SEQUENCE LISTING (79 sequences)

SEQ ID NO: 1 - DNA, 62 bp, Artificial Sequence
  Description of Artificial Sequence Synthetic oligonucleotide
  modified_base (51)..(52): Thymidine-succinyl hexamide CED phosphoramidite
agacaatcaaccatttggggtggacagccttgacctctagacttcggcattttttttttttt

SEQ ID NO: 2 - DNA, 112 bp, Artificial Sequence
  Description of Artificial Sequence Synthetic polynucleotide
  modified_base (101)..(102): Thymidine-succinyl hexamide CED phosphoramidite
cgggatccttatcgtcatcgtcgtacagatcccgacccatttgctgtccaccagtcatgctagccataccatgatgatgatgatgatgagaaccccgcattttttttttttt

SEQ ID NO: 3 - DNA, 19 bp, Artificial Sequence
  Description of Artificial Sequence Synthetic primer
atgcggggttctcatcatc

SEQ ID NO: 4 - DNA, 20 bp, Artificial Sequence
  Description of Artificial Sequence Synthetic primer
cgggatccttatcgtcatcg

SEQ ID NO: 5 - DNA, 22 bp, Homo sapiens
cagaacgttgttttcaggaagt

SEQ ID NO: 6 - DNA, 13 bp, Homo sapiens
aatacacaaagat

SEQ ID NO: 7 - protein, 4 aa, Homo sapiens
YVMA

SEQ ID NO: 8 - DNA, 13 bp, Homo sapiens
catacgtgatggc

SEQ ID NO: 9 - DNA, 10 bp, Homo sapiens
aggctcacca

SEQ ID NO: 10 - DNA, 10 bp, Homo sapiens
aggcaaccgt

SEQ ID NO: 11 - DNA, 16 bp, Homo sapiens
gcccagatggacatga

SEQ ID NO: 12 - protein, 5 aa, Homo sapiens
ELREA

SEQ ID NO: 13 - DNA, 16 bp, Homo sapiens
ggaattaagagaagca

SEQ ID NO: 14 - DNA, 10 bp, Homo sapiens
tatgggattg

SEQ ID NO: 15 - DNA, 16 bp, Homo sapiens
ttgggagatcttgcac

SEQ ID NO: 16 - protein, 6 aa, Homo sapiens
LGDLAR

SEQ ID NO: 17 - DNA, 25 bp, Homo sapiens
gcagacctctaggcaggcccaggcc

SEQ ID NO: 18 - DNA, 10 bp, Homo sapiens
tgtggccgag

SEQ ID NO: 19 - DNA, 28 bp, Homo sapiens
actgcttccctgaggaggagaagtgctt

SEQ ID NO: 20 - DNA, 13 bp, Homo sapiens
gagatgtttatga

SEQ ID NO: 21 - DNA, 17 bp, Homo sapiens
tgctttcaccctcagcg

SEQ ID NO: 22 - DNA, 17 bp, Homo sapiens
gaagctgtacttcacaa

SEQ ID NO: 23 - DNA, 20 bp, Homo sapiens
catgagtctagtacttaatg

SEQ ID NO: 24 - DNA, 23 bp, Homo sapiens
ctgtcttctgggattatcagaac

SEQ ID NO: 25 - DNA, 18 bp, Homo sapiens
gtaaaagtttagtaagta

SEQ ID NO: 26 - DNA, 15 bp, Homo sapiens
ttgattctagcacgc

SEQ ID NO: 27 - DNA, 18 bp, Homo sapiens
actacacaaatattgagg

SEQ ID NO: 28 - DNA, 26 bp, Homo sapiens
tgaaggactaaaggatcttcgaagac

SEQ ID NO: 29 - DNA, 23 bp, Homo sapiens
taagaaaagtatggatgatcaag

SEQ ID NO: 30 - DNA, 17 bp, Homo sapiens
aaatctggtgactatac

SEQ ID NO: 31 - DNA, 19 bp, Homo sapiens
actgtccccattggtgaat

SEQ ID NO: 32 - DNA, 17 bp, Homo sapiens
tgagacagttcctttta

SEQ ID NO: 33 - DNA, 20 bp, Homo sapiens
tgctccgccacctctaccag

SEQ ID NO: 34 - DNA, 30 bp, Homo sapiens
gatgaggagggcgcatgccagccttgcccc

SEQ ID NO: 35 - DNA, 29 bp, Homo sapiens
tctccactggcaccctccgaaggggctgg

SEQ ID NO: 36 - DNA, 28 bp, Homo sapiens
agatcccgactccgggacagcaccagcc

SEQ ID NO: 37 - DNA, 13 bp, Homo sapiens
caccccctccgaa

SEQ ID NO: 38 - DNA, 17 bp, Homo sapiens
ccttggcgaatccacca

SEQ ID NO: 39 - DNA, 31 bp, Homo sapiens
agaagatttgctgaaccctattggtgttact

SEQ ID NO: 40 - DNA, 14 bp, Homo sapiens
ctaaaatggcagtg

SEQ ID NO: 41 - DNA, 13 bp, Homo sapiens
tctgacagagtta

SEQ ID NO: 42 - DNA, 19 bp, Homo sapiens
gctgaaaaacaatctgact

SEQ ID NO: 43 - DNA, 17 bp, Homo sapiens
aatgaaactttcatttg

SEQ ID NO: 44 - DNA, 12 bp, Homo sapiens
cattgcagaatt

SEQ ID NO: 45 - DNA, 22 bp, Homo sapiens
attcactttgtcttagaggagt

SEQ ID NO: 46 - DNA, 25 bp, Homo sapiens
caatacaagcgactatagaggaaaa

SEQ ID NO: 47 - DNA, 16 bp, Homo sapiens
atttccagtcctgcag

SEQ ID NO: 48 - DNA, 26 bp, Homo sapiens
ctagagttctccttggaaatgagagc

SEQ ID NO: 49 - DNA, 13 bp, Homo sapiens
gacatgtccccca

SEQ ID NO: 50 - DNA, 16 bp, Homo sapiens
catgtgaacgctactt

SEQ ID NO: 51 - DNA, 14 bp, Homo sapiens
ataaccgaggagtt

SEQ ID NO: 52 - DNA, 14 bp, Homo sapiens
taattgtgcatgag

SEQ ID NO: 53 - DNA, 23 bp, Homo sapiens
ggcgtcccgcttgtccctgcgga

SEQ ID NO: 54 - DNA, 13 bp, Homo sapiens
agcatacgtgatg

SEQ ID NO: 55 - protein, 6 aa, Homo sapiens
SPDGHE

SEQ ID NO: 56 - DNA, 20 bp, Artificial Sequence
  Description of Artificial Sequence Synthetic primer
aatgatacggcgaccaccga

SEQ ID NO: 57 - DNA, 24 bp, Artificial Sequence
  Description of Artificial Sequence Synthetic primer
caagcagaagacggcatacgagat

SEQ ID NO: 58 - DNA, 23 bp, Artificial Sequence
  Description of Artificial Sequence Synthetic primer
gaagtgccattccgcgctcttca

SEQ ID NO: 59 - DNA, 24 bp, Artificial Sequence
  Description of Artificial Sequence Synthetic primer
tgaagagcatcgtacagctgctcg

SEQ ID NO: 60 - DNA, 17 bp, Artificial Sequence
  Description of Artificial Sequence Synthetic primer
ccattccgcgctcttca

SEQ ID NO: 61 - DNA, 19 bp, Artificial Sequence
  Description of Artificial Sequence Synthetic primer
tgaagagcatcgtacagct

SEQ ID NO: 62 - DNA, 22 bp, Artificial Sequence
  Description of Artificial Sequence Synthetic primer
gaagtgccattccgcctgacct

SEQ ID NO: 63 - DNA, 24 bp, Artificial Sequence
  Description of Artificial Sequence Synthetic primer
cgagcagctgtacgatgctcttca

SEQ ID NO: 64 - DNA, 24 bp, Artificial Sequence
  Description of Artificial Sequence Synthetic primer
cgctgacgatgtcagtgctcttca

SEQ ID NO: 65 - DNA, 19 bp, Artificial Sequence
  Description of Artificial Sequence Synthetic primer
agctgtacgatgctcttca

SEQ ID NO: 66 - DNA, 18 bp, Artificial Sequence
  Description of Artificial Sequence Synthetic primer
ccatgtgctcacgtctca

SEQ ID NO: 67 - DNA, 18 bp, Artificial Sequence
  Description of Artificial Sequence Synthetic primer
agtcaggatgtcgtctcg

SEQ ID NO: 68 - DNA, 18 bp, Artificial Sequence
  Description of Artificial Sequence Synthetic primer
cgagacgacatcctgact

SEQ ID NO: 69 - DNA, 73 bp, Artificial Sequence
  Description of Artificial Sequence Synthetic oligonucleotide
  Description of Combined DNA/RNA Molecule Synthetic oligonucleotide
  modified_base (33)..(42): a, c, t, g, u, unknown or other
gaagtgccattccgcctgacctgctcttccgunnnnnnnnnnacggaagagctccgatccacctccgagtcac

SEQ ID NO: 70 - DNA, 73 bp, Artificial Sequence
  Description of Artificial Sequence Synthetic oligonucleotide
  Description of Combined DNA/RNA Molecule Synthetic oligonucleotide
  modified_base (32)..(41): a, c, t, g, u, unknown or other
gtgactcggaggtggatcggagctcttccgunnnnnnnnnnacggaagagcaggtcaggcggaatggcacttc

SEQ ID NO: 71 - DNA, 72 bp, Artificial Sequence
  Description of Artificial Sequence Synthetic oligonucleotide
  modified_base (32)..(41): a, c, t, g, unknown or other
gaagtgccattccgcctgacctgctcttccgnnnnnnnnnnacggaagagctccgatccacctccgagtcac

SEQ ID NO: 72 - DNA, 72 bp, Artificial Sequence
  Description of Artificial Sequence Synthetic oligonucleotide
  modified_base (31)..(40): a, c, t, g, unknown or other
gtgactcggaggtggatcggagctcttccgnnnnnnnnnnacggaagagcaggtcaggcggaatggcacttc

SEQ ID NO: 73 - DNA, 41 bp, Artificial Sequence
  Description of Artificial Sequence Synthetic oligonucleotide
  modified_base (1)..(10): a, c, t, g, unknown or other
nnnnnnnnnnacggaagagctccgatccacctccgagtcac

SEQ ID NO: 74 - DNA, 42 bp, Artificial Sequence
  Description of Artificial Sequence Synthetic oligonucleotide
  modified_base (1)..(10): a, c, t, g, unknown or other
nnnnnnnnnnacggaagagcaggtcaggcggaatggcacttc

SEQ ID NO: 75 - DNA, 32 bp, Artificial Sequence
  Description of Artificial Sequence Synthetic primer
  Description of Combined DNA/RNA Molecule Synthetic primer
gaagtgccattccgcctgacctgctcttccgu

SEQ ID NO: 76 - DNA, 31 bp, Artificial Sequence
  Description of Artificial Sequence Synthetic primer
  Description of Combined DNA/RNA Molecule Synthetic primer
gtgactcggaggtggatcggagctcttccgu

SEQ ID NO: 77 - DNA, 32 bp, Artificial Sequence; identical to SEQ ID NO: 75
  Description of Artificial Sequence Synthetic primer
  Description of Combined DNA/RNA Molecule Synthetic primer

SEQ ID NO: 78 - DNA, 320 bp, Artificial Sequence
  Description of Artificial Sequence Synthetic polynucleotide
cagtggccagaggccatcaaagctcaggttaatgaaatgctctttattttgtagccatccagtccaatggatcagatgggcaagatgagacctcagccatatggcgggactaacccatactcgcagcaacagggacctccgtcaggaccgcagcaaggacctgggtacccagggcagccatacgggtcccagaccccgcagcggtacccgatgaccatgcagggccgggcgcagagtgccatgggcggcctctcttatacacagcaggtagatggtgattgtgattaccttgacccttgttgctgtccaaaatctgatct

SEQ ID NO: 79 - DNA, 3444 bp, Artificial Sequence
  Description of Artificial Sequence Synthetic polynucleotide
tcaggccatcaaagctcaggttaatgaaatgctctttattttgtagccatccagtccaatggatcagatgggcaagatgagacctcagccatatggcgggactaacccatactcgcagcaacagggacctccgtcaggaccgcagcaaggacctgggtaccctctacaaagctcaggttaatgaaatgctctttattttgtagccatccagtccaatggatcagatgggcaagatgagacctcagccatatggcgggactaacccatactcgcagcaacagggacctccgtcaggaccgcagcaaggacctgggtacccagggcaggggacaggttaatgaaatgctctttattttgtagccatccagtccaatggatcagatgggcaagatgagacctcagccatatggcgggactaacccatactcgcagcaacagggacctccgtcaggaccgcagcaaggacctgggtacccagggcagccatacgccatatgaaatgctctttattttgtagccatccagtccaatggatcagatgggcaagatgagacctcagccatatggcgggactaacccatactcgcagcaacagggacctccgtcaggaccgcagcaaggacctgggtacccagggcagccatacgggtcccacttagctctttattttgtagccatccagtccaatggatcagatgggcaagatgagacctcagccatatggcgggactaacccatactcgcagcaacagggacctccgtcaggaccgcagcaaggacctgggtacccagggcagccatacgggtcccagaccccggtaaattttgtagccatccagtccaatggatcagatgggcaagatgagacctcagccatatggcgggactaacccatactcgcagcaacagggacctccgtcaggaccgcagcaaggacctgggtacccagggcagccatacgggtcccagaccccgcagcggttgcaagccatccagtccaatggatcagatgggcaagatgagacctcagccatatggcgggactaacccatactcgcagcaacagggacctccgtcaggaccgcagcaaggacctgggtacccagggcagccatacgggtcccagaccccgcagcggtacccgatctagcagtccaatggatcagatgggcaagatgagacctcagccatatggcgggactaacccatactcgcagcaacagggacctccgtcaggaccgcagcaaggacctgggtacccagggcagccatacgggtcccagaccccgcagcggtacccgatgaccatggtgtatggatcagatgggcaagatgagacctcagccatatggcgggactaacccatactcgcagcaacagggacctccgtcaggaccgcagcaaggacctgggtacccagggcagccatacgggtcccagaccccgcagcggtacccgatgaccatgcagggccccagagatgggcaagatgagacctcagccatatggcgggactaacccatactcgcagcaacagggacctccgtcaggaccgcagcaaggacctgggtacccagggcagccatacgggtcccagaccccgcagcggtacccgatgaccatgcagggccgggcgcactcccaagatgagacctcagccatatggcgggactaacccatactcgcagcaacagggacctccgtcaggaccgcagcaaggacctgggtacccagggcagccatacgggtcccagaccccgcagcggtacccgatgaccatgcagggccgggcgcagagtgcctaacagacctcagccatatggcgggactaacccatactcgcagcaacagggacctccgtcaggaccgcagcaaggacctgggtacccagggcagccatacgggtcccagaccccgcagcggtacccgatgaccatgcagggccgggcgcagagtgccatgggcgcgagagccatatggcgggactaacccatactcgcagcaacagggacctccgtcaggaccgcagcaaggacctgggtacccagggcagccatacgggtcccagaccccgcagcggtacccgatgaccatgcagggccgggcgcagagtgccatgggcggcctctcaaattggcgggactaacccatactcgcagcaacagggacctccgtcaggaccgcagcaaggacctgggtacccagggcagccatacgggtcccagaccccgcagcggtacccgatgaccatgcagggccgggcgcagagtgccatgggcggcctctcttatacagtccactaacccatactcgcagcaacagggacctccgtcaggaccgcagcaaggacctgggtacccagggcagccatacgggtcccagaccccgcagcggtacccgatgaccatgcagggccgggcgcagagtgccatgggcggcctctcttatacacagcaggatggcatactcgcagcaacagggacctccgtcaggaccgcagcaaggacctgggtacccagggcagccatacgggtcccagaccccgcagcggtacccgatgaccatgcagggccgggcgcagagtgccatgggcggcctctcttatacacagcaggtagatggacaggcagcaacagggacctccgtcaggaccgcagcaaggacctgggtacccagggcagccatacgggtcccagaccccgcagcggtacccgatgaccatgcagggccgggcgcagagtgccatgggcggcctctcttatacacagcaggtagatggtgattgtctttcagggacctccgtcaggaccgcagcaaggacctgggtacccagggcagccatacgggtcccagaccccgcagcggtacccgatgaccatgcagggccgggcgcagagtgccatgggcggcctctcttatacacagcaggtagatggtgattgtgattaccaatgctccgtcaggaccgcagcaaggacctgggtacccagggcagccatacgggtcccagaccccgcagcggtacccgatgaccatgcagggccgggcgcagagtgccatgggcggcctctcttatacacagcaggtagatggtgattgtgattaccttgacccaaagaggaccgcagcaaggacctgggtacccagggcagccatacgggtcccagaccccgcagcggtacccgatgaccatgcagggccgggcgcagagtgccatgggcggcctctcttatacacagcaggtagatggtgattgtgattaccttgacccttgttgcacgccagcaaggacctgggtacccagggcagccatacgggtcccagaccccgcagcggtacccgatgaccatgcagggccgggcgcagagtgccatgggcggcctctcttatacacagcaggtagatggtgattgtgattaccttgacccttgttgctgtccaatt

(19) United States
(12) Patent Application Publication
(10) Pub. No.: US 2022/0356463 A1
(43) Pub. Date: Nov. 10, 2022

(54) LIBRARIES FOR MUTATIONAL ANALYSIS

(71) Applicant: Twist Bioscience Corporation, South San Francisco, CA (US)

(72) Inventors: Jinfeng SHEN, Redwood City, CA (US); Michael BOCEK, South San Francisco, CA (US); David LIN, South San Francisco, CA (US); Alonzo LEE, San Jose, CA (US); Patrick CHERRY, San Francisco, CA (US); Siyuan CHEN, San Mateo, CA (US); Esteban TORO, Fremont, CA (US); Leslie QUINTANILLA-ZARINAN, South San Francisco, CA (US)

(21) Appl. No.: 17/715,890

(22) Filed: Apr. 7, 2022

Related U.S. Application Data
(60) Provisional application No. 63/173,306, filed on Apr. 9, 2021.
(60) Provisional application No. 63/278,873, filed on Nov. 12, 2021.
(60) Provisional application No. 63/309,212, filed on Feb. 11, 2022.

(51) Int. Cl. C12N 15/10

(52) U.S. Cl. CPC C12N 15/1065; C12N 15/1093

(57) ABSTRACT

Provided herein are compositions and methods for identifying genomic variants. Further provided herein are standards useful for determining the analytical sensitivity and/or accuracy of instruments configured to measure nucleic acid variant frequencies.

### CROSS-REFERENCE

This application claims the benefit of U.S. provisional patent application No. 63/173,306 filed on Apr. 9, 2021; U.S. provisional patent application No. 63/278,873 filed on Nov. 12, 2021; and U.S. provisional patent application No. 63/309,212 filed on Feb. 11, 2022, each of which are incorporated by reference in its entirety.

### SEQUENCE LISTING

The instant application contains a Sequence Listing which has been submitted electronically in ASCII format and is hereby incorporated by reference in its entirety. Said ASCII copy, created on Jun. 13, 2022, is named 44854-823_201_SL.txt and is 20,388 bytes in size.

### BACKGROUND

Identification of genomic variants with high fidelity and low cost has a central role in biotechnology and medicine, and in basic biomedical research. While various methods are known for identification of genomic variants in complex nucleic acid samples, these techniques often suffer from scalability, automation, speed, sensitivity, accuracy, and cost.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF SUMMARY

Provided herein are compositions and methods for determination of genomic variants.

Provided herein are polynucleotide libraries comprising: a sample polynucleotide set comprising at least 100 polynucleotides derived from genomic sequences; and a background set comprising background polynucleotides, wherein the background set comprises cell-free DNA (cfDNA), wherein each of the least 100 polynucleotides comprises of the sample polynucleotide set comprises at least one variant, wherein the at least one variant comprises one or more changes compared to a background polynucleotide; and at least 2 polynucleotides of the at least 100 polynucleotides are tiled across each of the at least one variant. Further provided herein are libraries wherein each of the least 100 polynucleotides comprises one variant. Further provided herein are libraries wherein the sample polynucleotide set comprises at least 150 variants. Further provided herein are libraries wherein the sample polynucleotide set comprises at least 400 variants. Further provided herein are libraries wherein the least at least 5 polynucleotides are tiled across the at least one variant. Further provided herein are libraries wherein the least at least 20 polynucleotides are tiled across the at least one variant. Further provided herein are libraries wherein the least at least 30 polynucleotides are tiled across the at least one variant. Further provided herein are libraries wherein the least at least 10 polynucleotides are tiled across the at least one variant with an offset of 1-8 bases. Further provided herein are libraries wherein the genomic sequences are derived from cell-free DNA (cfDNA). Further provided herein are libraries wherein the sample polynucleotide set comprises no more than 10% of the total amount of polynucleotides in the library. Further provided herein are libraries wherein the at least one variant is present at a frequency of 0.01-5% relative to a wild-type genomic sequence. Further provided herein are libraries wherein the at least one variant is present at a frequency of 1-5% relative to a wild-type genomic sequence. Further provided herein are libraries wherein the at least one variant is present at a frequency of 0.1% 0.25%, 0.5%, 1%, or 2% relative to a wild-type genomic sequence. Further provided herein are libraries wherein at least 90% of the at least one variants is present at a frequency of no more than 10% relative to the frequency of other variants. Further provided herein are libraries wherein at least 99% of the at least one variants is present at a frequency of no more than 20% relative to the frequency of other variants. Further provided herein are libraries wherein at least some of the least 100 polynucleotides are double stranded. Further provided herein are libraries wherein at least 90% of the least 100 polynucleotides are double stranded. Further provided herein are libraries wherein the length of at least some of the least 100 polynucleotides is 125-200 bases. Further provided herein are libraries wherein the length of at least 90% of the least 100 polynucleotides is 125-200 bases. Further provided herein are libraries wherein the at least one variant comprises an insertion, deletion, fusion, duplication, frameshift, repeat expansion, or substitution. Further provided herein are libraries wherein the at least one variant comprises a copy number variant (CNV), microsatellite instability, loss of heterozygosity (LOH), DNA methylation, premature stop codon, trinucleotide repeat, translocation, somatic rearrangement, allelomorph, single nucleotide variant (SNV), indel, splice variant, regulator variant, copy number variant, or fusion. Further provided herein are libraries wherein the at least one variant comprises a single nucleotide variant, indel, fusion, or structural variant. Further provided herein are libraries wherein the indel is 1-15 bases in length. Further provided herein are libraries wherein the at least one variant comprises a modification to an tumor suppressor or oncogene. Further provided herein are libraries wherein the library comprises variants located in at least 50 genes. Further provided herein are libraries wherein the library comprises variants located in at least 75 genes. Further provided herein are libraries wherein the at least one variant is located in one or more of genes ABL1, ABL2, AKT1, ALK, APC, AR, ARAF, ARID1A, ATM, ATR, BAP1, BRAF, BRCA1, BRCA2, CCND1, CDC6, CDH1, CDK12, CDK4, CDX2, CTNNB1, DDR2, EGFR, EML4, ERBB2, ERBB3, ERG, ESR1, EZH2, FBXW7, FGFR1, FGFR2, FGFR3, FLT3, FOXA1, FOXL2, GATA3, GNA11, GNAQ, GNAS, HNF1A, HRAS, IDH1, IDH2, JAK2, KDM5C, KDM6A, KIF5B, KIT, KRAS, MAP2K1, MAPK1, MET, MIR4728, ERBB2, MLH1, MPL, MYCN, MYD88, NCOA4, NF1, NF2, NFE2L2, NOTCHI, NPM1, NRAS, PBRM1, PDGFRA, PIK3CA, PTEN, PTPN11, RET, RHEB, RHOA, RIT1, ROS1, SETD2, SMAD4, SMO, SPOP, TERT, TMPRSS2, TP53, TPR, TSC1, and VHL. Further provided herein are libraries wherein the at least one variant is located in ten or more of genes ABL1, ABL2, AKT1, ALK, APC, AR, ARAF, ARID1A, ATM, ATR, BAP1, BRAF, BRCA1, BRCA2, CCND1, CDC6, CDH1, CDK12, CDK4, CDX2, CTNNB1, DDR2, EGFR, EML4, ERBB2, ERBB3, ERG, ESR1, EZH2, FBXW7, FGFR1, FGFR2, FGFR3, FLT3, FOXA1, FOXL2, GATA3, GNA11, GNAQ, GNAS, HNF1A, HRAS, IDH1, IDH2, JAK2, KDM5C, KDM6A, KIF5B, KIT, KRAS, MAP2K1, MAPK1, MET, MIR4728, ERBB2, MLH1, MPL, MYCN, MYD88, NCOA4, NF1, NF2, NFE2L2, NOTCHI, NPM1, NRAS, PBRM1, PDGFRA, PIK3CA, PTEN, PTPN11, RET, RHEB, RHOA, RIT1, ROS1, SETD2, SMAD4, SMO, SPOP, TERT, TMPRSS2, TP53, TPR, TSC1, and VHL. Further provided herein are libraries wherein the sample polynucleotide set is substantially free of biological contamination. Further provided herein are libraries wherein the biological contamination comprises cellular components or biomolecules derived from plasma. Further provided herein are libraries wherein the library further comprises a buffer. Further provided herein are libraries wherein the buffer comprises tris-EDTA. Further provided herein are libraries wherein the background polynucleotide set comprises wild-type regions corresponding to locations of the at least one variant. Further provided herein are libraries wherein the wild-type regions are represented within 10% of the variant frequency of the variant set. Further provided herein are libraries wherein the background polynucleotide set comprises two or more polynucleotides. Further provided herein are libraries wherein highest abundance of polynucleotides in the background set are 125-200 bases in length. Further provided herein are libraries wherein highest abundance of polynucleotides in the background set are 150-185 bases in length. Further provided herein are libraries wherein at least 90% of the polynucleotides in the background set are mononucleosomal or dinucleosomal. Further provided herein are libraries wherein the ratio of mononucleosomal to dinucleosomal is 70:30 to 90:10. Further provided herein are libraries wherein the background polynucleotide set is derived from a healthy human. Further provided herein are libraries wherein the background polynucleotide set is isolated from a healthy human. Further provided herein are libraries wherein the human is male. Further provided herein are libraries wherein the human is no more than 30 years old. Further provided herein are libraries wherein at least one background polynucleotide comprises a variant present at a frequency of 0.001%, 0.01%, 0.1% 0.25%, 0.5%, 1%, or 2% relative to a wild-type genomic sequence.

Provided herein are kits for measuring variant detection limits comprising: a library described herein; instructions for use of the kit; and packaging configured to hold and describe the kit contents. Further provided herein are kits wherein the kit comprises at least two libraries described herein. Further provided herein are kits wherein the at least two libraries each comprise variants present at a frequency of 0%, 0.1% 0.25%, 0.5%, 1%, or 2% relative to a wild-type genomic sequence. Further provided herein are kits wherein the kit comprises five libraries, each comprising variants present at a frequency of 0%, 0.1% 0.25%, 0.5%, 1%, or 2% relative to a wild-type genomic sequence.

Provided herein are methods of preparing a library described herein comprising: providing the background polynucleotide set; synthesizing the sample polynucleotide set from predetermined sequences; and mixing the variant set and the background set in a buffer. Further provided herein are methods wherein synthesizing comprises chemical synthesis. Further provided herein are methods wherein synthesizing comprises synthesis on a surface. Further provided herein are methods wherein synthesizing comprises coupling of nucleoside phosphoramidites. Further provided herein are methods further comprising sequencing the library. Further provided herein are methods further comprising ddPCR measurement of the library. Further provided herein are methods further comprising fluorescence/UV DNA quantification and size distribution of the library. Further provided herein are methods further comprising determining the variant frequency in the background polynucleotide set, where the variants correspond to the at least one variant in the sample polynucleotide set. Further provided herein are methods further comprising fluorescence/UV DNA quantification of the sample polynucleotide set prior to mixing. Further provided herein are methods further comprising ZAG fragment analysis of the sample polynucleotide set prior to mixing. Provided herein are methods of preparing a nucleic acid test sample useful for determining the detection limit of genomic variants comprising: providing a library described herein; obtaining at least one test sample from a patient suspected of having a disease or condition; detecting the presence or absence of the one or more variants in the library; and detecting the presence or absence of the one or more variants in the at least one test sample. Further provided herein are methods wherein detecting comprises sequencing. Further provided herein are methods wherein detecting comprises Next Generation Sequencing. Further provided herein are methods wherein sequencing comprises sequencing by synthesis, nanopore sequencing, or SMRT sequencing. Further provided herein are methods wherein detecting comprises ddPCR or specific hybridization to an array. Further provided herein are methods wherein the at least one test sample comprises a liquid biopsy. Further provided herein are methods wherein the at least one test sample comprises circulating tumor DNA (ctDNA). Further provided herein are methods wherein the at least one test sample is obtained from blood. Further provided herein are methods wherein the at least one test sample is substantially cell-free. Further provided herein are methods wherein the method comprises at least 5 test samples. Further provided herein are methods wherein the method further comprises detection of minimal residual disease (MRD). Further provided herein are methods wherein the patient is suspected of having a disease or condition. Further provided herein are methods wherein the disease or condition is a proliferative disease. Further provided herein are methods wherein the disease or condition is cancer. Further provided herein are methods wherein the patient was previously treated, is currently treated, or has received a clinical diagnosis for cancer. Further provided herein are methods wherein the method further comprises ligating sequencing adapters to at least some polynucleotides in the test sample, the library, or both. Further provided herein are methods wherein the method further comprises amplifying at least some polynucleotides in the test sample, the library, or both. Further provided herein are methods wherein if one or more variants are not detected in the library, then results obtained from the at least one test sample is discarded or re-analyzed. Further provided herein are methods wherein detecting comprises addition of one or more adapters to at least some sample polynucleotides in the library. Further provided herein are methods wherein the adapters comprise at least one barcode. Further provided herein are methods wherein the at least one barcode comprises one or more of a unique molecular identifier and a sample index. Further provided herein are methods wherein the at least one adapter comprises a duplex adapter. Further provided herein are methods wherein at least one adapter comprises at least two unique molecular identifiers. Further provided herein are methods wherein at least one adapter comprises a first unique molecular identifier and a second unique molecular identifier. Further provided herein are methods wherein the first unique molecular identifier or the second unique molecular identifier comprises a sequence of one or more of AAGGA, ACAAC, ATACG, CACTG, CATGA, CGATA, CGTGT, GCCAT, GCTGT, GTCAC, GTCGT, TACGA, TCCTA, TCGTG, TGTCG, TTGGC, AACAC, AATGC, ACTAG, AGCAT, AGTAC, ATCTC, CAGAC, CAGTA, CGAAT, CGGTT, CTTGG, GCATA, GCTAA, GTGAG, GTGTC, and TGTGC. Further provided herein are methods wherein the first unique molecular identifier or the second unique molecular identifier comprises a sequences of 10 or more of AAGGA, ACAAC, ATACG, CACTG, CATGA, CGATA, CGTGT, GCCAT, GCTGT, GTCAC, GTCGT, TACGA, TCCTA, TCGTG, TGTCG, TTGGC, AACAC, AATGC, ACTAG, AGCAT, AGTAC, ATCTC, CAGAC, CAGTA, CGAAT, CGGTT, CTTGG, GCATA, GCTAA, GTGAG, GTGTC, and TGTGC.

### BRIEF DESCRIPTION OF THE DRAWINGS

FIG. 1A depicts a design of synthetic ctDNA to target a variant site. Multiple overlapping or “tiled” polynucleotides are configured to contain the variant site (indicated with a star). The x-axis is labeled genome coordinate from 0-300 at 100 unit intervals; the y-axis is labeled oligos.

FIG. 1B depicts a distribution of indel sizes for a synthetic ctDNA library, including short, medium (5-10 bp), and large size variants (˜30 bp). Positive numbers are insertions, and negative numbers are deletions. The y-axis is labeled number of variants from 0 to 40 at 20 unit intervals; the x-axis is labeled indel size (bp) from −30 to 10 at 10 unit intervals.

FIG. 1C depicts a plot of signal (representative of abundance) vs. size for background cell-free DNA (cfDNA). The background cfDNA was obtained from healthy donor plasma. The y-axis is labeled fluorescence units (FU) from 0 to 400 at 50 unit intervals; the x-axis is labeled base pairs (bp) at 35, 100, 150, 200, 300, 400, 500, 600, 1000, 2000, 10380. Peak 1 and peak 2 are labeled.

FIG. 2 depicts an image of a plate having 256 clusters, each cluster having 121 loci with polynucleotides extending therefrom.

FIG. 3A depicts a plot of polynucleotide representation (polynucleotide frequency versus abundance, as measured absorbance) across a plate from synthesis of 29,040 unique polynucleotides from 240 clusters, each cluster having 121 polynucleotides.

FIG. 3B depicts a plot of measurement of polynucleotide frequency versus abundance absorbance (as measured absorbance) across each individual cluster, with control clusters identified by a box.

FIG. 4 illustrates a computer system.

FIG. 5 is a block diagram illustrating an architecture of a computer system.

FIG. 6 is a diagram demonstrating a network configured to incorporate a plurality of computer systems, a plurality of cell phones and personal data assistants, and Network Attached Storage (NAS).

FIG. 7 is a block diagram of a multiprocessor computer system using a shared virtual address memory space.

FIG. 8A-1 depicts a cfDNA library target (white region “GACCTGG”) in a genomic region. Figure discloses SEQ ID NO: 78.

FIG. 8A-2 depicts a cfDNA library design without the flanks added, to show the location of each of the variants (white regions) across each molecule in the library. The dashed line separates the left and right sections of the figure. Figure discloses SEQ ID NO: 79.

FIG. 8B depicts sequencing results for original and expanded cfDNA libraries as a function of reads vs. template length. Data series are Supplier_v1_noexpansion; Supplier_v2; v2_1_exoIII; and v_2_2. The y-axis is labeled number of reads from 0 to 60,000 at 10,000 unit intervals; the x-axis is labeled template length from 100-600 at 100 unit intervals.

FIG. 8C depicts sequencing results for original and expanded cfDNA libraries as a function of the percent of reads with no soft-clipping. The y-axis is labeled percent of reads with no soft-clip; the x-axis is labeled sample name (left to right): Supplier_v2; v_2_2; v2_1_exoIII; and Supplier vi No expansion.

FIG. 9A depicts a graph showing the size distribution of cfDNA fragments generated using uracil-containing adapters. The y-axis is labeled fluorescence units (FU) from 0 to 400 at 50 unit intervals; the x-axis is labeled base pairs (bp) at 35, 100, 150, 200, 300, 400, 500, 600, 1000, 2000, 10380. Peak 1 and peak 2 are labeled.

FIG. 9B depicts a graph showing the size distribution of cfDNA fragments generated using uracil-containing adapters having a 3′ phosphorothioate bond. The y-axis is labeled fluorescence units (FU) from 0 to 400 at 50 unit intervals; the x-axis is labeled base pairs (bp) at 35, 100, 150, 200, 300, 400, 500, 600, 1000, 2000, 10380. Peak 1 and peak 2 are labeled.

FIG. 9C depicts a graph showing the size distribution of cfDNA fragments generated using uracil-containing adapters having three 3′ phosphorothioate bonds. The y-axis is labeled fluorescence units (FU) from 0 to 400 at 50 unit intervals; the x-axis is labeled base pairs (bp) at 35, 100, 150, 200, 300, 400, 500, 600, 1000, 2000, 10380. Peak 1 and peak 2 are labeled.

FIG. 10A depicts a workflow for attachment of adapters comprising unique molecular identifiers (UMIs) to a polynucleotide to form an adapter-ligated polynucleotide.

FIG. 10B depicts a workflow for amplification of adapter-ligated polynucleotides to form a library for sequencing.

FIG. 10C depicts a workflow for synthesis of a polynucleotide adapter comprising a UMI.

FIG. 10D depicts a workflow for synthesis of a polynucleotide adapter comprising a UMI, wherein the method comprises PCR extension of one strand of the adapter.

FIG. 10E depicts a workflow for synthesis of a polynucleotide adapter comprising a UMI, wherein the method comprises PCR extension of one strand of the adapter, followed by restriction enzyme cleavage.

FIG. 10F depicts a workflow for synthesis of a polynucleotide adapter comprising a UMI, wherein the method comprises restriction enzyme cleavage.

FIG. 11 depicts a workflow for duplex sequencing analysis to identify variants. “*” indicates potential errors introduced by PCR or sequencing, and “+” indicates true variants.

FIG. 12 depicts a plot of UMI performance (32 UMIs) for a ctDNA sample. Two different UMI sources were used.

FIG. 13A depicts a plot of UMI performance for each UMI barcode. Two different UMI sources were used.

FIG. 13B depicts a plot of UMI performance for each UMI barcode. Two different UMI sources were used, for two different runs (circles vs. squares).

FIG. 14A depicts a plot of UMI performance using Fold-80 base penalty. Two different runs were conducted.

FIG. 14B depicts a plot of UMI performance using HS library size. Two different runs were conducted.

FIG. 14C depicts a plot of UMI performance using percent off bait. Two different runs were conducted.

FIG. 15A depicts a plot of UMI performance using percent duplex family size for a number of samples.

FIG. 15B depicts a plot of UMI performance using family size for a first experiment.

FIG. 15C depicts a plot of UMI performance using family size for a second experiment.

FIG. 15D depicts a plot of UMI performance using family size for a first UMI library source.

FIG. 15E depicts a plot of UMI performance using family size for a second UMI library source.

FIG. 16 depicts a plot of UMI duplex efficiency as a function of different UMI blends.

FIG. 17A depicts plots of precision (left) and recall (right) with filtering recurrent variants.

FIG. 17B depicts plots of precision (left) and recall (right) without filtering recurrent variants.

FIG. 17C depicts a plot of recall for single base substitution variants (SBS).

FIG. 17D depicts plots of precision (left) and recall (right) with a 2-1-1 filter.

FIG. 18 depicts a plot of recall for single base substitution variants (SBS). The left set of bars in each set are variant calls (Mutect2) and the right set are raw pileups.

FIG. 19A depicts a plot of recall using 20000× downsampling and a 2-2-1 filter. The left set of bars in each set are calls and the right set are pileups.

FIG. 19B depicts a plot of recall using no downsampling and a 1-0-0 filter. The left set of bars in each set are calls and the right set are pileups.

FIG. 19C depicts a plot of variant calls for unfiltered reads for various indel lengths.

FIG. 19D depicts a plot of raw pileups for unfiltered reads and various indel lengths (left to right for each set: 0, 1, 2-4, 5-9, 10+).

FIG. 19E depicts a plot of variant calls for various indel lengths (left to right for each set: 0, 1, 2-4, 5-9, 10+) using no downsampling and a 1-1-0 filter.

FIG. 19F depicts a plot of raw pileups for various indel lengths (left to right for each set: 0, 1, 2-4, 5-9, 10+) using 20000× downsampling and a 1-1-0 filter.

### DETAILED DESCRIPTION

Described herein are compositions and methods for identification of genomic variants. Further provided herein are polynucleotide libraries configured as references or controls to measure detection sensitivity. Further described herein are methods of identifying variants using adapters which comprise unique molecular identifiers (UMIs). UMIs in some instances provide for uniquely identification of individual members of a polynucleotide library, which enables molecular counting and identification of potential errors generated during preparation of a polynucleotide library prior to sequencing.

### Definitions

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

As used herein, the terms “preselected sequence”, “predefined sequence” or “predetermined sequence” are used interchangeably. The terms mean that the sequence of the polymer is known and chosen before synthesis or assembly of the polymer. In particular, various aspects of the invention are described herein primarily with regard to the preparation of nucleic acids molecules, the sequence of the oligonucleotide or polynucleotide being known and chosen before the synthesis or assembly of the nucleic acid molecules.

The term nucleic acid encompasses double- or triple-stranded nucleic acids, as well as single-stranded molecules. In double- or triple-stranded nucleic acids, the nucleic acid strands need not be coextensive (i.e., a double-stranded nucleic acid need not be double-stranded along the entire length of both strands). Nucleic acid sequences, when provided, are listed in the 5′ to 3′ direction, unless stated otherwise. Methods described herein provide for the generation of isolated nucleic acids. Methods described herein additionally provide for the generation of isolated and purified nucleic acids. The length of polynucleotides, when provided, are described as the number of bases and abbreviated, such as nt (nucleotides), bp (bases), kb (kilobases), Mb (megabases) or Gb (gigabases).

Provided herein are methods and compositions for production of synthetic (i.e. de novo synthesized or chemically synthesizes) polynucleotides. The term oligonucleic acid, oligonucleotide, oligo, and polynucleotide are defined to be synonymous throughout. Libraries of synthesized polynucleotides described herein may comprise a plurality of polynucleotides collectively encoding for one or more genes or gene fragments. In some instances, the polynucleotide library comprises coding or non-coding sequences. In some instances, the polynucleotide library encodes for a plurality of cDNA sequences. Reference gene sequences from which the cDNA sequences are based may contain introns, whereas cDNA sequences exclude introns. Polynucleotides described herein may encode for genes or gene fragments from an organism. Exemplary organisms include, without limitation, prokaryotes (e.g., bacteria) and eukaryotes (e.g., mice, rabbits, humans, and non-human primates). In some instances, the polynucleotide library comprises one or more polynucleotides, each of the one or more polynucleotides encoding sequences for multiple exons. Each polynucleotide within a library described herein may encode a different sequence, i.e., non-identical sequence. In some instances, each polynucleotide within a library described herein comprises at least one portion that is complementary to sequence of another polynucleotide within the library. Polynucleotide sequences described herein may be, unless stated otherwise, comprise DNA or RNA. A polynucleotide library described herein may comprise at least 10, 20, 50, 100, 200, 500, 1,000, 2,000, 5,000, 10,000, 20,000, 30,000, 50,000, 100,000, 200,000, 500,000, 1,000,000, or more than 1,000,000 polynucleotides. A polynucleotide library described herein may have no more than 10, 20, 50, 100, 200, 500, 1,000, 2,000, 5,000, 10,000, 20,000, 30,000, 50,000, 100,000, 200,000, 500,000, or no more than 1,000,000 polynucleotides. A polynucleotide library described herein may comprise 10 to 500, 20 to 1000, 50 to 2000, 100 to 5000, 500 to 10,000, 1,000 to 5,000, 10,000 to 50,000, 100,000 to 500,000, or 50,000 to 1,000,000 polynucleotides. A polynucleotide library described herein may comprise about 370,000; 400,000; 500,000 or more different polynucleotides.

Libraries of Variants

Provided herein are polynucleotide libraries configured to measure the sensitivity of variant measurements. In some instances, these libraries are used as references or controls. Known methods of generating such libraries may comprise isolating nucleic acids from biological sources (blood, plasma, cells, or patients) with an established disease or condition. However, such methods in some instances provide libraries which contain contamination from their biological source. In some instances, libraries are produced from biological samples to mimic cell-free DNA (cfDNA) by restriction digestion, sonication, or other method of generating short nucleic acid fragments. These methods may not mimic the natural fragmentation profile of cfDNA. Additionally, low abundance variants may not be detected from biologically-derived libraries. Provided herein are methods comprising design and de-novo synthesis of polynucleotide libraries (or sample sets) which are useful for measuring variant frequencies. Such libraries in some instances provide enhanced accuracy for diagnosing diseases or conditions, and are substantially free of biological contamination. Synthetic polynucleotide libraries in some instances provide additional control over library content, reliability/reproducibility, lack of reliance on fragmentation methods, or provide other advantages over traditional cell-derived libraries. These libraries (sample libraries or variant libraries) are in some instances mixed with control nucleic acids (e.g., cfDNA) to generate reference standards at specific VAFs (variant allele frequencies). In some instances, a polynucleotide library comprises a sample polynucleotide set comprising polynucleotides derived from genomic sequences. In some instances, a polynucleotide library comprises a background set comprising background polynucleotides, wherein the background set comprises cell-free DNA (cfDNA). In some instances, at least some of the polynucleotides of the sample polynucleotide set comprise at least one variant, wherein the at least one variant comprises one or more changes compared to a background polynucleotide. In some instances, at least some of the polynucleotides of the sample set are tiled across each of the at least one variant. In some instances, background cfDNA is obtained, derived, or expanded from a cell line or patient sample.

Provided herein are libraries of polynucleotides comprising pre-determined variant sequences (e.g., variants). In some instances, libraries comprise at least 1, 5, 10, 15, 20, 25, 50, 75, 100, 150, 200, 250, 300, 350, 400, 450, 500, 750, 1000, or at least 2000 variants. In some instances, libraries comprise about 1, 5, 10, 15, 20, 25, 50, 75, 100, 150, 200, 250, 300, 350, 400, 450, 500, 750, 1000, or about 2000 variants. In some instances, libraries comprise no more than 1, 5, 10, 15, 20, 25, 50, 75, 100, 150, 200, 250, 300, 350, 400, 450, 500, 750, 1000, or no more than 2000 variants. In some instances, libraries comprise 1-500, 5-500, 10-500, 10-2000, 10-150, 15-500, 20-1000, 50-500, 50-750, 50-1000, 100-1000, 100-500, 100-750, 250-800, 400-1000, or 400-2000 variants.

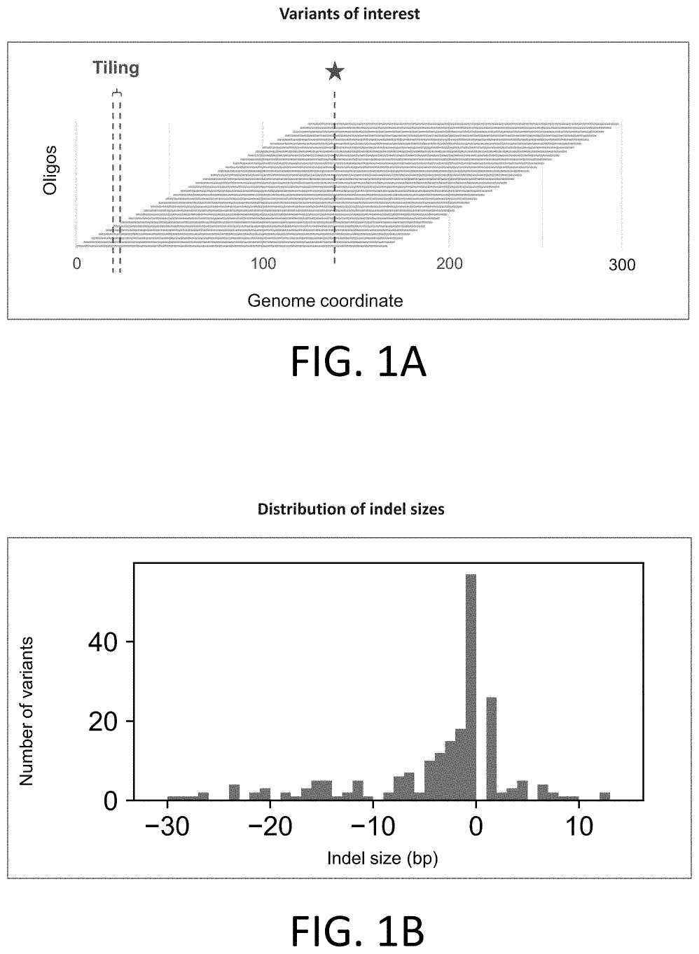

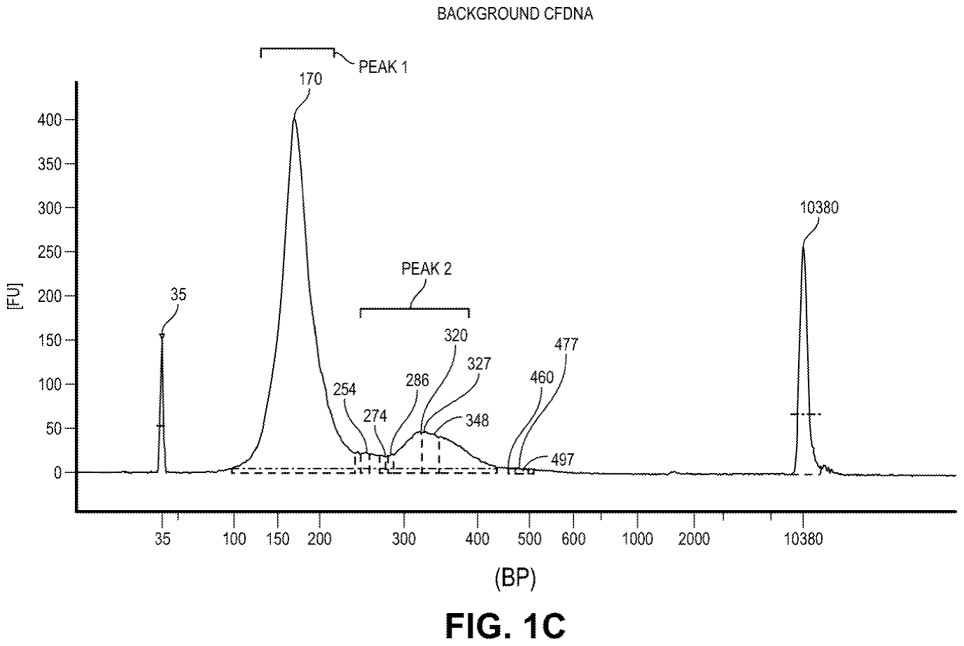

Polynucleotides provided herein may be tiled across a nucleic acid region. In some instances tiling describes the design of polynucleotides (or complements or reverse complements thereof) which cover or span a target area (such as a variant). An example of a tiling arrangement is shown in FIG. 1A. In some instances, tiling results in increases in sensitivity for detection either for probes targeting the variant, or in the design of corresponding standards, controls, or references. This is in some instances beneficial for regions of low abundance or comprising difficult sequences to sequence (repeating, high/low GC, or other challenge). In some instances, tiled polynucleotides for a target region are each different. Such tiling designs in some instances comprise about 2, 3, 4, 5, 6, 7, 8, 9, 10, 11, 12, 15, 20, 25, 27, 30, 32, 35, 40, 45, or about 50 polynucleotides tiled across a region (e.g., variant). Tiling designs in some instances comprise at least 2, 3, 4, 5, 6, 7, 8, 9, 10, 11, 12, 15, 20, 25, 30, 35, 40, 45, or at least 50 polynucleotides tiled across a region. Tiling designs in some instances comprise 10-100, 5-50, 2-50, 25-50, 30-40, or 30-60 polynucleotides tiled across a region. In some instances, tiled polynucleotides comprise at least one overlap region with another polynucleotide. In some instances, both 5′ and 3′ termini of a tiled polynucleotide overlap with an adjacent tiled polynucleotide. In some instances, one or more tiled polynucleotides are tiled with an offset value, such that a first polynucleotide starts at a different position than the next tiled polynucleotide. In some instances, the offset is about 1, 2, 3, 4, 5, 6, 7, 8, 9, 10, 12, 15, 17, 20, 25, or 30 bases. In some instances, the offset is 1-30, 1-20, 1-10, 1-8, or 2-5 bases. In some instances, the length of at least some of the polynucleotides is 20-500, 50-500, 75-500, 100-200, 100-500, 200-500, 100-250, 100-200, 100-1000, 250-500, or 250-1000. In some instances, the length of at least some of the polynucleotides is about 50, 75, 100, 125, 150, 155, 160, 165, 170, 175, 180, 190, 200, or 225 bases. In some instances, the length of at least 80% of the polynucleotides is 20-500, 50-500, 75-500, 100-200, 100-500, 200-500, 100-250, 100-200, 100-1000, 250-500, or 250-1000. In some instances, the length of at least 80% of the polynucleotides is about 50, 75, 100, 125, 150, 155, 160, 165, 170, 175, 180, 190, 200, or 225 bases. In some instances, the length of at least 90% of the polynucleotides is 20-500, 50-500, 75-500, 100-200, 100-500, 200-500, 100-250, 100-200, 100-1000, 250-500, or 250-1000. In some instances, the length of at least 90% of the polynucleotides is about 50, 75, 100, 125, 150, 155, 160, 165, 170, 175, 180, 190, 200, or 225 bases. In some instances, at least some of the polynucleotides are double stranded. In some instances, at least 50%, 60%, 70%, 75%, 80%, 90%, 95%, or at least 98% of the polynucleotides are double stranded.

Variants may be present at a predetermined frequency relative to other variants in a library (e.g., sample library). In some instances, at least 80% of the at least one variants are present at frequencies that differ by no more than 20%, 15%, 12%, 10%, 8% or no more than 5% relative to the expected frequency for uniformly pooled variants. In some instances, at least 90% of the at least one variants are present at frequencies that differ by no more than 20%, 15%, 12%, 10%, 8% or no more than 5% relative to the expected frequency for uniformly pooled variants. In some instances, at least 95% of the at least one variants are present at frequencies that differ by no more than 20%, 15%, 12%, 10%, 8% or no more than 5% relative to the expected frequency for uniformly pooled variants. In some instances, at least 99% of the at least one variants are present at frequencies that differ by no more than 20%, 15%, 12%, 10%, 8% or no more than 5% relative to the expected frequency for uniformly pooled variants.

Compositions described herein may comprise a background set (or library) of polynucleotides. The background set in some instances mimics the background cfDNA that would be present in a patient sample. In some instances, background polynucleotides are mixed with sample polynucleotides (e.g., polynucleotides comprising variants, variant polynucleotide libraries) to generate reference standards or controls. Standards or controls in some instances comprise variants having a VAF of 0%, 0.1% 0.25%, 0.5%, 1%, 2%, 5%, 10%, 15%, or 20% relative to a wild-type genomic sequence. In some instances, the background polynucleotide set comprises wild-type regions corresponding to locations of the at least one variant. In some instances, wild-type sequences are derived from a reference database or sample. In some instances, the background polynucleotide set comprises wild-type regions corresponding to locations of the at least 1, 2, 5, 10, 15, 20, 25, 50, 75, 100, 125, 150, 200, 250, 300, 350, 400, 450, 500, or at least 500 variants. In some instances, the wild-type regions are represented within 30%, 25%, 20%, 15%, 12%, 10%, 9%, 8%, 7%, or within 5% of the variant frequency of the variant set. In some instances, the background set comprises a low level amount of variations. In some instances, least one background polynucleotide comprises a variant present at a frequency of 0.001%, 0.01%, 0.1% 0.25%, 0.5%, 1%, or 2% relative to a wild-type genomic sequence. In some instances, least 1% of the background polynucleotides comprise a variant present at a frequency of 0.001%, 0.01%, 0.1% 0.25%, 0.5%, 1%, or 2% relative to a wild-type genomic sequence. In some instances, a background set is synthesized from pre-determined sequences. In some instances, the pre-determined sequences reflect desired variant frequencies. In some instances, synthetic background sets are used to calibrate instruments or methods by providing control over variant frequencies. In some instances, synthetic background sets are configured to mimic variant frequencies corresponding to specific samples or disease states.

In some instances, a background set comprises background polynucleotides. In some instances, a background set comprises background polynucleotides which substantially consist of wild-type sequences. In some instances, background sets are derived or isolated from healthy individuals. In some instances, the individual is a male. In some instances, the individual is a female. In some instances, the individual is no more than 40, 35, 30, 25, 20, or 15 years old. In some instances, background sets are obtained from a biological sample. In some instances, the biological sample comprises blood, plasma, or other source of nucleic acids. In some instances, the background set comprises cfDNA. In some instances, background sets comprises at least 2, 5, 10, 100, 200, 500, 1000, 10,000, 100,000, 500,000 polynucleotides, 1 million, 5 million, 10 million, 50 million, 100 million, 200 million, or more than 500 million polynucleotides. In some instances, the highest abundance of polynucleotides in the background set are 100-500, 50-500, 75-250, 50-750, 50-300, 100-300, 100-200, 125-300, 150-175, 150-185, or 125-200 bases in length. In some instances, at least 50%, 60%, 70%, 75%, 80%, 85%, 90%, 95%, or at least 97% of the polynucleotides in the background set are mononucleosomal or dinucleosomal. In some instances, the ratio of mononucleosomal to dinucleosomal is 50:50 to 90:10, 60:40 to 90:10, 60:40 to 95:5, 70:30 to 95:5, 70:30 to 90:10, or 80:20 to 95:5.

Polynucleotide libraries described herein may be mixed to form standards. In some instances, a (reference) standard comprises both a sample (variant) polynucleotide set and control polynucleotide. In some instances, standards comprising both a sample (variant) polynucleotide set and control polynucleotide set further comprise a liquid buffer. In some instances, the buffer comprises TE or TBE buffer. In some instances, standards comprise no more than 50%, 40%, 30%, 25%, 20%, 15%, or no more than 10% sample (variant) polynucleotides relative to background polynucleotides. Standards or controls in some instances comprise variants having a VAF of 0%, 0.1% 0.25%, 0.5%, 1%, or 2% relative to a wild-type genomic sequence. In some instances, a standard is subjected to one or more quality control operations including one or more of fluorescence/UV DNA quantification, electrophoretic size analysis, sequencing, ddPCR analysis, or other analysis technique. In some instances, a sample polynucleotide set is subjected to one or more quality control operations including one or more of fluorescence/UV DNA quantification, electrophoretic size analysis, sequencing, ddPCR analysis, or other analysis technique prior to mixing with a background polynucleotide set. In some instances, adapters comprising UMIs are ligated to sample polynucleotides.

Synthetic libraries (e.g., sample libraries/sets) comprising variants may have fewer contaminants (less contamination) than libraries derived from biological samples. A lower level of contaminants in some instances results in improved performance as a reference standard. In some instances, contamination includes but is not limited to cellular components, lipids, RNA, proteins, or other biomolecules derived from the biological source. In some instances, the biological source comprises plasma, cells, blood, or other source of nucleic acids. In some instances, synthetic libraries are prepared or stored in a buffer. In some instances, a synthetic library is at least 95%, 96%, 97%, 98%, 99%, 99.5%, or at least 99.7% free from biological contaminants.

Genomic Variants

Genetic variants (“variants” in nucleic acids) among populations of individuals may provide information regarding risk for diseases, identification of individuals, response to drug treatments, or susceptibility to environmental factors such as toxins. Compositions described herein in some instances involve synthesis of polynucleotide libraries which contain these variants. In some instances variants comprise a single nucleotide polymorphism (SNP), a single nucleotide variation (SNV), an indel, a copy number variation, a translocation, fusion, inversion, or structural variant. In some instances, a SNP differs between individuals in the same population. In some instance, an SNP differs between individuals in a different population. In some instances, an SNV comprises a variation in a single nucleotide without any limitations of frequency. Polynucleotide libraries (e.g., probe libraries) described herein are in some instances used to identify such variants after sequencing. In some instances, polynucleotide libraries are configured to enrich for nucleic acids (e.g., fragments of a genome) which comprise variants. Such nucleic acids in some instances are captured using the polynucleotide libraries and sequenced for calling variants. In some instances, variant calls may be assessed comparing to known variants using metrics such as recall and/or precision for one or all of the variants. In some instances, an SNP or SNV is heterozygous. In some instances, an SNP or SNV is homozygous. In some instances, an SNP or SNV is homozygous in matching a reference sequence. In some instances a variant is homozygous for a state other than that observed in the human reference genome. In some instances, variants are identified after sequencing by comparison to a reference database. In some instances the reference database comprises GiAB, dbSNP, DoGSD, dbGaP, clinvar, ncbi, refseq, refSNP, COSMIC, or other database which comprises known variants. In some instances, variants comprise an insertion, deletion, fusion, duplication, frameshift, repeat expansion, or substitution. In some instances, variants comprise a copy number variant (CNV), microsatellite instability, loss of heterozygosity (LOH), DNA methylation, premature stop codon, trinucleotide repeat, translocation, somatic rearrangement, allelomorph, single nucleotide variant (SNV), indel, splice variant, regulator variant, copy number variant, or fusion. In some instances indels are 1-50, 1-25, 1-20, 1-15, 2-20, 5-25, 5-15, or 5-10 bases in length. In some instances indels are not more than 1, 2, 3, 5, 7, 8, 10, 12, 15, 17, 20, 25, or no more than 50 bases in length. In some instances, a variant described herein is located in a gene. In some instances, a library described herein comprises variants found in at least 2, 5, 10, 15, 20, 25, 30, 50, 60, 75, 100, 125, 150, 200, 250, 300, 400, or at least 500 genes. In some instances, a library described herein comprises variants found in about 2, 5, 10, 15, 20, 25, 30, 50, 60, 75, 100, 125, 150, 200, 250, 300, 400, or about 500 genes. In some instances, a library described herein comprises variants found in 5-500, 5-100, 5-50, 10-200, 10-100, 25-500, 25-250, 25-150, 50-150, 50-250, 50-500, or 75-500 genes.

Identification of variants in some instances is accomplished using imputed data. In some instances, identification of variants near a known or detected variant inform the identity of a variant no measured, or which lacks sequencing data to accurately call. In some instances, the unmeasured (or unknown) genomic variant is within 100 bases, 500 bases, 1,000 bases, 10,000 bases, 100,000 bases, or 1,000,000 bases of a measured (or identified) genomic variant or variants, or more, depending on linkage disequilibrium (the non-random association of alleles for different variants within a population) between the measured and unmeasured variants. In some instances linkage disequilibrium may be inferred by making use of information about recombination rates observed in a genome or population otherwise known genetic distance. In some instances recombination rates, genetic distance maps, and variants themselves in some instances vary between different populations.

Variants may be present in a population of individuals, a single individual, tissue, or other group at different frequencies, such as in a genome. In some instances, genomic variants are co-occurring in less than 0.001, 0.01, 0.1, 0.5, 1, 1.5, 2, 5, 10, 20, 25, 50, or 75% of individuals in a group. In some instances, genomic variants are co-occurring in more than 0.001, 0.01, 0.1, 0.5, 1, 1.5, 2, 5, 10, 20, 25, 50, or 75% of individuals in a group. In some instances, genomic variants are co-occurring in about 0.001, 0.01, 0.1, 0.5, 1, 1.5, 2, 5, 10, 20, 25, 50, or 75% of individuals in a group. In some instances, genomic variants are co-occurring in 0.1-10%, 0.001-10%, 0.01-10%, 0.01-1%, 0.001-1%, 0.1-25%, 0.1-10%, or 0.1-5% of individuals in a group. In some instances, the occurrence of a variant is called a variant allele frequency (VAF).

Described herein are variants for detecting a disease or condition. In some instances, the disease or condition is a proliferative disease. In some instances, the disease or condition is cancer. In some instances, a variant is present in an oncogene or tumor suppressor gene. In some instances, a variant is present in one or more of genes ABL1, ABL2, AKT1, ALK, APC, AR, ARAF, ARID1A, ATM, ATR, BAP1, BRAF, BRCA1, BRCA2, CCND1, CDC6, CDH1, CDK12, CDK4, CDX2, CTNNB1, DDR2, EGFR, EML4, ERBB2, ERBB3, ERG, ESR1, EZH2, FBXW7, FGFR1, FGFR2, FGFR3, FLT3, FOXA1, FOXL2, GATA3, GNA11, GNAQ, GNAS, HNF1A, HRAS, IDH1, IDH2, JAK2, KDMSC, KDM6A, KIFSB, KIT, KRAS, MAP2K1, MAPK1, MET, MIR4728, ERBB2, MLH1, MPL, MYCN, MYD88, NCOA4, NF1, NF2, NFE2L2, NOTCHI, NPM1, NRAS, PBRM1, PDGFRA, PIK3CA, PTEN, PTPN11, RET, RHEB, RHOA, RIT1, ROS1, SETD2, SMAD4, SMO, SPOP, TERT, TMPRSS2, TP53, TPR, TSC1, and VHL. In some instances, a variant is present in one, two, three, five, seven, ten, 15, 20, 25 or more of genes ABL1, ABL2, AKT1, ALK, APC, AR, ARAF, ARID1A, ATM, ATR, BAP1, BRAF, BRCA1, BRCA2, CCND1, CDC6, CDH1, CDK12, CDK4, CDX2, CTNNB1, DDR2, EGFR, EML4, ERBB2, ERBB3, ERG, ESR1, EZH2, FBXW7, FGFR1, FGFR2, FGFR3, FLT3, FOXA1, FOXL2, GATA3, GNA11, GNAQ, GNAS, HNF1A, HRAS, IDH1, IDH2, JAK2, KDM5C, KDM6A, KIF5B, KIT, KRAS, MAP2K1, MAPK1, MET, MIR4728, ERBB2, MLH1, MPL, MYCN, MYD88, NCOA4, NF1, NF2, NFE2L2, NOTCHI, NPM1, NRAS, PBRM1, PDGFRA, PIK3CA, PTEN, PTPN11, RET, RHEB, RHOA, RIT1, ROS1, SETD2, SMAD4, SMO, SPOP, TERT, TMPRSS2, TP53, TPR, TSC1, and VHL. In some instances, multiple variants are present in a single gene. In some instances, a variant is present in one, two, three, five, seven, ten, 15, 20, 25 or more of genes. In some instances, a variant is present in one, two, three, five, seven, ten, 15, 20, 25 or more of genes which are associated with a disease or condition.

In some instances, the disease or condition is breast cancer. In some instances, a variant is present in one or more of genes TP53, PIK3CA, ERBB2, MYC, FGFR1/ZNF703, GATA3, CCND1, and CHD1 (e.g., CDH1*).

In some instances, the disease or condition is lung cancer. In some instances, a variant is present in one or more of genes KRAS (e.g., K117N), EGFR, ROS, ALK, and BRAF.

In some instances, the disease or condition is colorectal cancer. In some instances, a variant is present in one or more of genes TP53 APC, KRAS, BRAF, PIK3CA, SMAD4, FBXW7 (e.g., R465C), and NF1.

In some instances, the disease or condition is bladder cancer. In some instances, a variant is present in one or more of TP53, FGFR3 (e.g., S249C), ARID1A and KDM6A.

In some instances, the disease or condition is prostate cancer. In some instances, a variant is present in one or more of genes ETS (e.g., ETS-TMPRSS2), SPOP (e.g., F133V), TP53, FOXA1 (e.g., R219), and PTEN.

In some instances, the disease or condition is kidney cancer. In some instances, a variant is present in one or more of genes PBRM1, SETD2, BAP1, KDM5C, MTOR, VHL, MET, NF2, KDM6A, SMARCB1, FH, and CDKN2A.

In some instances, the disease or condition is melanoma. In some instances, a variant is present in one or more of genes NRAS, BRAF, PTEN, CDKN2A, MAP2K1, MAP2K2, GNAQ, GNA11, BAP (e.g., W196X).

In some instances, a variant is described in Table 1.

TABLE 1

| | mutation | | | | | | |
|---|---|---|---|---|---|---|---|
| Gene | description | COSMIC_id | chrom | pos | shift | ref | alt |
| ARID1A | Q1401* | COSM51417 | chr1 | 26774428 | | C | T |
| ARID1A | M1564Hfs*8 | COSM211769 | chr1 | 26774915 | 487 | C | CC |
| MPL | p.W515L | COSM18918 | chr1 | 43349338 | 16574423 | G | T |
| NRAS | Q61H | COSM586 | chr1 | 114713907 | 71364569 | T | G |
| NRAS | G12D | COSM564 | chr1 | 114716126 | 2219 | C | T |
| RIT1 | M90I | COSM3 57927 | chr1 | 155904470 | 41188344 | C | T |
| ABL2 | P986Hfs*4 | COSM2095020 | chr1 | 179108313 | 23203843 | GG | G |
| DDR2 | I638F | COSM7363943 | chr1 | 162775707 | −16332606 | A | T |
| GATA3 | P408fs | COSM166059 | chr10 | 8073911 | −154701796 | C | CG |
| RET | p.M918T | COSM965 | chr10 | 43121968 | 35048057 | T | C |
| PTEN | R130G | COSM5033 | chr10 | 87933148 | 44811180 | G | A |
| PTEN | p.D268Gfs*30 | COSM5012 | chr10 | 87958018 | 24870 | A | AA |
| PTEN | p.N323Kfs*2 | COSM4990 | chr10 | 87961060 | 3042 | A | AA |
| FGFR2 | K659E | COSM36909 | chr10 | 121488002 | 33526942 | T | C |
| FGFR2 | N549K | COSM36912 | chr10 | 121498520 | 10518 | A | T |
| FGFR2 | C382R | COSM36906 | chr10 | 121515260 | 16740 | A | G |
| FGFR2 | S252W | COSM36903 | chr10 | 121520163 | 4903 | G | C |
| HRAS | G12V | COSM483 | chr11 | 534288 | −120985875 | C | A |
| CCND1 | T286I | COSM931395 | chr11 | 69651251 | 69116963 | C | T |
| CCND1 | E275*fs | COSM931393 | chr11 | 69651217 | 69116929 | G | T |
| ATM | S214Pfs*16 | COSM1350740 | chr11 | 108244089 | 38592838 | CT | C |
| KRAS | K117N | COSM19940 | chr12 | 25225713 | −83018376 | T | G |
| KRAS | Q61H | COSM554 | chr12 | 25227341 | 1628 | T | G |
| KRAS | G13D | COSM532 | chr12 | 25245347 | 18006 | C | T |
| KRAS | p.G12D | COSM521 | chr12 | 25245350 | 3 | C | T |
| ERBB3 | v104m | COSM20710 | chr12 | 56085070 | 30839723 | G | A |
| CDK4 | R24C | COSM1677139 | chr12 | 57751648 | 1666578 | G | A |
| PTPN11 | E76K | COSM13000 | chr12 | 112450406 | 54698758 | G | A |
| PTPN11 | G503R | COSM14259 | chr12 | 112489083 | 38677 | G | A |
| HNF1A | P289fs | COSM1476243 | chr12 | 120994313 | 8505230 | G | GC |
| CDX2 | V306Cfs*2 | COSM1366182 | chr13 | 27963146 | −93031167 | CC | C |
| FLT3 | p.D835Y | COSM783 | chr13 | 28018505 | 55359 | C | A |
| BRCA2 | N1784Tfs*7 | COSM18607 | chr13 | 32339699 | 4321194 | CA | C |
| BRCA2 | T3033Lfs*29 | COSM1366491 | chr13 | 32379885 | 40186 | CA | C |
| FOXA1 | R219S | COSM3738526 | chr14 | 37592129 | 5212244 | G | T |
| AKT1 | Q79K | COSM159008 | chr14 | 104776711 | 67184582 | G | T |
| AKT1 | L52R | COSM93893 | chr14 | 104780108 | 3397 | A | C |
| AKT1 | p.E17K | COSM33765 | chr14 | 104780214 | 3503 | C | T |
| MAP2K1 | K57N | COSM1235478 | chr15 | 66435117 | −38345097 | G | T |
| IDH2 | R140Q | COSM41590 | chr15 | 90088702 | 23653585 | C | T |
| IDH2 | R172K | COSM33733 | chr15 | 90088606 | 23653489 | C | T |
| CDH1 | Q23* | COSM19503 | chr16 | 68738315 | −21350387 | C | T |
| CDH1 | A634V | COSM19822 | chr16 | 68822190 | 83875 | C | T |
| CDH1 | R732Q | COSM972800 | chr16 | 68828204 | 6014 | G | A |
| TP53 | R282W | COSM10704 | chr17 | 7673776 | −61154428 | G | A |
| TP53 | R342Efs*3 | COSM18597 | chr17 | 7670685 | −61157519 | GG | G |
| TP53 | p.R273H | COSM10660 | chr17 | 7673802 | 3117 | C | T |
| TP53 | G245C | COSM11081 | chr17 | 7674230 | 428 | C | A |
| TP53 | p.R175H | COSM10648 | chr17 | 7675088 | 858 | C | T |
| TP53 | p.L26Pfs*11 | COSM45386 | chr17 | 7676382 | 1294 | CAGAAC | C |
| | | | | | | GTTGTT | |
| | | | | | | TTCAGG | |
| | | | | | | AAGT | |
| | | | | | | (SEQ ID | |
| | | | | | | NO: 5) | |
| TP53 | R209Kfs*6 | COSM6482 | chr17 | 7674903 | 673 | TTC | T |
| TP53 | P152Rfs*18 | COSM43792 | chr17 | 7675156 | 68 | CG | C |
| TP53 | V73Rfs*76 | COSM128714 | chr17 | 7676152 | 996 | C | C |
| NF1 | I679Dfs*21 | COSM24504 | chr17 | 31226465 | 23550083 | C | CC |
| NF1 | F1247Ifs*18 | COSM436320 | chr17 | 31235638 | 9173 | CTGTT | C |
| NF1 | Y2285Tfs*5 | COSM39161 | chr17 | 31338733 | 103095 | CACTT | C |
| CDK12 | W719* | COSM118018 | chr17 | 39492798 | 8154065 | G | A |
| CDK12 | E928fs27* | COSM6965693 | chr17 | 39515745 | 22947 | A | AATACA |
| | | | | | | | CAAAGA |
| | | | | | | | T (SEQ ID |
| | | | | | | | NO: 6) |
| ERBB2 | L755S | COSM14060 | chr17 | 39723967 | 208222 | T | C |
| ERBB2 | p.A775_G776i | COSM20959 | chr17 | 39724730 | 763 | C | CATACG |
| | nsYVMA | | | | | | TGATGG |
| | (“YVMA” | | | | | | C (SEQ ID |
| | disclosed as | | | | | | NO: 8) |
| | SEQ ID NO: | | | | | | |
| | 7) | | | | | | |
| ERBB2 | P780_Y781ins | COSM21607 | chr17 | 39724758 | 28 | A | AGGCTC |
| | GSP | | | | | | ACCA |
| | | | | | | | (SEQ ID |
| | | | | | | | NO: 9) |
| ERBB2 | V842I | COSM14065 | chr17 | 39725079 | 349 | G | A |
| BRCA1 | R1443* | COSM979730 | chr17 | 43082434 | 3357355 | G | A |
| BRCA1 | K654Sfs*47 | COSM219054 | chr17 | 43093569 | 11135 | CT | C |
| BRCA1 | E23Vfs*17 | COSM35893 | chr17 | 43124027 | 30458 | ACT | A |
| SPOP | F133V | COSM219965 | chr17 | 49619064 | 6495037 | A | C |
| SMAD4 | D52Rfs*2 | COSM14091 | chr18 | 51047198 | 1428134 | A | AA |
| SMAD4 | R361H | COSM14122 | chr18 | 51065549 | 18351 | G | A |
| NFE2L2 | G333C | COSM1193323 | chr19 | 10491905 | −40573644 | C | A |
| MYCN | P44L | COSM35624 | chr2 | 15942195 | 5450290 | C | T |
| ALK | P1543S | COSM2941442 | chr2 | 29193460 | 13251265 | G | A |
| ALK | R1275Q | COSM28056 | chr2 | 29209798 | 16338 | C | T |
| ALK | R1192P | COSM7340824 | chr2 | 29220776 | 10978 | C | G |
| ALK | F1174L | COSM28055 | chr2 | 29220829 | 11031 | G | T |
| ALK | G1128A | COSM98475 | chr2 | 29222584 | 1755 | C | G |
| IDH1 | p.R132C | COSM28747 | chr2 | 208248389 | 179025805 | G | A |
| GNAS | p.R201C | COSM27887 | chr20 | 58909365 | −149339024 | C | T |
| MAPK1 | E322K | COSM461148 | chr22 | 21772875 | −37136490 | C | T |
| NF2 | L14Qfs*34 | COSM22312 | chr22 | 29604033 | 7831158 | GCT | G |
| NF2 | P275Tfs*4 | COSM6951489 | chr22 | 29665000 | 60967 | A | AA |
| NF2 | R341* | COSM21990 | chr22 | 29671847 | 6847 | C | T |
| NF2 | E445Gfs*9 | COSM22271 | chr22 | 29673477 | 1630 | CAGAG | C |
| VHL | F148Lfs*11 | COSM14410 | chr3 | 10146612 | −19526865 | AT | A |
| VHL | R161* | COSM17612 | chr3 | 10149804 | 3192 | C | T |
| MLH1 | R498fs | COSM5895322 | chr3 | 37028864 | 26879060 | GG | G |
| MYD88 | L265P | COSM85940 | chr3 | 38141150 | 1112286 | T | C |
| CTNNB1 | p.T41A | COSM5664 | chr3 | 41224633 | 3083483 | A | G |
| CTNNB1 | G34E | COSM5671 | chr3 | 41224613 | 3083463 | G | A |
| SETD2 | S2382Lfs*29 | COSM3068849 | chr3 | 47042655 | 5818022 | AG | A |
| SETD2 | R1407Gfs*5 | COSM3069036 | chr3 | 47120416 | 77761 | CT | C |
| SETD2 | S203Ifs*33 | COSM1161887 | chr3 | 47124027 | 3611 | TGA | T |
| RHOA | Y42C | COSM2849892 | chr3 | 49375465 | 2251438 | T | C |
| BAP1 | W196* | COSM51977 | chr3 | 52406900 | 3031435 | C | T |
| PBRM1 | p.I279Yfs*4 | COSM52863 | chr3 | 52644767 | 237867 | AT | A |
| FOXL2 | p.C134W | COSM33661 | chr3 | 138946321 | 86301554 | G | C |
| ATR | I774Yfs*5 | COSM214499 | chr3 | 142555906 | 3609585 | TT | T |
| PIK3CA | G106_R108del | COSM13475 | chr3 | 179199140 | 36643234 | AGGCAA | A |
| | | | | | | CCGT | |
| | | | | | | (SEQ ID | |
| | | | | | | NO: 10) | |
| PIK3CA | N345K | COSM754 | chr3 | 179203765 | 4625 | T | A |
| PIK3CA | p.E545K | COSM763 | chr3 | 179218303 | 14538 | G | A |
| PIK3CA | p.S553Tfs*20 | COSM27488 | chr3 | 179218327 | 24 | AGT | A |
| PIK3CA | p.H1047R | COSM775 | chr3 | 179234297 | 15994 | A | G |
| FGFR3 | p.S249C | COSM715 | chr4 | 1801841 | −177432456 | C | G |
| FGFR3 | Y375C | COSM718 | chr4 | 1804372 | 2531 | A | G |
| FGFR3 | K652E | COSM719 | chr4 | 1806162 | 1790 | A | G |
| FGFR3 | S249C | COSM715 | chr4 | 1801841 | −177432456 | C | G |
| PDGFRA | S566_ | COSM30546 | chr4 | 54274884 | 52468722 | GCCCAG | G |
| | E571delinsR | | | | | ATGGAC | |
| | | | | | | ATGA | |
| | | | | | | (SEQ ID | |
| | | | | | | NO: 11) | |
| PDGFRA | N659K | COSM22414 | chr4 | 54277981 | 3097 | C | G |
| PDGFRA | p.D842V | COSM736 | chr4 | 54285926 | 7945 | A | T |
| PDGFRA | V561D | COSM739 | chr4 | 54274869 | 52468707 | T | A |
| KIT | L576P | COSM1290 | chr4 | 54727495 | 441569 | T | C |
| KIT | del557-558 | COSM1210 | chr4 | 54727434 | 441508 | CAGTGG | C |
| | | | | | | A | |
| KIT | K642E | COSM1304 | chr4 | 54728055 | 560 | A | G |
| KIT | p.D816V | COSM1314 | chr4 | 54733155 | 5100 | A | T |
| FBXW7 | R465C | COSM22932 | chr4 | 152328233 | 97595078 | G | A |
| TERT | C228G | tert_c228g | chr5 | 1295229 | −151033004 | G | A |
| TERT | C250T | None | chr5 | 1295250 | 22 | C | T |
| APC | p.R213* | COSM13134 | chr5 | 112780895 | 111485666 | C | T |
| APC | A1002Gfs*6 | COSM5748894 | chr5 | 112838598 | 57703 | G | GG |
| APC | p.E1309Dfs*4 | COSM13113 | chr5 | 112839514 | 916 | TAAAAG | T |
| APC | p.R1450* | COSM13127 | chr5 | 112839942 | 428 | C | T |
| APC | R2714C | COSM2991126 | chr5 | 112843734 | 3792 | C | T |
| APC | p.S1465Wfs*3 | COSM13864 | chr5 | 112839978 | 36 | AAG | A |
| NPM1 | p.W288fs*12 | COSM17559 | chr5 | 171410539 | 58566805 | C | CTCTG |
| ROS1 | G2032R | COSM1651690 | chr6 | 117317184 | −54093355 | C | T |
| ESR1 | D538G | COSM94250 | chr6 | 152098791 | 34781607 | A | G |
| EGFR | L718Q | COSM6503269 | chr7 | 55174012 | −96924779 | T | A |
| EGFR | p.E746_A750d | COSM6225 | chr7 | 55174772 | 760 | GGAATT | G |
| | elELREA | | | | | AAGAG | |
| | (“ELREA” | | | | | AAGCA | |
| | disclosed as | | | | | (SEQ ID | |
| | SEQ ID NO: | | | | | NO: 13) | |
| | 12) | | | | | | |
| EGFR | S768I | COSM6241 | chr7 | 55181312 | 6540 | G | T |
| EGFR | p.D770_ | COSM12378 | chr7 | 55181319 | 7 | C | CGGT |
| | N771insG | | | | | | |
| EGFR | p.T790M | COSM6240 | chr7 | 55181378 | 6606 | C | T |
| EGFR | p.L858R | COSM6224 | chr7 | 55191822 | 10444 | T | G |
| EGFR | G724S | COSM13979 | chr7 | 55174029 | 17 | G | A |
| EGFR | L792H | COSM6493934 | chr7 | 55181384 | 6 | T | A |
| MET | exon_14_skip | metexon14_skip | chr7 | 116771990 | 61580168 | G | A |
| MET | d1246n | COSM5015794 | chr7 | 116783353 | 11363 | G | A |
| SMO | D473H | COSM34198 | chr7 | 129209348 | 12425995 | G | C |
| BRAF | p.V600E | COSM476 | chr7 | 140753336 | 11543988 | A | T |
| EZH2 | Y641F | COSM37028 | chr7 | 148811635 | 8058299 | T | A |
| RHEB | Y35N | COSM485065 | chr7 | 151490964 | 2679329 | A | T |
| FGFR1 | K656E | COSM35673 | chr8 | 38414790 | −113076174 | T | C |
| FGFR1 | N546K | COSM19176 | chr8 | 38417331 | 2541 | G | T |
| JAK2 | p.V617F | COSM12600 | chr9 | 5073770 | −33343561 | G | T |
| GNAQ | p.Q209P | COSM28758 | chr9 | 77794572 | 72720802 | T | G |
| GNAQ | T96S | COSM404628 | chr9 | 77922196 | 127624 | T | A |
| ABL1 | F317V | COSM211607 | chr9 | 130872901 | 52950705 | T | G |
| TSC1 | Q794 | COSM753312 | chr9 | 132902616 | 2029715 | G | A |
| NOTCH1 | P2514Rfs*4 | COSM12774 | chr9 | 136496196 | 3593580 | CAG | C |
| NOTCH1 | p.L1600Pfs*10 | COSM5751249 | chr9 | 136504893 | 8697 | G | GG |
| KDM6A | K1097Sfs*6 | COSM7211707 | chrX | 45083464 | −91421429 | AAGTT | A |
| ARAF | S214C | COSM5044705 | chrX | 47566722 | 2483258 | C | G |
| KDM5C | p.D1407Tfs*5 | COSM1161909 | chrX | 53193534 | 5626812 | TC | T |
| AR | W742C | COSM5944171 | chrX | 67717530 | 14523996 | G | C |
| AR | T878A | COSM236693 | chrX | 67723710 | 6180 | A | G |

In some instances, a variant is described in Table 2.

TABLE 2

| | | region_ | | | | | LEGACY_ | | |
|---|---|---|---|---|---|---|---|---|---|
| gene | COSMIC_id | str | chrom | pos | ref | alt | MUTATION_ID | HGVSP | name |
| RET | COSM9358963 | chr10: | chr10 | 43112867 | T | TTCC | COSM9358963 | ENSP00000347942.3: | T > TTCC |
| | | 43112852- | | | | | | p.Phe555_ | |
| | | 43112963 | | | | | | Ser556insLeu | |
| PTEN | COSM7350864 | chr10: | chr10 | 87864536 | T | TCGG | COSM7350864 | ENSP00000361021.3: | T > TCGG |
| | | 87864469- | | | | GAGC | | p.Leu23SerfsTer23 | GAGC |
| | | 87864548 | | | | | | | |
| PTEN | COSM5346960 | chr10: | chr10 | 87894056 | T | TATG | COSM5346960 | ENSP00000361021.3: | T > TATG |
| | | 87894024- | | | | GGAT | | p.Phe37_ | GGATTG |
| | | 87894109 | | | | TG | | Pro38insMetGlyLeu | (SEQ |
| | | | | | | (SEQ | | | ID NO: |
| | | | | | | ID | | | 14) |
| | | | | | | NO: | | | |
| | | | | | | 14) | | | |
| PTEN | COSM5882 | chr10: | chr10 | 87925549 | A | ATAT | COSM5882 | ENSP00000361021.3: | A > ATAT |
| | | 87925512- | | | | | | p.Tyr68dup | |
| | | 87925557 | | | | | | | |
| PTEN | COSM1173605 | chr10: | chr10 | 87931069 | C | CTA | COSM1173605 | ENSP00000361021.3: | C > CTA |
| | | 87931045- | | | | | | p.Ala79ThrfsTer21 | |
| | | 87931089 | | | | | | | |
| PTEN | COSM7347202 | chr10: | chr10 | 87952180 | T | TCAC | COSM7347202 | ENSP00000361021.3: | T > TCAC |
| | | 87952117- | | | | CGA | | p.His185_ | CGA |
| | | 87952259 | | | | | | Leu186insHisArg | |
| ATM | COSM1235448 | chr11: | chr11 | 108247062 | T | TA | COSM1235448 | ENSP00000278616.4: | T > TA |
| | | 108246963- | | | | | | p.Ser334TyrfsTer5 | |
| | | 108247127 | | | | | | | |
| ATM | COSM6928114 | chr11: | chr11 | 108256298 | TGAA | TAAA | COSM6928114 | ENSP00000278616.4: | TGAA |
| | | 108256214- | | | G | AA | | p.Glu737LysfsTer6 | G > TA |
| | | 108256340 | | | | | | | AAAA |
| ATM | COSM9312366 | chr11: | chr11 | 108292758 | C | CATA | COSM9312366 | ENSP00000278616.4: | C > CAT |
| | | 108292618- | | | | A | | p.Pro1526HisfsTer6 | AA |
| | | 108292793 | | | | | | | |
| ATM | COSM9358682 | chr11: | chr11 | 108293339 | G | GGAT | COSM9358682 | ENSP00000278616.4: | G > GG |
| | | 108293312- | | | | A | | p.Ile1547AspfsTer4 | ATA |
| | | 108293477 | | | | | | | |
| ATM, | COSM6853938 | chr11: | chr11 | 108331534 | G | GA | COSM6853938 | ENSP00000278616.4: | G > GA |
| C11orf65 | | 108331443- | | | | | | p.Gly2536GlufsTer4 | |
| | | 108331557 | | | | | | | |
| ATM, | COSM6936524 | chr11: | chr11 | 108331960 | T | TA | COSM6936524 | ENSP00000278616.4: | T > TA |
| C11orf65 | | 108331878- | | | | | | p.Phe2571TyrfsTer4 | |
| | | 108332037 | | | | | | | |
| ATM, | COSM6854263 | chr11: | chr11 | 108345767 | G | GT | COSM6854263 | ENSP00000278616.4: | G > GT |
| C11orf65 | | 108345742- | | | | | | p.Glu2815ValfsTer4 | |
| | | 108345908 | | | | | | | |
| AKT1 | COSM7345039 | chr14: | chr14 | 104772908 | G | GC | COSM7345039 | ENSP00000451828.1: | G > GC |
| | | 104772877- | | | | | | p.Ser381CysfsTer54 | |
| | | 104773092 | | | | | | | |
| AKT1 | COSM5751911 | chr14: | chr14 | 104792618 | T | TC | COSM5751911 | ENSP00000451828.1: | T > TC |
| | | 104792597- | | | | | | p.Glu9GlyfsTer24 | |
| | | 104792643 | | | | | | | |
| ERBB2 | COSM9494270 | chr17: | chr17 | 39727873 | C | CAGA | COSM9494270 | ENSP00000269571.4: | C > CA |
| | | 39727688- | | | | G | | p.Gln1200ArgfsTer78 | GAG |
| | | 39728044 | | | | | | | |
| TP53 | COSM6503572 | chr17: | chr17 | 7669655 | C | CT | COSM6503572 | ENSP00000269305.4: | C > CT |
| | | 7669608- | | | | | | p.Arg379GlnfsTer3 | |
| | | 7669690 | | | | | | | |
| ALK | COSM7347227 | chr2: | chr2 | 29320797 | T | TG | COSM7347227 | ENSP00000373700.3: | T > TG |
| | | 29320750- | | | | | | p.Gln500HisfsTer26 | |
| | | 29320882 | | | | | | | |
| PIK3CA | COSM5751700 | chr3: | chr3 | 179219582 | G | GT | COSM5751700 | ENSP00000263967.3: | G > GT |
| | | 179219570- | | | | | | p.Val587CysfsTer10 | |
| | | 179219735 | | | | | | | |
| CTNNB1 | COSM6853630 | chr3: | chr3 | 41233407 | G | GGGA | COSM6853630 | ENSP00000495360.1: | G > GG |
| | | 41233340- | | | | | | p.Trp383_ | GA |
| | | 41233444 | | | | | | Thr384insGlu | |
| FGFR3 | COSM13248 | chr4: | chr4 | 1804823 | G | GGTA | COSM13248 | ENSP00000339824.4: | G > GG |
| | | 1804823- | | | | ACA | | p.Val425_ | TAAC |
| | | 1804969 | | | | | | Ser426insThrVal | A |
| FGFR3 | COSM7448276 | chr4: | chr4 | 1806568 | T | TTGG | COSM7448276 | ENSP00000339824.4: | T > TTG |
| | | 1806545- | | | | GAGA | | p.Trp687delinsLeu | GGAG |
| | | 1806683 | | | | TCTT | | GlyAspLeuAlaArg | ATCTT |
| | | | | | | GCAC | | (“LeuGlyAspLeuAla | GCAC |
| | | | | | | (SEQ | | Arg” | (SEQ |
| | | | | | | ID | | disclosed as | ID NO: |
| | | | | | | NO: | | SEQ ID NO: 16) | 15) |
| | | | | | | 15) | | | |
| FGFR3 | COSM729 | chr4: | chr4 | 1807221 | CT | CGA | COSM729 | ENSP00000339824.4: | CT > CG |
| | | 1807115- | | | | | | p.Leu796ArgfsTer23 | A |
| | | 1807262 | | | | | | | |
| PDGFRA | COSM7345286 | chr4: | chr4 | 54261354 | C | CA | COSM7345286 | ENSP00000257290.5: | C > CA |
| | | 54261094- | | | | | | p.His104ThrfsTer8 | |
| | | 54261412 | | | | | | | |
| PDGFRA | COSM9358924 | chr4: | chr4 | 54270721 | A | AAGC | COSM9358924 | ENSP00000257290.5: | A > AA |
| | | 54270632- | | | | T | | p.Ser404LysfsTer6 | GCT |
| | | 54270748 | | | | | | | |
| KIT | COSM6008883 | chr4: | chr4 | 54723605 | A | ACGA | COSM6008883 | ENSP00000288135.5: | A > AC |
| | | 54723583- | | | | TTTT | | p.Arg420PhefsTer30 | GATT |
| | | 54723698 | | | | | | | TT |
| KIT | COSM53306 | chr4: | chr4 | 54726012 | C | CTGC | COSM53306 | ENSP00000288135.5: | C > CTG |
| | | 54725856- | | | | CTT | | p.Ala502_ | CCTT |
| | | 54726050 | | | | | | Tyr503insPheAla | |
| APC | COSM9113053 | chr5: | chr5 | 112767380 | G | GGAG | COSM9113053 | ENSP00000257430.4: | G > GG |
| | | 112767188- | | | | AAAG | | p.Glu138GlyfsTer35 | AGAA |
| | | 112767390 | | | | A | | | AGA |
| APC | COSM5010340 | chr5: | chr5 | 112815593 | G | GATG | COSM5010340 | ENSP00000257430.4: | G > GA |
| | | 112815494- | | | | TTT | | p.Lys311_ | TGTTT |
| | | 112815593 | | | | | | Val312insMetPhe | |
| APC | COSM25155 | chr5: | chr5 | 112819174 | C | CG | COSM25155 | ENSP00000257430.4: | C > CG |
| | | 112818965- | | | | | | p.Arg382GlnfsTer15 | |
| | | 112819344 | | | | | | | |
| APC | COSM6854200 | chr5: | chr5 | 112835141 | T | TA | COSM6854200 | ENSP00000257430.4: | T > TA |
| | | 112834950- | | | | | | p.Ile646AspfsTer5 | |
| | | 112835165 | | | | | | | |
| ROS1 | COSM6967149 | chr6: | chr6 | 117319975 | G | GA | COSM6967149 | ENSP00000357494.3: | G > GA |
| | | 117319867- | | | | | | p.Leu1945SerfsTer17 | |
| | | 117320030 | | | | | | | |
| ROS1 | COSM9499684 | chr6: | chr6 | 117359964 | C | CAA | COSM9499684 | ENSP00000357494.3: | C > CA |
| | | 117359808- | | | | | | p.Val1165LeufsTer4 | A |
| | | 117360011 | | | | | | | |
| MET | COSM6957131 | chr7: | chr7 | 116763127 | C | CA | COSM6957131 | ENSP00000317272.6: | C > CA |
| | | 116763049- | | | | | | p.Leu833ThrfsTer18 | |
| | | 116763268 | | | | | | | |
| RET | COSM7341796 | chr10: | chr10 | 43077301 | TTGC | T | COSM7341796 | ENSP00000347942.3: | TTG |
| | | 43077258- | | | | | | p.Leu19del | C > T |
| | | 43077331 | | | | | | | |
| RET | COSM7351211 | chr10: | chr10 | 43102447 | CCTT | C | COSM7351211 | ENSP00000347942.3: | CCT |
| | | 43102341- | | | | | | p.Phe150del | T > C |
| | | 43102629 | | | | | | | |
| RET | COSM4989957 | chr10: | chr10 | 43105037 | CGAG | C | COSM4989957 | ENSP00000347942.3: | CGAG |
| | | 43104951- | | | CTGG | | | p.Glu238GlyfsTer113 | CTGG |
| | | 43105193 | | | T | | | | T > C |
| RET | COSM9277092 | chr10: | chr10 | 43111413 | GCAG | G | COSM9277092 | ENSP00000347942.3: | GCAG |
| | | 43111206- | | | ACCT | | | p.Thr492_Gln499del | ACCT |
| | | 43111465 | | | CTAG | | | | CTAG |
| | | | | | GCAG | | | | GCAG |
| | | | | | GCCC | | | | GCCC |
| | | | | | AGGC | | | | AGGC |
| | | | | | C | | | | C (SEQ |
| | | | | | (SEQ | | | | ID NO: |
| | | | | | ID | | | | 17) > G |
| | | | | | NO: | | | | |
| | | | | | 17) | | | | |
| RET | COSM1237681 | chr10: | chr10 | 43112100 | TGTG | T | COSM1237681 | ENSP00000347942.3: | TGTG |
| | | 43112098- | | | GCCG | | | p.Val509_Glu511del | GCCG |
| | | 43112224 | | | AG | | | | AG |
| | | | | | (SEQ | | | | (SEQ |
| | | | | | ID | | | | ID NO: |
| | | | | | NO: | | | | 18) > T |
| | | | | | 18) | | | | |
| RET | COSM984 | chr10: | chr10 | 43113625 | ACTG | A | COSM984 | ENSP00000347942.3: | ACTG |
| | | 43113555- | | | CTTC | | | p.Phe612_Cys620del | CTTCC |
| | | 43113675 | | | CCTG | | | | CTGA |
| | | | | | AGGA | | | | GGAG |
| | | | | | GGAG | | | | GAGA |
| | | | | | AAGT | | | | AGTG |
| | | | | | GCTT | | | | CTT |
| | | | | | (SEQ | | | | (SEQ |
| | | | | | ID | | | | ID NO: |
| | | | | | NO: | | | | 19) > A |
| | | | | | 19) | | | | |
| RET | COSM962 | chr10: | chr10 | 43120163 | GAGA | G | COSM962 | ENSP00000347942.3: | GAGA |
| | | 43120080- | | | TGTT | | | p.Asp898_Glu901del | TGTTT |
| | | 43120203 | | | TATG | | | | ATGA |
| | | | | | A | | | | (SEQ |
| | | | | | (SEQ | | | | ID NO: |
| | | | | | ID | | | | 20) > G |
| | | | | | NO: | | | | |
| | | | | | 20) | | | | |
| RET | COSM6929334 | chr10: | chr10 | 43123710 | AG | A | COSM6929334 | ENSP00000347942.3: | AG > A |
| | | 43123670- | | | | | | p.Gly949GlufsTer16 | |
| | | 43123808 | | | | | | | |
| RET | COSM7449721 | chr10: | chr10 | 43128223 | TGCT | T | COSM7449721 | ENSP00000347942.3: | TGCTT |
| | | 43128111- | | | TTCA | | | p.Leu1101GlnfsTer3 | TCAC |
| | | 43128269 | | | CCCT | | | | CCTC |
| | | | | | CAGC | | | | AGCG |
| | | | | | G | | | | (SEQ |
| | | | | | (SEQ | | | | ID NO: |
| | | | | | ID | | | | 21) > T |
| | | | | | NO: | | | | |
| | | | | | 21) | | | | |
| PTEN | COSM6942496 | chr10: | chr10 | 87965289 | GAAG | G | COSM6942496 | ENSP00000361021.3: | GAAG |
| | | 87965286- | | | CTGT | | | p.Leu345GlnfsTer2 | CTGT |
| | | 87965472 | | | ACTT | | | | ACTT |
| | | | | | CACA | | | | CACA |
| | | | | | A | | | | A (SEQ |
| | | | | | (SEQ | | | | ID NO: |
| | | | | | ID | | | | 22) > G |
| | | | | | NO: | | | | |
| | | | | | 22) | | | | |
| ATM | COSM1315819 | chr11: | chr11 | 108227624 | CATG | C | COSM1315819 | ENSP00000278616.4: | CATG |
| | | 108227624- | | | AGTC | | | p.Met1? | AGTC |
| | | 108227696 | | | TAGT | | | | TAGT |
| | | | | | ACTT | | | | ACTT |
| | | | | | AATG | | | | AATG |
| | | | | | (SEQ | | | | (SEQ |
| | | | | | ID | | | | ID NO: |
| | | | | | NO: | | | | 23) > C |
| | | | | | 23) | | | | |
| ATM | COSM6978979 | chr11: | chr11 | 108229315 | CAAA | C | COSM6978979 | ENSP00000278616.4: | CAAA |
| | | 108229177- | | | CAGA | | | p.Asn109SerfsTer3 | CAGA |
| | | 108229323 | | | A | | | | A > C |
| ATM | COSM758337 | chr11: | chr11 | 108235814 | TATC | T | COSM758337 | ENSP00000278616.4: | TATCT |
| | | 108235669- | | | TC | | | p.Ser160AlafsTer23 | C > T |
| | | 108235834 | | | | | | | |
| ATM | COSM3733253 | chr11: | chr11 | 108244952 | AAG | A | COSM3733253 | ENSP00000278616.4: | AAG > A |
| | | 108244787- | | | | | | p.Glu277SerfsTer4 | |
| | | 108245026 | | | | | | | |
| ATM | COSM1235427 | chr11: | chr11 | 108249045 | GGGA | G | COSM1235427 | ENSP00000278616.4: | GGGAA |
| | | 108248932- | | | AGTA | | | p.Trp393Ter | GTA > G |
| | | 108249102 | | | | | | | |
| ATM | COSM6945044 | chr11: | chr11 | 108250727 | CAAA | CT | COSM6945044 | ENSP00000278616.4: | CAAA |
| | | 108250700- | | | G | | | p.Lys422del | G > CT |
| | | 108251072 | | | | | | | |
| ATM | COSM6935895 | chr11: | chr11 | 108251945 | AAAG | A | COSM6935895 | ENSP00000278616.4: | AAAG |
| | | 108251836- | | | GAAT | | | p.Lys573AsnfsTer13 | GAAT |
| | | 108252031 | | | C | | | | C > A |
| ATM | COSM6958308 | chr11: | chr11 | 108252838 | GGA | G | COSM6958308 | ENSP00000278616.4: | GGA > G |
| | | 108252816- | | | | | | p.Lys610AsnfsTer11 | |
| | | 108252912 | | | | | | | |
| ATM | COSM22531 | chr11: | chr11 | 108253991 | CTGT | CG | COSM22531 | ENSP00000278616.4: | CTGT |
| | | 108253813- | | | CTTC | | | p.Cys693_ | CTTCT |
| | | 108254039 | | | TGGG | | | Gln700delinsGlu | GGGA |
| | | | | | ATTA | | | | TTATC |
| | | | | | TCAG | | | | AGAA |
| | | | | | AAC | | | | C (SEQ |
| | | | | | (SEQ | | | | ID NO: |
| | | | | | ID | | | | 24) > C |
| | | | | | NO: | | | | G |
| | | | | | 24) | | | | |
| ATM | COSM6986880 | chr11: | chr11 | 108257598 | TGTA | T | COSM6986880 | ENSP00000278616.4: | TGTA |
| | | 108257480- | | | CCA | | | p.Cys790_ | CCA > T |
| | | 108257606 | | | | | | Lys792delinsTer | |
| ATM | COSM9179264 | chr11: | chr11 | 108259065 | GTAA | G | COSM9179264 | | GTAA |
| | | 108258985- | | | AAGT | | | | AAGT |
| | | 108259075 | | | TTAG | | | | TTAG |
| | | | | | TAAG | | | | TAAG |
| | | | | | TA | | | | TA |
| | | | | | (SEQ | | | | (SEQ |
| | | | | | ID | | | | ID NO: |
| | | | | | NO: | | | | 25) > G |
| | | | | | 25) | | | | |
| ATM | COSM6856770 | chr11: | chr11 | 108267334 | GTAC | G | COSM6856770 | ENSP00000278616.4: | GTAC |
| | | 108267170- | | | CA | | | p.Thr878ArgfsTer4 | CA > G |
| | | 108267342 | | | | | | | |
| ATM | COSM6906886 | chr11: | chr11 | 108268563 | TTGA | T | COSM6906886 | ENSP00000278616.4: | TTGA |
| | | 108268409- | | | TTCT | | | p.Asp932ArgfsTer32 | TTCTA |
| | | 108268609 | | | AGCA | | | | GCAC |
| | | | | | CGC | | | | GC |
| | | | | | (SEQ | | | | (SEQ |
| | | | | | ID | | | | ID NO: |
| | | | | | NO: | | | | 26) > T |
| | | | | | 26) | | | | |
| ATM | COSM7345428 | chr11: | chr11 | 108271283 | ATGT | A | COSM7345428 | ENSP00000278616.4: | ATGT |
| | | 108271250- | | | T | | | p.Cys987LysfsTer4 | T > A |
| | | 108271406 | | | | | | | |
| ATM | COSM7345432 | chr11: | chr11 | 108272761 | GGA | G | COSM7345432 | ENSP00000278616.4: | GGA > G |
| | | 108272721- | | | | | | p.Gly1065GlufsTer7 | |
| | | 108272852 | | | | | | | |
| ATM | COSM1235411 | chr11: | chr11 | 108279519 | TC | T | COSM1235411 | ENSP00000278616.4: | TC > T |
| | | 108279490- | | | | | | p.Arg1106GlyfsTer3 | |
| | | 108279608 | | | | | | | |
| ATM | COSM9493731 | chr11: | chr11 | 108281162 | GA | G | COSM9493731 | ENSP00000278616.4: | GA > G |
| | | 108280994- | | | | | | p.Lys1192ArgfsTer3 | |
| | | 108281168 | | | | | | | |
| ATM | COSM758341 | chr11: | chr11 | 108282852 | ACTA | A | COSM758341 | ENSP00000278616.4: | ACTA |
| | | 108282709- | | | CACA | | | p.Asn1240LysfsTer4 | CACA |
| | | 108282879 | | | AATA | | | | AATA |
| | | | | | TTGA | | | | TTGA |
| | | | | | GG | | | | GG |
| | | | | | (SEQ | | | | (SEQ |
| | | | | | ID | | | | ID NO: |
| | | | | | NO: | | | | 27) > A |
| | | | | | 27) | | | | |
| ATM | COSM21638 | chr11: | chr11 | 108284389 | CAGA | C | COSM21638 | ENSP00000278616.4: | CAGAG |
| | | 108284226- | | | GACA | | | p.Arg1304ValfsTer43 | ACA > C |
| | | 108284473 | | | | | | | |
| ATM | COSM6958310 | chr11: | chr11 | 108287644 | GTTA | G | COSM6958310 | ENSP00000278616.4: | GTT |
| | | 108287599- | | | | | | p.Leu1348del | A > G |
| | | 108287715 | | | | | | | |
| ATM | COSM6971320 | chr11: | chr11 | 108289010 | TC | T | COSM6971320 | ENSP00000278616.4: | TC > T |
| | | 108288976- | | | | | | p.Pro1382HisfsTer4 | |
| | | 108289103 | | | | | | | |
| ATM | COSM6956709 | chr11: | chr11 | 108289695 | CTGT | C | COSM6956709 | ENSP00000278616.4: | CTG |
| | | 108289601- | | | T | | | p.Phe1445LeufsTer5 | TT > C |
| | | 108289801 | | | | | | | |
| ATM | COSM22532 | chr11: | chr11 | 108294983 | TGAA | TT | COSM22532 | ENSP00000278616.4: | TGAA |
| | | 108294926- | | | GGAC | | | p.Glu1612_ | GGAC |
| | | 108295059 | | | TAAA | | | Gln1620delinsTer | TAAA |
| | | | | | GGAT | | | | GGAT |
| | | | | | CTTC | | | | CTTC |
| | | | | | GAAG | | | | GAAG |
| | | | | | AC | | | | AC |
| | | | | | (SEQ | | | | (SEQ |
| | | | | | ID | | | | ID NO: |
| | | | | | NO: | | | | 28) > TT |
| | | | | | 28) | | | | |
| ATM | COSM4745906 | chr11: | chr11 | 108297369 | AAAA | A | COSM4745906 | ENSP00000278616.4: | AAAA |
| | | 108297286- | | | G | | | p.Glu1666PhefsTer2 | G > A |
| | | 108297382 | | | | | | | |
| ATM | COSM22533 | chr11: | chr11 | 108299754 | TTTC | T | COSM22533 | ENSP00000278616.4: | TTTCT |
| | | 108299713- | | | TC | | | p.Phe1683TyrfsTer7 | C > T |
| | | 108299885 | | | | | | | |
| ATM | COSM22526 | chr11: | chr11 | 108301670 | GTTA | G | COSM22526 | ENSP00000278616.4: | GTTA |
| | | 108301647- | | | CCTG | | | p.Thr1735GlufsTer11 | CCTG |
| | | 108301789 | | | T | | | | T > G |
| ATM | COSM7347299 | chr11: | chr11 | 108302857 | TAGA | T | COSM7347299 | ENSP00000278616.4: | TAG |
| | | 108302852- | | | | | | p.Glu1776del | A > T |
| | | 108303029 | | | | | | | |
| ATM | COSM9358193 | chr11: | chr11 | 108304685 | AC | A | COSM9358193 | ENSP00000278616.4: | AC > A |
| | | 108304674- | | | | | | p.Cys1838ValfsTer8 | |
| | | 108304852 | | | | | | | |
| ATM | COSM1315822 | chr11: | chr11 | 108307969 | TGAG | T | COSM1315822 | ENSP00000278616.4: | TGA |
| | | 108307896- | | | | | | p.Met1916_ | G > T |
| | | 108307984 | | | | | | Arg1917delinsIle | |
| ATM, | COSM5967541 | chr11: | chr11 | 108310286 | TAAG | T | COSM5967541 | ENSP00000278616.4: | TAAG |
| C11orf65 | | 108310159- | | | AAAA | | | p.Lys1964ArgfsTer19 | AAAA |
| | | 108310315 | | | GTAT | | | | GTAT |
| | | | | | GGAT | | | | GGAT |
| | | | | | GATC | | | | GATC |
| | | | | | AAG | | | | AAG |
| | | | | | (SEQ | | | | (SEQ |
| | | | | | ID | | | | ID NO: |
| | | | | | NO: | | | | 29) > T |
| | | | | | 29) | | | | |
| ATM, | COSM1235422 | chr11: | chr11 | 108316060 | TA | T | COSM1235422 | ENSP00000278616.4: | TA > T |
| C11orf65 | | 108316010- | | | | | | p.Tyr2049LeufsTer33 | |
| | | 108316113 | | | | | | | |
| ATM, | COSM6944878 | chr11: | chr11 | 108317460 | GAAG | GTC | COSM6944878 | ENSP00000278616.4: | GAAG |
| C11orf65 | | 108317372- | | | AACT | | | p.Glu2096ValfsTer29 | AAC |
| | | 108317521 | | | | | | | T > GTC |
| ATM, | COSM6911065 | chr11: | chr11 | 108319958 | GA | G | COSM6911065 | ENSP00000278616.4: | GA > G |
| C11orf65 | | 108319953- | | | | | | p.Val2119Ter | |
| | | 108320058 | | | | | | | |
| ATM, | COSM21644 | chr11: | chr11 | 108325443 | GAA | G | COSM21644 | ENSP00000278616.4: | GAA > G |
| C11orf65 | | 108325309- | | | | | | p.Lys2237GlyfsTer11 | |
| | | 108325544 | | | | | | | |
| ATM, | COSM6933908 | chr11: | chr11 | 108326152 | CA | C | COSM6933908 | ENSP00000278616.4: | CA > C |
| C11orf65 | | 108326057- | | | | | | p.Lys2303ArgfsTer7 | |
| | | 108326225 | | | | | | | |
| ATM, | COSM758343 | chr11: | chr11 | 108327657 | CTAA | C | COSM758343 | ENSP00000278616.4: | CTAA |
| C11orf65 | | 108327644- | | | AACT | | | p.Lys2331HisfsTer6 | AAC |
| | | 108327758 | | | | | | | T > C |
| ATM, | COSM6977654 | chr11: | chr11 | 108329028 | AAG | A | COSM6977654 | ENSP00000278616.4: | AAG > A |
| C11orf65 | | 108329020- | | | | | | p.Glu2366AspfsTer6 | |
| | | 108329238 | | | | | | | |
| ATM, | COSM6986181 | chr11: | chr11 | 108330215 | TACA | T | COSM6986181 | ENSP00000278616.4: | TACA |
| C11orf65 | | 108330213- | | | C | | | p.Tyr2437Ter | C > T |
| | | 108330421 | | | | | | | |
| ATM, | COSM4745907 | chr11: | chr11 | 108332850 | CTTA | C | COSM4745907 | ENSP00000278616.4: | CTTAT |
| C11orf65 | | 108332761- | | | TA | | | p.Ile2629SerfsTer25 | A > C |
| | | 108332900 | | | | | | | |
| ATM, | COSM6853895 | chr11: | chr11 | 108333892 | TA | T | COSM6853895 | ENSP00000278616.4: | TA > T |
| C11orf65 | | 108333885- | | | | | | p.Asn2646IlefsTer14 | |
| | | 108333968 | | | | | | | |
| ATM, | COSM6986871 | chr11: | chr11 | 108334992 | AAAT | A | COSM6986871 | ENSP00000278616.4: | AAAT |
| C11orf65 | | 108334968- | | | CTGG | | | p.Asn2679SerfsTer9 | CTGG |
| | | 108335109 | | | TGAC | | | | TGAC |
| | | | | | TATA | | | | TATA |
| | | | | | C | | | | C(SEQ |
| | | | | | (SEQ | | | | ID NO: |
| | | | | | ID | | | | 30) > A |
| | | | | | NO: | | | | |
| | | | | | 30) | | | | |
| ATM, | COSM1235408 | chr11: | chr11 | 108343270 | ACTG | AA | COSM1235408 | ENSP00000278616.4: | ACTG |
| C11orf65 | | 108343221- | | | TCCC | | | p.Thr2773AsnfsTer4 | TCCC |
| | | 108343371 | | | CATT | | | | CATT |
| | | | | | GGTG | | | | GGTG |
| | | | | | AAT | | | | AAT |
| | | | | | (SEQ | | | | (SEQ |
| | | | | | ID | | | | ID NO: |
| | | | | | NO: | | | | 31) > AA |
| | | | | | 31) | | | | |
| ATM, | COSM22484 | chr11: | chr11 | 108347306 | GACA | G | COSM22484 | ENSP00000278616.4: | GAC |
| C11orf65 | | 108347278- | | | | | | p.Arg2871_ | A > G |
| | | 108347365 | | | | | | His2872delinsSer | |
| ATM, | COSM6933059 | chr11: | chr11 | 108353803 | TGAG | T | COSM6933059 | ENSP00000278616.4: | TGAG |
| C11orf65 | | 108353765- | | | ACAG | | | p.Glu2904AspfsTer29 | ACAG |
| | | 108353880 | | | TTCC | | | | TTCCT |
| | | | | | TTTT | | | | TTTA |
| | | | | | A | | | | (SEQ |
| | | | | | (SEQ | | | | ID NO: |
| | | | | | ID | | | | 32) > T |
| | | | | | NO: | | | | |
| | | | | | 32) | | | | |
| ATM, | COSM6930780 | chr11: | chr11 | 108354854 | ACT | A | COSM6930780 | ENSP00000278616.4: | ACT > A |
| C11orf65 | | 108354810- | | | | | | p.Leu2945ValfsTer10 | |
| | | 108354874 | | | | | | | |
| ATM, | COSM3733420 | chr11: | chr11 | 108365362 | GTCT | G | COSM3733420 | ENSP00000278616.4: | GTC |
| C11orf65 | | 108365324- | | | | | | p.Leu3010del | T > G |
| | | 108365508 | | | | | | | |
| AKT1 | COSM9358172 | chr14: | chr14 | 104773279 | CA | C | COSM9358172 | ENSP00000451828.1: | CA > C |
| | | 104773250- | | | | | | p.Cys310AlafsTer33 | |
| | | 104773379 | | | | | | | |
| ERBB2 | COSM5967125 | chr17: | chr17 | 39707050 | TGCT | T | COSM5967125 | ENSP00000269571.4: | TGCT |
| | | 39706989- | | | CCGC | | | p.Leu46AlafsTer40 | CCGC |
| | | 39707141 | | | CACC | | | | CACC |
| | | | | | TCTA | | | | TCTA |
| | | | | | CCAG | | | | CCAG |
| | | | | | (SEQ | | | | (SEQ |
| | | | | | ID | | | | ID NO: |
| | | | | | NO: | | | | 33) > T |
| | | | | | 33) | | | | |
| ERBB2 | COSM7345562 | chr17: | chr17 | 39708492 | TCC | T | COSM7345562 | ENSP00000269571.4: | TCC > T |
| | | 39708320- | | | | | | p.Pro134ArgfsTer66 | |
| | | 39708534 | | | | | | | |
| ERBB2 | COSM7345564 | chr17: | chr17 | 39710383 | GC | G | COSM7345564 | ENSP00000269571.4: | GC > G |
| | | 39710339- | | | | | | p.Pro269GlnfsTer28 | |
| | | 39710481 | | | | | | | |
| ERBB2 | COSM6961097 | chr17: | chr17 | 39712401 | CAAG | C | COSM6961097 | ENSP00000269571.4: | CAA |
| | | 39712321- | | | | | | p.Lys369del | G > C |
| | | 39712448 | | | | | | | |
| ERBB2 | COSM9494227 | chr17: | chr17 | 39715878 | GCT | G | COSM9494227 | ENSP00000269571.4: | GCT > G |
| | | 39715739- | | | | | | p.Phe486SerfsTer80 | |
| | | 39715939 | | | | | | | |
| ERBB2 | COSM6974323 | chr17: | chr17 | 39717434 | GATG | G | COSM6974323 | ENSP00000269571.4: | GATG |
| | | 39717319- | | | AGGA | | | p.Glu619GlnfsTer11 | AGGA |
| | | 39717484 | | | GGGC | | | | GGGC |
| | | | | | GCAT | | | | GCAT |
| | | | | | GCCA | | | | GCCA |
| | | | | | GCCT | | | | GCCT |
| | | | | | TGCC | | | | TGCC |
| | | | | | CC | | | | CC |
| | | | | | (SEQ | | | | (SEQ |
| | | | | | ID | | | | ID NO: |
| | | | | | NO: | | | | 34) > G |
| | | | | | 34) | | | | |
| ERBB2 | COSM7345566 | chr17: | chr17 | 39719798 | TG | T | COSM7345566 | ENSP00000269571.4: | TG > T |
| | | 39719786- | | | | | | p.Asp638MetfsTer14 | |
| | | 39719834 | | | | | | | |
| ERBB2 | COSM6973838 | chr17: | chr17 | 39723542 | TGGA | T | COSM6973838 | ENSP00000269571.4: | TGG |
| | | 39723537- | | | | | | p.Glu698del | A > T |
| | | 39723660 | | | | | | | |
| ERBB2 | COSM7345570 | chr17: | chr17 | 39725766 | CG | C | COSM7345570 | ENSP00000269571.4: | CG > C |
| | | 39725706- | | | | | | p.Glu930ArgfsTer24 | |
| | | 39725853 | | | | | | | |
| MIR4728, | COSM6865894 | chr17: | chr17 | 39726611 | TG | T | COSM6865894 | ENSP00000269571.4: | TG > T |
| ERBB2 | | 39726561- | | | | | | p.Glu975AsnfsTer85 | |
| | | 39726659 | | | | | | | |
| ERBB2 | COSM6865896 | chr17: | chr17 | 39727352 | TCTC | T | COSM6865896 | ENSP00000269571.4: | TCTCC |
| | | 39727294- | | | CACT | | | p.Leu1075MetfsTer48 | ACTG |
| | | 39727547 | | | GGCA | | | | GCAC |
| | | | | | CCCT | | | | CCTC |
| | | | | | CCGA | | | | CGAA |
| | | | | | AGGG | | | | GGGG |
| | | | | | GCTG | | | | CTGG |
| | | | | | G | | | | (SEQ |
| | | | | | (SEQ | | | | ID NO: |
| | | | | | ID | | | | 35) > T |
| | | | | | NO: | | | | |
| | | | | | 35) | | | | |
| GNA11 | COSM9232870 | chr19: | chr19 | 3114989 | CA | C | COSM9232870 | ENSP00000078429.3: | CA > C |
| | | 3114943- | | | | | | p.Thr175ProfsTer49 | |
| | | 3115072 | | | | | | | |
| GNA11 | COSM1392334 | chr19: | chr19 | 3118935 | TG | T | COSM1392334 | ENSP00000078429.3: | TG > T |
| | | 3118923- | | | | | | p.Gly208AlafsTer16 | |
| | | 3119053 | | | | | | | |
| GNA11 | COSM6342228 | chr19: | chr19 | 3121130 | TG | T | COSM6342228 | ENSP00000078429.3: | TG > T |
| | | 3120988- | | | | | | p.Lys345ArgfsTer108 | |
| | | 3121179 | | | | | | | |
| GNAS | COSM9277149 | chr20: | chr20 | 58854492 | AGAT | A | COSM9277149 | ENSP00000360141.3: | AGAT |
| | | 58853265- | | | CCCG | | | p.Thr415_Gly423del | CCCG |
| | | 58855333 | | | ACTC | | | | ACTC |
| | | | | | CGGG | | | | CGGG |
| | | | | | ACAG | | | | ACAG |
| | | | | | CACC | | | | CACC |
| | | | | | AGCC | | | | AGCC |
| | | | | | (SEQ | | | | (SEQ |
| | | | | | ID | | | | ID NO: |
| | | | | | NO: | | | | 36) > A |
| | | | | | 36) | | | | |
| GNAS | COSM6984215 | chr20: | chr20 | 58905438 | ACGA | AG | COSM6984215 | ENSP00000360141.3: | ACG |
| | | 58905382- | | | | | | p.Tyr806Ter | A > AG |
| | | 58905480 | | | | | | | |
| ALK | COSM7347365 | chr2: | chr2 | 29228926 | CACC | C | COSM7347365 | ENSP00000373700.3: | CACC |
| | | 29228883- | | | CCCT | | | p.Phe921_Gly924del | CCCT |
| | | 29229066 | | | CCGA | | | | CCGA |
| | | | | | A | | | | A (SEQ |
| | | | | | (SEQ | | | | ID NO: |
| | | | | | ID | | | | 37) > C |
| | | | | | NO: | | | | |
| | | | | | 37) | | | | |
| ALK | COSM2941501 | chr2: | chr2 | 29233586 | AC | A | COSM2941501 | ENSP00000373700.3: | AC > A |
| | | 29233564- | | | | | | p.Gly822ValfsTer9 | |
| | | 29233696 | | | | | | | |
| ALK | COSM6926372 | chr2: | chr2 | 29275466 | TC | T | COSM6926372 | ENSP00000373700.3: | TC > T |
| | | 29275401- | | | | | | p.Gly616AspfsTer49 | |
| | | 29275496 | | | | | | | |
| ALK | COSM6922292 | chr2: | chr2 | 29318345 | TG | T | COSM6922292 | ENSP00000373700.3: | TG > T |
| | | 29318303- | | | | | | p.Ser536ValfsTer25 | |
| | | 29318404 | | | | | | | |
| ALK | COSM9358093 | chr2: | chr2 | 29920118 | CCTT | CG | COSM9358093 | ENSP00000373700.3: | CCTT |
| | | 29919992- | | | GGCG | | | p.Trp176_ | GGCG |
| | | 29920659 | | | AATC | | | Gly181delinsArg | AATC |
| | | | | | CACC | | | | CACC |
| | | | | | A | | | | A (SEQ |
| | | | | | (SEQ | | | | ID NO: |
| | | | | | ID | | | | 38) > CG |
| | | | | | NO: | | | | |
| | | | | | 38) | | | | |
| PIK3CA | COSM5613085 | chr3: | chr3 | 179209674 | GT | G | COSM5613085 | ENSP00000263967.3: | GT > G |
| | | 179209594- | | | | | | p.Gly411AlafsTer17 | |
| | | 179209700 | | | | | | | |
| PIK3CA | COSM6940128 | chr3: | chr3 | 179210290 | AGAA | A | COSM6940128 | ENSP00000263967.3: | AGAA |
| | | 179210185- | | | GATT | | | p.Glu453_Thr462del | GATT |
| | | 179210338 | | | TGCT | | | | TGCT |
| | | | | | GAAC | | | | GAAC |
| | | | | | CCTA | | | | CCTA |
| | | | | | TTGG | | | | TTGG |
| | | | | | TGTT | | | | TGTT |
| | | | | | ACT | | | | ACT |
| | | | | | (SEQ | | | | (SEQ |
| | | | | | ID | | | | ID NO: |
| | | | | | NO: | | | | 39) > A |
| | | | | | 39) | | | | |
| PIK3CA | COSM6911769 | chr3: | chr3 | 179221134 | GAGA | G | COSM6911769 | ENSP00000263967.3: | GAG |
| | | 179220985- | | | | | | p.Lys724del | A > G |
| | | 179221157 | | | | | | | |
| CTNNB1 | COSM6845286 | chr3: | chr3 | 41225063 | TCAT | T | COSM6845286 | ENSP00000495360.1: | TCAT |
| | | 41224953- | | | CCCA | | | p.His118LeufsTer13 | CCC |
| | | 41225207 | | | | | | | A > T |
| CTNNB1 | COSM6963932 | chr3: | chr3 | 41225731 | CTAA | CA | COSM6963932 | ENSP00000495360.1: | CTAA |
| | | 41225659- | | | AATG | | | p.Lys270_Val273del | AATG |
| | | 41225861 | | | GCAG | | | | GCAG |
| | | | | | TG | | | | TG |
| | | | | | (SEQ | | | | (SEQ |
| | | | | | ID | | | | ID NO: |
| | | | | | NO: | | | | 40) > CA |
| | | | | | 40) | | | | |
| CTNNB1 | COSM5608170 | chr3: | chr3 | 41227274 | AAAC | A | COSM5608170 | ENSP00000495360.1: | AAAC |
| | | 41227207- | | | T | | | p.Lys335AsnfsTer9 | T > A |
| | | 41227352 | | | | | | | |
| CTNNB1 | COSM6939570 | chr3: | chr3 | 41235755 | GTTG | G | COSM6939570 | ENSP00000495360.1: | GTTG |
| | | 41235723- | | | TACC | | | p.Cys573GlufsTer6 | TAC |
| | | 41235843 | | | | | | | C > G |
| CTNNB1 | COSM6853546 | chr3: | chr3 | 41236462 | TCTG | T | COSM6853546 | ENSP00000495360.1: | TCTG |
| | | 41236348- | | | ACAG | | | p.Thr641_Leu644del | ACAG |
| | | 41236499 | | | AGTT | | | | AGTT |
| | | | | | A | | | | A (SEQ |
| | | | | | (SEQ | | | | ID NO: |
| | | | | | ID | | | | 41) > T |
| | | | | | NO: | | | | |
| | | | | | 41) | | | | |
| FGFR3 | COSM4616014 | chr4: | chr4 | 1799410 | TG | T | COSM4616014 | ENSP00000339824.4: | TG > T |
| | | 1799253- | | | | | | p.Gln92SerfsTer6 | |
| | | 1799523 | | | | | | | |
| FGFR3 | COSM4992106 | chr4: | chr4 | 1803754 | TGAG | T | COSM4992106 | | TGAG |
| | | 1803691- | | | GACG | | | | GACG |
| | | 1803836 | | | C | | | | C > T |
| PDGFRA | COSM6906234 | chr4: | chr4 | 54264999 | GT | G | COSM6906234 | ENSP00000257290.5: | GT > G |
| | | 54264918- | | | | | | p.Phe238LeufsTer16 | |
| | | 54265049 | | | | | | | |
| PDGFRA | COSM6964190 | chr4: | chr4 | 54267667 | GCTG | G | COSM6964190 | ENSP00000257290.5: | GCTG |
| | | 54267551- | | | AAAA | | | p.Lys351_Leu356del | AAAA |
| | | 54267741 | | | ACAA | | | | ACAA |
| | | | | | TCTG | | | | TCTG |
| | | | | | ACT | | | | ACT |
| | | | | | (SEQ | | | | (SEQ |
| | | | | | ID | | | | ID NO: |
| | | | | | NO: | | | | 42) > G |
| | | | | | 42) | | | | |
| PDGFRA | COSM3301372 | chr4: | chr4 | 54285479 | CA | C | COSM3301372 | ENSP00000257290.5: | CA > C |
| | | 54285370- | | | | | | p.Asn813IlefsTer20 | |
| | | 54285486 | | | | | | | |
| PDGFRA | COSM7346029 | chr4: | chr4 | 54287473 | AC | A | COSM7346029 | ENSP00000257290.5: | AC > A |
| | | 54287429- | | | | | | p.Asp869GlufsTer7 | |
| | | 54287541 | | | | | | | |
| PDGFRA | COSM6972086 | chr4: | chr4 | 54289073 | AGT | AC | COSM6972086 | ENSP00000257290.5: | AGT > AC |
| | | 54289008- | | | | | | p.Ser947ThrfsTer24 | |
| | | 54289114 | | | | | | | |
| PDGFRA | COSM6956086 | chr4: | chr4 | 54290548 | GAC | G | COSM6956086 | ENSP00000257290.5: | GAC > G |
| | | 54290312- | | | | | | p.His1040GlnfsTer6 | |
| | | 54290554 | | | | | | | |
| PDGFRA | COSM7346028 | chr4: | chr4 | 54295233 | CAT | C | COSM7346028 | ENSP00000257290.5: | CAT > C |
| | | 54295124- | | | | | | p.Ile1078ArgfsTer41 | |
| | | 54295272 | | | | | | | |
| KIT | COSM6951399 | chr4: | chr4 | 54658073 | TC | T | COSM6951399 | ENSP00000288135.5: | TC > T |
| | | 54658014- | | | | | | p.Gln21ArgfsTer9 | |
| | | 54658081 | | | | | | | |
| KIT | COSM7345631 | chr4: | chr4 | 54695772 | TTTG | T | COSM7345631 | ENSP00000288135.5: | TTT |
| | | 54695511- | | | | | | p.Val111del | G > T |
| | | 54695781 | | | | | | | |
| KIT | COSM7345632 | chr4: | chr4 | 54698515 | AG | A | COSM7345632 | ENSP00000288135.5: | AG > A |
| | | 54698283- | | | | | | p.Glu191ArgfsTer12 | |
| | | 54698565 | | | | | | | |
| KIT | COSM1305 | chr4: | chr4 | 54729451 | TATA | T | COSM1305 | ENSP00000288135.5: | TATA |
| | | 54729334- | | | AGA | | | p.Lys704_Asn705del | AGA > T |
| | | 54729485 | | | | | | | |
| KIT | COSM1306 | chr4: | chr4 | 54731324 | CCAG | C | COSM1306 | ENSP00000288135.5: | CCA |
| | | 54731327- | | | | | | p.Ser715del | G > C |
| | | 54731419 | | | | | | | |
| KIT | COSM28578 | chr4: | chr4 | 54731967 | CA | C | COSM28578 | ENSP00000288135.5: | CA > C |
| | | 54731870- | | | | | | p.Lys778ArgfsTer36 | |
| | | 54731998 | | | | | | | |
| KIT | COSM6965292 | chr4: | chr4 | 54738465 | CAG | C | COSM6965292 | ENSP00000288135.5: | CAG > C |
| | | 54738428- | | | | | | p.Lys948AlafsTer100 | |
| | | 54738557 | | | | | | | |
| APC | COSM6963650 | chr5: | chr5 | 112755023 | AAGG | A | COSM6963650 | | AAGG |
| | | 112754890- | | | TATC | | | | TAT |
| | | 112755025 | | | | | | | C > A |
| APC | COSM6853815 | chr5: | chr5 | 112766390 | AT | A | COSM6853815 | ENSP00000257430.4: | AT > A |
| | | 112766325- | | | | | | p.Leu68TyrfsTer2 | |
| | | 112766410 | | | | | | | |
| APC | COSM6854236 | chr5: | chr5 | 112775710 | AATA | A | COSM6854236 | ENSP00000257430.4: | AATA |
| | | 112775628- | | | G | | | p.Asp170ValfsTer4 | G > A |
| | | 112775737 | | | | | | | |
| APC | COSM6976104 | chr5: | chr5 | 112792468 | AAAT | A | COSM6976104 | ENSP00000257430.4: | AAAT |
| | | 112792445- | | | CG | | | p.Ile224LysfsTer26 | CG > A |
| | | 112792529 | | | | | | | |
| APC | COSM6984704 | chr5: | chr5 | 112801284 | ATC | A | COSM6984704 | ENSP00000257430.4: | ATC > A |
| | | 112801278- | | | | | | p.Gln247GlufsTer4 | |
| | | 112801383 | | | | | | | |
| APC | COSM6971752 | chr5: | chr5 | 112821942 | AATG | A | COSM6971752 | ENSP00000257430.4: | AATG |
| | | 112821895- | | | AAAC | | | p.Leu456SerfsTer6 | AAAC |
| | | 112821991 | | | TTTC | | | | TTTCA |
| | | | | | ATTT | | | | TTTG |
| | | | | | G | | | | (SEQ |
| | | | | | (SEQ | | | | ID NO: |
| | | | | | ID | | | | 43) > A |
| | | | | | NO: | | | | |
| | | | | | 43) | | | | |
| APC | COSM4169285 | chr5: | chr5 | 112827121 | CATT | C | COSM4169285 | ENSP00000257430.4: | CATT |
| | | 112827107- | | | GCAG | | | p.Glu477SerfsTer4 | GCAG |
| | | 112827247 | | | AATT | | | | AATT |
| | | | | | (SEQ | | | | (SEQ |
| | | | | | ID | | | | ID NO: |
| | | | | | NO: | | | | 44) > C |
| | | | | | 44) | | | | |
| APC | COSM1169625 | chr5: | chr5 | 112827937 | ATGC | A | COSM1169625 | ENSP00000257430.4: | ATGC |
| | | 112827928- | | | TC | | | p.Cys520TyrfsTer15 | TC > A |
| | | 112828006 | | | | | | | |
| APC | COSM4169180 | chr5: | chr5 | 112828863 | CGAG | C | COSM4169180 | ENSP00000257430.4: | CGAG |
| | | 112828855- | | | T | | | p.Ser546PhefsTer2 | T > C |
| | | 112828972 | | | | | | | |
| ROS1 | COSM6921151 | chr6: | chr6 | 117310082 | ATAC | A | COSM6921151 | ENSP00000357494.3: | ATAC |
| | | 117310080- | | | AT | | | p.Asp2143ValfsTer25 | AT > A |
| | | 117310281 | | | | | | | |
| ROS1 | COSM6959063 | chr6: | chr6 | 117326294 | TCTG | T | COSM6959063 | ENSP00000357494.3: | TCTG |
| | | 117326223- | | | AA | | | p.Phe1828SerfsTer6 | AA > T |
| | | 117326414 | | | | | | | |
| ROS1 | COSM6968834 | chr6: | chr6 | 117341465 | ATTC | A | COSM6968834 | ENSP00000357494.3: | ATTC |
| | | 117341399- | | | ACTT | | | p.Thr1606_ | ACTTT |
| | | 117341632 | | | TGTC | | | Glu1612del | GTCTT |
| | | | | | TTAG | | | | AGAG |
| | | | | | AGGA | | | | GAGT |
| | | | | | GT | | | | (SEQ |
| | | | | | (SEQ | | | | ID NO: |
| | | | | | ID | | | | 45) > A |
| | | | | | NO: | | | | |
| | | | | | 45) | | | | |
| ROS1 | COSM9225153 | chr6: | chr6 | 117342404 | AT | A | COSM9225153 | ENSP00000357494.3: | AT > A |
| | | 117342399- | | | | | | p.Asn1555MetfsTer48 | |
| | | 117342544 | | | | | | | |
| ROS1 | COSM6978532 | chr6: | chr6 | 117356904 | CAAT | C | COSM6978532 | | CAAT |
| | | 117356628- | | | ACAA | | | | ACAA |
| | | 117356915 | | | GCGA | | | | GCGA |
| | | | | | CTAT | | | | CTAT |
| | | | | | AGAG | | | | AGAG |
| | | | | | GAAA | | | | GAAA |
| | | | | | A | | | | A (SEQ |
| | | | | | (SEQ | | | | ID NO: |
| | | | | | ID | | | | 46) > C |
| | | | | | NO: | | | | |
| | | | | | 46) | | | | |
| ROS1 | COSM6984106 | chr6: | chr6 | 117357806 | TA | T | COSM6984106 | ENSP00000357494.3: | TA > T |
| | | 117357803- | | | | | | p.Leu1284Ter | |
| | | 117358009 | | | | | | | |
| ROS1 | COSM5977598 | chr6: | chr6 | 117360400 | CCT | C | COSM5977598 | ENSP00000357494.3: | CCT > C |
| | | 117360341- | | | | | | p.Arg1129GlyfsTer5 | |
| | | 117360405 | | | | | | | |
| ROS1 | COSM5576297 | chr6: | chr6 | 117362635 | CT | C | COSM5576297 | ENSP00000357494.3: | CT > C |
| | | 117362602- | | | | | | p.Gly1117AlafsTer2 | |
| | | 117362865 | | | | | | | |
| ROS1 | COSM7409277 | chr6: | chr6 | 117387804 | AG | A | COSM7409277 | ENSP00000357494.3: | AG > A |
| | | 117387779- | | | | | | p.Ser664GlnfsTer18 | |
| | | 117388034 | | | | | | | |
| ROS1 | COSM6940064 | chr6: | chr6 | 117393245 | AT | A | COSM6940064 | ENSP00000357494.3: | AT > A |
| | | 117393223- | | | | | | p.Ile414LeufsTer14 | |
| | | 117393321 | | | | | | | |
| ROS1 | COSM6916198 | chr6: | chr6 | 117396979 | AT | A | COSM6916198 | ENSP00000357494.3: | AT > A |
| | | 117396914- | | | | | | p.Lys238AsnfsTer2 | |
| | | 117397116 | | | | | | | |
| MET | COSM6912457 | chr7: | chr7 | 116699692 | CTTC | C | COSM6912457 | ENSP00000317272.6: | CTTC |
| | | 116699084- | | | T | | | p.Ser204IlefsTer12 | T > C |
| | | 116700284 | | | | | | | |
| MET | COSM6975700 | chr7: | chr7 | 116740889 | ATTT | A | COSM6975700 | ENSP00000317272.6: | ATTTC |
| | | 116740851- | | | CCAG | | | p.Phe523_Ser527del | CAGT |
| | | 116741025 | | | TCCT | | | | CCTG |
| | | | | | GCAG | | | | CAG |
| | | | | | (SEQ | | | | (SEQ |
| | | | | | ID | | | | ID NO: |
| | | | | | NO: | | | | 47) > A |
| | | | | | 47) | | | | |
| MET | COSM6976259 | chr7: | chr7 | 116755455 | CTAG | CG | COSM6976259 | ENSP00000317272.6: | CTAG |
| | | 116755354- | | | AGTT | | | p.Arg602_Ser609del | AGTT |
| | | 116755515 | | | CTCC | | | | CTCCT |
| | | | | | TTGG | | | | TGGA |
| | | | | | AAAT | | | | AATG |
| | | | | | GAGA | | | | AGAG |
| | | | | | GC | | | | C(SEQ |
| | | | | | (SEQ | | | | ID NO: |
| | | | | | ID | | | | 48) > CG |
| | | | | | NO: | | | | |
| | | | | | 48) | | | | |
| MET | COSM5977594 | chr7: | chr7 | 116758487 | AC | A | COSM5977594 | ENSP00000317272.6: | AC > A |
| | | 116758458- | | | | | | p.Pro712GlnfsTer13 | |
| | | 116758620 | | | | | | | |
| MET | COSM6937367 | chr7: | chr7 | 116759394 | TG | T | COSM6937367 | ENSP00000317272.6: | TG > T |
| | | 116759336- | | | | | | p.Ser776AlafsTer3 | |
| | | 116759490 | | | | | | | |
| MET | COSM6984036 | chr7: | chr7 | 116774940 | GACA | G | COSM6984036 | ENSP00000317272.6: | GACA |
| | | 116774880- | | | TGTC | | | p.Asp1048_ | TGTC |
| | | 116775111 | | | CCCC | | | Ile1052delinsVal | CCCC |
| | | | | | A | | | | A (SEQ |
| | | | | | (SEQ | | | | ID NO: |
| | | | | | ID | | | | 49) > G |
| | | | | | NO: | | | | |
| | | | | | 49) | | | | |
| MET | COSM1579075 | chr7: | chr7 | 116782056 | CA | C | COSM1579075 | ENSP00000317272.6: | CA > C |
| | | 116781987- | | | | | | p.Lys1217SerfsTer49 | |
| | | 116782097 | | | | | | | |
| MET | COSM7345743 | chr7: | chr7 | 116796002 | CATG | C | COSM7345743 | ENSP00000317272.6: | CATG |
| | | 116795886- | | | TGAA | | | p.Ala1372_ | TGAA |
| | | 116796124 | | | CGCT | | | Asn1376del | CGCT |
| | | | | | ACTT | | | | ACTT |
| | | | | | (SEQ | | | | (SEQ |
| | | | | | ID | | | | ID NO: |
| | | | | | NO: | | | | 50) > C |
| | | | | | 50) | | | | |
| EGFR | COSM6973876 | chr7: | chr7 | 55142292 | AAGG | AC | COSM6973876 | ENSP00000275493.2: | AAGG |
| | | 55142285- | | | CACG | | | p.Gln32HisfsTer46 | CACG |
| | | 55142437 | | | A | | | | A > AC |
| EGFR | COSM9494233 | chr7: | chr7 | 55154103 | CCCC | C | COSM9494233 | ENSP00000275493.2: | CCCC |
| | | 55154010- | | | GAGG | | | p.Pro281GlnfsTer15 | GAGG |
| | | 55154152 | | | G | | | | G > C |
| EGFR | COSM6962235 | chr7: | chr7 | 55155837 | TGTG | T | COSM6962235 | ENSP00000275493.2: | TGTG > T |
| | | 55155829- | | | | | | p.Val301del | |
| | | 55155946 | | | | | | | |
| EGFR | COSM7343128 | chr7: | chr7 | 55161538 | AG | A | COSM7343128 | ENSP00000275493.2: | AG > A |
| | | 55161498- | | | | | | p.Gly514AlafsTer54 | |
| | | 55161631 | | | | | | | |
| EGFR | COSM6196864 | chr7: | chr7 | 55165374 | TG | T | COSM6196864 | ENSP00000275493.2: | TG > T |
| | | 55165279- | | | | | | p.Val607SerfsTer98 | |
| | | 55165437 | | | | | | | |
| EGFR | COSM9110951 | chr7: | chr7 | 55170527 | GCCT | G | COSM9110951 | | GCC |
| | | 55170306- | | | | | | | T > G |
| | | 55170544 | | | | | | | |
| EGFR | COSM6909028 | chr7: | chr7 | 55173048 | TG | T | COSM6909028 | ENSP00000275493.2: | TG > T |
| | | 55172982- | | | | | | p.Ile664SerfsTer41 | |
| | | 55173124 | | | | | | | |
| GNAQ | COSM6342235 | chr9: | chr9 | 77721491 | TG | T | COSM6342235 | ENSP00000286548.4: | TG > T |
| | | 77721322- | | | | | | p.Ala304GlufsTer7 | |
| | | 77721513 | | | | | | | |
| GNAQ | COSM28414 | chr9: | chr9 | 77728594 | ATAA | A | COSM28414 | ENSP00000286548.4: | ATAA |
| | | 77728513- | | | CCGA | | | p.Asn266PhefsTer4 | CCGA |
| | | 77728667 | | | GGAG | | | | GGAG |
| | | | | | TT | | | | TT |
| | | | | | (SEQ | | | | (SEQ |
| | | | | | ID | | | | ID NO: |
| | | | | | NO: | | | | 51) > A |
| | | | | | 51) | | | | |
| GNAQ | COSM7347398 | chr9: | chr9 | 77815756 | TAAT | T | COSM7347398 | ENSP00000286548.4: | TAAT |
| | | 77815615- | | | TGTG | | | p.Ala108GlufsTer18 | TGTG |
| | | 77815770 | | | CATG | | | | CATG |
| | | | | | AG | | | | AG |
| | | | | | (SEQ | | | | (SEQ |
| | | | | | ID | | | | ID NO: |
| | | | | | NO: | | | | 52) > T |
| | | | | | 52) | | | | |
| GNAQ | COSM9113869 | chr9: | chr9 | 78031128 | GGCG | G | COSM9113869 | ENSP00000286548.4: | GGCG |
| | | 78031099- | | | TCCC | | | p.Leu29ProfsTer24 | TCCC |
| | | 78031235 | | | GCTT | | | | GCTT |
| | | | | | GTCC | | | | GTCC |
| | | | | | CTGC | | | | CTGC |
| | | | | | GGA | | | | GGA |
| | | | | | (SEQ | | | | (SEQ |
| | | | | | ID | | | | ID NO: |
| | | | | | NO: | | | | 53) > G |
| | | | | | 53) | | | | |
| RET | COSM4989947 | chr10: | chr10 | 43100532 | C | T | COSM4989947 | ENSP00000347942.3: | C > T |
| | | 43100458- | | | | | | p.Pro49= | |
| | | 43100722 | | | | | | | |
| RET | COSM6947065 | chr10: | chr10 | 43106469 | G | A | COSM6947065 | ENSP00000347942.3: | G > A |
| | | 43106375- | | | | | | p.Gly321Arg | |
| | | 43106571 | | | | | | | |
| RET | COSM9277606 | chr10: | chr10 | 43109132 | C | T | COSM9277606 | ENSP00000347942.3: | C > T |
| | | 43109030- | | | | | | p.Leu389Phe | |
| | | 43109230 | | | | | | | |
| RET | COSM4418405 | chr10: | chr10 | 43118395 | G | T | COSM4418405 | ENSP00000347942.3: | G > T |
| | | 43118372- | | | | | | p.Leu769= | |
| | | 43118480 | | | | | | | |
| RET | COSM6945831 | chr10: | chr10 | 43119624 | G | T | COSM6945831 | ENSP00000347942.3: | G > T |
| | | 43119530- | | | | | | p.Ser829Ile | |
| | | 43119745 | | | | | | | |
| RET | COSM3997965 | chr10: | chr10 | 43124887 | C | T | COSM3997965 | ENSP00000347942.3: | C > T |
| | | 43124882- | | | | | | p.Arg982Cys | |
| | | 43124982 | | | | | | | |
| RET | COSM6914657 | chr10: | chr10 | 43126707 | G | A | COSM6914657 | ENSP00000347942.3: | G > A |
| | | 43126574- | | | | | | p.Glu1058Lys | |
| | | 43126754 | | | | | | | |
| ATM, | COSM21325 | chr11: | chr11 | 108312465 | A | C | COSM21325 | ENSP00000278616.4: | A > C |
| C11orf65 | | 108312410- | | | | | | p.Glu1991Asp | |
| | | 108312498 | | | | | | | |
| ATM, | COSM7343670 | chr11: | chr11 | 108321330 | G | A | COSM7343670 | ENSP00000278616.4: | G > A |
| C11orf65 | | 108321300- | | | | | | p.Arg2161His | |
| | | 108321420 | | | | | | | |
| ATM, | COSM200673 | chr11: | chr11 | 108335854 | G | A | COSM200673 | ENSP00000278616.4: | G > A |
| C11orf65 | | 108335844- | | | | | | p.Asp2721Asn | |
| | | 108335961 | | | | | | | |
| AKT1 | COSM5044338 | chr14: | chr14 | 104770406 | A | T | COSM5044338 | ENSP00000451828.1: | A > T |
| | | 104770340- | | | | | | p.Cys460Ser | |
| | | 104770420 | | | | | | | |
| AKT1 | COSM6966503 | chr14: | chr14 | 104770769 | T | A | COSM6966503 | ENSP00000451828.1: | T > A |
| | | 104770744- | | | | | | p.Ile447Phe | |
| | | 104770847 | | | | | | | |
| AKT1 | COSM5020215 | chr14: | chr14 | 104772446 | G | A | COSM5020215 | ENSP00000451828.1: | G > A |
| | | 104772364- | | | | | | p.Gly393= | |
| | | 104772452 | | | | | | | |
| AKT1 | COSM6924152 | chr14: | chr14 | 104773963 | G | C | COSM6924152 | ENSP00000451828.1: | G > C |
| | | 104773911- | | | | | | p.Phe217Leu | |
| | | 104773980 | | | | | | | |
| AKT1 | COSM9102250 | chr14: | chr14 | 104775003 | C | T | COSM9102250 | ENSP00000451828.1: | C > T |
| | | 104774937- | | | | | | p.Asp190Asn | |
| | | 104775003 | | | | | | | |
| AKT1 | COSM6986817 | chr14: | chr14 | 104775763 | G | C | COSM6986817 | ENSP00000451828.1: | G > C |
| | | 104775651- | | | | | | p.Asp108Glu | |
| | | 104775799 | | | | | | | |
| ERBB2 | COSM5414789 | chr17: | chr17 | 39700281 | C | T | COSM5414789 | ENSP00000269571.4: | C > T |
| | | 39700238- | | | | | | p.Leu15Phe | |
| | | 39700311 | | | | | | | |
| ERBB2 | COSM9102609 | chr17: | chr17 | 39709385 | G | A | COSM9102609 | ENSP00000269571.4: | G > A |
| | | 39709317- | | | | | | p.Trp169Ter | |
| | | 39709452 | | | | | | | |
| ERBB2 | COSM6913537 | chr17: | chr17 | 39709822 | G | A | COSM6913537 | ENSP00000269571.4: | G > A |
| | | 39709812- | | | | | | p.Cys195Tyr | |
| | | 39709881 | | | | | | | |
| ERBB2 | COSM94225 | chr17: | chr17 | 39711955 | C | A | COSM94225 | ENSP00000269571.4: | C > A |
| | | 39711927- | | | | | | p.Ser310Tyr | |
| | | 39712047 | | | | | | | |
| ERBB2 | COSM9110847 | chr17: | chr17 | 39715294 | C | A | COSM9110847 | ENSP00000269571.4: | C > A |
| | | 39715285- | | | | | | p.Ala386Asp | |
| | | 39715359 | | | | | | | |
| ERBB2 | COSM7343981 | chr17: | chr17 | 39716431 | G | T | COSM7343981 | ENSP00000269571.4: | G > T |
| | | 39716300- | | | | | | p.Gln548His | |
| | | 39716433 | | | | | | | |
| TP53 | COSM9312241 | chr17: | chr17 | 7673240 | G | A | COSM9312241 | | G > A |
| | | 7673218- | | | | | | | |
| | | 7673266 | | | | | | | |
| GNA11 | COSM5611295 | chr19: | chr19 | 3110238 | ACC | ATT | COSM5611295 | ENSP00000078429.3: | ACC > A |
| | | 3110148- | | | | | | p.Thr76Ile | TT |
| | | 3110333 | | | | | | | |
| GNA11 | COSM6939602 | chr19: | chr19 | 3113334 | A | G | COSM6939602 | ENSP00000078429.3: | A > G |
| | | 3113329- | | | | | | p.Asn109Ser | |
| | | 3113484 | | | | | | | |
| GNAS | COSM6939725 | chr20: | chr20 | 58891769 | G | T | COSM6939725 | | G > T |
| | | 58891726- | | | | | | | |
| | | 58891865 | | | | | | | |
| GNAS | COSM6965756 | chr20: | chr20 | 58895644 | A | G | COSM6965756 | ENSP00000360141.3: | A > G |
| | | 58895611- | | | | | | p.Lys701Glu | |
| | | 58895684 | | | | | | | |
| GNAS | COSM9312081 | chr20: | chr20 | 58898948 | G | A | COSM9312081 | ENSP00000360141.3: | G > A |
| | | 58898940- | | | | | | p.Glu717Lys | |
| | | 58898985 | | | | | | | |
| GNAS | COSM3758661 | chr20: | chr20 | 58903752 | C | T | COSM3758661 | ENSP00000360141.3: | C > T |
| | | 58903671- | | | | | | p.Ile774= | |
| | | 58903791 | | | | | | | |
| GNAS | COSM6977578 | chr20: | chr20 | 58910048 | C | T | COSM6977578 | ENSP00000360141.3: | C > T |
| | | 58909950- | | | | | | p.Pro956Ser | |
| | | 58910081 | | | | | | | |
| GNAS | COSM4485625 | chr20: | chr20 | 58910387 | C | T | COSM4485625 | ENSP00000360141.3: | C > T |
| | | 58910333- | | | | | | p.Arg985Ter | |
| | | 58910401 | | | | | | | |
| GNAS | COSM6907299 | chr20: | chr20 | 58910782 | C | T | COSM6907299 | ENSP00000360141.3: | C > T |
| | | 58910682- | | | | | | p.Arg1023Cys | |
| | | 58910829 | | | | | | | |
| ALK | COSM7408659 | chr2: | chr2 | 29196823 | C | T | COSM7408659 | ENSP00000373700.3: | C > T |
| | | 29196769- | | | | | | p.Glu1371Lys | |
| | | 29196860 | | | | | | | |
| ALK | COSM6924954 | chr2: | chr2 | 29197575 | C | T | COSM6924954 | ENSP00000373700.3: | C > T |
| | | 29197541- | | | | | | p.Arg1347Gln | |
| | | 29197676 | | | | | | | |
| ALK | COSM6939221 | chr2: | chr2 | 29207181 | T | C | COSM6939221 | ENSP00000373700.3: | T > C |
| | | 29207170- | | | | | | p.Thr1310Ala | |
| | | 29207272 | | | | | | | |
| ALK | COSM28617 | chr2: | chr2 | 29214027 | C | T | COSM28617 | ENSP00000373700.3: | C > T |
| | | 29213983- | | | | | | p.Ala1234Thr | |
| | | 29214081 | | | | | | | |
| ALK | COSM6949625 | chr2: | chr2 | 29223444 | G | A | COSM6949625 | ENSP00000373700.3: | G > A |
| | | 29223341- | | | | | | p.Ser1086Leu | |
| | | 29223528 | | | | | | | |
| ALK | COSM6948461 | chr2: | chr2 | 29227060 | C | T | COSM6948461 | ENSP00000373700.3: | C > T |
| | | 29226921- | | | | | | p.Gly977Arg | |
| | | 29227074 | | | | | | | |
| ALK | COSM6908629 | chr2: | chr2 | 29227669 | C | T | COSM6908629 | ENSP00000373700.3: | C > T |
| | | 29227573- | | | | | | p.Gly940Asp | |
| | | 29227672 | | | | | | | |
| ALK | COSM148825 | chr2: | chr2 | 29232401 | A | G | COSM148825 | ENSP00000373700.3: | A > G |
| | | 29232303- | | | | | | p.Gly845= | |
| | | 29232448 | | | | | | | |
| ALK | COSM50296 | chr2: | chr2 | 29239766 | C | T | COSM50296 | ENSP00000373700.3: | C > T |
| | | 29239679- | | | | | | p.Val757Met | |
| | | 29239830 | | | | | | | |
| ALK | COSM6940013 | chr2: | chr2 | 29251115 | C | T | COSM6940013 | ENSP00000373700.3: | C > T |
| | | 29251104- | | | | | | p.Asp732Asn | |
| | | 29251267 | | | | | | | |
| ALK | COSM5019540 | chr2: | chr2 | 29275101 | G | A | COSM5019540 | ENSP00000373700.3: | G > A |
| | | 29275098- | | | | | | p.Thr680Ile | |
| | | 29275227 | | | | | | | |
| ALK | COSM6963778 | chr2: | chr2 | 29296995 | C | G | COSM6963778 | ENSP00000373700.3: | C > G |
| | | 29296887- | | | | | | p.Glu570Asp | |
| | | 29297057 | | | | | | | |
| ALK | COSM6947853 | chr2: | chr2 | 29328379 | G | T | COSM6947853 | ENSP00000373700.3: | G > T |
| | | 29328349- | | | | | | p.Ala462Asp | |
| | | 29328481 | | | | | | | |
| ALK | COSM1172867 | chr2: | chr2 | 29383830 | C | T | COSM1172867 | ENSP00000373700.3: | C > T |
| | | 29383731- | | | | | | p.Arg395His | |
| | | 29383859 | | | | | | | |
| ALK | COSM6598514 | chr2: | chr2 | 29532024 | C | T | COSM6598514 | ENSP00000373700.3: | C > T |
| | | 29531914- | | | | | | p.Val349Ile | |
| | | 29532116 | | | | | | | |
| ALK | COSM1236664 | chr2: | chr2 | 29694870 | C | T | COSM1236664 | ENSP00000373700.3: | C > T |
| | | 29694849- | | | | | | p.Arg311His | |
| | | 29695014 | | | | | | | |
| ALK | COSM4416269 | chr2: | chr2 | 29717663 | A | T | COSM4416269 | ENSP00000373700.3: | A > T |
| | | 29717577- | | | | | | p.Pro234= | |
| | | 29717697 | | | | | | | |
| PIK3CA | COSM3205605 | chr3: | chr3 | 179199822 | G | A | COSM3205605 | ENSP00000263967.3: | G > A |
| | | 179199689- | | | | | | p.Arg162Lys | |
| | | 179199899 | | | | | | | |
| PIK3CA | COSM6931303 | chr3: | chr3 | 179201476 | A | T | COSM6931303 | ENSP00000263967.3: | A > T |
| | | 179201289- | | | | | | p.Tyr250Phe | |
| | | 179201540 | | | | | | | |
| PIK3CA | COSM21450 | chr3: | chr3 | 179204576 | G | T | COSM21450 | ENSP00000263967.3: | G > T |
| | | 179204502- | | | | | | p.Cys378Phe | |
| | | 179204588 | | | | | | | |
| PIK3CA | COSM1716809 | chr3: | chr3 | 179219228 | C | T | COSM1716809 | ENSP00000263967.3: | C > T |
| | | 179219195- | | | | | | p.Pro566Leu | |
| | | 179219277 | | | | | | | |
| PIK3CA | COSM250052 | chr3: | chr3 | 179219950 | T | C | COSM250052 | ENSP00000263967.3: | T > C |
| | | 179219948- | | | | | | p.Val638Ala | |
| | | 179220052 | | | | | | | |
| PIK3CA | COSM6475729 | chr3: | chr3 | 179224123 | T | C | COSM6475729 | ENSP00000263967.3: | T > C |
| | | 179224080- | | | | | | p.Phe744Leu | |
| | | 179224187 | | | | | | | |
| PIK3CA | COSM6981846 | chr3: | chr3 | 179224740 | C | A | COSM6981846 | ENSP00000263967.3: | C > A |
| | | 179224699- | | | | | | p.Leu779Met | |
| | | 179224821 | | | | | | | |
| PIK3CA | COSM1041507 | chr3: | chr3 | 179225997 | C | T | COSM1041507 | ENSP00000263967.3: | C > T |
| | | 179225961- | | | | | | p.Arg818Cys | |
| | | 179226040 | | | | | | | |
| PIK3CA | COSM39499 | chr3: | chr3 | 179229374 | G | C | COSM39499 | ENSP00000263967.3: | G > C |
| | | 179229271- | | | | | | p.Leu866Phe | |
| | | 179229442 | | | | | | | |
| PIK3CA | COSM769 | chr3: | chr3 | 179230039 | G | T | COSM769 | ENSP00000263967.3: | G > T |
| | | 179230003- | | | | | | p.Cys901Phe | |
| | | 179230121 | | | | | | | |
| PIK3CA | COSM6475740 | chr3: | chr3 | 179230373 | A | G | COSM6475740 | ENSP00000263967.3: | A > G |
| | | 179230224- | | | | | | p.Glu978Gly | |
| | | 179230376 | | | | | | | |
| PIK3CA, | COSM9111593 | chr3: | chr3 | 179240022 | T | C | COSM9111593 | | T > C |
| KCNMB3 | | 179239995- | | | | | | | |
| | | 179240064 | | | | | | | |
| CTNNB1 | COSM4117539 | chr3: | chr3 | 41224075 | A | G | COSM4117539 | ENSP00000495360.1: | A > G |
| | | 41224068- | | | | | | p.Thr3Ala | |
| | | 41224081 | | | | | | | |
| CTNNB1 | COSM5576265 | chr3: | chr3 | 41234157 | C | T | COSM5576265 | ENSP00000495360.1: | C > T |
| | | 41234138- | | | | | | p.Arg515Ter | |
| | | 41234297 | | | | | | | |
| CTNNB1 | COSM1044608 | chr3: | chr3 | 41238068 | G | A | COSM1044608 | ENSP00000495360.1: | G > A |
| | | 41238015- | | | | | | p.Arg710His | |
| | | 41238076 | | | | | | | |
| CTNNB1 | COSM1485172 | chr3: | chr3 | 41239208 | G | A | COSM1485172 | ENSP00000495360.1: | G > A |
| | | 41239133- | | | | | | p.Glu738Lys | |
| | | 41239342 | | | | | | | |
| FGFR3 | COSM6968758 | chr4: | chr4 | 1794007 | T | A | COSM6968758 | ENSP00000339824.4: | T > A |
| | | 1793934- | | | | | | p.Leu25Met | |
| | | 1794043 | | | | | | | |
| FGFR3 | COSM9213245 | chr4: | chr4 | 1799784 | C | T | COSM9213245 | ENSP00000339824.4: | C > T |
| | | 1799746- | | | | | | p.Asp139= | |
| | | 1799812 | | | | | | | |
| FGFR3 | COSM6942045 | chr4: | chr4 | 1801370 | C | T | COSM6942045 | ENSP00000339824.4: | C > T |
| | | 1801366- | | | | | | p.Ala150Val | |
| | | 1801536 | | | | | | | |
| FGFR3 | COSM7342301 | chr4: | chr4 | 1802941 | G | A | COSM7342301 | ENSP00000339824.4: | G > A |
| | | 1802913- | | | | | | p.Asp320Asn | |
| | | 1803064 | | | | | | | |
| FGFR3 | COSM6919387 | chr4: | chr4 | 1805402 | T | C | COSM6919387 | ENSP00000339824.4: | T > C |
| | | 1805354- | | | | | | p.Val489Ala | |
| | | 1805476 | | | | | | | |
| PDGFRA | COSM4383728 | chr4: | chr4 | 54258785 | C | T | COSM4383728 | ENSP00000257290.5: | C > T |
| | | 54258768- | | | | | | p.Pro6Leu | |
| | | 54258817 | | | | | | | |
| PDGFRA | COSM4416371 | chr4: | chr4 | 54263911 | T | C | COSM4416371 | ENSP00000257290.5: | T > C |
| | | 54263666- | | | | | | p.Asn204= | |
| | | 54263927 | | | | | | | |
| PDGFRA | COSM6938810 | chr4: | chr4 | 54272497 | G | A | COSM6938810 | ENSP00000257290.5: | G > A |
| | | 54272393- | | | | | | p.Trp447Ter | |
| | | 54272520 | | | | | | | |
| PDGFRA | COSM2155032 | chr4: | chr4 | 54273575 | A | G | COSM2155032 | ENSP00000257290.5: | A > G |
| | | 54273536- | | | | | | p.Asn468Ser | |
| | | 54273730 | | | | | | | |
| PDGFRA | COSM4417622 | chr4: | chr4 | 54277410 | G | A | COSM4417622 | ENSP00000257290.5: | G > A |
| | | 54277387- | | | | | | p.Ala603= | |
| | | 54277492 | | | | | | | |
| PDGFRA | COSM6958142 | chr4: | chr4 | 54278422 | A | C | COSM6958142 | ENSP00000257290.5: | A > C |
| | | 54278361- | | | | | | p.Lys688Thr | |
| | | 54278515 | | | | | | | |
| PDGFRA | COSM4383732 | chr4: | chr4 | 54280374 | A | G | COSM4383732 | ENSP00000257290.5: | A > G |
| | | 54280315- | | | | | | p.Thr739Ala | |
| | | 54280482 | | | | | | | |
| KIT | COSM6909371 | chr4: | chr4 | 54699686 | G | T | COSM6909371 | ENSP00000288135.5: | G > T |
| | | 54699629- | | | | | | p.Gly226Trp | |
| | | 54699766 | | | | | | | |
| KIT | COSM3301432 | chr4: | chr4 | 54703760 | G | A | COSM3301432 | ENSP00000288135.5: | G > A |
| | | 54703723- | | | | | | p.Gly265Ser | |
| | | 54703892 | | | | | | | |
| KIT | COSM9500507 | chr4: | chr4 | 54707133 | A | G | COSM9500507 | ENSP00000288135.5: | A > G |
| | | 54707097- | | | | | | p.Thr321Ala | |
| | | 54707287 | | | | | | | |
| KIT | COSM6005552 | chr4: | chr4 | 54709427 | C | T | COSM6005552 | ENSP00000288135.5: | C > T |
| | | 54709423- | | | | | | p.Tyr373= | |
| | | 54709539 | | | | | | | |
| KIT | COSM1325 | chr4: | chr4 | 54736599 | G | C | COSM1325 | ENSP00000288135.5: | G > C |
| | | 54736497- | | | | | | p.Leu862= | |
| | | 54736609 | | | | | | | |
| KIT | COSM6945539 | chr4: | chr4 | 54737225 | C | A | COSM6945539 | ENSP00000288135.5: | C > A |
| | | 54737174- | | | | | | p.Thr916Lys | |
| | | 54737280 | | | | | | | |
| ROS1 | COSM249317 | chr6: | chr6 | 117288728 | C | T | COSM249317 | ENSP00000357494.3: | C > T |
| | | 117288491- | | | | | | p.Glu2270Lys | |
| | | 117288802 | | | | | | | |
| ROS1 | COSM150168 | chr6: | chr6 | 117301021 | G | C | COSM150168 | ENSP00000357494.3: | G > C |
| | | 117300973- | | | | | | p.Ser2229Cys | |
| | | 117301137 | | | | | | | |
| ROS1 | COSM5576148 | chr6: | chr6 | 117308866 | G | T | COSM5576148 | ENSP00000357494.3: | G > T |
| | | 117308793- | | | | | | p.Ser2166Tyr | |
| | | 117308928 | | | | | | | |
| ROS1 | COSM6950684 | chr6: | chr6 | 117311094 | C | A | COSM6950684 | ENSP00000357494.3: | C > A |
| | | 117311019- | | | | | | p.Leu2053Phe | |
| | | 117311117 | | | | | | | |
| ROS1 | COSM6950893 | chr6: | chr6 | 117318188 | C | T | COSM6950893 | ENSP00000357494.3: | C > T |
| | | 117318187- | | | | | | p.Ser2002Asn | |
| | | 117318252 | | | | | | | |
| ROS1 | COSM9513198 | chr6: | chr6 | 117321391 | C | A | COSM9513198 | ENSP00000357494.3: | C > A |
| | | 117321258- | | | | | | p.Trp1882Leu | |
| | | 117321394 | | | | | | | |
| ROS1 | COSM6941244 | chr6: | chr6 | 117324337 | G | A | COSM6941244 | ENSP00000357494.3: | G > A |
| | | 117324331- | | | | | | p.Thr1879Ile | |
| | | 117324415 | | | | | | | |
| ROS1 | COSM6965416 | chr6: | chr6 | 117329359 | C | A | COSM6965416 | ENSP00000357494.3: | C > A |
| | | 117329328- | | | | | | p.Cys1779Phe | |
| | | 117329446 | | | | | | | |
| ROS1 | COSM9125580 | chr6: | chr6 | 117337327 | T | C | COSM9125580 | ENSP00000357494.3: | T > C |
| | | 117337171- | | | | | | p.Glu1698Gly | |
| | | 117337340 | | | | | | | |
| ROS1 | COSM6969339 | chr6: | chr6 | 117344154 | C | T | COSM6969339 | ENSP00000357494.3: | C > T |
| | | 117344059- | | | | | | p.Trp1477Ter | |
| | | 117344262 | | | | | | | |
| ROS1 | COSM4992412 | chr6: | chr6 | 117353036 | C | T | COSM4992412 | ENSP00000357494.3: | C > T |
| | | 117352989- | | | | | | p.Arg1425= | |
| | | 117353169 | | | | | | | |
| ROS1 | COSM6951463 | chr6: | chr6 | 117365132 | G | C | COSM6951463 | ENSP00000357494.3: | G > C |
| | | 117365059- | | | | | | p.Leu1016Val | |
| | | 117365204 | | | | | | | |
| ROS1 | COSM6954777 | chr6: | chr6 | 117365633 | G | A | COSM6954777 | ENSP00000357494.3: | G > A |
| | | 117365580- | | | | | | p.Ala974Val | |
| | | 117365741 | | | | | | | |
| ROS1 | COSM95208 | chr6: | chr6 | 117366216 | T | C | COSM95208 | ENSP00000357494.3: | T > C |
| | | 117366075- | | | | | | p.Tyr891Cys | |
| | | 117366290 | | | | | | | |
| ROS1 | COSM4992418 | chr6: | chr6 | 117379095 | C | T | COSM4992418 | ENSP00000357494.3: | C > T |
| | | 117379058- | | | | | | p.Cys854Tyr | |
| | | 117379159 | | | | | | | |
| ROS1 | COSM7342701 | chr6: | chr6 | 117383402 | G | T | COSM7342701 | ENSP00000357494.3: | G > T |
| | | 117383316- | | | | | | p.Thr804Asn | |
| | | 117383508 | | | | | | | |
| ROS1 | COSM9277478 | chr6: | chr6 | 117385755 | G | C | COSM9277478 | ENSP00000357494.3: | G > C |
| | | 117385682- | | | | | | p.Ser744Arg | |
| | | 117385861 | | | | | | | |
| ROS1 | COSM6977455 | chr6: | chr6 | 117386990 | G | A | COSM6977455 | ENSP00000357494.3: | G > A |
| | | 117386888- | | | | | | p.Pro675Leu | |
| | | 117386999 | | | | | | | |
| ROS1 | COSM6912204 | chr6: | chr6 | 117389389 | G | C | COSM6912204 | ENSP00000357494.3: | G > C |
| | | 117389349- | | | | | | p.Leu574Val | |
| | | 117389846 | | | | | | | |
| ROS1 | COSM6912725 | chr6: | chr6 | 117394313 | T | A | COSM6912725 | ENSP00000357494.3: | T > A |
| | | 117394161- | | | | | | p.Tyr338Phe | |
| | | 117394346 | | | | | | | |
| ROS1 | COSM6968764 | chr6: | chr6 | 117394705 | C | T | COSM6968764 | ENSP00000357494.3: | C > T |
| | | 117394615- | | | | | | p.Arg297Lys | |
| | | 117394738 | | | | | | | |
| ROS1 | COSM6921289 | chr6: | chr6 | 117396196 | G | C | COSM6921289 | ENSP00000357494.3: | G > C |
| | | 117396187- | | | | | | p.Ser283Cys | |
| | | 117396264 | | | | | | | |
| ROS1 | COSM5019315 | chr6: | chr6 | 117403216 | C | T | COSM5019315 | ENSP00000357494.3: | C > T |
| | | 117403138- | | | | | | p.Arg167Gln | |
| | | 117403277 | | | | | | | |
| ROS1 | COSM3761460 | chr6: | chr6 | 117404415 | T | A | COSM3761460 | ENSP00000357494.3: | T > A |
| | | 117404279- | | | | | | p.Leu101= | |
| | | 117404428 | | | | | | | |
| ROS1 | COSM6952051 | chr6: | chr6 | 117409597 | C | T | COSM6952051 | ENSP00000357494.3: | C > T |
| | | 117409581- | | | | | | p.Glu92Lys | |
| | | 117409642 | | | | | | | |
| ROS1 | COSM4604501 | chr6: | chr6 | 117416316 | C | T | COSM4604501 | ENSP00000357494.3: | C > T |
| | | 117416257- | | | | | | p.Cys57Tyr | |
| | | 117416317 | | | | | | | |
| ROS1 | COSM6355496 | chr6: | chr6 | 117418506 | c | T | COSM6355496 | ENSP00000357494.3: | C > T |
| | | 117418461- | | | | | | p.Gly42Ser | |
| | | 117418506 | | | | | | | |
| ROS1 | COSM6910632 | chr6: | chr6 | 117425580 | T | C | COSM6910632 | ENSP00000357494.3: | T > C |
| | | 117425533- | | | | | | p.Gln26Arg | |
| | | 117425656 | | | | | | | |
| MET | COSM5945634 | chr7: | chr7 | 116731717 | G | A | COSM5945634 | ENSP00000317272.6: | G > A |
| | | 116731667- | | | | | | p.Arg417Gln | |
| | | 116731859 | | | | | | | |
| MET | COSM6927005 | chr7: | chr7 | 116739966 | C | T | COSM6927005 | ENSP00000317272.6: | C > T |
| | | 116739949- | | | | | | p.Ser470Leu | |
| | | 116740084 | | | | | | | |
| MET | COSM3632213 | chr7: | chr7 | 116757727 | G | A | COSM3632213 | ENSP00000317272.6: | G > A |
| | | 116757637- | | | | | | p.Gly685= | |
| | | 116757774 | | | | | | | |
| MET | COSM5047343 | chr7: | chr7 | 116769789 | G | C | COSM5047343 | ENSP00000317272.6: | G > C |
| | | 116769644- | | | | | | p.Glu928Gln | |
| | | 116769791 | | | | | | | |
| MET | COSM5609378 | chr7: | chr7 | 116771624 | C | T | COSM5609378 | ENSP00000317272.6: | C > T |
| | | 116771497- | | | | | | p.Leu971= | |
| | | 116771654 | | | | | | | |
| MET | COSM6438054 | chr7: | chr7 | 116777440 | A | T | COSM6438054 | ENSP00000317272.6: | A > T |
| | | 116777388- | | | | | | p.Lys1122Ile | |
| | | 116777469 | | | | | | | |
| MET | COSM6983877 | chr7: | chr7 | 116778811 | ACC | ATT | COSM6983877 | ENSP00000317272.6: | ACC > A |
| | | 116778775- | | | | | | p.Thr1144Ile | TT |
| | | 116778957 | | | | | | | |
| EGFR | COSM6937748 | chr7: | chr7 | 55019353 | G | A | COSM6937748 | ENSP00000275493.2: | G > A |
| | | 55019277- | | | | | | p.Glu26Lys | |
| | | 55019365 | | | | | | | |
| EGFR | COSM9233361 | chr7: | chr7 | 55143416 | G | T | COSM9233361 | ENSP00000275493.2: | G > T |
| | | 55143304- | | | | | | p.Ala118Ser | |
| | | 55143488 | | | | | | | |
| EGFR | COSM42978 | chr7: | chr7 | 55146655 | C | T | COSM42978 | ENSP00000275493.2: | C > T |
| | | 55146605- | | | | | | p.Asn158= | |
| | | 55146740 | | | | | | | |
| EGFR | COSM7002280 | chr7: | chr7 | 55151308 | C | G | COSM7002280 | ENSP00000275493.2: | C > G |
| | | 55151293- | | | | | | p.Pro192Ala | |
| | | 55151362 | | | | | | | |
| EGFR | COSM4166393 | chr7: | chr7 | 55152627 | C | T | COSM4166393 | ENSP00000275493.2: | C > T |
| | | 55152545- | | | | | | p.Ala237Val | |
| | | 55152664 | | | | | | | |
| EGFR | COSM6970489 | chr7: | chr7 | 55156802 | G | A | COSM6970489 | ENSP00000275493.2: | G > A |
| | | 55156758- | | | | | | p.Asp393Asn | |
| | | 55156843 | | | | | | | |
| EGFR | COSM7002279 | chr7: | chr7 | 55157735 | G | A | COSM7002279 | ENSP00000275493.2: | G > A |
| | | 55157662- | | | | | | p.Arg427His | |
| | | 55157753 | | | | | | | |
| EGFR | COSM236670 | chr7: | chr7 | 55160316 | C | A | COSM236670 | ENSP00000275493.2: | C > A |
| | | 55160138- | | | | | | p.Ser492Arg | |
| | | 55160338 | | | | | | | |
| EGFR | COSM5530405 | chr7: | chr7 | 55163734 | G | A | COSM5530405 | ENSP00000275493.2: | G > A |
| | | 55163732- | | | | | | p.Glu545Lys | |
| | | 55163823 | | | | | | | |
| EGFR | COSM3762772 | chr7: | chr7 | 55171181 | T | A | COSM3762772 | ENSP00000275493.2: | T > A |
| | | 55171174- | | | | | | p.Thr629= | |
| | | 55171213 | | | | | | | |
| EGFR | COSM6976991 | chr7: | chr7 | 55192784 | G | A | COSM6976991 | ENSP00000275493.2: | G > A |
| | | 55192765- | | | | | | p.Ala882Thr | |
| | | 55192841 | | | | | | | |
| EGFR | COSM6932208 | chr7: | chr7 | 55198789 | C | T | COSM6932208 | ENSP00000275493.2: | C > T |
| | | 55198716- | | | | | | p.Ser925Phe | |
| | | 55198863 | | | | | | | |
| EGFR | COSM5762244 | chr7: | chr7 | 55200351 | C | T | COSM5762244 | ENSP00000275493.2: | C > T |
| | | 55200315- | | | | | | p.Arg962Cys | |
| | | 55200413 | | | | | | | |
| EGFR | COSM3762773 | chr7: | chr7 | 55201223 | C | T | COSM3762773 | ENSP00000275493.2: | C > T |
| | | 55201187- | | | | | | p.Asp994= | |
| | | 55201355 | | | | | | | |
| EGFR | COSM6925302 | chr7: | chr7 | 55201765 | T | C | COSM6925302 | ENSP00000275493.2: | T > C |
| | | 55201734- | | | | | | p.Cys1049Arg | |
| | | 55201782 | | | | | | | |
| EGFR | COSM7410173 | chr7: | chr7 | 55202527 | G | A | COSM7410173 | ENSP00000275493.2: | G > A |
| | | 55202516- | | | | | | p.Cys1058Tyr | |
| | | 55202625 | | | | | | | |
| EGFR | COSM9496259 | chr7: | chr7 | 55205525 | G | A | COSM9496259 | ENSP00000275493.2: | G > A |
| | | 55205255- | | | | | | p.Ala1181Thr | |
| | | 55205617 | | | | | | | |
| GNAQ | COSM52975 | chr9: | chr9 | 77797577 | C | T | COSM52975 | ENSP00000286548.4: | C > T |
| | | 77797519- | | | | | | p.Arg183Gln | |
| | | 77797648 | | | | | | | |

In some instances, a variant is described in Table 3.

TABLE 3

| | | | | | genome_ |
|---|---|---|---|---|---|
| fusion_name | chrom_5 | pos_5 | chrom_3 | pos_3 | build |
| TPR-ALK | chr1 | 186,356,039 | chr2 | 29,224,077 | hg38 |
| NCOA4-RET | chr10 | 46,011,368 | chr10 | 43,116,070 | hg38 |
| EML4-ALK1 | chr2 | 42296684 | chr2 | 29223819 | hg38 |
| EML4-ALK2 | chr2 | 42274039 | chr2 | 29225364 | hg38 |
| EML4-ALK3 | chr2 | 42299217 | chr2 | 29224971 | hg38 |
| KIF5B-RET1 | chr10 | 32024672 | chr10 | 43115128 | hg38 |
| KIF5B-RET2 | chr10 | 32017899 | chr10 | 43116571 | hg38 |
| KIF5B-RET3 | chr10 | 32016770 | chr10 | 43111682 | hg38 |
| CDC6-RET_1 | chr10 | 59902926 | chr10 | 43116231 | hg38 |
| CDC6-RET_2 | chr10 | 59856493 | chr10 | 43115739 | hg38 |
| CDC6-RET_3 | chr10 | 59878856 | chr10 | 43114499 | hg38 |
| TMPRSS2- | chr21 | 41500529 | chr21 | 38459804 | hg38 |
| ERG_1 | | | | | |
| TMPRSS2- | chr21 | 41498978 | chr21 | 38498963 | hg38 |
| ERG_2 | | | | | |
| TMPRSS2- | chr21 | 41492789 | chr21 | 38454919 | hg38 |
| ERG_3 | | | | | |
| TMPRSS2- | chr21 | 41491740 | chr21 | 38504508 | hg38 |
| ERG_4 | | | | | |

In some instances, a variant is described in Table 4.

TABLE 4

| | | | | | | | | cosmic_ | ddpcr_ | Var_ |
|---|---|---|---|---|---|---|---|---|---|---|
| chrom | start | end | gene | protein | dna | ref | alt | id | quant | length |
| 1 | 11471 | 11471 | NRAS/ | p.Q61R | c.182A > G | T | C | COSM58 | 0.0221 | 1 |
| | 3907 | 3908 | CSDE1 | | | | | 4 | | |
| 3 | 17923 | 17923 | PIK3C | p.N1068fs*4 | c.3204_320 | c | C + 1A | COSM12 | 0.0187 | 1 |
| | 4360 | 4361 | A | | 5insA | | | 464 | | |
| 4 | 54274 | 54274 | PDGFR | p.S566fs*6 | C.1694_169 | T | T + 1A | COSM28 | 0.0224 | 1 |
| | 880 | 881 | A | | 5insA | | | 053 | | |
| 5 | 11284 | 11284 | APC | p.T1556fs*3 | c.4666_466 | G | G + 1A | COSM18 | 0.0181 | 1 |
| | 0253 | 0254 | | | 7insA | | | 561 | | |
| 10 | 87957 | 87957 | PTEN | p.P248fs*5 | c.741_742 | T | T + 1A | COSM49 | 0.0143 | 1 |
| | 957 | 958 | | | insA | | | 86 | | |
| 10 | 87958 | 87958 | PTEN | p.K267fs*9 | c.800delA | A | * | COSM58 | 0.0143 | 1 |
| | 012 | 013 | | | | | | 09 | | |
| 11 | 10824 | 10824 | ATM | p.C353fs*5 | c.1058_105 | | | COSM21 | 0.025 | 1 |
| | 7120 | 7121 | | | 9delGT | | | 924 | | |
| 17 | 76742 | 76742 | TP53 | p.R248Q | c.743G > A | C | T | COSM10 | 0.0204 | 1 |
| | 19 | 20 | | | | | | 662 | | |
| 17 | 76742 | 76742 | TP53 | p.C242fs*5 | c.723delC | G | * | COSM65 | 0.0202 | 1 |
| | 39 | 40 | | | | | | 30 | | |
| 17 | 76761 | 76761 | TP53 | p.S90fs*33 | c.263delC | G | * | COSM18 | NaN | 1 |
| | 01 | 02 | | | | | | 610 | | |
| 18 | 51076 | 51076 | SMAD4 | p.A466fs*28 | c.1394_139 | G | G + 1T | COSM14 | 0.0197 | 1 |
| | 721 | 722 | | | 5insT | | | 105 | | |
| 1 | 43349 | 43349 | MPL | P.W515L | c.1544G > T | G | T | COSM18 | 0.0219 | 1 |
| | 337 | 338 | | | | | | 918 | | |
| 2 | 20824 | 20824 | IDH1 | P.R132C | c.394C > T | G | A | COSM28 | 0.0253 | 1 |
| | 8388 | 8389 | | | | | | 747 | | |
| 3 | 41224 | 41224 | CTNNB1 | p.T41A | c.121A > G | A | G | COSM56 | 0.0226 | 1 |
| | 632 | 633 | | | | | | 64 | | |
| 3 | 13894 | 13894 | FOXL2 | P.C134W | c.402C > G | G | C | COSM33 | 0.0189 | 1 |
| | 6320 | 6321 | | | | | | 661 | | |
| 3 | 17921 | 17921 | PIK3C | p.E545K | c.1633G > A | G | A | COSM76 | 0.0177 | 1 |
| | 8302 | 8303 | A | | | | | 3 | | |
| 3 | 17923 | 17923 | PIK3C | p.H1047R | c.3140A > G | A | G | COSM77 | 0.0204 | 1 |
| | 4296 | 4297 | A | | | | | 5 | | |
| 4 | 18018 | 18018 | FGFR3 | P.S249C | c.746C > G | C | G | COSM71 | 0.0199 | 1 |
| | 40 | 41 | | | | | | 5 | | |
| 4 | 54285 | 54285 | PDGFR | p.D842V | c.2525A > T | A | T | COSM73 | 0.0211 | 1 |
| | 925 | 926 | A | | | | | 6 | | |
| 4 | 54733 | 54733 | KIT | P.D816V | c.2447A > T | A | T | COSM13 | 0.0223 | 1 |
| | 154 | 155 | | | | | | 14 | | |
| 5 | 11283 | 11283 | APC | p.R1450* | c.4348C > T | C | T | COSM13 | 0.0175 | 1 |
| | 9941 | 9942 | | | | | | 127 | | |
| 5 | 17141 | 17141 | NPMI | p.W288fs*12 | c.863_864 | C | C + | COSM17 | 0.015 | 1 |
| | 0538 | 0539 | | | insTCTG | | 4TCTG | 559 | | |
| 7 | 55174 | 55174 | EGFR | p.E746_A750 | c.2236_225 | | | COSM62 | 0.0243 | 15 |
| | 772 | 787 | | delELREA | 0del15 | | | 25 | | |
| | | | | (“ELREA” | | | | | | |
| | | | | disclosed as | | | | | | |
| | | | | SEQ ID NO: | | | | | | |
| | | | | 12) | | | | | | |
| 7 | 55181 | 55181 | EGFR | p.D770_N771 | c.2310_231 | C | C+3GGT | COSM12 | 0.0214 | 1 |
| | 318 | 319 | | insG | linsGGT | | | 378 | | |
| 7 | 55181 | 55181 | EGFR | p.T790M | c.2369C > T | C | T | COSM62 | 0.0214 | 1 |
| | 377 | 378 | | | | | | 40 | | |
| 7 | 55191 | 55191 | EGFR | p.L858R | c.2573T > G | T | G | COSM62 | 0.0261 | 1 |
| | 821 | 822 | | | | | | 24 | | |
| 7 | 14075 | 14075 | BRAF | P.V600E | c,1799T > A | A | T | COSM47 | 0.0213 | 1 |
| | 3335 | 3336 | | | | | | 6 | | |
| 9 | 50737 | 50737 | JAK2 | P.V617F | c,1849G > T | G | T | COSM12 | 0.0198 | 1 |
| | 69 | 70 | | | | | | 600 | | |
| 9 | 77794 | 77794 | GNAQ | P.Q209P | c.626A > C | T | G | COSM28 | 0.0193 | 1 |
| | 571 | 572 | | | | | | 758 | | |
| 10 | 43121 | 43121 | RET | p.M918T | c.2753T > C | T | C | COSM96 | 0.0204 | 1 |
| | 967 | 968 | | | | | | 5 | | |
| 12 | 25245 | 25245 | KRAS | p.G12D | c.35G> A | C | T | COSM52 | 0.0203 | 1 |
| | 349 | 350 | | | | | | 1 | | |
| 13 | 28018 | 28018 | FLT3 | p.D835Y | c.2503G > T | C | A | COSM78 | 0.021 | 1 |
| | 504 | 505 | | | | | | 3 | | |
| 14 | 10478 | 10478 | AKTI | p.E17K | c.49G > A | C | T | COSM33 | 0.022 | 1 |
| | 0213 | 0214 | | | | | | 765 | | |
| 17 | 76738 | 76738 | TP53 | p.R273H | c.818G > A | c | T | COSM10 | 0.0196 | 1 |
| | 01 | 02 | | | | | | 660 | | |
| 17 | 76750 | 76750 | TP53 | p.R175H | c.524G > A | c | T | COSM10 | 0.0209 | 1 |
| | 87 | 88 | | | | | | 648 | | |
| 17 | 39724 | 39724 | ERBB2 | p.A775_G776 | c.2324_232 | A | A + | COSM68 | 0.0227 | 1 |
| | | | | insYVMA | 5ins12 | | 12GCATA | 2/20959 | | |
| | 727 | 728 | | (“YVMA” | | | CGTGATG | | | |
| | | | | disclosed as | | | (SEQ ID | | | |
| | | | | SEQ ID NO: | | | NO: | | | |
| | | | | 7) | | | 54) | | | |
| 20 | 58854 | 58854 | GNAS | P.R201C | c.601C > T | c | T | COSM27 | 0.0206 | 1 |
| | 052 | 053 | | | | | | 887 | | |

In some instances, a variant is described in Table 5.

TABLE 5

| Chromosome | Gene | Mutation |
|---|---|---|
| 7q34 | BRAF | V600E |
| 4q11-q12 | cKIT | D816V |
| 7p12 | EGFR | ΔE746 - A750 |
| 7p12 | EGFR | L858R |
| 7p12 | EGFR | T790M |
| 7p12 | EGFR | G719S |
| 12p12.1 | KRAS | G13D |
| 12p12.1 | KRAS | G12D |
| 1p13.2 | NRAS | Q61K |
| 3q26.3 | PIK3CA | h2047R |
| 3q26.3 | PIK3CA | E545K |
| p23 | ALK | P1543S |
| 1q25.2 | ABL2 | P986fs |
| 5q21-q22 | ARC | R2714C |
| 1p35.3 | ARID1A | p.M1564fs*1 |
| 13q12.3 | BRCA2 | A1689fs |
| 13q12.3 | CDX2 | V306fs |
| 22q13.2 | EP300 | K291fs |
| 4q31.3 | FBXW7 | G667fs |
| 8p12 | FGFR1 | P150L |
| 13q12 | FLT3 | V197A |
| 2q33.3 | IDh2 | S261L |
| 7q31 | MET | V237fs |
| 3p21.3 | MLh2 | L323M |
| 17q11.2 | NF1 | L626fs |
| 22q12.2 | NF2 | P275fs |
| 9q34.3 | NOTCh2 | P668S |
| 1q21-q22 | NTRK1 | 5′UTR |
| 4q12 | PDGFRA | G426D |

In some instances, a variant is described in Table 6.

TABLE 6

| Gene | Variant description |
|---|---|
| NRAS | G12D |
| NRAS | Q61H |
| IDH2 | R172K |
| IDH2 | R140Q |
| CTNNB1 | G34E |
| FOXL2 | — |
| PIK3CA | N345K |
| FGFR1 | N546K |
| FGFR1 | K656E |
| FGFR2 | S252W |
| FGFR2 | N549K |
| FGFR2 | C382R |
| FGFR2 | K659E |
| FGFR3 | Y373C |
| FGFR3 | K650E |
| PDGFRA | V561D |
| PDGFRA | N659K |
| PDGFRA | SPDGHE566- (“SPDGHE” disclosed as SEQ ID NO: 55) |
| KIT | L576P |
| KIT | V560G |
| KIT | del547-555 |
| KIT | K642E |
| EGFR | S768I |
| EGFR | G724S |
| EGFR | L792H |
| EGFR | L718Q |
| BRAF | None |
| JAK2 | None |
| GNAQ | T96S |
| RET | None |
| PTEN | None |
| KRAS | Q61H |
| FLT3 | None |
| AKT1 | L52R |
| AKT1 | Q79K |
| TP53 | G245C |
| TP53 | R282W |
| ERBB2 | L755S |
| ERBB2 | P780_Y781insGSP |
| ERBB2 | V842I |
| SMAD4 | R361H |
| GNA11 | None |
| GNAS1 | None |
| ATM | None |
| ALK | G1128A, F1174L, R1192P, R1275Q |
| AR | T878A, W742C, structural variants |
| ARAF | S214C |
| BRCA1 | c.4964_4982del19 - p.(Ser1655Tyrfs*16)/5, |
| | c.5266dupC - p.(Gln1756Profs*74)/3, |
| BRCA2 | c.5351dupA - p.(Asn1784Lysfs*3)/4 |
| CCND1 | E275*fs, T286I |
| CDH1 | R732Q, A634V |
| CDK12 | W719*, E928fs27* |
| CDK4 | R24C |
| CDK6 | — |
| CDKN2A | — |
| DDR2 | I638F, L239R |
| ESR1 | D538G |
| EZH2 | Y641F |
| FGFR2 | S252W, N550K |
| HRAS | G12V |
| JAK3 | — |
| MAP2K1 | K57 N |
| MAP2K2 | — |
| MET | d1246n |
| MTOR | — |
| NF1 | — |
| NTRK1 | Fusions |
| PTPN11 | E76K, G503R |
| RAF1 | — |
| RB1 | — |
| ROS1 | G2032R |
| SMO | D473H |
| STK11 | — |
| TERT | C228T and C250T in promoter |
| ABL1 | F317V |
| ARID1A | Q1401* |
| ATR | — |
| BAP1 | W196* |
| CCND2 | None |
| CCNE1 | None |
| CD274 | None |
| CHD1 | Q23* |
| CHEK2 | None |
| CRKL | None |
| ERBB3 | v104m |
| ERRFI1 | None |
| FBXW7 | R465C |
| FGFR4 | None |
| FH | None |
| FOXA1 | R219S |
| GATA3 | P408fs |
| HNF1A | P289fs |
| KDM5C | S1222P |
| KDM6A | p.I598fsX6 |
| MAPK1 | E322K |
| MAPK3 | None |
| MLH1 | R498fs |
| MYC | T58A |
| MYCN | P44L |
| MYD88 | L265P |
| NF2 | None |
| NFE2L2 | G333C |
| NOTCH1 | LOF frameshift mutations, E124*, W1843* |
| NTRK3 | None |
| PALB2 | — |
| PBRM1 | p.F116fs*7 |
| PDCD1LG2 | None |
| PDGFRB | None |
| RHEB | Y35N |
| RHOA | Y42C |
| RIT1 | M90I |
| SETD2 | — |
| SF3B1 | None |
| SMARCB1 | None |
| SPOP | F133V |
| TSC1 | p.Q794 |
| VEGFA | — |
| VHL | None |
| ZNF703 | None |

In some instances, a variant described herein is from one or more of Tables 1-6.

Variants (e.g., genomic variants) may be detected from a sample (e.g., genomic sample) with varying degrees of recall and precision. In some instances, the upper limit on detection is determined by performance of a reference standard described herein. In some instances, reference standards have pre-selected variant frequencies for comparison to patient samples. In some instances, recall represents the number of variants detected out of all that variants expected to be detectable. In some instances, precision represents the number of variants that are called correctly out of everything detected as a variant. In some instances, the variant is detected with a recall of at least 30%, 50%, 75%, 80%, 85%, 90%, 95%, 97%, 98%, or at least 99%. In some instances, the variant is detected with a recall of about 30%, 50%, 75%, 80%, 85%, 90%, 95%, 97%, 98%, or about 99%. In some instances, the variant is detected with a recall of about 10%-99%, 25-99%, 30-90%, 45-80%, 50-99%, 75-99%, or 90-99%. In some instances, the variant is detected with a precision of at least 30%, 50%, 75%, 80%, 85%, 90%, 95%, 97%, 98%, or at least 99%. In some instances, the variant is detected with a precision of about 30%, 50%, 75%, 80%, 85%, 90%, 95%, 97%, 98%, or about 99%. In some instances, the variant is detected with a precision of about 10%-99%, 25-99%, 30-90%, 45-80%, 50-99%, 75-99%, or 90-99%.

Polynucleotide libraries may be designed to comprise sequences which are identical to or complementary (to target, hybridize) to one or more variants. In some instances, at least some of the polynucleotides are each configured to hybridize to genomic regions which comprise at least two variants. In some instances, at least some of the polynucleotides are each configured to hybridize to genomic regions which comprise at least one, two, three, four, five, six, or more than six variants. In some instances, at least some of the polynucleotides are each configured to hybridize to genomic regions which comprise one to four variants. In some instances, at least some of the polynucleotides are each configured to hybridize to genomic regions which comprise one to two or three variants. In some instances, at least 50% of the polynucleotides are each configured to hybridize to genomic regions which comprise at least two variants. In some instances, at least 50% of the polynucleotides are each configured to hybridize to genomic regions which comprise at least one, two, three, four, five, six, or more than six variants. In some instances, at least 50% of the polynucleotides are each configured to hybridize to genomic regions which comprise one to four variants. In some instances, at least 50% of the polynucleotides are each configured to hybridize to genomic regions which comprise one to two or three variants. In some instances, at least 25% of the polynucleotides are each configured to hybridize to genomic regions which comprise at least two variants. In some instances, at least 25% of the polynucleotides are each configured to hybridize to genomic regions which comprise at least one, two, three, four, five, six, or more than six variants. In some instances, at least 25% of the polynucleotides are each configured to hybridize to genomic regions which comprise one to four variants. In some instances, at least 25% of the polynucleotides are each configured to hybridize to genomic regions which comprise one to two or three variants. In some instances, at least 5% of the polynucleotides are each configured to hybridize to genomic regions which comprise at least two variants. In some instances, at least 5% of the polynucleotides are each configured to hybridize to genomic regions which comprise at least one, two, three, four, five, six, or more than six variants. In some instances, at least 5% of the polynucleotides are each configured to hybridize to genomic regions which comprise one to four variants. In some instances, at least 5% of the polynucleotides are each configured to hybridize to genomic regions which comprise one to two or three variants.

Polynucleotide libraries may be configured to bind to many variants. In some instances, a polynucleotide library is collectively configured to bind to genomic regions comprising about 50, 100, 200, 500, 800, 1000, 2000, 5000, 8000, 10,000, 20,000, 50,000, 80,000, 100,000, 250,000, 500,000, 750,000, 1 million, 1.5 million, 2 million, 2.5 million, 3 million, 3.5 million, 4 million, 4.5 million, or about 5 million variants. In some instances, a polynucleotide library is collectively configured to bind to genomic regions comprising at least 50, 100, 200, 500, 800, 1000, 2000, 5000, 8000, 10,000, 20,000, 50,000, 80,000, 100,000, 250,000, 500,000, 750,000, 1 million, 1.5 million, 2 million, 2.5 million, 3 million, 3.5 million, 4 million, 4.5 million, or at least 5 million variants. In some instances, a polynucleotide library is collectively configured to bind to genomic regions comprising 100-1000, 50-100, 50-500, 50-5000, 50-10,000, 100,000-5 million, 250,000-3 million, 500,000-2 million, 750,000-4 million, 1 million-5 million, 1 million-3 million, 1 million-4 million, or 4 million to 6 million variants.

Polynucleotide libraries for identifying variants may be optimized. In some instances, the library is uniform (each unique polynucleotide is equally represented). In some instances, the library is not uniform. In some instances, polynucleotides are represented in an amount within at least about 1.5 times the mean representation for the polynucleotide library. In some instances, polynucleotides are represented in an amount within at least about 2 times the mean representation for the polynucleotide library. In some instances, polynucleotides are represented in an amount within at least about 1.2 times the mean representation for the polynucleotide library. In some instances, polynucleotides are represented in an amount within at least about 1.7 times the mean representation for the polynucleotide library. In some instances, at least 80% polynucleotides are represented in an amount within at least about 1.5 times the mean representation for the polynucleotide library. In some instances, at least 80% polynucleotides are represented in an amount within at least about 2 times the mean representation for the polynucleotide library. In some instances, at least 80% polynucleotides are represented in an amount within at least about 1.7 times the mean representation for the polynucleotide library. In some instances, at least 80% polynucleotides are represented in an amount within at least about 2 times the mean representation for the polynucleotide library. In some instances, at least 90% polynucleotides are represented in an amount within at least about 1.5 times the mean representation for the polynucleotide library. In some instances, at least 90% polynucleotides are represented in an amount within at least about 2 times the mean representation for the polynucleotide library. In some instances, at least 80% polynucleotides are represented in an amount within at least about 1.7 times the mean representation for the polynucleotide library. In some instances, at least 90% polynucleotides are represented in an amount within at least about 2 times the mean representation for the polynucleotide library. In some instances, at least 95% polynucleotides are represented in an amount within at least about 1.5 times the mean representation for the polynucleotide library. In some instances, at least 95% polynucleotides are represented in an amount within at least about 2 times the mean representation for the polynucleotide library. In some instances, at least 95% polynucleotides are represented in an amount within at least about 1.7 times the mean representation for the polynucleotide library. In some instances, at least 95% polynucleotides are represented in an amount within at least about 2 times the mean representation for the polynucleotide library. Polynucleotide libraries in some instances comprise at least some polynucleotides which each comprise an overlap region with another polynucleotide in the library. In some instances at least 10%, 20%, 30%, 40%, 50%, 60%, 70%, 80%, or at least 90% of the polynucleotides each comprise an overlap region with another polynucleotide in the library. In some instances about 10%, 20%, 30%, 40%, 50%, 60%, 70%, 80%, or about 90% of the polynucleotides each comprise an overlap region with another polynucleotide in the library. In some instances 10%-90%, 10-80%, 10-75%, 25%-50%, 25-90%, 50-90%, 15-35%, or 80-99% of the polynucleotides each comprise an overlap region with another polynucleotide in the library. In some instances, the amount of at least some of the polynucleotides in the library is 5, 10, 20, 25, 50, 75, 100, 150, 200, 250, 300, 400, 500, or 600 times higher than the mean representation for the polynucleotide library. In some instances, the amount of at least 1% of the polynucleotides in the library is 5, 10, 20, 25, 50, 75, 100, 150, 200, 250, 300, 400, 500, or 600 times higher than the mean representation for the polynucleotide library. In some instances, the amount of at least 2% of the polynucleotides in the library is 5, 10, 20, 25, 50, 75, 100, 150, 200, 250, 300, 400, 500, or 600 times higher than the mean representation for the polynucleotide library. In some instances, the amount of at least 5% of the polynucleotides in the library is 5, 10, 20, 25, 50, 75, 100, 150, 200, 250, 300, 400, 500, or 600 times higher than the mean representation for the polynucleotide library. In some instances, the amount of no more than 5% of the polynucleotides in the library is 5, 10, 20, 25, 50, 75, 100, 150, 200, 250, 300, 400, 500, or 600 times higher than the mean representation for the polynucleotide library. In some instances, the amount of no more than 10% of the polynucleotides in the library is 5, 10, 20, 25, 50, 75, 100, 150, 200, 250, 300, 400, 500, or 600 times higher than the mean representation for the polynucleotide library. In some instances, the amount of at least 1%-10% of the polynucleotides in the library is 5, 10, 20, 25, 50, 75, 100, 150, 200, 250, 300, 400, 500, or 600 times higher than the mean representation for the polynucleotide library. In some instances, the amount of at least 1%-20% of the polynucleotides in the library is 5, 10, 20, 25, 50, 75, 100, 150, 200, 250, 300, 400, 500, or 600 times higher than the mean representation for the polynucleotide library. In some instances, the relative amount of a polynucleotide library is adjusted based on high or low GC content.

Polynucleotide libraries for identifying variants may collectively target a desired number of bases (bait territory). In some instances, a polynucleotide library comprise a bait territory of at least 5, 10, 15, 20, 25, 30, 40, 50, 60, 70, 80, 90 or at least 100 million bases. In some instances, a polynucleotide library comprise a bait territory of about 5, 10, 15, 20, 25, 30, 40, 50, 60, 70, 80, 90 or about 100 million bases. In some instances, a polynucleotide library comprise a bait territory of no more than 5, 10, 15, 20, 25, 30, 40, 50, 60, 70, 80, 90 or no more than 100 million bases.

Unique Molecular Identifiers

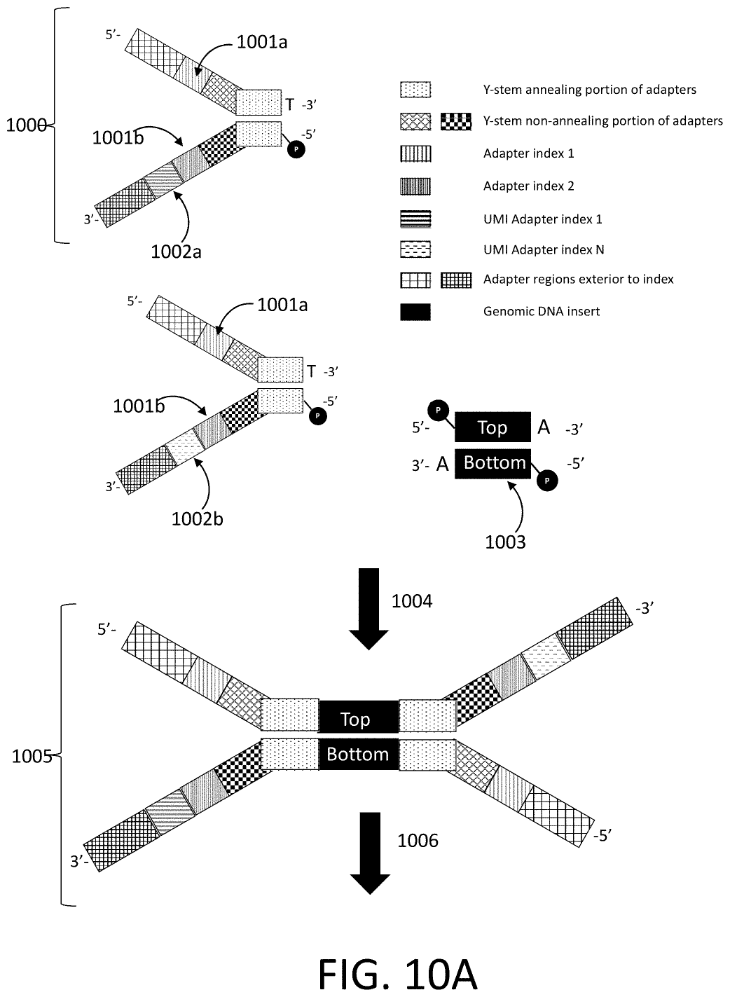

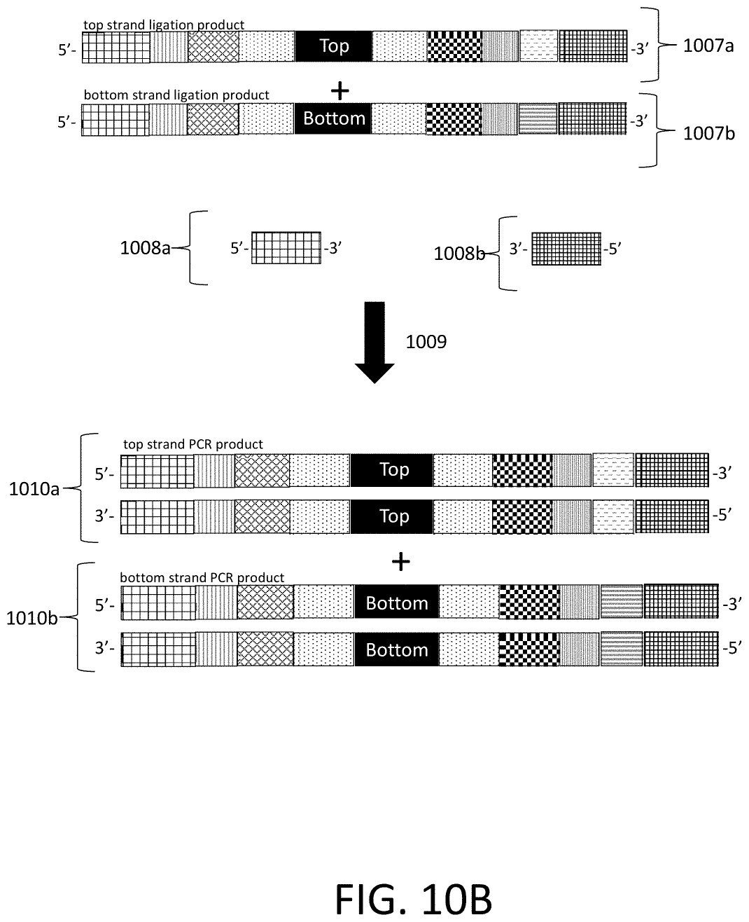

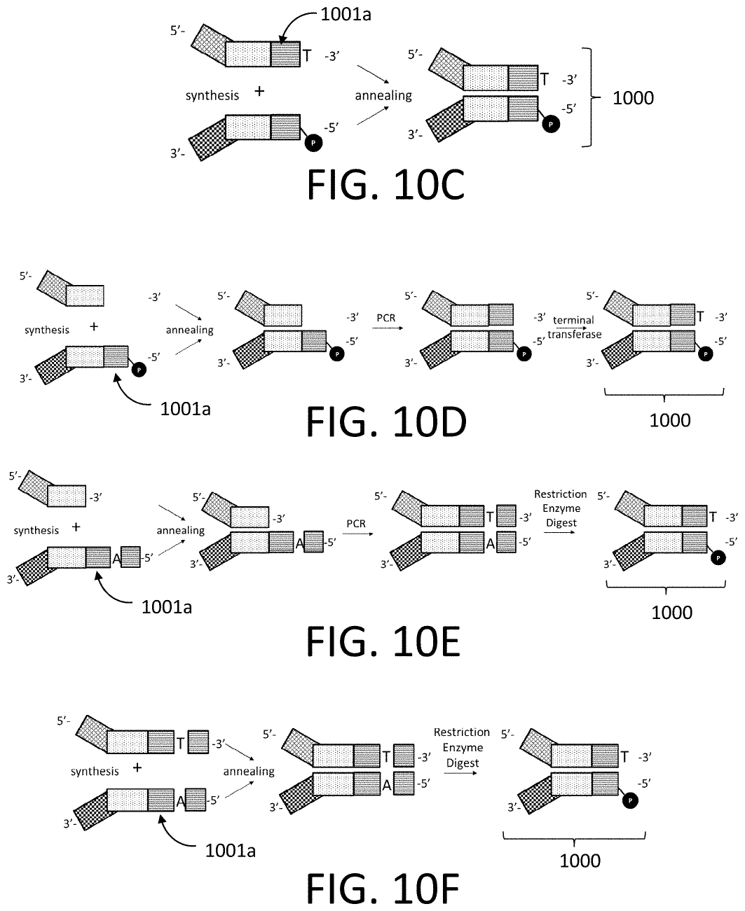

Described herein are adapters comprising unique molecular identifiers (UMIs). Adapters in some instances comprise a structure 1000 of FIG. 10. In some instances, adapters comprise universal adapters. In some instances adapters comprise a Y-annealing region (anneals to form yoke), one or more Y-step non-annealing regions, a first index region 1001a, a second index region 1001b, a first UMI (index) region 1002a, a second UMI (index) region 1002b, and one or more regions exterior to the index. In some instances, adapters 1000 are ligated 1004 to sample polynucleotides 1003 to form an adapter-ligated polynucleotide 1005. After denaturation 1006 of 1005 (FIG. 10A), top 1007a and bottom 1007b strand ligation products are formed. In some instances, each strand is labeled with a different UML. After amplification 1009 with forward 1008a and backward 1008b primers, top strand 1010a and bottom strand 1010b PCR products are generated. In some instances, adapter ligated polynucleotides generated with universal adapters are further amplified with barcoded primers. In some instances adapters described herein comprise “in-line” UMIs, wherein at least one of a 5′ or 3′ UMI is not complementary to the other corresponding strand of the adapter (1001a and 1001b are not complementary). In some instances adapters described herein comprise “duplex” UMIs, wherein at least one of a 5′ or 3′ UMI is complementary to the other corresponding strand of the adapter (1001a and 1001b are complementary).

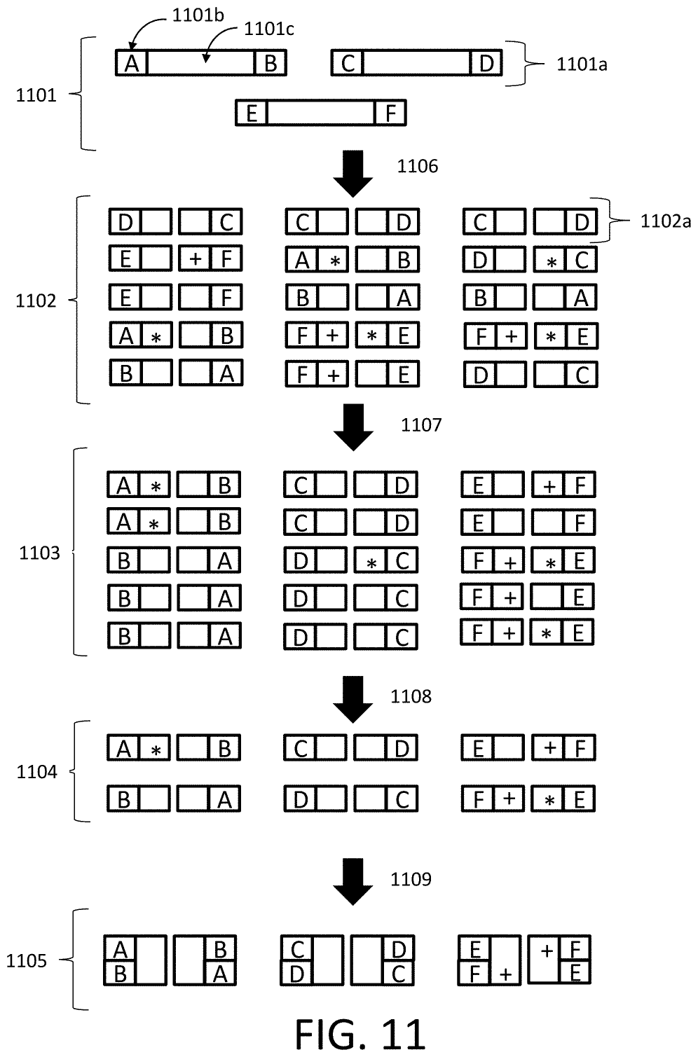

Adapter-ligated libraries comprising unique molecular identifiers may be used to distinguish between “true” mutations from a polynucleotide sample library and artifacts generated during sequencing library preparation (e.g., PCR errors, sequencing errors, or other erroneous base call). In some instances, a workflow as shown in FIG. 11 is used to analyze a library of adapter-ligated sample polynucleotides 1101. Adapter-ligated sample polynucleotides 1101 each comprise two distinct UMIs 1101b represented by letters (A-F; six combinations of barcodes are shown for simplicity), and are attached to a sample polynucleotide 1101c. After sequencing 1106, forward and reverse read pairs 1102 from sequencing are sorted into read pair groups 1102a. Potential PCR-based errors are designated with “*”, and true polymorphisms are designated as “+”. Next, read pairs 1103 are grouped 1107 by barcode and barcode position. Single-stranded consensus sequences 1104 are then generated 1108 from each group of barcode-grouped read pairs. Errors from D-C, and F-E are identified, although the error in A-B remains. Finally, duplex consensus sequences 1105 are generated 1109 by comparing each set of single stranded consensus sequences. The error in A-B can be identified, and true mutation E-F can be confirmed. In some instances, errors include substitutions, deletions, or insertions. In some instances, an error is present in the sample polynucleotide portion of an adapter-ligated polynucleotide. In some instances, an error is present in a barcode configured to identify a sample origin (e.g., index) or to uniquely identify a sample polynucleotide. In some instances, an error is present in a UML. In some instances, an error is present in a sample index. Compositions and methods described herein in some instances are used to identify such errors.

Described herein are sets of UMIs, wherein the set has defined properties. In some instances, a UMI set comprises a plurality of different polynucleotides having unique sequences. In some instances, a UMI set is 8, 12, 16, 20, 24, 30, 32, 36, 39, 48, or 64 unique sequences. In some instances, the sequences of a UMI set differ by a Hamming distance of no more than 1, 2, 3, 4, or 5. In some instances, the sequences of a UMI set differ by a Hamming distance of at least 1, 2, 3, 4, or 5. In some instances, the sequences of a UMI set differ by a Hamming distance of at least 2. In some instances, the sequences of a UMI set differ by a Hamming distance of at least 1.

UMIs may be any length, depending on the desired application. In some instances, a UMI is no more than 15, 12, 10, 8, 7, 6, 5, 4, or not more than 3 bases in length. In some instances, a UMI is about 15, 12, 10, 8, 7, 6, 5, 4, or about 3 bases in length. In some instances, a UMI is about 3-12, 3-10, 3-8. 4-12, 4-10, 4-8, 6-12, or 8-12 bases in length. UMIs in a set may comprise more than one length. In some instances, 10, 20, 25, 30, 40, 50, 60, or 70 percent of UMIs in the set are a first length, and 90, 80, 75, 70, 60, 50, 40, or 30 percent are a second length. In some instances, the first length is 3-5 bases, and the second length is 3-5 bases. In some instances, UMIs comprise lengths of 5 or 6 bases.

After addition of UMI-containing adapters to sample polynucleotides, at least some of the sample polynucleotides may be uniquely labeled. In some instances, at least 30%, 50%, 75%, 80%, 90%, 95%, or at least 98% of the sample polynucleotides are ligated to adapters comprising UMIs. In some instances, at least 1%, 2%, 5%, 10%, 15%, 20%, 30%, 50%, 75%, 80%, 90%, 95%, or at least 98% of the sample polynucleotides are labeled with a unique UMI sequence. In some instances, no more than 1%, 2%, 5%, 10%, 15%, 20%, 30%, 50%, 75%, 80%, 90%, 95%, or no more than 98% of the sample polynucleotides are labeled with a unique UMI sequence. In some instances, at least 1%, 2%, 5%10%, 15%, 20%, 30%, 50%, 75%, 80%, 90%, 95%, or at least 98% of the sample polynucleotides are uniquely identifiable after labeling with a UMI.

UMIs described herein in some instances comprise sequences of one or more of AAGGA, ACAAC, ATACG, CACTG, CATGA, CGATA, CGTGT, GCCAT, GCTGT, GTCAC, GTCGT, TACGA, TCCTA, TCGTG, TGTCG, TTGGC, AACAC, AATGC, ACTAG, AGCAT, AGTAC, ATCTC, CAGAC, CAGTA, CGAAT, CGGTT, CTTGG, GCATA, GCTAA, GTGAG, GTGTC, and TGTGC. UMIs described herein in some instances comprise sequences of two or more of AAGGA, ACAAC, ATACG, CACTG, CATGA, CGATA, CGTGT, GCCAT, GCTGT, GTCAC, GTCGT, TACGA, TCCTA, TCGTG, TGTCG, TTGGC, AACAC, AATGC, ACTAG, AGCAT, AGTAC, ATCTC, CAGAC, CAGTA, CGAAT, CGGTT, CTTGG, GCATA, GCTAA, GTGAG, GTGTC, and TGTGC. UMIs described herein in some instances comprise sequences of five or more of AAGGA, ACAAC, ATACG, CACTG, CATGA, CGATA, CGTGT, GCCAT, GCTGT, GTCAC, GTCGT, TACGA, TCCTA, TCGTG, TGTCG, TTGGC, AACAC, AATGC, ACTAG, AGCAT, AGTAC, ATCTC, CAGAC, CAGTA, CGAAT, CGGTT, CTTGG, GCATA, GCTAA, GTGAG, GTGTC, and TGTGC. UMIs described herein in some instances comprise sequences of ten or more of AAGGA, ACAAC, ATACG, CACTG, CATGA, CGATA, CGTGT, GCCAT, GCTGT, GTCAC, GTCGT, TACGA, TCCTA, TCGTG, TGTCG, TTGGC, AACAC, AATGC, ACTAG, AGCAT, AGTAC, ATCTC, CAGAC, CAGTA, CGAAT, CGGTT, CTTGG, GCATA, GCTAA, GTGAG, GTGTC, and TGTGC.

UMIs may be represented at pre-selected percentages among a library of UMIs. In some instances at least 90% of the UMIs are present at fraction of 1-5%. In some instances at least 90% of the UMIs are present at fraction of 0.5%, 1%, 1.5%, 2%, 2.5%, 3%, 3.5%, 4%, 4.5%, 5%, 5.5% 6%, 7%, or 8%. In some instances at least 90% of the UMIs are present at fraction of 0.5-8%, 1-7%, 1.5-7%, 2-7%, 2.5-6%, 3-8%, 3-6%, 1-5%, 0.5-5.5%, 1-4%, 1-6%, or 1-8%.

Any amount of sample polynucleotides (e.g., input DNA or other nucleic acid) may be ligated to adapters described herein. In some instances, the amount of sample polynucleotides is about 1, 5, 8, 10, 15, 20, 25, 30, 50, 75, or about 100 ng. In some instances, the amount of sample polynucleotides is no more than 1, 5, 8, 10, 15, 20, 25, 30, 50, 75, or no more than 100 ng. In some instances, the amount of sample polynucleotides is at least 1, 5, 8, 10, 15, 20, 25, 30, 50, 75, or at least 100 ng. In some instances, the amount of sample polynucleotides 1-10 ng, 1-100 ng, 3-10 ng, 5-100 ng, 5-75 ng, 5-50 ng, 10-100 ng, 10-50 ng, 25-100 ng, or 25-75 ng.

Provided herein are methods of generating adapters comprising UMIs. In a first method of adapter synthesis comprising synthesis of a top strand of an adapter comprising at least one UMI and a complementary bottom strand. After annealing the top and bottom adapter strands, an adapter comprising the structure of adapter 1000 is formed (FIG. 10C). In a second method of adapter synthesis, a top strand is synthesized without a UMI, and a bottom strand comprising a complementary region and a UMI (FIG. 10D). After, annealing, PCR is used to generate a complementary UMI on the top strand, and a terminal transferase adds a T to the 3′ end of top strand to generate adapter 1000. In a third method of synthesis, a top strand which does not comprise a UMI, and a bottom strand comprising a UMI, a restrictions site, and a 5′ overhang are synthesized (FIG. 10E). After annealing, the top strand is extended with PCR, and a restriction endonuclease is used to cleave a portion of the 3′ top strand and 5′ bottom strand to generate adapter 1000. In a fourth method of adapter synthesis, two complementary strands each comprising a UMI, a restriction site, and an overhang portion (3′ top strand, 5′ bottom strand) are synthesized, annealed, and cleaved with a restriction enzyme to generate adapter 1000. More than one UMIs may be present per adapter. In some instances, an adapter comprises 1, 2, 3, 4, 5, or more UMIs. In some instances, adapters comprise a first UMI and a second UML. In some instances, a first UMI and a second UMI are complementary. In some instances, adapters comprise a first UMI and a second UMI. In some instances, a first UMI and a second UMI are not complementary. In some instances adapters are combined into libraries of adapters. In some instances adapters in a library comprise UMIs. In some instances adapters in a library comprise unique combinations of a first UMI and a second UMI.

Universal Adapters

Provided herein are universal adapters. In some instances, universal adapters comprise one or more unique molecular identifiers. In some instances, the universal adapters disclosed herein may comprise a universal polynucleotide adapter comprising a first strand and a second strand. In some instances, a first strand comprises a first primer binding region, a first non-complementary region, and a first yoke region. In some instances, a second strand comprises a second primer binding region, a second non-complementary region, and a second yoke region. In some instances, a primer binding region allows for PCR amplification of a polynucleotide adapter. In some instances, a primer binding region allows for PCR amplification of a polynucleotide adapter and concurrent addition of one or more barcodes to the polynucleotide adapter. In some instances, the first yoke region is complementary to the second yoke region. In some instances, the first non-complementary region is not complementary to the second non-complementary region. In some instances, the universal adapter is a Y-shaped or forked adapter. In some instances, one or more yoke regions comprise nucleobase analogues that raise the Tₘ between a first yoke region and a second yoke region. Primer binding regions as described herein may be in the form of a terminal adapter region of a polynucleotide. In some instances, a universal adapter comprises one index sequence. In some instances, a universal adapter comprises one unique molecular identifier. In some instances, universal adapters are configured for use with barcoded primers, wherein after ligation, barcoded primers are added via PCR.

A universal (polynucleotide) adapter may be shortened relative to a typical barcoded adapter (e.g., full-length “Y adapter”). For example, a universal adapter strand is 20-45 bases in length. In some instances, a universal adapter strand is 25-40 bases in length. In some instances, a universal adapter strand is 30-35 bases in length. In some instances, a universal adapter strand is no more than 50 bases in length, no more than 45 bases in length, no more than 40 bases in length, no more than 35 bases in length, no more than 30 bases in length, or no more than 25 bases in length. In some instances, a universal adapter strand is about 25, 27, 30, 32, 34, 36, 38, 40, 42, 44, 46, 48, 50, 52, 54, 56, 58, or about 60 bases in length. In some instances, a universal adapter strand is about 60 base pairs in length. In some instances, a universal adapter strand is about 58 base pairs in length. In some instances, a universal adapter strand is about 52 base pairs in length. In some instances, a universal adapter strand is about 33 base pairs in length.

A universal adapter may be modified to facilitate ligation with a sample polynucleotide. For example, the 5′ terminus is phosphorylated. In some instances, a universal adapter comprises one or more non-native nucleobase linkages such as a phosphorothioate linkage. For example, a universal adapter comprises a phosphorothioate between the 3′ terminal base, and the base adjacent to the 3′ terminal base. A sample polynucleotide in some instances comprises nucleic acid from a variety of sources, such as DNA or RNA of human, bacterial, plant, animal, fungal, or viral origin. An adapter-ligated sample polynucleotide in some instances comprises a sample polynucleotide (e.g., sample nucleic acid) with adapters universal adapters ligated to both the 5′ and 3′ end of the sample polynucleotide to form an adapter-ligated polynucleotide. A duplex sample polynucleotide comprises both a first strand (forward) and a second strand (reverse).

Universal adapters may contain any number of different nucleobases (DNA, RNA, etc.), nucleobase analogues, or non-nucleobase linkers or spacers. For example, an adapter comprises one or more nucleobase analogues or other groups that enhance hybridization (Tₘ) between two strands of the adapter. In some instances, nucleobase analogues are present in the yoke region of an adapter. Nucleobase analogues and other groups include but are not limited to locked nucleic acids (LNAs), bicyclic nucleic acids (BNAs), C5-modified pyrimidine bases, 2′-O-methyl substituted RNA, peptide nucleic acids (PNAs), glycol nucleic acid (GNAs), threose nucleic acid (TNAs), xenonucleic acids (XNAs) morpholino backbone-modified bases, minor grove binders (MGBs), spermine, G-clamps, or a anthraquinone (Uaq) caps.

Universal adapters may comprise any number of nucleobase analogues (such as LNAs or BNAs), depending on the desired hybridization Tₘ. For example, an adapter comprises 1 to 20 nucleobase analogues. In some instances, an adapter comprises 1 to 8 nucleobase analogues. In some instances, an adapter comprises at least 1, 2, 3, 4, 5, 6, 7, 8, 9, 10, 11, 12, or at least 12 nucleobase analogues. In some instances, an adapter comprises about 1, 2, 3, 4, 5, 6, 7, 8, 9, 10, 11, 12, 13, 14, 15, or about 16 nucleobase analogues. In some instances, the number of nucleobase analogous is expressed as a percent of the total bases in the adapter. For example, an adapter comprises at least 1%, 2%, 5%, 10%, 12%, 18%, 24%, 30%, or more than 30% nucleobase analogues. In some instances, adapters (e.g., universal adapters) described herein comprise methylated nucleobases, such as methylated cytosine.

### Barcodes

Polynucleotide primers may comprise defined sequences, such as barcodes (or indices). Adapters in some instances comprise one or more barcodes. In some instances, an adapter comprises at least one indexing barcode and at least one unique molecular identifier barcode. Barcodes can be attached to universal adapters, for example, using PCR and barcoded primers to generate barcoded adapter-ligated sample polynucleotides. Primer binding sites, such as universal primer binding sites, facilitate simultaneous amplification of all members of a barcode primer library, or a subpopulation of members. In some instances, a primer binding site comprises a region that binds to a flow cell or other solid support during next generation sequencing. In some instances, a barcoded primer comprises a P5 (5′-AATGATACGGCGACCACCGA-3′ (SEQ ID NO: 56)) or P7 (5′-CAAGCAGAAGACGGCATACGAGAT-3′ (SEQ ID NO: 57)) sequence. In some instances, primer binding sites are configured to bind to universal adapter sequences, and facilitate amplification and generation of barcoded adapters. In some instances, barcoded primers are no more than 60 bases in length. In some instances, barcoded primers are no more than 55 bases in length. In some instances, barcoded primers are 50-60 bases in length. In some instances, barcoded primers are about 60 bases in length. In some instances, barcodes described herein comprise methylated nucleobases, such as methylated cytosine.

The number of unique barcodes available for a barcode set (collection of unique barcodes or barcode combinations configured to be used together to unique define samples) may depend on the barcode length. In some instances, a Hamming distance is defined by the number of base differences between any two barcodes. In some instances, a Levenshtein distance is defined by the number changes needed to change one barcode into another (insertions, substitutions, or deletions). In some instances, barcode sets described herein comprise a Levenshtein distance of at least 2, 3, 4, 5, 6, 7, or at least 8. In some instances, barcode sets described herein comprise a Hamming distance of at least 2, 3, 4, 5, 6, 7, or at least 8.

Barcodes may be incorrectly associated with a different sample than they were assigned. In some instances, incorrect barcodes are occur from PCR errors (e.g., substitution) during library amplification. In some instances, entire barcodes “hop” or are transferred from one sample polynucleotide to another. Such transfers in some instances result from cross-contamination of free adapters or primers during a library generation workflow. In some instances a group of barcodes (barcode set) is chosen to minimize “barcode hopping”. In some instances, barcode hopping (for a single barcode) for a barcode set described herein is no more than 7%, 5%, 4%, 3%, 2%, 1%, 0.5%, or no more than 0.1%. In some instances, barcode hopping (for a single barcode) for a barcode set described herein is 0.1-6%, 0.1-5%, 0.2-5%, 0.5-5%, 1-7%, 1-5%, or 0.5-7%. In some instances, barcode hopping (for two barcodes) for a barcode set described herein is no more than 0.7%, 0.5%, 0.4%, 0.3%, 0.2%, 0.1%, 0.05%, or no more than 0.1%. In some instances, barcode hopping (for two barcodes) for a barcode set described herein is 0.01-0.6%, 0.01-0.5%, 0.02-0.5%, 0.05-0.5%, 0.1-0.7%, 0.1-0.5%, or 0.05-0.7%.

Barcoded primers comprise one or more barcodes. In some instances, the barcodes are added to universal adapters through PCR reaction. Barcodes are nucleic acid sequences that allow some feature of a polynucleotide with which the barcode is associated to be identified. In some instances, a barcode comprises an index sequence. In some instances, index sequences allow for identification of a sample, or unique source of nucleic acids to be sequenced. A barcode or combination of barcodes in some instances identifies a specific patient. A barcode or combination of barcodes in some instances identifies a specific sample from a patient among other samples from the same patient. After sequencing, the barcode (or barcode region) provides an indicator for identifying a characteristic associated with the coding region or sample source. Barcodes can be designed at suitable lengths to allow sufficient degree of identification, e.g., at least about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, or more bases in length. Multiple barcodes, such as about 2, 3, 4, 5, 6, 7, 8, 9, 10, or more barcodes, may be used on the same molecule, optionally separated by non-barcode sequences. In some instances, a barcode is positioned on the 5′ and the 3′ sides of a sample polynucleotide. In some instances, each barcode in a plurality of barcodes differ from every other barcode in the plurality at least three base positions, such as at least about 3, 4, 5, 6, 7, 8, 9, 10, or more positions. Use of barcodes allows for the pooling and simultaneous processing of multiple libraries for downstream applications, such as sequencing (multiplex). In some instances, at least 4, 8, 16, 32, 48, 64, 128, or more 512 barcoded libraries are used. In some instances, at least 400, 500, 800, 1000, 2000, 5000, 10,000, 12,000, 15,000, 18,000, 20,000, or at 25,000 barcodes are used. Barcoded primers or adapters may comprise unique molecular identifiers (UMI). Such UMIs in some instances uniquely tag all nucleic acids in a sample. In some instances, at least 60%, 70%, 80%, 90%, 95%, or more than 95% of the nucleic acids in a sample are tagged with a UML. In some instances, at least 85%, 90%, 95%, 97%, or at least 99% of the nucleic acids in a sample are tagged with a unique barcode, or UML. Barcoded primers in some instances comprise an index sequence and one or more UMI. UMIs allow for internal measurement of initial sample concentrations or stoichiometry prior to downstream sample processing (e.g., PCR or enrichment steps) which can introduce bias. In some instances, UMIs comprise one or more barcode sequences. In some instances, each strand (forward vs. reverse) of an adapter-ligated sample polynucleotide possesses one or more unique barcodes. Such barcodes are optionally used to uniquely tag each strand of a sample polynucleotide. In some instances, a barcoded primer comprises an index barcode and a UMI barcode. In some instances, after amplification with at least two barcoded primers, the resulting amplicons comprise two index sequences and two UMIs. In some instances, after amplification with at least two barcoded primers, the resulting amplicons comprise two index barcodes and one UMI barcode. In some instances, each strand of a universal adapter-sample polynucleotide duplex is tagged with a unique barcode, such as a UMI or index barcode.

Barcoded primers in a library comprise a region that is complementary to a primer binding region on a universal adapter. For example, universal adapter binding region is complementary to primer region of the universal adapter, and universal adapter binding region is complementary to primer region of the universal adapter. Such arrangements facilitate extension of universal adapters during PCR, and attach barcoded primers. In some instances, the Tₘ between the primer and the primer binding region is 40-65 degrees C. In some instances, the Tₘ between the primer and the primer binding region is 42-63 degrees C. In some instances, the Tₘ between the primer and the primer binding region is 50-60 degrees C. In some instances, the Tₘ between the primer and the primer binding region is 53-62 degrees C. In some instances, the Tₘ between the primer and the primer binding region is 54-58 degrees C. In some instances, the Tₘ between the primer and the primer binding region is 40-57 degrees C. In some instances, the Tₘ between the primer and the primer binding region is 40-50 degrees C. In some instances, the Tₘ between the primer and the primer binding region is about 40, 45, 47, 50, 52, 53, 55, 57, 59, 61, or 62 degrees C.

Hybridization Blockers

Blockers may contain any number of different nucleobases (DNA, RNA, etc.), nucleobase analogues (non-canonical), or non-nucleobase linkers or spacers. In some instances, blockers comprise universal blockers. Such blockers may in some instances are described as a “set”, wherein the set comprises two or more blockers configured to prevent unwanted interactions with the same adapter sequence. In some instances, universal blockers prevent adapter-adapter interactions independent of one or more barcodes present on at least one of the adapters. For example, a blocker comprises one or more nucleobase analogues or other groups that enhance hybridization (Tₘ) between the blocker and the adapter. In some instances, a blocker comprises one or more nucleobases which decrease hybridization (Tₘ) between the blocker and the adapter (e.g., “universal” bases). In some instances, a blocker described herein comprises both one or more nucleobases which increase hybridization (Tₘ) between the blocker and the adapter and one or more nucleobases which decrease hybridization (Tₘ) between the blocker and the adapter.

Described herein are hybridization blockers comprising one or more regions which enhance binding to targeted sequences (e.g., adapter), and one or more regions which decrease binding to target sequences (e.g., adapter). In some instances, each region is tuned for a given desired level of off-bait activity during target enrichment applications. In some instances, each region can be altered with either a single type of chemical modification/moiety or multiple types to increase or decrease overall affinity of a molecule for a targeted sequence. In some instances, the melting temperature of all individual members of a blocker set are held above a specified temperature (e.g., with the addition of moieties such as LNAs and/or BNAs). In some instances, a given set of blockers will improve off bait performance independent of index length, independent of index sequence, and independent of how many adapter indices are present in hybridization.

Blockers may comprise moieties which increase and/or decrease affinity for a target sequencing, such as an adapter. In some instances, such specific regions can be thermodynamically tuned to specific melting temperatures to either avoid or increase the affinity for a particular targeted sequence. This combination of modifications is in some instances designed to help increase the affinity of the blocker molecule for specific and unique adapter sequence and decrease the affinity of the blocker molecule for repeated adapter sequence (e.g., Y-stem annealing portion of adapter). In some instances, blockers comprise moieties which decrease binding of a blocker to the Y-stem region of an adapter. In some instances, blockers comprise moieties which decrease binding of a blocker to the Y-stem region of an adapter, and moieties which increase binding of a blocker to non-Y-stem regions of an adapter.

Blockers (e.g., universal blockers) and adapters may form a number of different populations during hybridization. In a population ‘A’ in some instances comprises blockers correctly bound to non-index regions of the adapters. In a population ‘B’, a region of the blockers is bound to the “yoke” region of the adapter, but a remaining portion of the blocker does not bind to an adjacent region of the adapter. In a population ‘C’, two blockers unproductively dimerize. In a population ‘D’, blockers are unbound to any other nucleic acids. In some instances, when the number of DNA modifications that decrease affinity in the Y-stem annealing region of the blocker are increased, the populations ‘A’ & ‘D’ dominate and either have the desired or minimal effect. In some instances, as the number of DNA modifications that decrease affinity in the Y-stem annealing region of the blocker are decreased, the populations ‘B’ & ‘C’ dominate and have undesired effects where daisy-chaining or annealing to other adapters can occur (‘B’) or sequester blockers where they are unable to function properly (‘C’).

The index on both single or dual index adapter designs may be either partially or fully covered by universal blockers that have been extended with specifically designed DNA modifications to cover adapter index bases. In some instances, such modifications comprise moieties which decrease annealing to the index, such as universal bases. In some instances, the index of a dual index adapter is partially covered (or is overlapped) by one or more blockers. In some instances, the index of a dual index adapter is fully covered by one or more blockers. In some instances, the index of a single index adapter is partially covered by one or more blockers. In some instances, the index of a single index adapter is fully covered by one or more blockers. In some instances, a blocker overlaps an index sequence by at least 1, 2, 3, 4, 5, 6, 7, 8, 9, 10, 11, 12, 13, 14, 15, 20 or more than 20 bases. In some instances, a blocker overlaps an index sequence by no more than 1, 2, 3, 4, 5, 6, 7, 8, 9, 10, 11, 12, 13, 14, 15, 20, or no more than 25 bases. In some instances, a blocker overlaps an index sequence by about 1, 2, 3, 4, 5, 6, 7, 8, 9, 10, 11, 12, 13, 14, 15, 20 or about 30 bases. In some instances, a blocker overlaps an index sequence by 1-5, 1-3, 2-5, 2-8, 2-10, 3-6, 3-10, 4-10, 4-15, 1-4 or 5-7 bases. In some instances, a region of a blocker which overlaps an index sequences comprises at least one 2-deoxyinosine or 5-nitroindole nucleobase.

One or two blockers may overlap with an index sequence present on an adapter. In some instances, one or two blockers combined overlap with at least 1, 2, 3, 4, 5, 6, 7, 8, 9, 10, 11, 12, 13, 14, 15, 20 or more than 20 bases of the index sequence. In some instances, one or two blockers combined overlap with no more than 1, 2, 3, 4, 5, 6, 7, 8, 9, 10, 11, 12, 13, 14, 15, 20 or no more than 20 bases of the index sequence. In some instances, one or two blockers combined overlap with about 1, 2, 3, 4, 5, 6, 7, 8, 9, 10, 11, 12, 13, 14, 15, 20 or about 20 bases of the index sequence. In some instances, one or two blockers combined overlap by 1-5, 1-3, 2-5, 2-8, 2-10, 3-6, 3-10, 4-10, 4-15, 1-4 or 5-7 bases of the index sequence. In some instances, a region of a blocker which overlaps an index sequences comprises at least one 2-deoxyinosine or 5-nitroindole nucleobase.

In a first arrangement, the length of the adapter index overhang may be varied. When designed from a single side, the adapter index overhang can be altered to cover from 0 to n of the adapter index bases from either side of the index. This allows for the ability to design such adapter blockers for both single and dual index adapter systems.

In a second arrangement, the adapter index bases are covered from both sides. When adapter index bases are covered from both sides, the length of the covering region of each blocker can be chosen such that a single pair of blockers is capable of interacting with a range of adapter index lengths while still covering a significant portion of the total number of index bases. As an example, take two blockers that have been designed with 3 bp overhangs that cover the adapter index. In the context of 6 bp, 8 bp, or 10 bp adapter index lengths, these blockers will leave 0 bp, 2 bp, or 4 bp exposed during hybridization, respectively.

In a third arrangement, modified nucleobases are selected to cover index adapter bases. Examples of these modifications that are currently commercially available include degenerate bases (i.e., mixed bases of A, T, C, G), 2′-deoxylnosine, & 5-nitroindole.

In a forth arrangement, blockers with adapter index overhangs bind to either the sense (i.e., ‘top’) or anti-sense (i.e., ‘bottom’) strand of a next generation sequencing library.

In a fifth arrangement, blockers are further extended to cover other polynucleotide sequences (e.g., a poly-A tail added in a previous biochemical step in order to facilitate ligation or other method to introduce a defined adapter sequence, unique molecular identifier for bioinformatic assignment following sequencing, etc.) in addition to the standard adapter index bases of defined length and composition. These types of sequences can be placed in multiple locations of an adapter and in this case the most widely utilized case (i.e., unique molecular index next to the genomic insert) is presented. Other positions for the unique molecular identifier (e.g., next to adapter index bases) could also be addressed with similar approaches.

In a sixth arrangement, all of the previous arrangements are utilized in various combinations to meet a targeted performance metric for off-bait performance during target enrichment under specified conditions.

Blockers may comprise moieties, such as nucleobase analogues. Nucleobase analogues and other groups include but are not limited to locked nucleic acids (LNAs), bicyclic nucleic acids (BNAs), C5-modified pyrimidine bases, 2′-O-methyl substituted RNA, peptide nucleic acids (PNAs), glycol nucleic acid (GNAs), threose nucleic acid (TNAs), inosine, 2′-deoxylnosine, 3-nitropyrrole, 5-nitroindole, xenonucleic acids (XNAs) morpholino backbone-modified bases, minor grove binders (MGBs), spermine, G-clamps, or a anthraquinone (Uaq) caps. In some instances, nucleobase analogues comprise universal bases, wherein the nucleobase has a lower Tₘ for binding to a cognate nucleobase. In some instances, universal bases comprise 5-nitroindole or 2′-deoxylnosine. In instances, blockers comprise spacer elements that connect two polynucleotide chains. In some instances, blockers comprise one or more nucleobase analogues. In some instances, such nucleobase analogues are added to control the Tₘ of a blocker. Blockers may comprise any number of nucleobase analogues (such as LNAs or BNAs), depending on the desired hybridization Tₘ. For example, a blocker comprises 20 to 40 nucleobase analogues. In some instances, a blocker comprises 8 to 16 nucleobase analogues. In some instances, a blocker comprises at least 1, 2, 3, 4, 5, 6, 7, 8, 9, 10, 11, 12, or at least 12 nucleobase analogues. In some instances, a blocker comprises about 1, 2, 3, 4, 5, 6, 7, 8, 9, 10, 11, 12, 13, 14, 15, or about 16 nucleobase analogues. In some instances, the number of nucleobase analogous is expressed as a percent of the total bases in the blocker. For example, a blocker comprises at least 1%, 2%, 5%, 10%, 12%, 18%, 24%, 30%, or more than 30% nucleobase analogues. In some instances, the blocker comprising a nucleobase analogue raises the Tₘ in a range of about 2° C. to about 8° C. for each nucleobase analogue. In some instances, the Tₘ is raised by at least or about 1° C., 2° C., 3° C., 4° C., 5° C., 6° C., 7° C., 8° C., 9° C., 10° C., 12° C., 14° C., or 16° C. for each nucleobase analogue. Such blockers in some instances are configured to bind to the top or “sense” strand of an adapter. Blockers in some instances are configured to bind to the bottom or “anti-sense” strand of an adapter. In some instances a set of blockers includes sequences which are configured to bind to both top and bottom strands of an adapter. Additional blockers in some instances are configured to the complement, reverse, forward, or reverse complement of an adapter sequence. In some instances, a set of blockers targeting a top (binding to the top) or bottom strand (or both) is designed and tested, followed by optimization, such as replacing a top blocker with a bottom blocker, or a bottom blocker with a top blocker. In some instances, a blocker is configured to overlap fully or partially with bases of an index or barcode on an adapter. A set of blockers in some instances comprise at least one blocker overlapping with an adapter index sequence. A set of blockers in some instances comprise at least one blocker overlapping with an adapter index sequence, and at least one blocker which does not overlap with an adapter sequence. A set of blockers in some instances comprise at least one blocker which does not overlap with a yoke region sequence. A set of blockers in some instances comprise at least one blocker which does not overlap with a yoke region sequence and at least one blocker which overlaps with a yoke region sequence. A sets of blockers in some instances comprises 2, 3, 4, 5, 6, 7, 8, 9, 10, or more than 10 blockers.

Blockers may be any length, depending on the size of the adapter or hybridization Tₘ. For example, blockers are 20 to 50 bases in length. In some instances, blockers are 25 to 45 bases, 30 to 40 bases, 20 to 40 bases, or 30 to 50 bases in length. In some instances, blockers are 25 to 35 bases in length. In some instances blockers are at least 25, 26, 27, 28, 29, 30, 31, 32, 33, 34, or at least 35 bases in length. In some instances, blockers are no more than 25, 26, 27, 28, 29, 30, 31, 32, 33, 34, or no more than 35 bases in length. In some instances, blockers are about 25, 26, 27, 28, 29, 30, 31, 32, 33, 34, or about 35 bases in length. In some instances, blockers are about 50 bases in length. A set of blockers targeting an adapter-tagged genomic library fragment in some instances comprises blockers of more than one length. Two blockers are in some instances tethered together with a linker. Various linkers are well known in the art, and in some instances comprise alkyl groups, polyether groups, amine groups, amide groups, or other chemical group. In some instances, linkers comprise individual linker units, which are connected together (or attached to blocker polynucleotides) through a backbone such as phosphate, thiophosphate, amide, or other backbone. In an exemplary arrangement, a linker spans the index region between a first blocker that each targets the 5′ end of the adapter sequence and a second blocker that targets the 3′ end of the adapter sequence. In some instances, capping groups are added to the 5′ or 3′ end of the blocker to prevent downstream amplification. Capping groups variously comprise polyethers, polyalcohols, alkanes, or other non-hybridizable group that prevents amplification. Such groups are in some instances connected through phosphate, thiophosphate, amide, or other backbone. In some instances, one or more blockers are used. In some instances, at least 4 non-identical blockers are used. In some instances, a first blocker spans a first 3′ end of an adaptor sequence, a second blocker spans a first 5′ end of an adaptor sequence, a third blocker spans a second 3′ end of an adaptor sequence, and a fourth blockers spans a second 5′ end of an adaptor sequence. In some instances a first blocker is at least 20, 21, 22, 23, 24, 25, 26, 27, 28, 29, 30, 31, 32, 33, 34, or at least 35 bases in length. In some instances a second blocker is at least 20, 21, 22, 23, 24, 25, 26, 27, 28, 29, 30, 31, 32, 33, 34, or at least 35 bases in length. In some instances a third blocker is at least 20, 21, 22, 23, 24, 25, 26, 27, 28, 29, 30, 31, 32, 33, 34, or at least 35 bases in length. In some instances a fourth blocker is at least 20, 21, 22, 23, 24, 25, 26, 27, 28, 29, 30, 31, 32, 33, 34, or at least 35 bases in length. In some instances, a first blocker, second blocker, third blocker, or fourth blocker comprises a nucleobase analogue. In some instances, the nucleobase analogue is LNA.

The design of blockers may be influenced by the desired hybridization Tₘ to the adapter sequence. In some instances, non-canonical nucleic acids (for example locked nucleic acids, bridged nucleic acids, or other non-canonical nucleic acid or analog) are inserted into blockers to increase or decrease the blocker's Tₘ. In some instances, the Tₘ of a blocker is calculated using a tool specific to calculating Tₘ for polynucleotides comprising a non-canonical amino acid. In some instances, a Tₘ is calculated using the Exiqon™ online prediction tool. In some instances, blocker Tₘ described herein are calculated in-silico. In some instances, the blocker Tₘ is calculated in-silico, and is correlated to experimental in-vitro conditions. Without being bound by theory, an experimentally determined Tₘ may be further influenced by experimental parameters such as salt concentration, temperature, presence of additives, or other factor. In some instances, Tₘ described herein are in-silico determined Tₘ that are used to design or optimize blocker performance. In some instances, Tₘ values are predicted, estimated, or determined from melting curve analysis experiments. In some instances, blockers have a Tₘ of 70 degrees C. to 99 degrees C. In some instances, blockers have a Tₘ of 75 degrees C. to 90 degrees C. In some instances, blockers have a Tₘ of at least 85 degrees C. In some instances, blockers have a Tₘ of at least 70, 72, 75, 77, 80, 82, 85, 88, 90, or at least 92 degrees C. In some instances, blockers have a Tₘ of about 70, 72, 75, 77, 80, 82, 85, 88, 90, 92, or about 95 degrees C. In some instances, blockers have a Tₘ of 78 degrees C. to 90 degrees C. In some instances, blockers have a Tₘ of 79 degrees C. to 90 degrees C. In some instances, blockers have a Tₘ of 80 degrees C. to 90 degrees C. In some instances, blockers have a Tₘ of 81 degrees C. to 90 degrees C. In some instances, blockers have a Tₘ of 82 degrees C. to 90 degrees C. In some instances, blockers have a Tₘ of 83 degrees C. to 90 degrees C. In some instances, blockers have a Tₘ of 84 degrees C. to 90 degrees C. In some instances, a set of blockers have an average Tₘ of 78 degrees C. to 90 degrees C. In some instances, a set of blockers have an average Tₘ of 80 degrees C. to 90 degrees C. In some instances, a set of blockers have an average Tₘ of at least 80 degrees C. In some instances, a set of blockers have an average Tₘ of at least 81 degrees C. In some instances, a set of blockers have an average Tₘ of at least 82 degrees C. In some instances, a set of blockers have an average Tₘ of at least 83 degrees C. In some instances, a set of blockers have an average Tₘ of at least 84 degrees C. In some instances, a set of blockers have an average Tₘ of at least 86 degrees C. Blocker Tₘ are in some instances modified as a result of other components described herein, such as use of a fast hybridization buffer and/or hybridization enhancer.

The molar ratio of blockers to adapter targets may influence the off-bait (and subsequently off-target) rates during hybridization. The more efficient a blocker is at binding to the target adapter, the less blocker is required. Blockers described herein in some instances achieve sequencing outcomes of no more than 20% off-target reads with a molar ratio of less than 20:1 (blocker:target). In some instances, no more than 20% off-target reads are achieved with a molar ratio of less than 10:1 (blocker:target). In some instances, no more than 20% off-target reads are achieved with a molar ratio of less than 5:1 (blocker:target). In some instances, no more than 20% off-target reads are achieved with a molar ratio of less than 2:1 (blocker:target). In some instances, no more than 20% off-target reads are achieved with a molar ratio of less than 1.5:1 (blocker:target). In some instances, no more than 20% off-target reads are achieved with a molar ratio of less than 1.2:1 (blocker:target). In some instances, no more than 20% off-target reads are achieved with a molar ratio of less than 1.05:1 (blocker:target).

The universal blockers may be used with panel libraries of varying size. In some embodiments, the panel libraries comprises at least or about 0.01, 0.02, 0.03, 0.04, 0.05, 0.06, 0.07, 0.08, 0.09, 1.0, 2.0, 4.0, 8.0, 10.0, 12.0, 14.0, 16.0, 18.0, 20.0, 22.0, 24.0, 26.0, 28.0, 30.0, 40.0, 50.0, 60.0, or more than 60.0 megabases (Mb).

Blockers as described herein may improve on-target performance. In some embodiments, on-target performance is improved by at least or about 5%, 10%, 15%, 20%, 25%, 30%, 35%, 40%, 45%, 50%, 55%, 60%, 65%, 70%, 75%, 80%, 85%, 90%, 95%, or more than 95%. In some embodiments, the on-target performance is improved by at least or about 5%, 10%, 15%, 20%, 25%, 30%, 35%, 40%, 45%, 50%, 55%, 60%, 65%, 70%, 75%, 80%, 85%, 90%, 95%, or more than 95% for various index designs. In some embodiments, the on-target performance is improved by at least or about 5%, 10%, 15%, 20%, 25%, 30%, 35%, 40%, 45%, 50%, 55%, 60%, 65%, 70%, 75%, 80%, 85%, 90%, 95%, or more than 95% is improved for various panel sizes.

De Novo Synthesis of Small Polynucleotide Populations for Amplification Reactions

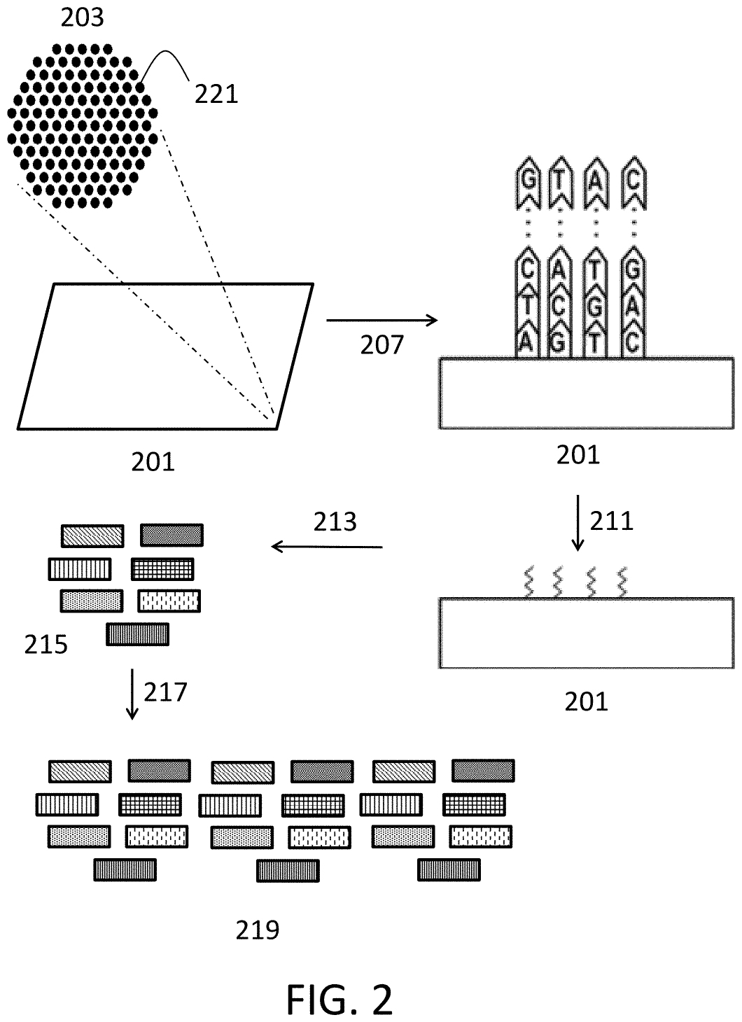

Described herein are methods of synthesis of polynucleotides from a surface, e.g., a plate (FIG. 2). In some instances, polynucleotide libraries comprise sample polynucleotide libraries. In some instances, the polynucleotides are synthesized on a cluster of loci for polynucleotide extension, released and then subsequently subjected to an amplification reaction, e.g., PCR. An exemplary workflow of synthesis of polynucleotides from a cluster is depicted in FIG. 2. A silicon plate 201 includes multiple clusters 203. Within each cluster are multiple loci 221. Polynucleotides are synthesized 207 de novo on a plate 201 from the cluster 203. Polynucleotides are cleaved 211 and removed 213 from the plate to form a population of released polynucleotides 215. The population of released polynucleotides 215 is then amplified 217 to form a library of amplified polynucleotides 219.

Provided herein are methods where amplification of polynucleotides synthesized on a cluster provide for enhanced control over polynucleotide representation compared to amplification of polynucleotides across an entire surface of a structure without such a clustered arrangement. In some instances, amplification of polynucleotides synthesized from a surface having a clustered arrangement of loci for polynucleotides extension provides for overcoming the negative effects on representation due to repeated synthesis of large polynucleotide populations. Exemplary negative effects on representation due to repeated synthesis of large polynucleotide populations include, without limitation, amplification bias resulting from high/low GC content, repeating sequences, trailing adenines, secondary structure, affinity for target sequence binding, or modified nucleotides in the polynucleotide sequence.

Cluster amplification as opposed to amplification of polynucleotides across an entire plate without a clustered arrangement can result in a tighter distribution around the mean. For example, if 100,000 reads are randomly sampled, an average of 8 reads per sequence would yield a library with a distribution of about 1.5× from the mean. In some cases, single cluster amplification results in at most about 1.5×, 1.6×, 1.7×, 1.8×, 1.9×, or 2.0× from the mean. In some cases, single cluster amplification results in at least about 1.0×, 1.2×, 1.3×, 1.5×1.6×, 1.7×, 1.8×, 1.9×, or 2.0× from the mean.

Cluster amplification methods described herein when compared to amplification across a plate can result in a polynucleotide library that requires less sequencing for equivalent sequence representation. In some instances at least 10%, at least 20%, at least 30%, at least 40%, at least 50%, at least 60%, at least 70%, at least 80%, at least 90%, or at least 95% less sequencing is required. In some instances up to 10%, up to 20%, up to 30%, up to 40%, up to 50%, up to 60%, up to 70%, up to 80%, up to 90%, or up to 95% less sequencing is required. Sometimes 30% less sequencing is required following cluster amplification compared to amplification across a plate. Sequencing of polynucleotides in some instances is verified by high-throughput sequencing such as by next generation sequencing. Sequencing of the sequencing library can be performed with any appropriate sequencing technology, including but not limited to single-molecule real-time (SMRT) sequencing, polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. The number of times a single nucleotide or polynucleotide is identified or “read” is defined as the sequencing depth or read depth. In some cases, the read depth is referred to as a fold coverage, for example, 55 fold (or 55×) coverage, optionally describing a percentage of bases.

In some instances, amplification from a clustered arrangement compared to amplification across a plate results in less dropouts, or sequences which are not detected after sequencing of amplification product. Dropouts can be of AT and/or GC. In some instances, a number of dropouts are at most about 1%, 2%, 3%, 4%, or 5% of a polynucleotide population. In some cases, the number of dropouts is zero.

A cluster as described herein comprises a collection of discrete, non-overlapping loci for polynucleotide synthesis. A cluster can comprise about 50-1000, 75-900, 100-800, 125-700, 150-600, 200-500, or 300-400 loci. In some instances, each cluster includes 121 loci. In some instances, each cluster includes about 50-500, 50-200, 100-150 loci. In some instances, each cluster includes at least about 50, 100, 150, 200, 500, 1000 or more loci. In some instances, a single plate includes 100, 500, 10000, 20000, 30000, 50000, 100000, 500000, 700000, 1000000 or more loci. A locus can be a spot, well, microwell, channel, or post. In some instances, each cluster has at least 1×, 2×, 3×, 4×, 5×, 6×, 7×, 8×, 9×, 10×, or more redundancy of separate features supporting extension of polynucleotides having identical sequence.

Generation of Polynucleotide Libraries with Controlled Stoichiometry of Sequence Content

In some instances, the polynucleotide library (such as a sample polynucleotide set for variant detection) is synthesized with a specified distribution of desired polynucleotide sequences. In some instances, adjusting polynucleotide libraries for enrichment of specific desired sequences results in improved downstream application outcomes.

One or more specific sequences can be selected based on their evaluation in a downstream application. In some instances, the evaluation is binding affinity to target sequences for amplification, enrichment, or detection, stability, melting temperature, biological activity, ability to assemble into larger fragments, or other property of polynucleotides. In some instances, the evaluation is empirical or predicted from prior experiments and/or computer algorithms. An exemplary application includes increasing sequences in a probe library which correspond to areas of a genomic target having less than average read depth.

Selected sequences in a polynucleotide library can be at least 10%, 20%, 30%, 40%, 50%, 60%, 70%, 80%, 90%, 95%, or more than 95% of the sequences. In some instances, selected sequences in a polynucleotide library are at most 10%, 20%, 30%, 40%, 50%, 60%, 70%, 80%, 90%, 95%, or at most 100% of the sequences. In some cases, selected sequences are in a range of about 5-95%, 10-90%, 30-80%, 40-75%, or 50-70% of the sequences.

Polynucleotide libraries can be adjusted for the frequency of each selected sequence. In some instances, polynucleotide libraries favor a higher number of selected sequences. For example, a library is designed where increased polynucleotide frequency of selected sequences is in a range of about 40% to about 90%. In some instances, polynucleotide libraries contain a low number of selected sequences. For example, a library is designed where increased polynucleotide frequency of the selected sequences is in a range of about 10% to about 60%. A library can be designed to favor a higher and lower frequency of selected sequences. In some instances, a library favors uniform sequence representation. For example, polynucleotide frequency is uniform with regard to selected sequence frequency, in a range of about 10% to about 90%. In some instances, a library comprises polynucleotides with a selected sequence frequency of about 10% to about 95% of the sequences.

Generation of polynucleotide libraries with a specified selected sequence frequency in some cases occurs by combining at least 2 polynucleotide libraries with different selected sequence frequency content. In some instances, at least 2, 3, 4, 5, 6, 7, 10, or more than 10 polynucleotide libraries are combined to generate a population of polynucleotides with a specified selected sequence frequency. In some cases, no more than 2, 3, 4, 5, 6, 7, or 10 polynucleotide libraries are combined to generate a population of non-identical polynucleotides with a specified selected sequence frequency.

In some instances, selected sequence frequency is adjusted by synthesizing fewer or more polynucleotides per cluster. For example, at least 25, 50, 100, 200, 300, 400, 500, 600, 700, 800, 900, 1000, or more than 1000 non-identical polynucleotides are synthesized on a single cluster. In some cases, no more than about 50, 100, 200, 300, 400, 500, 600, 700, 800, 900, 1000 non-identical polynucleotides are synthesized on a single cluster. In some instances, 50 to 500 non-identical polynucleotides are synthesized on a single cluster. In some instances, 100 to 200 non-identical polynucleotides are synthesized on a single cluster. In some instances, about 100, about 120, about 125, about 130, about 150, about 175, or about 200 non-identical polynucleotides are synthesized on a single cluster.

In some cases, selected sequence frequency is adjusted by synthesizing non-identical polynucleotides of varying length. For example, the length of each of the non-identical polynucleotides synthesized may be at least or about at least 10, 15, 20, 25, 30, 35, 40, 45, 50, 100, 150, 200, 300, 400, 500, 2000 nucleotides, or more. The length of the non-identical polynucleotides synthesized may be at most or about at most 2000, 500, 400, 300, 200, 150, 100, 50, 45, 35, 30, 25, 20, 19, 18, 17, 16, 15, 14, 13, 12, 11, 10 nucleotides, or less. The length of each of the non-identical polynucleotides synthesized may fall from 10-2000, 10-500, 9-400, 11-300, 12-200, 13-150, 14-100, 15-50, 16-45, 17-40, 18-35, and 19-25.

Use of Polynucleotide Libraries as Standards

Provided herein are methods of using polynucleotide libraries to improve the sensitivity and accuracy of nucleic acid variant detection. In some instances, the method comprises preparing a nucleic acid sample useful for determining the detection limit of genomic variants. In some instances, the method comprises one or more of the steps of providing a polynucleotide library described herein (e.g., reference standard); obtaining at least one sample from a patient suspected of having a disease or condition; detecting the presence or absence of the one or more variants in the library; and detecting the presence or absence of the one or more variants in the at least one sample. In some instances, detecting comprises sequencing. In some instances, detecting comprises Next Generation Sequencing. In some instances, sequencing comprises sequencing by synthesis, nanopore sequencing, SMRT sequencing, or other sequencing method described herein. In some instances, detecting comprises ddPCR or specific hybridization to an array.

Samples (test samples) may be obtained from any source. In some instances, the source is a human. In some instances, the source is a human (or patient) suspected of having a disease or condition. In some instances, the test sample comprises a liquid biopsy. In some instances, the test sample comprises circulating tumor DNA (ctDNA). In some instances, the test sample comprises circulating tumor DNA (ctDNA). In some instances, the test sample is obtained from blood. In some instances, the test sample is substantially cell-free. In some instances, more than one test sample is analyzed sequentially or in parallel. In some instances, at least 1, 2, 3, 4, 5, 10, 20, 50, 100, 200, 500, 1000, or more than 2000 test samples are analyzed. In some instances, the method further comprises detection of minimal residual disease (MRD). In some instances, the patient is suspected of having a disease or condition. In some instances, the disease or condition is a proliferative disease. In some instances, the disease or condition is cancer. In some instances, the patient was previously treated, is currently treated, or has received a clinical diagnosis for cancer. In some instances, the method further comprises ligating sequencing adapters to at least some polynucleotides in the sample, the library, or both. In some instances, the method further comprises amplifying at least some polynucleotides in the sample, the library, or both. In some instances, if one or more variants are not detected in the library, then results obtained from the at least one sample is discarded or re-analyzed.

Kits

Provided herein are kits comprising libraries of polynucleotides. In some instances, a kit comprises one or more of a reference standards (controls), wherein the reference standard comprises a sample polynucleotide set and a background set; instructions for use of the kit contents; and packaging to hold and describe the kit contents. In some instances, a kit comprises at least two standards selected from sample polynucleotides having a VAF of 0%, 0.1% 0.25%, 0.5%, 1%, or 2% relative to a wild-type genomic sequence. In some instances, a kit comprises five standards each having a VAF of 0%, 0.1% 0.25%, 0.5%, 1%, or 2% relative to a wild-type genomic sequence. In some instances, kits comprise instructions of use of reference standards with one or more sequencing instruments or other instrument which is configured to measure genomic variants. In some instances, the reference standard is packaged in a buffer. In some instances, the reference standard is packaged in a tube. In some instances, the reference standard is not packaged in a plasma-like format. In some instances, the reference standard comprises 500 ng to 5 micrograms of total DNA.

Next Generation Sequencing Applications

Downstream applications of polynucleotide libraries (such as sample polynucleotide sets or reference standards) may include next generation sequencing. For example, enrichment of target sequences with a controlled stoichiometry polynucleotide probe library results in more efficient sequencing. The performance of a polynucleotide library for capturing or hybridizing to targets may be defined by a number of different metrics describing efficiency, accuracy, and precision. For example, Picard metrics comprise variables such as HS library size (the number of unique molecules in the library that correspond to target regions, calculated from read pairs), mean target coverage (the percentage of bases reaching a specific coverage level), depth of coverage (number of reads including a given nucleotide) fold enrichment (sequence reads mapping uniquely to the target/reads mapping to the total sample, multiplied by the total sample length/target length), percent off-bait bases (percent of bases not corresponding to bases of the probes/baits), percent off-target (percent of bases not corresponding to bases of interest), usable bases on target, AT or GC dropout rate, fold 80 base penalty (fold over-coverage needed to raise 80 percent of non-zero targets to the mean coverage level), percent zero coverage targets, PF reads (the number of reads passing a quality filter), percent selected bases (the sum of on-bait bases and near-bait bases divided by the total aligned bases), percent duplication, or other variable consistent with the specification.

Read depth (sequencing depth, or sampling) represents the total number of times a sequenced nucleic acid fragment (a “read”) is obtained for a sequence. Theoretical read depth is defined as the expected number of times the same nucleotide is read, assuming reads are perfectly distributed throughout an idealized genome. Read depth is expressed as function of % coverage (or coverage breadth). For example, 10 million reads of a 1 million base genome, perfectly distributed, theoretically results in 10× read depth of 100% of the sequences. In practice, a greater number of reads (higher theoretical read depth, or oversampling) may be needed to obtain the desired read depth for a percentage of the target sequences. Enrichment of target sequences with a controlled stoichiometry probe library increases the efficiency of downstream sequencing, as fewer total reads will be required to obtain an outcome with an acceptable number of reads over a desired % of target sequences. For example, in some instances 55× theoretical read depth of target sequences results in at least 30× coverage of at least 90% of the sequences. In some instances no more than 55× theoretical read depth of target sequences results in at least 30× read depth of at least 80% of the sequences. In some instances no more than 55× theoretical read depth of target sequences results in at least 30× read depth of at least 95% of the sequences. In some instances no more than 55× theoretical read depth of target sequences results in at least 10× read depth of at least 98% of the sequences. In some instances, 55× theoretical read depth of target sequences results in at least 20× read depth of at least 98% of the sequences. In some instances no more than 55× theoretical read depth of target sequences results in at least 5× read depth of at least 98% of the sequences. Increasing the concentration of probes during hybridization with targets can lead to an increase in read depth. In some instances, the concentration of probes is increased by at least 1.5×, 2.0×, 2.5×, 3×, 3.5×, 4×, 5×, or more than 5×. In some instances, increasing the probe concentration results in at least a 1000% increase, or a 20%, 30%, 40%, 50%, 60%, 70%, 80%, 90%, 100%, 200%, 300%, 500%, 750%, 1000%, or more than a 1000% increase in read depth. In some instances, increasing the probe concentration by 3× results in a 1000% increase in read depth. In some instances, sequencing is performed to achieve a theoretical read depth of at least 30×, 50×, 100×, 150×, 200×, 250×, 300×, 500×, or at least 1000×. In some instances, sequencing is performed to achieve a theoretical read depth of about 30×, 50×, 100×, 150×, 200×, 250×, 300×, 500×, or about 1000×. In some instances, sequencing is performed to achieve a theoretical read depth of no more than 30×, 50×, 100×, 150×, 200×, 250×, 300×, 500×, or no more than 1000×. In some instances, sequencing is performed to achieve an actual read depth of at least 30×, 50×, 100×, 150×, 200×, 250×, 300×, 500×, or at least 1000×. In some instances, sequencing is performed to achieve an actual read depth of no more than 30×, 50×, 100×, 150×, 200×, 250×, 300×, 500×, or no more than 1000×. In some instances, sequencing is performed to achieve an actual read depth of about 30×, 50×, 100×, 150×, 200×, 250×, 300×, 500×, or about 1000×.

On-target rate represents the percentage of sequencing reads that correspond with the desired target sequences. In some instances, a controlled stoichiometry polynucleotide probe library results in an on-target rate of at least 30%, or at least 35%, 40%, 45%, 50%, 55%, 60%, 65%, 70%, 75%, 80%, 85%, or at least 90%. Increasing the concentration of polynucleotide probes during contact with target nucleic acids leads to an increase in the on-target rate. In some instances, the concentration of probes is increased by at least 1.5×, 2.0×, 2.5×, 3×, 3.5×, 4×, 5×, or more than 5×. In some instances, increasing the probe concentration results in at least a 20% increase, or a 10%, 20%, 30%, 40%, 50%, 60%, 70%, 80%, 90%, 100%, 200%, 300%, or at least a 500% increase in on-target binding. In some instances, increasing the probe concentration by 3× results in a 20% increase in on-target rate.

Coverage uniformity is in some cases calculated as the read depth as a function of the target sequence identity. Higher coverage uniformity results in a lower number of sequencing reads needed to obtain the desired read depth. For example, a property of the target sequence may affect the read depth, for example, high or low GC or AT content, repeating sequences, trailing adenines, secondary structure, affinity for target sequence binding (for amplification, enrichment, or detection), stability, melting temperature, biological activity, ability to assemble into larger fragments, sequences containing modified nucleotides or nucleotide analogues, or any other property of polynucleotides. Enrichment of target sequences with controlled stoichiometry polynucleotide probe libraries results in higher coverage uniformity after sequencing. In some instances, 95% of the sequences have a read depth that is within 1× of the mean library read depth, or about 0.05, 0.1, 0.2, 0.5, 0.7, 1, 1.2, 1.5, 1.7 or about within 2× the mean library read depth. In some instances, 80%, 85%, 90%, 95%, 97%, or 99% of the sequences have a read depth that is within 1× of the mean.

Enrichment of Target Nucleic Acids with a Polynucleotide Probe Library

A probe library described herein may be used to enrich target polynucleotides present in a population of sample polynucleotides, for a variety of downstream applications. In one some instances, a sample is obtained from one or more sources, and the population of sample polynucleotides is isolated. Samples are obtained (by way of non-limiting example) from biological sources such as saliva, blood, tissue, skin, or completely synthetic sources. The plurality of polynucleotides obtained from the sample are fragmented, end-repaired, and adenylated to form a double stranded sample nucleic acid fragment. In some instances, end repair is accomplished by treatment with one or more enzymes, such as T4 DNA polymerase, klenow enzyme, and T4 polynucleotide kinase in an appropriate buffer. A nucleotide overhang to facilitate ligation to adapters is added, in some instances with 3′ to 5′ exo minus klenow fragment and dATP.

Adapters (such as universal adapters) may be ligated to both ends of the sample polynucleotide fragments with a ligase, such as T4 ligase, to produce a library of adapter-tagged polynucleotide strands, and the adapter-tagged polynucleotide library is amplified with primers, such as universal primers. In some instances, the adapters are Y-shaped adapters comprising one or more primer binding sites, one or more grafting regions, and one or more index (or barcode) regions. In some instances, the one or more index region is present on each strand of the adapter. In some instances, grafting regions are complementary to a flowcell surface, and facilitate next generation sequencing of sample libraries. In some instances, Y-shaped adapters comprise partially complementary sequences. In some instances, Y-shaped adapters comprise a single thymidine overhang which hybridizes to the overhanging adenine of the double stranded adapter-tagged polynucleotide strands. Y-shaped adapters may comprise modified nucleic acids, that are resistant to cleavage. For example, a phosphorothioate backbone is used to attach an overhanging thymidine to the 3′ end of the adapters. If universal primers are used, amplification of the library is performed to add barcoded primers to the adapters. A library of double stranded adapter-tagged polynucleotide strands is contacted with polynucleotide probes, to form hybrid pairs. Such pairs are separated from unhybridized fragments, and isolated from probes to produce an enriched library. The enriched library may then be sequenced.

The library of double stranded sample nucleic acid fragments is then denatured in the presence of adapter blockers. Adapter blockers minimize off-target hybridization of probes to the adapter sequences (instead of target sequences) present on the adapter-tagged polynucleotide strands, and/or prevent intermolecular hybridization of adapters (i.e., “daisy chaining”). Denaturation is carried out in some instances at 96° C., or at about 85, 87, 90, 92, 95, 97, 98 or about 99° C. A polynucleotide targeting library (probe library) is denatured in a hybridization solution, in some instances at 96° C., at about 85, 87, 90, 92, 95, 97, 98 or 99° C. The denatured adapter-tagged polynucleotide library and the hybridization solution are incubated for a suitable amount of time and at a suitable temperature to allow the probes to hybridize with their complementary target sequences. In some instances, a suitable hybridization temperature is about 45 to 80° C., or at least 45, 50, 55, 60, 65, 70, 75, 80, 85, or 90° C. In some instances, the hybridization temperature is 70° C. In some instances, a suitable hybridization time is 16 hours, or at least 4, 6, 8, 10, 12, 14, 16, 18, 20, 22, or more than 22 hours, or about 12 to 20 hours. Binding buffer is then added to the hybridized adapter-tagged-polynucleotide probes, and a solid support comprising a capture moiety is used to selectively bind the hybridized adapter-tagged polynucleotide-probes. The solid support is washed with buffer to remove unbound polynucleotides before an elution buffer is added to release the enriched, tagged polynucleotide fragments from the solid support. In some instances, the solid support is washed 2 times, or 1, 2, 3, 4, 5, or 6 times. The enriched library of adapter-tagged polynucleotide fragments is amplified and the enriched library is sequenced.

A plurality of nucleic acids (i.e. genomic sequence) may obtained from a sample, and fragmented, optionally end-repaired, and adenylated. Adapters are ligated to both ends of the polynucleotide fragments to produce a library of adapter-tagged polynucleotide strands, and the adapter-tagged polynucleotide library is amplified. The adapter-tagged polynucleotide library is then denatured at high temperature, preferably 96° C., in the presence of adapter blockers. A polynucleotide targeting library (probe library) is denatured in a hybridization solution at high temperature, preferably about 90 to 99° C., and combined with the denatured, tagged polynucleotide library in hybridization solution for about 10 to 24 hours at about 45 to 80° C. Binding buffer is then added to the hybridized tagged polynucleotide probes, and a solid support comprising a capture moiety are used to selectively bind the hybridized adapter-tagged polynucleotide-probes. The solid support is washed one or more times with buffer, preferably about 2 and 5 times to remove unbound polynucleotides before an elution buffer is added to release the enriched, adapter-tagged polynucleotide fragments from the solid support. The enriched library of adapter-tagged polynucleotide fragments is amplified and then the library is sequenced. Alternative variables such as incubation times, temperatures, reaction volumes/concentrations, number of washes, or other variables consistent with the specification are also employed in the method.

In any of the instances, the detection or quantification analysis of the oligonucleotides can be accomplished by sequencing. The subunits or entire synthesized oligonucleotides can be detected via full sequencing of all oligonucleotides by any suitable methods known in the art, e.g., Illumina sequencing by synthesis, PacBio nanopore sequencing, or BGI/MGI nanoball sequencing, including the sequencing methods described herein.

Sequencing can be accomplished through classic Sanger sequencing methods which are well known in the art. Sequencing can also be accomplished using high-throughput systems some of which allow detection of a sequenced nucleotide immediately after or upon its incorporation into a growing strand, i.e., detection of sequence in red time or substantially real time. In some cases, high throughput sequencing generates at least 1,000, at least 5,000, at least 10,000, at least 20,000, at least 30,000, at least 40,000, at least 50,000, at least 100,000 or at least 500,000 sequence reads per hour; with each read being at least 50, at least 60, at least 70, at least 80, at least 90, at least 100, at least 120 or at least 150 bases per read.

In some instances, high-throughput sequencing involves the use of technology available by Illumina's Genome Analyzer IIX, MiSeq personal sequencer, or HiSeq systems, such as those using HiSeq 2500, HiSeq 1500, HiSeq 2000, HiSeq 1000, iSeq 100, Mini Seq, MiSeq, NextSeq 550, NextSeq 2000, NextSeq 550, or NovaSeq 6000. These machines use reversible terminator-based sequencing by synthesis chemistry. These machines can generate 6000 Gb or more reads in 13-44 hours. Smaller systems may be utilized for runs within 3, 2, 1 days or less time. Short synthesis cycles may be used to minimize the time it takes to obtain sequencing results.

In some instances, high-throughput sequencing involves the use of technology available by ABI Solid System. This genetic analysis platform that enables massively parallel sequencing of clonally-amplified DNA fragments linked to beads. The sequencing methodology is based on sequential ligation with dye-labeled oligonucleotides.

The next generation sequencing can comprise ion semiconductor sequencing (e.g., using technology from Life Technologies (Ion Torrent)). Ion semiconductor sequencing can take advantage of the fact that when a nucleotide is incorporated into a strand of DNA, an ion can be released. To perform ion semiconductor sequencing, a high density array of micromachined wells can be formed. Each well can hold a single DNA template. Beneath the well can be an ion sensitive layer, and beneath the ion sensitive layer can be an ion sensor. When a nucleotide is added to a DNA, H+ can be released, which can be measured as a change in pH. The H+ ion can be converted to voltage and recorded by the semiconductor sensor. An array chip can be sequentially flooded with one nucleotide after another. No scanning, light, or cameras can be required. In some cases, an IONPROTON™ Sequencer is used to sequence nucleic acid. In some cases, an IONPGM™ Sequencer is used. The Ion Torrent Personal Genome Machine (PGM) can do 10 million reads in two hours.

In some instances, high-throughput sequencing involves the use of technology available by Helicos BioSciences Corporation (Cambridge, Mass.) such as the Single Molecule Sequencing by Synthesis (SMSS) method. SMSS is unique because it allows for sequencing the entire human genome in up to 24 hours. Finally, SMSS is powerful because, like the MW technology, it does not require a pre amplification step prior to hybridization. In fact, SMSS does not require any amplification.

In some instances, high-throughput sequencing involves the use of technology available by 454 Lifesciences, Inc. (Branford, Conn.) such as the Pico Titer Plate device which includes a fiber optic plate that transmits chemiluminescent signal generated by the sequencing reaction to be recorded by a CCD camera in the instrument. This use of fiber optics allows for the detection of a minimum of 20 million base pairs in 4.5 hours.

Methods for using bead amplification followed by fiber optics detection are described in Marguiles, M., et al. “Genome sequencing in microfabricated high-density picolitre reactors”, Nature, doi: 10.1038/nature03959.

In some instances, high-throughput sequencing is performed using Clonal Single Molecule Array (Solexa, Inc.) or sequencing-by-synthesis (SBS) utilizing reversible terminator chemistry. Constans, A., The Scientist 2003, 17(13):36. High-throughput sequencing of oligonucleotides can be achieved using any suitable sequencing method known in the art, such as those commercialized by Pacific Biosciences, Complete Genomics, Genia Technologies, Halcyon Molecular, Oxford Nanopore Technologies and the like. Overall such systems involve sequencing a target oligonucleotide molecule having a plurality of bases by the temporal addition of bases via a polymerization reaction that is measured on a molecule of oligonucleotide, i e., the activity of a nucleic acid polymerizing enzyme on the template oligonucleotide molecule to be sequenced is followed in real time. Sequence can then be deduced by identifying which base is being incorporated into the growing complementary strand of the target oligonucleotide by the catalytic activity of the nucleic acid polymerizing enzyme at each step in the sequence of base additions. A polymerase on the target oligonucleotide molecule complex is provided in a position suitable to move along the target oligonucleotide molecule and extend the oligonucleotide primer at an active site. A plurality of labeled types of nucleotide analogs are provided proximate to the active site, with each distinguishably type of nucleotide analog being complementary to a different nucleotide in the target oligonucleotide sequence. The growing oligonucleotide strand is extended by using the polymerase to add a nucleotide analog to the oligonucleotide strand at the active site, where the nucleotide analog being added is complementary to the nucleotide of the target oligonucleotide at the active site. The nucleotide analog added to the oligonucleotide primer as a result of the polymerizing step is identified. The steps of providing labeled nucleotide analogs, polymerizing the growing oligonucleotide strand, and identifying the added nucleotide analog are repeated so that the oligonucleotide strand is further extended and the sequence of the target oligonucleotide is determined.

The next generation sequencing technique can comprises real-time (SMRT™) technology by Pacific Biosciences. In SMRT, each of four DNA bases can be attached to one of four different fluorescent dyes. These dyes can be phospho linked. A single DNA polymerase can be immobilized with a single molecule of template single stranded DNA at the bottom of a zero-mode waveguide (ZMW). A ZMW can be a confinement structure which enables observation of incorporation of a single nucleotide by DNA polymerase against the background of fluorescent nucleotides that can rapidly diffuse in an out of the ZMW (in microseconds). It can take several milliseconds to incorporate a nucleotide into a growing strand. During this time, the fluorescent label can be excited and produce a fluorescent signal, and the fluorescent tag can be cleaved off. The ZMW can be illuminated from below. Attenuated light from an excitation beam can penetrate the lower 20-30 nm of each ZMW. A microscope with a detection limit of 20 zepto liters (10″ liters) can be created. The tiny detection volume can provide 1000-fold improvement in the reduction of background noise. Detection of the corresponding fluorescence of the dye can indicate which base was incorporated. The process can be repeated.

In some cases, the next generation sequencing is nanopore sequencing {See e.g., Soni G V and Meller A. (2007) Clin Chem 53: 1996-2001). A nanopore can be a small hole, of the order of about one nanometer in diameter. Immersion of a nanopore in a conducting fluid and application of a potential across it can result in a slight electrical current due to conduction of ions through the nanopore. The amount of current which flows can be sensitive to the size of the nanopore. As a DNA molecule passes through a nanopore, each nucleotide on the DNA molecule can obstruct the nanopore to a different degree. Thus, the change in the current passing through the nanopore as the DNA molecule passes through the nanopore can represent a reading of the DNA sequence. The nanopore sequencing technology can be from Oxford Nanopore Technologies; e.g., a GridION system. A single nanopore can be inserted in a polymer membrane across the top of a microwell. Each microwell can have an electrode for individual sensing. The microwells can be fabricated into an array chip, with 100,000 or more microwells (e.g., more than 200,000, 300,000, 400,000, 500,000, 600,000, 700,000, 800,000, 900,000, or 1,000,000) per chip. An instrument (or node) can be used to analyze the chip. Data can be analyzed in real-time. One or more instruments can be operated at a time. The nanopore can be a protein nanopore, e.g., the protein alpha-hemolysin, a heptameric protein pore. The nanopore can be a solid-state nanopore made, e.g., a nanometer sized hole formed in a synthetic membrane (e.g., SiNₓ, or SiO₂). The nanopore can be a hybrid pore (e.g., an integration of a protein pore into a solid-state membrane). The nanopore can be a nanopore with an integrated sensors (e.g., tunneling electrode detectors, capacitive detectors, or graphene based nano-gap or edge state detectors (see e.g., Garaj et al. (2010) Nature vol. 67, doi: 10.1038/nature09379)). A nanopore can be functionalized for analyzing a specific type of molecule (e.g., DNA, RNA, or protein). Nanopore sequencing can comprise “strand sequencing” in which intact DNA polymers can be passed through a protein nanopore with sequencing in real time as the DNA translocates the pore. An enzyme can separate strands of a double stranded DNA and feed a strand through a nanopore. The DNA can have a hairpin at one end, and the system can read both strands. In some cases, nanopore sequencing is “exonuclease sequencing” in which individual nucleotides can be cleaved from a DNA strand by a processive exonuclease, and the nucleotides can be passed through a protein nanopore. The nucleotides can transiently bind to a molecule in the pore (e.g., cyclodextran). A characteristic disruption in current can be used to identify bases.

Nanopore sequencing technology from GENIA can be used. An engineered protein pore can be embedded in a lipid bilayer membrane. “Active Control” technology can be used to enable efficient nanopore-membrane assembly and control of DNA movement through the channel. In some cases, the nanopore sequencing technology is from NABsys. Genomic DNA can be fragmented into strands of average length of about 100 kb. The 100 kb fragments can be made single stranded and subsequently hybridized with a 6-mer probe. The genomic fragments with probes can be driven through a nanopore, which can create a current-versus-time tracing. The current tracing can provide the positions of the probes on each genomic fragment. The genomic fragments can be lined up to create a probe map for the genome. The process can be done in parallel for a library of probes. A genome-length probe map for each probe can be generated. Errors can be fixed with a process termed “moving window Sequencing By Hybridization (mwSBH).” In some cases, the nanopore sequencing technology is from IBM/Roche. An electron beam can be used to make a nanopore sized opening in a microchip. An electrical field can be used to pull or thread DNA through the nanopore. A DNA transistor device in the nanopore can comprise alternating nanometer sized layers of metal and dielectric. Discrete charges in the DNA backbone can get trapped by electrical fields inside the DNA nanopore. Turning off and on gate voltages can allow the DNA sequence to be read.

The next generation sequencing can comprise DNA nanoball sequencing (as performed, e.g., by Complete Genomics; see e.g., Drmanac et al. (2010) Science 327: 78-81). DNA can be isolated, fragmented, and size selected. For example, DNA can be fragmented (e.g., by sonication) to a mean length of about 500 bp. Adaptors (Adl) can be attached to the ends of the fragments. The adaptors can be used to hybridize to anchors for sequencing reactions. DNA with adaptors bound to each end can be PCR amplified. The adaptor sequences can be modified so that complementary single strand ends bind to each other forming circular DNA. The DNA can be methylated to protect it from cleavage by a type IIS restriction enzyme used in a subsequent step. An adaptor (e.g., the right adaptor) can have a restriction recognition site, and the restriction recognition site can remain non-methylated. The non-methylated restriction recognition site in the adaptor can be recognized by a restriction enzyme (e.g., Acul), and the DNA can be cleaved by Acul 13 bp to the right of the right adaptor to form linear double stranded DNA. A second round of right and left adaptors (Ad2) can be ligated onto either end of the linear DNA, and all DNA with both adapters bound can be PCR amplified (e.g., by PCR). Ad2 sequences can be modified to allow them to bind each other and form circular DNA. The DNA can be methylated, but a restriction enzyme recognition site can remain non-methylated on the left Ad1 adapter. A restriction enzyme (e.g., Acul) can be applied, and the DNA can be cleaved 13 bp to the left of the Ad1 to form a linear DNA fragment. A third round of right and left adaptor (Ad3) can be ligated to the right and left flank of the linear DNA, and the resulting fragment can be PCR amplified. The adaptors can be modified so that they can bind to each other and form circular DNA. A type III restriction enzyme (e.g., EcoP15) can be added; EcoP15 can cleave the DNA 26 bp to the left of Ad3 and 26 bp to the right of Ad2. This cleavage can remove a large segment of DNA and linearize the DNA once again. A fourth round of right and left adaptors (Ad4) can be ligated to the DNA, the DNA can be amplified (e.g., by PCR), and modified so that they bind each other and form the completed circular DNA template.

Rolling circle replication (e.g., using Phi 29 DNA polymerase) can be used to amplify small fragments of DNA. The four adaptor sequences can contain palindromic sequences that can hybridize and a single strand can fold onto itself to form a DNA nanoball (DNB™) which can be approximately 200-300 nanometers in diameter on average. A DNA nanoball can be attached (e.g., by adsorption) to a microarray (sequencing flowcell). The flow cell can be a silicon wafer coated with silicon dioxide, titanium and hexamethyldisilazane (HMDS) and a photoresist material. Sequencing can be performed by unchained sequencing by ligating fluorescent probes to the DNA. The color of the fluorescence of an interrogated position can be visualized by a high resolution camera. The identity of nucleotide sequences between adaptor sequences can be determined.

A population of polynucleotides may be enriched prior to adapter ligation. In one example, a plurality of polynucleotides is obtained from a sample, fragmented, optionally end-repaired, and denatured at high temperature, preferably 90-99° C. A polynucleotide targeting library (probe library) is denatured in a hybridization solution at high temperature, preferably about 90 to 99° C., and combined with the denatured, tagged polynucleotide library in hybridization solution for about 10 to 24 hours at about 45 to 80° C. Binding buffer is then added to the hybridized tagged polynucleotide probes, and a solid support comprising a capture moiety are used to selectively bind the hybridized adapter-tagged polynucleotide-probes. The solid support is washed one or more times with buffer, preferably about 2 and 5 times to remove unbound polynucleotides before an elution buffer is added to release the enriched, adapter-tagged polynucleotide fragments from the solid support. The enriched polynucleotide fragments are then polyadenylated, adapters are ligated to both ends of the polynucleotide fragments to produce a library of adapter-tagged polynucleotide strands, and the adapter-tagged polynucleotide library is amplified. The adapter-tagged polynucleotide library is then sequenced.

A polynucleotide targeting library may also be used to filter undesired sequences from a plurality of polynucleotides, by hybridizing to undesired fragments. For example, a plurality of polynucleotides is obtained from a sample, and fragmented, optionally end-repaired, and adenylated. Adapters are ligated to both ends of the polynucleotide fragments to produce a library of adapter-tagged polynucleotide strands, and the adapter-tagged polynucleotide library is amplified. Alternatively, adenylation and adapter ligation steps are instead performed after enrichment of the sample polynucleotides. The adapter-tagged polynucleotide library is then denatured at high temperature, preferably 90-99° C., in the presence of adapter blockers. A polynucleotide filtering library (probe library) designed to remove undesired, non-target sequences is denatured in a hybridization solution at high temperature, preferably about 90 to 99° C., and combined with the denatured, tagged polynucleotide library in hybridization solution for about 10 to 24 hours at about 45 to 80° C. Binding buffer is then added to the hybridized tagged polynucleotide probes, and a solid support comprising a capture moiety are used to selectively bind the hybridized adapter-tagged polynucleotide-probes. The solid support is washed one or more times with buffer, preferably about 1 and 5 times to elute unbound adapter-tagged polynucleotide fragments. The enriched library of unbound adapter-tagged polynucleotide fragments is amplified and then the amplified library is sequenced.

Highly Parallel De Novo Nucleic Acid Synthesis

Described herein is a platform approach utilizing miniaturization, parallelization, and vertical integration of the end-to-end process from polynucleotide synthesis to gene assembly within Nano wells on silicon to create a revolutionary synthesis platform. Devices described herein provide, with the same footprint as a 96-well plate, a silicon synthesis platform is capable of increasing throughput by a factor of 100 to 1,000 compared to traditional synthesis methods, with production of up to approximately 1,000,000 polynucleotides in a single highly-parallelized run. In some instances, a single silicon plate described herein provides for synthesis of about 6,100 non-identical polynucleotides. In some instances, each of the non-identical polynucleotides is located within a cluster. A cluster may comprise 50 to 500 non-identical polynucleotides.

Methods described herein provide for synthesis of a library of polynucleotides each encoding for a predetermined variant of at least one predetermined reference nucleic acid sequence. In some cases, the predetermined reference sequence is nucleic acid sequence encoding for a protein, and the variant library comprises sequences encoding for variation of at least a single codon such that a plurality of different variants of a single residue in the subsequent protein encoded by the synthesized nucleic acid are generated by standard translation processes. The synthesized specific alterations in the nucleic acid sequence can be introduced by incorporating nucleotide changes into overlapping or blunt ended polynucleotide primers. Alternatively, a population of polynucleotides may collectively encode for a long nucleic acid (e.g., a gene) and variants thereof. In this arrangement, the population of polynucleotides can be hybridized and subject to standard molecular biology techniques to form the long nucleic acid (e.g., a gene) and variants thereof. When the long nucleic acid (e.g., a gene) and variants thereof are expressed in cells, a variant protein library is generated. Similarly, provided here are methods for synthesis of variant libraries encoding for RNA sequences (e.g., miRNA, shRNA, and mRNA) or DNA sequences (e.g., enhancer, promoter, UTR, and terminator regions). Also provided here are downstream applications for variants selected out of the libraries synthesized using methods described here. Downstream applications include identification of variant nucleic acid or protein sequences with enhanced biologically relevant functions, e.g., biochemical affinity, enzymatic activity, changes in cellular activity, and for the treatment or prevention of a disease state.

Substrates

Provided herein are substrates comprising a plurality of clusters, wherein each cluster comprises a plurality of loci that support the attachment and synthesis of polynucleotides. The term “locus” as used herein refers to a discrete region on a structure which provides support for polynucleotides encoding for a single predetermined sequence to extend from the surface. In some instances, a locus is on a two dimensional surface, e.g., a substantially planar surface. In some instances, a locus refers to a discrete raised or lowered site on a surface e.g., a well, micro well, channel, or post. In some instances, a surface of a locus comprises a material that is actively functionalized to attach to at least one nucleotide for polynucleotide synthesis, or preferably, a population of identical nucleotides for synthesis of a population of polynucleotides. In some instances, polynucleotide refers to a population of polynucleotides encoding for the same nucleic acid sequence. In some instances, a surface of a device is inclusive of one or a plurality of surfaces of a substrate.

Provided herein are structures that may comprise a surface that supports the synthesis of a plurality of polynucleotides having different predetermined sequences at addressable locations on a common support. In some instances, a device provides support for the synthesis of more than 2,000; 5,000; 10,000; 20,000; 30,000; 50,000; 75,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more non-identical polynucleotides. In some instances, the device provides support for the synthesis of more than 2,000; 5,000; 10,000; 20,000; 30,000; 50,000; 75,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more polynucleotides encoding for distinct sequences. In some instances, at least a portion of the polynucleotides have an identical sequence or are configured to be synthesized with an identical sequence.

Provided herein are methods and devices for manufacture and growth of polynucleotides about 5, 10, 20, 30, 40, 50, 60, 70, 80, 90, 100, 125, 150, 175, 200, 225, 250, 275, 300, 325, 350, 375, 400, 425, 450, 475, 500, 600, 700, 800, 900, 1000, 1100, 1200, 1300, 1400, 1500, 1600, 1700, 1800, 1900, or 2000 bases in length. In some instances, the length of the polynucleotide formed is about 5, 10, 20, 30, 40, 50, 60, 70, 80, 90, 100, 125, 150, 175, 200, or 225 bases in length. A polynucleotide may be at least 5, 10, 20, 30, 40, 50, 60, 70, 80, 90, or 100 bases in length. A polynucleotide may be from 10 to 225 bases in length, from 12 to 100 bases in length, from 20 to 150 bases in length, from 20 to 130 bases in length, or from 30 to 100 bases in length.

In some instances, polynucleotides are synthesized on distinct loci of a substrate, wherein each locus supports the synthesis of a population of polynucleotides. In some instances, each locus supports the synthesis of a population of polynucleotides having a different sequence than a population of polynucleotides grown on another locus. In some instances, the loci of a device are located within a plurality of clusters. In some instances, a device comprises at least 10, 500, 1000, 2000, 3000, 4000, 5000, 6000, 7000, 8000, 9000, 10000, 11000, 12000, 13000, 14000, 15000, 20000, 30000, 40000, 50000 or more clusters. In some instances, a device comprises more than 2,000; 5,000; 10,000; 100,000; 200,000; 300,000; 400,000; 500,000; 600,000; 700,000; 800,000; 900,000; 1,000,000; 1,100,000; 1,200,000; 1,300,000; 1,400,000; 1,500,000; 1,600,000; 1,700,000; 1,800,000; 1,900,000; 2,000,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; or 10,000,000 or more distinct loci. In some instances, a device comprises about 10,000 distinct loci. The amount of loci within a single cluster is varied in different instances. In some instances, each cluster includes 1, 2, 3, 4, 5, 6, 7, 8, 9, 10, 20, 30, 40, 50, 60, 70, 80, 90, 100, 120, 130, 150, 200, 300, 400, 500, 1000 or more loci. In some instances, each cluster includes about 50-500 loci. In some instances, each cluster includes about 100-200 loci. In some instances, each cluster includes about 100-150 loci. In some instances, each cluster includes about 109, 121, 130 or 137 loci. In some instances, each cluster includes about 19, 20, 61, 64 or more loci.

The number of distinct polynucleotides synthesized on a device may be dependent on the number of distinct loci available in the substrate. In some instances, the density of loci within a cluster of a device is at least or about 1 locus per mm², 10 loci per mm², 25 loci per mm², 50 loci per mm², 65 loci per mm², 75 loci per mm², 100 loci per mm², 130 loci per mm², 150 loci per mm², 175 loci per mm², 200 loci per mm², 300 loci per mm², 400 loci per mm², 500 loci per mm², 1,000 loci per mm² or more. In some instances, a device comprises from about 10 loci per mm² to about 500 mm², from about 25 loci per mm² to about 400 mm², from about 50 loci per mm² to about 500 mm², from about 100 loci per mm² to about 500 mm², from about 150 loci per mm² to about 500 mm², from about 10 loci per mm² to about 250 mm², from about 50 loci per mm² to about 250 mm², from about 10 loci per mm² to about 200 mm², or from about 50 loci per mm² to about 200 mm². In some instances, the distance from the centers of two adjacent loci within a cluster is from about 10 um to about 500 um, from about 10 um to about 200 um, or from about 10 um to about 100 um. In some instances, the distance from two centers of adjacent loci is greater than about 10 um, 20 um, 30 um, 40 um, 50 um, 60 um, 70 um, 80 um, 90 um or 100 um. In some instances, the distance from the centers of two adjacent loci is less than about 200 um, 150 um, 100 um, 80 um, 70 um, 60 um, 50 um, 40 um, 30 um, 20 um or 10 um. In some instances, each locus has a width of about 0.5 um, 1 um, 2 um, 3 um, 4 um, 5 um, 6 um, 7 um, 8 um, 9 um, 10 um, 20 um, 30 um, 40 um, 50 um, 60 um, 70 um, 80 um, 90 um or 100 um. In some instances, each locus is has a width of about 0.5 um to 100 um, about 0.5 um to 50 um, about 10 um to 75 um, or about 0.5 um to 50 um.

In some instances, the density of clusters within a device is at least or about 1 cluster per 100 mm², 1 cluster per 10 mm², 1 cluster per 5 mm², 1 cluster per 4 mm², 1 cluster per 3 mm², 1 cluster per 2 mm², 1 cluster per 1 mm², 2 clusters per 1 mm², 3 clusters per 1 mm², 4 clusters per 1 mm², 5 clusters per 1 mm², 10 clusters per 1 mm², 50 clusters per 1 mm² or more. In some instances, a device comprises from about 1 cluster per 10 mm² to about 10 clusters per 1 mm². In some instances, the distance from the centers of two adjacent clusters is less than about 50 um, 100 um, 200 um, 500 um, 1000 um, or 2000 um or 5000 um. In some instances, the distance from the centers of two adjacent clusters is from about 50 um and about 100 um, from about 50 um and about 200 um, from about 50 um and about 300 um, from about 50 um and about 500 um, and from about 100 um to about 2000 um. In some instances, the distance from the centers of two adjacent clusters is from about 0.05 mm to about 50 mm, from about 0.05 mm to about 10 mm, from about 0.05 mm and about 5 mm, from about 0.05 mm and about 4 mm, from about 0.05 mm and about 3 mm, from about 0.05 mm and about 2 mm, from about 0.1 mm and 10 mm, from about 0.2 mm and 10 mm, from about 0.3 mm and about 10 mm, from about 0.4 mm and about 10 mm, from about 0.5 mm and 10 mm, from about 0.5 mm and about 5 mm, or from about 0.5 mm and about 2 mm. In some instances, each cluster has a diameter or width along one dimension of about 0.5 to 2 mm, about 0.5 to 1 mm, or about 1 to 2 mm. In some instances, each cluster has a diameter or width along one dimension of about 0.5, 0.6, 0.7, 0.8, 0.9, 1, 1.1, 1.2, 1.3, 1.4, 1.5, 1.6, 1.7, 1.8, 1.9 or 2 mm. In some instances, each cluster has an interior diameter or width along one dimension of about 0.5, 0.6, 0.7, 0.8, 0.9, 1, 1.1, 1.15, 1.2, 1.3, 1.4, 1.5, 1.6, 1.7, 1.8, 1.9 or 2 mm.

A device may be about the size of a standard 96 well plate, for example from about 100 and 200 mm by from about 50 and 150 mm. In some instances, a device has a diameter less than or equal to about 1000 mm, 500 mm, 450 mm, 400 mm, 300 mm, 250 nm, 200 mm, 150 mm, 100 mm or 50 mm. In some instances, the diameter of a device is from about 25 mm and 1000 mm, from about 25 mm and about 800 mm, from about 25 mm and about 600 mm, from about 25 mm and about 500 mm, from about 25 mm and about 400 mm, from about 25 mm and about 300 mm, or from about 25 mm and about 200. Non-limiting examples of device size include about 300 mm, 200 mm, 150 mm, 130 mm, 100 mm, 76 mm, 51 mm and 25 mm. In some instances, a device has a planar surface area of at least about 100 mm²; 200 mm²; 500 mm²; 1,000 mm²; 2,000 mm²; 5,000 mm²; 10,000 mm²; 12,000 mm²; 15,000 mm²; 20,000 mm²; 30,000 mm²; 40,000 mm²; 50,000 mm² or more. In some instances, the thickness of a device is from about 50 mm and about 2000 mm, from about 50 mm and about 1000 mm, from about 100 mm and about 1000 mm, from about 200 mm and about 1000 mm, or from about 250 mm and about 1000 mm. Non-limiting examples of device thickness include 275 mm, 375 mm, 525 mm, 625 mm, 675 mm, 725 mm, 775 mm and 925 mm. In some instances, the thickness of a device varies with diameter and depends on the composition of the substrate. For example, a device comprising materials other than silicon has a different thickness than a silicon device of the same diameter. Device thickness may be determined by the mechanical strength of the material used and the device must be thick enough to support its own weight without cracking during handling. In some instances, a structure comprises a plurality of devices described herein.

Surface Materials

Provided herein is a device comprising a surface, wherein the surface is modified to support polynucleotide synthesis at predetermined locations and with a resulting low error rate, a low dropout rate, a high yield, and a high oligo representation. In some instances, surfaces of a device for polynucleotide synthesis provided herein are fabricated from a variety of materials capable of modification to support a de novo polynucleotide synthesis reaction. In some cases, the devices are sufficiently conductive, e.g., are able to form uniform electric fields across all or a portion of the device. A device described herein may comprise a flexible material. Exemplary flexible materials include, without limitation, modified nylon, unmodified nylon, nitrocellulose, and polypropylene. A device described herein may comprise a rigid material. Exemplary rigid materials include, without limitation, glass, fuse silica, silicon, silicon dioxide, silicon nitride, plastics (for example, polytetrafluoroethylene, polypropylene, polystyrene, polycarbonate, and blends thereof, and metals (for example, gold, platinum). Device disclosed herein may be fabricated from a material comprising silicon, polystyrene, agarose, dextran, cellulosic polymers, polyacrylamides, polydimethylsiloxane (PDMS), glass, or any combination thereof. In some cases, a device disclosed herein is manufactured with a combination of materials listed herein or any other suitable material known in the art.

A listing of tensile strengths for exemplary materials described herein is provides as follows: nylon (70 MPa), nitrocellulose (1.5 MPa), polypropylene (40 MPa), silicon (268 MPa), polystyrene (40 MPa), agarose (1-10 MPa), polyacrylamide (1-10 MPa), polydimethylsiloxane (PDMS) (3.9-10.8 MPa). Solid supports described herein can have a tensile strength from 1 to 300, 1 to 40, 1 to 10, 1 to 5, or 3 to 11 MPa. Solid supports described herein can have a tensile strength of about 1, 1.5, 2, 3, 4, 5, 6, 7, 8, 9, 10, 11, 20, 25, 40, 50, 60, 70, 80, 90, 100, 150, 200, 250, 270, or more MPa. In some instances, a device described herein comprises a solid support for polynucleotide synthesis that is in the form of a flexible material capable of being stored in a continuous loop or reel, such as a tape or flexible sheet.

Young's modulus measures the resistance of a material to elastic (recoverable) deformation under load. A listing of Young's modulus for stiffness of exemplary materials described herein is provides as follows: nylon (3 GPa), nitrocellulose (1.5 GPa), polypropylene (2 GPa), silicon (150 GPa), polystyrene (3 GPa), agarose (1-10 GPa), polyacrylamide (1-10 GPa), polydimethylsiloxane (PDMS) (1-10 GPa). Solid supports described herein can have a Young's moduli from 1 to 500, 1 to 40, 1 to 10, 1 to 5, or 3 to 11 GPa. Solid supports described herein can have a Young's moduli of about 1, 1.5, 2, 3, 4, 5, 6, 7, 8, 9, 10, 11, 20, 25, 40, 50, 60, 70, 80, 90, 100, 150, 200, 250, 400, 500 GPa, or more. As the relationship between flexibility and stiffness are inverse to each other, a flexible material has a low Young's modulus and changes its shape considerably under load.

In some cases, a device disclosed herein comprises a silicon dioxide base and a surface layer of silicon oxide. Alternatively, the device may have a base of silicon oxide. Surface of the device provided here may be textured, resulting in an increase overall surface area for polynucleotide synthesis. Device disclosed herein may comprise at least 5%, 10%, 25%, 50%, 80%, 90%, 95%, or 99% silicon. A device disclosed herein may be fabricated from a silicon on insulator (SOI) wafer.

Surface Architecture

Provided herein are devices comprising raised and/or lowered features. One benefit of having such features is an increase in surface area to support polynucleotide synthesis. In some instances, a device having raised and/or lowered features is referred to as a three-dimensional substrate. In some instances, a three-dimensional device comprises one or more channels. In some instances, one or more loci comprise a channel. In some instances, the channels are accessible to reagent deposition via a deposition device such as a polynucleotide synthesizer. In some instances, reagents and/or fluids collect in a larger well in fluid communication one or more channels. For example, a device comprises a plurality of channels corresponding to a plurality of loci with a cluster, and the plurality of channels are in fluid communication with one well of the cluster. In some methods, a library of polynucleotides is synthesized in a plurality of loci of a cluster.

In some instances, the structure is configured to allow for controlled flow and mass transfer paths for polynucleotide synthesis on a surface. In some instances, the configuration of a device allows for the controlled and even distribution of mass transfer paths, chemical exposure times, and/or wash efficacy during polynucleotide synthesis. In some instances, the configuration of a device allows for increased sweep efficiency, for example by providing sufficient volume for a growing a polynucleotide such that the excluded volume by the growing polynucleotide does not take up more than 50, 45, 40, 35, 30, 25, 20, 15, 14, 13, 12, 11, 10, 9, 8, 7, 6, 5, 4, 3, 2, 1%, or less of the initially available volume that is available or suitable for growing the polynucleotide. In some instances, a three-dimensional structure allows for managed flow of fluid to allow for the rapid exchange of chemical exposure.

Provided herein are methods to synthesize an amount of DNA of 1 fM, 5 fM, 10 fM, 25 fM, 50 fM, 75 fM, 100 fM, 200 fM, 300 fM, 400 fM, 500 fM, 600 fM, 700 fM, 800 fM, 900 fM, 1 pM, 5 pM, 10 pM, 25 pM, 50 pM, 75 pM, 100 pM, 200 pM, 300 pM, 400 pM, 500 pM, 600 pM, 700 pM, 800 pM, 900 pM, or more. In some instances, a polynucleotide library may span the length of about 1%, 20%, 3%, 40%, 5%, 100%, 15%, 200%, 300%, 400%, 500%, 600%, 700%, 800%, 900%, 95%, or 100% of a gene. A gene may be varied up to about 1%, 2%, 3%, 4%, 5%, 10%, 15%, 20%, 30%, 40%, 50%, 60%, 70%, 80%, 85%, 90%, 95%, or 100%.

Non-identical polynucleotides may collectively encode a sequence for at least 1%, 2%, 3%, 4%, 5%, 10%, 15%, 20%, 30%, 40%, 50%, 60%, 70%, 800%, 85%, 90%, 95%, or 100% of a gene. In some instances, a polynucleotide may encode a sequence of 50%, 60%, 70%, 80%, 85%, 90%, 95%, or more of a gene. In some instances, a polynucleotide may encode a sequence of 80%, 85%, 90%, 95%, or more of a gene.

In some instances, segregation is achieved by physical structure. In some instances, segregation is achieved by differential functionalization of the surface generating active and passive regions for polynucleotide synthesis. Differential functionalization is also be achieved by alternating the hydrophobicity across the device surface, thereby creating water contact angle effects that cause beading or wetting of the deposited reagents. Employing larger structures can decrease splashing and cross-contamination of distinct polynucleotide synthesis locations with reagents of the neighboring spots. In some instances, a device, such as a polynucleotide synthesizer, is used to deposit reagents to distinct polynucleotide synthesis locations. Substrates having three-dimensional features are configured in a manner that allows for the synthesis of a large number of polynucleotides (e.g., more than about 10,000) with a low error rate (e.g., less than about 1:500, 1:1000, 1:1500, 1:2,000; 1:3,000; 1:5,000; or 1:10,000). In some instances, a device comprises features with a density of about or greater than about 1, 5, 10, 20, 30, 40, 50, 60, 70, 80, 100, 110, 120, 130, 140, 150, 160, 170, 180, 190, 200, 300, 400 or 500 features per mm².

A well of a device may have the same or different width, height, and/or volume as another well of the substrate. A channel of a device may have the same or different width, height, and/or volume as another channel of the substrate. In some instances, the width of a cluster is from about 0.05 mm to about 50 mm, from about 0.05 mm to about 10 mm, from about 0.05 mm and about 5 mm, from about 0.05 mm and about 4 mm, from about 0.05 mm and about 3 mm, from about 0.05 mm and about 2 mm, from about 0.05 mm and about 1 mm, from about 0.05 mm and about 0.5 mm, from about 0.05 mm and about 0.1 mm, from about 0.1 mm and 10 mm, from about 0.2 mm and 10 mm, from about 0.3 mm and about 10 mm, from about 0.4 mm and about 10 mm, from about 0.5 mm and 10 mm, from about 0.5 mm and about 5 mm, or from about 0.5 mm and about 2 mm. In some instances, the width of a well comprising a cluster is from about 0.05 mm to about 50 mm, from about 0.05 mm to about 10 mm, from about 0.05 mm and about 5 mm, from about 0.05 mm and about 4 mm, from about 0.05 mm and about 3 mm, from about 0.05 mm and about 2 mm, from about 0.05 mm and about 1 mm, from about 0.05 mm and about 0.5 mm, from about 0.05 mm and about 0.1 mm, from about 0.1 mm and 10 mm, from about 0.2 mm and 10 mm, from about 0.3 mm and about 10 mm, from about 0.4 mm and about 10 mm, from about 0.5 mm and 10 mm, from about 0.5 mm and about 5 mm, or from about 0.5 mm and about 2 mm. In some instances, the width of a cluster is less than or about 5 mm, 4 mm, 3 mm, 2 mm, 1 mm, 0.5 mm, 0.1 mm, 0.09 mm, 0.08 mm, 0.07 mm, 0.06 mm or 0.05 mm. In some instances, the width of a cluster is from about 1.0 and 1.3 mm. In some instances, the width of a cluster is about 1.150 mm. In some instances, the width of a well is less than or about 5 mm, 4 mm, 3 mm, 2 mm, 1 mm, 0.5 mm, 0.1 mm, 0.09 mm, 0.08 mm, 0.07 mm, 0.06 mm or 0.05 mm. In some instances, the width of a well is from about 1.0 and 1.3 mm. In some instances, the width of a well is about 1.150 mm. In some instances, the width of a cluster is about 0.08 mm. In some instances, the width of a well is about 0.08 mm. The width of a cluster may refer to clusters within a two-dimensional or three-dimensional substrate.

In some instances, the height of a well is from about 20 um to about 1000 um, from about 50 um to about 1000 um, from about 100 um to about 1000 um, from about 200 um to about 1000 um, from about 300 um to about 1000 um, from about 400 um to about 1000 um, or from about 500 um to about 1000 um. In some instances, the height of a well is less than about 1000 um, less than about 900 um, less than about 800 um, less than about 700 um, or less than about 600 um.

In some instances, a device comprises a plurality of channels corresponding to a plurality of loci within a cluster, wherein the height or depth of a channel is from about 5 um to about 500 um, from about 5 um to about 400 um, from about 5 um to about 300 um, from about 5 um to about 200 um, from about 5 um to about 100 um, from about 5 um to about 50 um, or from about 10 um to about 50 um. In some instances, the height of a channel is less than 100 um, less than 80 um, less than 60 um, less than 40 um or less than 20 um.

In some instances, the diameter of a channel, locus (e.g., in a substantially planar substrate) or both channel and locus (e.g., in a three-dimensional device wherein a locus corresponds to a channel) is from about 1 um to about 1000 um, from about 1 um to about 500 um, from about 1 um to about 200 um, from about 1 um to about 100 um, from about 5 um to about 100 um, or from about 10 um to about 100 um, for example, about 90 um, 80 um, 70 um, 60 um, 50 um, 40 um, 30 um, 20 um or 10 um. In some instances, the diameter of a channel, locus, or both channel and locus is less than about 100 um, 90 um, 80 um, 70 um, 60 um, 50 um, 40 um, 30 um, 20 um or 10 um. In some instances, the distance from the center of two adjacent channels, loci, or channels and loci is from about 1 um to about 500 um, from about 1 um to about 200 um, from about 1 um to about 100 um, from about 5 um to about 200 um, from about 5 um to about 100 um, from about 5 um to about 50 um, or from about 5 um to about 30 um, for example, about 20 um.

Surface Modifications

In various instances, surface modifications are employed for the chemical and/or physical alteration of a surface by an additive or subtractive process to change one or more chemical and/or physical properties of a device surface or a selected site or region of a device surface. For example, surface modifications include, without limitation, (1) changing the wetting properties of a surface, (2) functionalizing a surface, i.e., providing, modifying or substituting surface functional groups, (3) defunctionalizing a surface, i.e., removing surface functional groups, (4) otherwise altering the chemical composition of a surface, e.g., through etching, (5) increasing or decreasing surface roughness, (6) providing a coating on a surface, e.g., a coating that exhibits wetting properties that are different from the wetting properties of the surface, and/or (7) depositing particulates on a surface.

In some instances, the addition of a chemical layer on top of a surface (referred to as adhesion promoter) facilitates structured patterning of loci on a surface of a substrate. Exemplary surfaces for application of adhesion promotion include, without limitation, glass, silicon, silicon dioxide and silicon nitride. In some instances, the adhesion promoter is a chemical with a high surface energy. In some instances, a second chemical layer is deposited on a surface of a substrate. In some instances, the second chemical layer has a low surface energy. In some instances, surface energy of a chemical layer coated on a surface supports localization of droplets on the surface. Depending on the patterning arrangement selected, the proximity of loci and/or area of fluid contact at the loci are alterable.

In some instances, a device surface, or resolved loci, onto which nucleic acids or other moieties are deposited, e.g., for polynucleotide synthesis, are smooth or substantially planar (e.g., two-dimensional) or have irregularities, such as raised or lowered features (e.g., three-dimensional features). In some instances, a device surface is modified with one or more different layers of compounds. Such modification layers of interest include, without limitation, inorganic and organic layers such as metals, metal oxides, polymers, small organic molecules and the like. Non-limiting polymeric layers include peptides, proteins, nucleic acids or mimetics thereof (e.g., peptide nucleic acids and the like), polysaccharides, phospholipids, polyurethanes, polyesters, polycarbonates, polyureas, polyamides, polyethyleneamines, polyarylene sulfides, polysiloxanes, polyimides, polyacetates, and any other suitable compounds described herein or otherwise known in the art. In some instances, polymers are heteropolymeric. In some instances, polymers are homopolymeric. In some instances, polymers comprise functional moieties or are conjugated.

In some instances, resolved loci of a device are functionalized with one or more moieties that increase and/or decrease surface energy. In some instances, a moiety is chemically inert. In some instances, a moiety is configured to support a desired chemical reaction, for example, one or more processes in a polynucleotide synthesis reaction. The surface energy, or hydrophobicity, of a surface is a factor for determining the affinity of a nucleotide to attach onto the surface. In some instances, a method for device functionalization may comprise: (a) providing a device having a surface that comprises silicon dioxide; and (b) silanizing the surface using, a suitable silanizing agent described herein or otherwise known in the art, for example, an organofunctional alkoxysilane molecule.

In some instances, the organofunctional alkoxysilane molecule comprises dimethylchloro-octodecyl-silane, methyldichloro-octodecyl-silane, trichloro-octodecyl-silane, trimethyl-octodecyl-silane, triethyl-octodecyl-silane, or any combination thereof. In some instances, a device surface comprises functionalized with polyethylene/polypropylene (functionalized by gamma irradiation or chromic acid oxidation, and reduction to hydroxyalkyl surface), highly crosslinked polystyrene-divinylbenzene (derivatized by chloromethylation, and aminated to benzylamine functional surface), nylon (the terminal aminohexyl groups are directly reactive), or etched with reduced polytetrafluoroethylene. Other methods and functionalizing agents are described in U.S. Pat. No. 5,474,796, which is herein incorporated by reference in its entirety.

In some instances, a device surface is functionalized by contact with a derivatizing composition that contains a mixture of silanes, under reaction conditions effective to couple the silanes to the device surface, typically via reactive hydrophilic moieties present on the device surface. Silanization generally covers a surface through self-assembly with organofunctional alkoxysilane molecules.

A variety of siloxane functionalizing reagents can further be used as currently known in the art, e.g., for lowering or increasing surface energy. The organofunctional alkoxysilanes can be classified according to their organic functions.

Provided herein are devices that may contain patterning of agents capable of coupling to a nucleoside. In some instances, a device may be coated with an active agent. In some instances, a device may be coated with a passive agent. Exemplary active agents for inclusion in coating materials described herein includes, without limitation, N-(3-triethoxysilylpropyl)-4-hydroxybutyramide (HAPS), 11-acetoxyundecyltriethoxysilane, n-decyltriethoxysilane, (3-aminopropyl)trimethoxysilane, (3-aminopropyl)triethoxysilane, 3-glycidoxypropyltrimethoxysilane (GOPS), 3-iodo-propyltrimethoxysilane, butyl-aldehydr-trimethoxysilane, dimeric secondary aminoalkyl siloxanes, (3-aminopropyl)-diethoxy-methylsilane, (3-aminopropyl)-dimethyl-ethoxysilane, and (3-aminopropyl)-trimethoxysilane, (3-glycidoxypropyl)-dimethyl-ethoxysilane, glycidoxy-trimethoxysilane, (3-mercaptopropyl)-trimethoxysilane, 3-4 epoxycyclohexyl-ethyltrimethoxysilane, and (3-mercaptopropyl)-methyl-dimethoxysilane, allyl trichlorochlorosilane, 7-oct-1-enyl trichlorochlorosilane, or bis (3-trimethoxysilylpropyl) amine.

Exemplary passive agents for inclusion in a coating material described herein includes, without limitation, perfluorooctyltrichlorosilane; tridecafluoro-1,1,2,2-tetrahydrooctyl)trichlorosilane; 1H, 1H, 2H, 2H-fluorooctyltriethoxysilane (FOS); trichloro(1H, 1H, 2H, 2H-perfluorooctyl)silane; tert-butyl-[5-fluoro-4-(4,4,5,5-tetramethyl-1,3,2-dioxaborolan-2-yl)indol-1-yl]-dimethyl-silane; CYTOP™; Fluorinert™; perfluoroctyltrichlorosilane (PFOTCS); perfluorooctyldimethylchlorosilane (PFODCS); perfluorodecyltriethoxysilane (PFDTES); pentafluorophenyl-dimethylpropylchloro-silane (PFPTES); perfluorooctyltriethoxysilane; perfluorooctyltrimethoxysilane; octylchlorosilane; dimethylchloro-octodecyl-silane; methyldichloro-octodecyl-silane; trichloro-octodecyl-silane; trimethyl-octodecyl-silane; triethyl-octodecyl-silane; or octadecyltrichlorosilane.

In some instances, a functionalization agent comprises a hydrocarbon silane such as octadecyltrichlorosilane. In some instances, the functionalizing agent comprises 11-acetoxyundecyltriethoxysilane, n-decyltriethoxysilane, (3-aminopropyl)trimethoxysilane, (3-aminopropyl)triethoxysilane, glycidyloxypropyl/trimethoxysilane and N-(3-triethoxysilylpropyl)-4-hydroxybutyramide.

Polynucleotide Synthesis

Methods of the current disclosure for polynucleotide synthesis may include processes involving phosphoramidite chemistry. In some instances, polynucleotide synthesis comprises coupling a base with phosphoramidite. Polynucleotide synthesis may comprise coupling a base by deposition of phosphoramidite under coupling conditions, wherein the same base is optionally deposited with phosphoramidite more than once, i.e., double coupling. Polynucleotide synthesis may comprise capping of unreacted sites. In some instances, capping is optional. Polynucleotide synthesis may also comprise oxidation or an oxidation step or oxidation steps. Polynucleotide synthesis may comprise deblocking, detritylation, and sulfurization. In some instances, polynucleotide synthesis comprises either oxidation or sulfurization. In some instances, between one or each step during a polynucleotide synthesis reaction, the device is washed, for example, using tetrazole or acetonitrile. Time frames for any one step in a phosphoramidite synthesis method may be less than about 2 minutes, 1 minute, 50 seconds, 40 seconds, 30 seconds, 20 seconds and 10 seconds.

Polynucleotide synthesis using a phosphoramidite method may comprise a subsequent addition of a phosphoramidite building block (e.g., nucleoside phosphoramidite) to a growing polynucleotide chain for the formation of a phosphite triester linkage. Phosphoramidite polynucleotide synthesis proceeds in the 3′ to 5′ direction. Phosphoramidite polynucleotide synthesis allows for the controlled addition of one nucleotide to a growing nucleic acid chain per synthesis cycle. In some instances, each synthesis cycle comprises a coupling step. Phosphoramidite coupling involves the formation of a phosphite triester linkage between an activated nucleoside phosphoramidite and a nucleoside bound to the substrate, for example, via a linker. In some instances, the nucleoside phosphoramidite is provided to the device activated. In some instances, the nucleoside phosphoramidite is provided to the device with an activator. In some instances, nucleoside phosphoramidites are provided to the device in a 1.5, 2, 3, 4, 5, 6, 7, 8, 9, 10, 11, 12, 13, 14, 15, 16, 17, 18, 19, 20, 25, 30, 35, 40, 50, 60, 70, 80, 90, 100-fold excess or more over the substrate-bound nucleosides. In some instances, the addition of nucleoside phosphoramidite is performed in an anhydrous environment, for example, in anhydrous acetonitrile. Following addition of a nucleoside phosphoramidite, the device is optionally washed. In some instances, the coupling step is repeated one or more additional times, optionally with a wash step between nucleoside phosphoramidite additions to the substrate. In some instances, a polynucleotide synthesis method used herein comprises 1, 2, 3 or more sequential coupling steps. Prior to coupling, in many cases, the nucleoside bound to the device is de-protected by removal of a protecting group, where the protecting group functions to prevent polymerization. A common protecting group is 4,4′-dimethoxytrityl (DMT).

Following coupling, phosphoramidite polynucleotide synthesis methods optionally comprise a capping step. In a capping step, the growing polynucleotide is treated with a capping agent. A capping step is useful to block unreacted substrate-bound 5′—OH groups after coupling from further chain elongation, preventing the formation of polynucleotides with internal base deletions. Further, phosphoramidites activated with 1H-tetrazole may react, to a small extent, with the 06 position of guanosine. Without being bound by theory, upon oxidation with I₂/water, this side product, possibly via O6-N7 migration, may undergo depurination. The apurinic sites may end up being cleaved in the course of the final deprotection of the polynucleotide thus reducing the yield of the full-length product. The O6 modifications may be removed by treatment with the capping reagent prior to oxidation with I₂/water. In some instances, inclusion of a capping step during polynucleotide synthesis decreases the error rate as compared to synthesis without capping. As an example, the capping step comprises treating the substrate-bound polynucleotide with a mixture of acetic anhydride and 1-methylimidazole. Following a capping step, the device is optionally washed.

In some instances, following addition of a nucleoside phosphoramidite, and optionally after capping and one or more wash steps, the device bound growing nucleic acid is oxidized. The oxidation step comprises the phosphite triester is oxidized into a tetracoordinated phosphate triester, a protected precursor of the naturally occurring phosphate diester internucleoside linkage. In some instances, oxidation of the growing polynucleotide is achieved by treatment with iodine and water, optionally in the presence of a weak base (e.g., pyridine, lutidine, collidine). Oxidation may be carried out under anhydrous conditions using, e.g. tert-Butyl hydroperoxide or (1S)-(+)-(10-camphorsulfonyl)-oxaziridine (CSO). In some methods, a capping step is performed following oxidation. A second capping step allows for device drying, as residual water from oxidation that may persist can inhibit subsequent coupling. Following oxidation, the device and growing polynucleotide is optionally washed. In some instances, the step of oxidation is substituted with a sulfurization step to obtain polynucleotide phosphorothioates, wherein any capping steps can be performed after the sulfurization. Many reagents are capable of the efficient sulfur transfer, including but not limited to 3-(Dimethylaminomethylidene)amino)-3H-1,2,4-dithiazole-3-thione, DDTT, 3H-1,2-benzodithiol-3-one 1,1-dioxide, also known as Beaucage reagent, and N,N,N′N′-Tetraethylthiuram disulfide (TETD).

In order for a subsequent cycle of nucleoside incorporation to occur through coupling, the protected 5′ end of the device bound growing polynucleotide is removed so that the primary hydroxyl group is reactive with a next nucleoside phosphoramidite. In some instances, the protecting group is DMT and deblocking occurs with trichloroacetic acid in dichloromethane. Conducting detritylation for an extended time or with stronger than recommended solutions of acids may lead to increased depurination of solid support-bound polynucleotide and thus reduces the yield of the desired full-length product. Methods and compositions of the disclosure described herein provide for controlled deblocking conditions limiting undesired depurination reactions. In some instances, the device bound polynucleotide is washed after deblocking. In some instances, efficient washing after deblocking contributes to synthesized polynucleotides having a low error rate.

Methods for the synthesis of polynucleotides typically involve an iterating sequence of the following steps: application of a protected monomer to an actively functionalized surface (e.g., locus) to link with either the activated surface, a linker or with a previously deprotected monomer; deprotection of the applied monomer so that it is reactive with a subsequently applied protected monomer; and application of another protected monomer for linking. One or more intermediate steps include oxidation or sulfurization. In some instances, one or more wash steps precede or follow one or all of the steps.

Methods for phosphoramidite-based polynucleotide synthesis comprise a series of chemical steps. In some instances, one or more steps of a synthesis method involve reagent cycling, where one or more steps of the method comprise application to the device of a reagent useful for the step. For example, reagents are cycled by a series of liquid deposition and vacuum drying steps. For substrates comprising three-dimensional features such as wells, microwells, channels and the like, reagents are optionally passed through one or more regions of the device via the wells and/or channels.

Methods and systems described herein relate to polynucleotide synthesis devices for the synthesis of polynucleotides. The synthesis may be in parallel. For example at least or about at least 2, 3, 4, 5, 6, 7, 8, 9, 10, 11, 12, 13, 14, 15, 16, 17, 18, 19, 20, 21, 22, 23, 24, 25, 30, 35, 40, 45, 50, 100, 150, 200, 250, 300, 350, 400, 450, 500, 550, 600, 650, 700, 750, 800, 850, 900, 1000, 10000, 50000, 75000, 100000 or more polynucleotides can be synthesized in parallel. The total number polynucleotides that may be synthesized in parallel may be from 2-100000, 3-50000, 4-10000, 5-1000, 6-900, 7-850, 8-800, 9-750, 10-700, 11-650, 12-600, 13-550, 14-500, 15-450, 16-400, 17-350, 18-300, 19-250, 20-200, 21-150,22-100, 23-50, 24-45, 25-40, 30-35. Those of skill in the art appreciate that the total number of polynucleotides synthesized in parallel may fall within any range bound by any of these values, for example 25-100. The total number of polynucleotides synthesized in parallel may fall within any range defined by any of the values serving as endpoints of the range. Total molar mass of polynucleotides synthesized within the device or the molar mass of each of the polynucleotides may be at least or at least about 10, 20, 30, 40, 50, 100, 250, 500, 750, 1000, 2000, 3000, 4000, 5000, 6000, 7000, 8000, 9000, 10000, 25000, 50000, 75000, 100000 picomoles, or more. The length of each of the polynucleotides or average length of the polynucleotides within the device may be at least or about at least 10, 15, 20, 25, 30, 35, 40, 45, 50, 100, 150, 200, 300, 400, 500 nucleotides, or more. The length of each of the polynucleotides or average length of the polynucleotides within the device may be at most or about at most 500, 400, 300, 200, 150, 100, 50, 45, 35, 30, 25, 20, 19, 18, 17, 16, 15, 14, 13, 12, 11, 10 nucleotides, or less. The length of each of the polynucleotides or average length of the polynucleotides within the device may fall from 10-500, 9-400, 11-300, 12-200, 13-150, 14-100, 15-50, 16-45, 17-40, 18-35, 19-25. Those of skill in the art appreciate that the length of each of the polynucleotides or average length of the polynucleotides within the device may fall within any range bound by any of these values, for example 100-300. The length of each of the polynucleotides or average length of the polynucleotides within the device may fall within any range defined by any of the values serving as endpoints of the range.

Methods for polynucleotide synthesis on a surface provided herein allow for synthesis at a fast rate. As an example, at least 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 70, 80, 90, 100, 125, 150, 175, 200 nucleotides per hour, or more are synthesized. Nucleotides include adenine, guanine, thymine, cytosine, uridine building blocks, or analogs/modified versions thereof. In some instances, libraries of polynucleotides are synthesized in parallel on substrate. For example, a device comprising about or at least about 100; 1,000; 10,000; 30,000; 75,000; 100,000; 1,000,000; 2,000,000; 3,000,000; 4,000,000; or 5,000,000 resolved loci is able to support the synthesis of at least the same number of distinct polynucleotides, wherein polynucleotide encoding a distinct sequence is synthesized on a resolved locus. In some instances, a library of polynucleotides are synthesized on a device with low error rates described herein in less than about three months, two months, one month, three weeks, 15, 14, 13, 12, 11, 10, 9, 8, 7, 6, 5, 4, 3, 2 days, 24 hours or less. In some instances, larger nucleic acids assembled from a polynucleotide library synthesized with low error rate using the substrates and methods described herein are prepared in less than about three months, two months, one month, three weeks, 15, 14, 13, 12, 11, 10, 9, 8, 7, 6, 5, 4, 3, 2 days, 24 hours or less.

In some instances, methods described herein provide for generation of a library of polynucleotides comprising variant polynucleotides differing at a plurality of codon sites. In some instances, a polynucleotide may have 1 site, 2 sites, 3 sites, 4 sites, 5 sites, 6 sites, 7 sites, 8 sites, 9 sites, 10 sites, 11 sites, 12 sites, 13 sites, 14 sites, 15 sites, 16 sites, 17 sites 18 sites, 19 sites, 20 sites, 30 sites, 40 sites, 50 sites, or more of variant codon sites.

In some instances, the one or more sites of variant codon sites may be adjacent. In some instances, the one or more sites of variant codon sites may be not be adjacent and separated by 1, 2, 3, 4, 5, 6, 7, 8, 9, 10, or more codons.

In some instances, a polynucleotide may comprise multiple sites of variant codon sites, wherein all the variant codon sites are adjacent to one another, forming a stretch of variant codon sites. In some instances, a polynucleotide may comprise multiple sites of variant codon sites, wherein none the variant codon sites are adjacent to one another. In some instances, a polynucleotide may comprise multiple sites of variant codon sites, wherein some the variant codon sites are adjacent to one another, forming a stretch of variant codon sites, and some of the variant codon sites are not adjacent to one another.

Large Polynucleotide Libraries Having Low Error Rates

Average error rates for polynucleotides synthesized within a library using the systems and methods provided may be less than 1 in 1000, less than 1 in 1250, less than 1 in 1500, less than 1 in 2000, less than 1 in 3000 or less often. In some instances, average error rates for polynucleotides synthesized within a library using the systems and methods provided are less than 1/500, 1/600, 1/700, 1/800, 1/900, 1/1000, 1/1100, 1/1200, 1/1250, 1/1300, 1/1400, 1/1500, 1/1600, 1/1700, 1/1800, 1/1900, 1/2000, 1/3000, or less. In some instances, average error rates for polynucleotides synthesized within a library using the systems and methods provided are less than 1/1000.

In some instances, aggregate error rates for polynucleotides synthesized within a library using the systems and methods provided are less than 1/500, 1/600, 1/700, 1/800, 1/900, 1/1000, 1/1100, 1/1200, 1/1250, 1/1300, 1/1400, 1/1500, 1/1600, 1/1700, 1/1800, 1/1900, 1/2000, 1/3000, or less compared to the predetermined sequences. In some instances, aggregate error rates for polynucleotides synthesized within a library using the systems and methods provided are less than 1/500, 1/600, 1/700, 1/800, 1/900, or 1/1000. In some instances, aggregate error rates for polynucleotides synthesized within a library using the systems and methods provided are less than 1/1000.

In some instances, an error correction enzyme may be used for polynucleotides synthesized within a library using the systems and methods provided can use. In some instances, aggregate error rates for polynucleotides with error correction can be less than 1/500, 1/600, 1/700, 1/800, 1/900, 1/1000, 1/1100, 1/1200, 1/1300, 1/1400, 1/1500, 1/1600, 1/1700, 1/1800, 1/1900, 1/2000, 1/3000, or less compared to the predetermined sequences. In some instances, aggregate error rates with error correction for polynucleotides synthesized within a library using the systems and methods provided can be less than 1/500, 1/600, 1/700, 1/800, 1/900, or 1/1000. In some instances, aggregate error rates with error correction for polynucleotides synthesized within a library using the systems and methods provided can be less than 1/1000.

Error rate may limit the value of gene synthesis for the production of libraries of gene variants. With an error rate of 1/300, about 0.7% of the clones in a 1500 base pair gene will be correct. As most of the errors from polynucleotide synthesis result in frame-shift mutations, over 99% of the clones in such a library will not produce a full-length protein. Reducing the error rate by 75% would increase the fraction of clones that are correct by a factor of 40. The methods and compositions of the disclosure allow for fast de novo synthesis of large polynucleotide and gene libraries with error rates that are lower than commonly observed gene synthesis methods both due to the improved quality of synthesis and the applicability of error correction methods that are enabled in a massively parallel and time-efficient manner. Accordingly, libraries may be synthesized with base insertion, deletion, substitution, or total error rates that are under 1/300, 1/400, 1/500, 1/600, 1/700, 1/800, 1/900, 1/1000, 1/1250, 1/1500, 1/2000, 1/2500, 1/3000, 1/4000, 1/5000, 1/6000, 1/7000, 1/8000, 1/9000, 1/10000, 1/12000, 1/15000, 1/20000, 1/25000, 1/30000, 1/40000, 1/50000, 1/60000, 1/70000, 1/80000, 1/90000, 1/100000, 1/125000, 1/150000, 1/200000, 1/300000, 1/400000, 1/500000, 1/600000, 1/700000, 1/800000, 1/900000, 1/1000000, or less, across the library, or across more than 80%, 85%, 90%, 93%, 95%, 96%, 97%, 98%, 99%, 99.5%, 99.8%, 99.9%, 99.95%, 99.98%, 99.99%, or more of the library. The methods and compositions of the disclosure further relate to large synthetic polynucleotide and gene libraries with low error rates associated with at least 30%, 40%, 50%, 60%, 70%, 75%, 80%, 85%, 90%, 93%, 95%, 96%, 97%, 98%, 99%, 99.5%, 99.8%, 99.9%, 99.95%, 99.98%, 99.99%, or more of the polynucleotides or genes in at least a subset of the library to relate to error free sequences in comparison to a predetermined/preselected sequence. In some instances, at least 30%, 40%, 50%, 60%, 70%, 75%, 80%, 85%, 90%, 93%, 95%, 96%, 97%, 98%, 99%, 99.5%, 99.8%, 99.9%, 99.95%, 99.98%, 99.99%, or more of the polynucleotides or genes in an isolated volume within the library have the same sequence. In some instances, at least 30%, 40%, 50%, 60%, 70%, 75%, 80%, 85%, 90%, 93%, 95%, 96%, 97%, 98%, 99%, 99.5%, 99.8%, 99.9%, 99.95%, 99.98%, 99.99%, or more of any polynucleotides or genes related with more than 95%, 96%, 97%, 98%, 99%, 99.5%, 99.6%, 99.7%, 99.8%, 99.9% or more similarity or identity have the same sequence. In some instances, the error rate related to a specified locus on a polynucleotide or gene is optimized. Thus, a given locus or a plurality of selected loci of one or more polynucleotides or genes as part of a large library may each have an error rate that is less than 1/300, 1/400, 1/500, 1/600, 1/700, 1/800, 1/900, 1/1000, 1/1250, 1/1500, 1/2000, 1/2500, 1/3000, 1/4000, 1/5000, 1/6000, 1/7000, 1/8000, 1/9000, 1/10000, 1/12000, 1/15000, 1/20000, 1/25000, 1/30000, 1/40000, 1/50000, 1/60000, 1/70000, 1/80000, 1/90000, 1/100000, 1/125000, 1/150000, 1/200000, 1/300000, 1/400000, 1/500000, 1/600000, 1/700000, 1/800000, 1/900000, 1/1000000, or less. In various instances, such error optimized loci may comprise at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500, 600, 700, 800, 900, 1000, 1500, 2000, 2500, 3000, 4000, 5000, 6000, 7000, 8000, 9000, 10000, 30000, 50000, 75000, 100000, 500000, 1000000, 2000000, 3000000 or more loci. The error optimized loci may be distributed to at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500, 600, 700, 800, 900, 1000, 1500, 2000, 2500, 3000, 4000, 5000, 6000, 7000, 8000, 9000, 10000, 30000, 75000, 100000, 500000, 1000000, 2000000, 3000000 or more polynucleotides or genes.

The error rates can be achieved with or without error correction. The error rates can be achieved across the library, or across more than 80%, 85%, 90%, 93%, 95%, 96%, 97%, 98%, 99%, 99.5%, 99.8%, 99.9%, 99.95%, 99.98%, 99.99%, or more of the library.

Computer Systems

Any of the systems described herein, may be operably linked to a computer and may be automated through a computer either locally or remotely. In various instances, the methods and systems of the disclosure may further comprise software programs on computer systems and use thereof. Accordingly, computerized control for the synchronization of the dispense/vacuum/refill functions such as orchestrating and synchronizing the material deposition device movement, dispense action and vacuum actuation are within the bounds of the disclosure. The computer systems may be programmed to interface between the user specified base sequence and the position of a material deposition device to deliver the correct reagents to specified regions of the substrate.

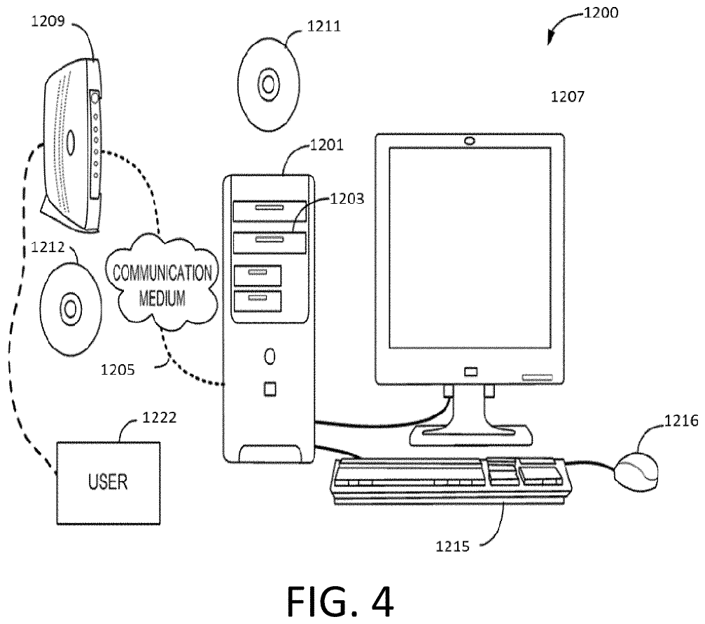

The computer system 1200 illustrated in FIG. 4 may be understood as a logical apparatus that can read instructions from media 1211 and/or a network port 1205, which can optionally be connected to server 1209 having fixed media 1212. The system, such as shown in FIG. 4 can include a CPU 1201, disk drives 1203, optional input devices such as keyboard 1215 and/or mouse 1216 and optional monitor 1207. Data communication can be achieved through the indicated communication medium to a server at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party 1222 as illustrated in FIG. 4.

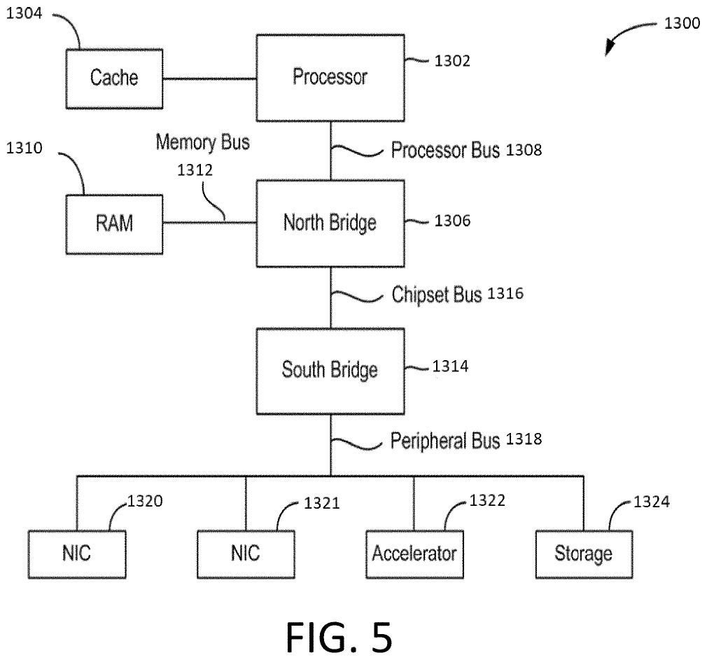

FIG. 5 is a block diagram illustrating a first example architecture of a computer system 1300 that can be used in connection with example instances of the present disclosure. As depicted in FIG. 5, the example computer system can include a processor 1302 for processing instructions. Non-limiting examples of processors include: Intel Xeon™ processor, AMD Opteron™ processor, Samsung 32-bit RISC ARM 1176JZ(F)-S v1.0™ processor, ARM Cortex-A8 Samsung S5PC100™ processor, ARM Cortex-A8 Apple A4™ processor, Marvell PXA 930™ processor, or a functionally-equivalent processor. Multiple threads of execution can be used for parallel processing. In some instances, multiple processors or processors with multiple cores can also be used, whether in a single computer system, in a cluster, or distributed across systems over a network comprising a plurality of computers, cell phones, and/or personal data assistant devices.

As illustrated in FIG. 5, a high speed cache 1304 can be connected to, or incorporated in, the processor 1302 to provide a high speed memory for instructions or data that have been recently, or are frequently, used by processor 1302. The processor 1302 is connected to a north bridge 1306 by a processor bus 1308. The north bridge 1306 is connected to random access memory (RAM) 1310 by a memory bus 1312 and manages access to the RAM 1310 by the processor 1302. The north bridge 1306 is also connected to a south bridge 1314 by a chipset bus 1316. The south bridge 1314 is, in turn, connected to a peripheral bus 1318. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 1318. In some alternative architectures, the functionality of the north bridge can be incorporated into the processor instead of using a separate north bridge chip. In some instances, system 1300 can include an accelerator card 1322 attached to the peripheral bus 1318. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 1324 and can be loaded into RAM 1310 and/or cache 1304 for use by the processor. The system 1300 includes an operating system for managing system resources; non-limiting examples of operating systems include: Linux, Windows™, MACOS™, BlackBerry OS™, iOS™, and other functionally-equivalent operating systems, as well as application software running on top of the operating system for managing data storage and optimization in accordance with example instances of the present disclosure. In this example, system 1300 also includes network interface cards (NICs) 1320 and 1321 connected to the peripheral bus for providing network interfaces to external storage, such as Network Attached Storage (NAS) and other computer systems that can be used for distributed parallel processing.

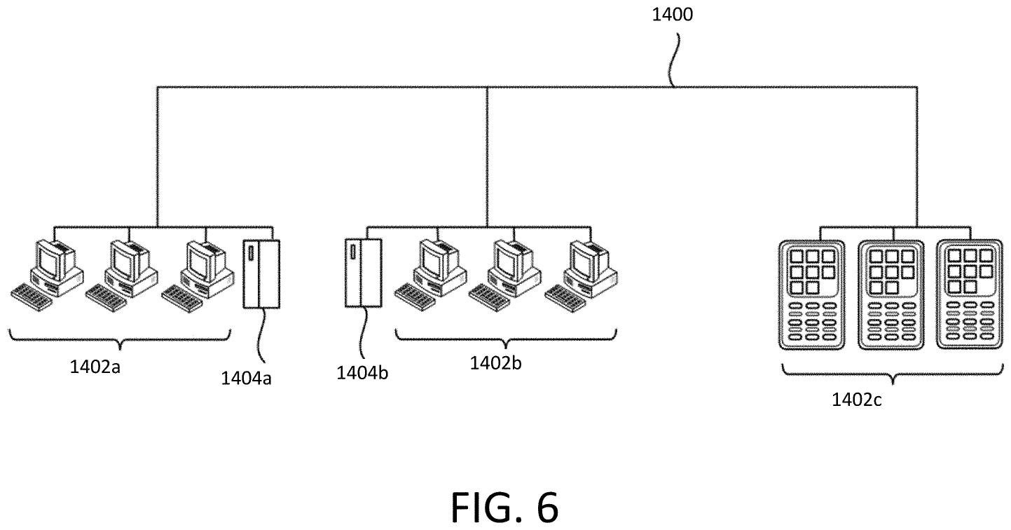

FIG. 6 is a diagram showing a network 1400 with a plurality of computer systems 1402a, and 1402b, a plurality of cell phones and personal data assistants 1402c, and Network Attached Storage (NAS) 1404a, and 1404b. In example instances, systems 1402a, 1402b, and 1402c can manage data storage and optimize data access for data stored in Network Attached Storage (NAS) 1404a and 1404b. A mathematical model can be used for the data and be evaluated using distributed parallel processing across computer systems 1402a, and 1402b, and cell phone and personal data assistant systems 1402c. Computer systems 1402a, and 1402b, and cell phone and personal data assistant systems 1402c can also provide parallel processing for adaptive data restructuring of the data stored in Network Attached Storage (NAS) 1404a and 1404b. FIG. 6 illustrates an example only, and a wide variety of other computer architectures and systems can be used in conjunction with the various instances of the present disclosure. For example, a blade server can be used to provide parallel processing. Processor blades can be connected through a back plane to provide parallel processing. Storage can also be connected to the back plane or as Network Attached Storage (NAS) through a separate network interface. In some example instances, processors can maintain separate memory spaces and transmit data through network interfaces, back plane or other connectors for parallel processing by other processors. In other instances, some or all of the processors can use a shared virtual address memory space.

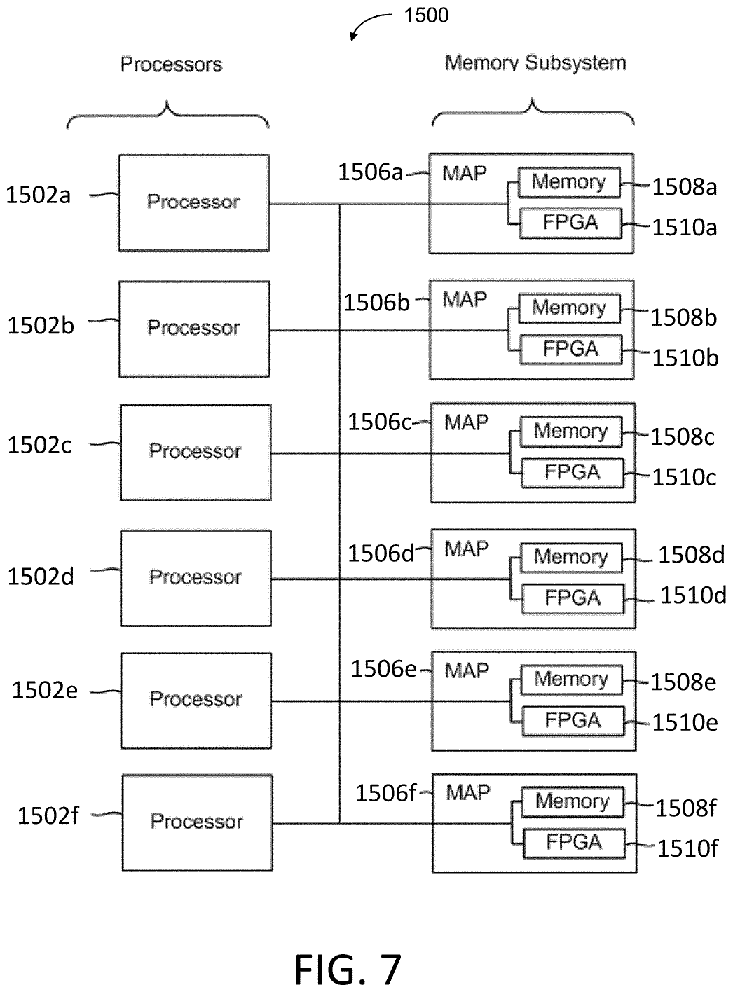

FIG. 7 is a block diagram of a multiprocessor computer system 1500 using a shared virtual address memory space in accordance with an example instance. The system includes a plurality of processors 1502a-f that can access a shared memory subsystem 1504. The system incorporates a plurality of programmable hardware memory algorithm processors (MAPs) 1506a-f in the memory subsystem 1504. Each MAP 1506a-f can comprise a memory 1508a-f and one or more field programmable gate arrays (FPGAs) 1510a-f. The MAP provides a configurable functional unit and particular algorithms or portions of algorithms can be provided to the FPGAs 1510a-f for processing in close coordination with a respective processor. For example, the MAPs can be used to evaluate algebraic expressions regarding the data model and to perform adaptive data restructuring in example instances. In this example, each MAP is globally accessible by all of the processors for these purposes. In one configuration, each MAP can use Direct Memory Access (DMA) to access an associated memory 1508a-f, allowing it to execute tasks independently of, and asynchronously from the respective microprocessor 1502a-f. In this configuration, a MAP can feed results directly to another MAP for pipelining and parallel execution of algorithms.

The above computer architectures and systems are examples only, and a wide variety of other computer, cell phone, and personal data assistant architectures and systems can be used in connection with example instances, including systems using any combination of general processors, co-processors, FPGAs and other programmable logic devices, system on chips (SOCs), application specific integrated circuits (ASICs), and other processing and logic elements. In some instances, all or part of the computer system can be implemented in software or hardware. Any variety of data storage media can be used in connection with example instances, including random access memory, hard drives, flash memory, tape drives, disk arrays, Network Attached Storage (NAS) and other local or distributed data storage devices and systems.

In example instances, the computer system can be implemented using software modules executing on any of the above or other computer architectures and systems. In other instances, the functions of the system can be implemented partially or completely in firmware, programmable logic devices such as field programmable gate arrays (FPGAs) as referenced in FIG. 7, system on chips (SOCs), application specific integrated circuits (ASICs), or other processing and logic elements. For example, the Set Processor and Optimizer can be implemented with hardware acceleration through the use of a hardware accelerator card, such as accelerator card 1322 illustrated in FIG. 5.

Numbered Embodiments

Provided herein are numbered embodiments 1-83. Embodiment 1. A polynucleotide library comprising: a sample polynucleotide set comprising at least 100 polynucleotides derived from genomic sequences; and a background set comprising background polynucleotides, wherein the background set comprises cell-free DNA (cfDNA), wherein each of the least 100 polynucleotides of the sample polynucleotide set comprises at least one variant, wherein the at least one variant comprises one or more changes compared to a background polynucleotide; and at least 2 polynucleotides of the at least 100 polynucleotides are tiled across each of the at least one variant. Embodiment 2. The library of embodiment 1, wherein each of the least 100 polynucleotides comprises one variant. Embodiment 3. The library of embodiment 2, wherein the sample polynucleotide set comprises at least 150 variants. Embodiment 4. The library of embodiment 2, wherein the sample polynucleotide set comprises at least 400 variants. Embodiment 5. The library of any one of embodiments 1-4, wherein at least 5 polynucleotides are tiled across each of the at least one variant. Embodiment 6. The library of embodiment 5, wherein at least 20 polynucleotides are tiled across the at least one variant. Embodiment 7. The library of embodiment 6, wherein at least 30 polynucleotides are tiled across the at least one variant. Embodiment 8. The library of any one of embodiments 1-7, wherein the least at least 10 polynucleotides are tiled across the at least one variant with an offset of 1-8 bases. Embodiment 9. The library of any one of embodiments 1-8, wherein the genomic sequences are derived from cell-free DNA (cfDNA). Embodiment 10. The library of any one of embodiments 1-9, wherein the sample polynucleotide set comprises no more than 10% of the total amount of polynucleotides in the library. Embodiment 11. The library of any one of embodiments 1-10, wherein the at least one variant is present at a frequency of 0.01-5% relative to a wild-type genomic sequence. Embodiment 12. The library of embodiment 11, wherein the at least one variant is present at a frequency of 1-5% relative to a wild-type genomic sequence. Embodiment 13. The library of embodiment 11, wherein the at least one variant is present at a frequency of 0.1% 0.25%, 0.5%, 1%, or 2% relative to a wild-type genomic sequence. Embodiment 14. The library of embodiment any one of embodiments 1-13, wherein at least 90% of the at least one variants is present at a frequency of no more than 10% relative to the frequency of other variants. Embodiment 15. The library of embodiment 14, wherein at least 99% of the at least one variants is present at a frequency of no more than 20% relative to the frequency of other variants. Embodiment 16. The library of any one of embodiments 1-15, wherein at least some of the least 100 polynucleotides are double stranded. Embodiment 17. The library of embodiment 16, wherein at least 90% of the least 100 polynucleotides are double stranded. Embodiment 18. The library of any one of embodiments 1-17, wherein the length of at least some of the least 100 polynucleotides is 125-200 bases. Embodiment 19. The library of embodiment 18, wherein the length of at least 90% of the least 100 polynucleotides is 125-200 bases. Embodiment 20. The library of any one of embodiments 1-19, wherein the at least one variant comprises an insertion, deletion, fusion, duplication, frameshift, repeat expansion, or substitution. Embodiment 21. The library of any one of embodiments 1-19, wherein the at least one variant comprises a copy number variant (CNV), microsatellite instability, loss of heterozygosity (LOH), DNA methylation, premature stop codon, trinucleotide repeat, translocation, somatic rearrangement, allelomorph, single nucleotide variant (SNV), indel, splice variant, regulator variant, copy number variant, or fusion. Embodiment 22. The library of any one of embodiments 1-19, wherein the at least one variant comprises a single nucleotide variant, indel, fusion, or structural variant. Embodiment 23. The library of embodiment 22, wherein the indel is 1-15 bases in length. Embodiment 24. The library of any one of embodiments 1-23, wherein the at least one variant comprises a modification to an tumor suppressor or oncogene. Embodiment 25. The library of any one of embodiments 1-24, wherein the library comprises variants located in at least 50 genes. Embodiment 26. The library of embodiment 25, wherein the library comprises variants located in at least 75 genes. Embodiment 27. The library of any one of embodiments 1-26, wherein the at least one variant is located in one or more of genes ABL1, ABL2, AKT1, ALK, APC, AR, ARAF, ARID1A, ATM, ATR, BAP1, BRAF, BRCA1, BRCA2, CCND1, CDC6, CDH1, CDK12, CDK4, CDX2, CTNNB1, DDR2, EGFR, EML4, ERBB2, ERBB3, ERG, ESR1, EZH2, FBXW7, FGFR1, FGFR2, FGFR3, FLT3, FOXA1, FOXL2, GATA3, GNA11, GNAQ, GNAS, HNF1A, HRAS, IDH1, IDH2, JAK2, KDM5C, KDM6A, KIF5B, KIT, KRAS, MAP2K1, MAPK1, MET, MIR4728, ERBB2, MLH1, MPL, MYCN, MYD88, NCOA4, NF1, NF2, NFE2L2, NOTCHI, NPM1, NRAS, PBRM1, PDGFRA, PIK3CA, PTEN, PTPN11, RET, RHEB, RHOA, RIT1, ROS1, SETD2, SMAD4, SMO, SPOP, TERT, TMPRSS2, TP53, TPR, TSC1, and VHL. Embodiment 28. The library of embodiment 27, wherein the at least one variant is located in ten or more of genes ABL1, ABL2, AKT1, ALK, APC, AR, ARAF, ARID1A, ATM, ATR, BAP1, BRAF, BRCA1, BRCA2, CCND1, CDC6, CDH1, CDK12, CDK4, CDX2, CTNNB1, DDR2, EGFR, EML4, ERBB2, ERBB3, ERG, ESR1, EZH2, FBXW7, FGFR1, FGFR2, FGFR3, FLT3, FOXA1, FOXL2, GATA3, GNA11, GNAQ, GNAS, HNF1A, HRAS, IDH1, IDH2, JAK2, KDM5C, KDM6A, KIF5B, KIT, KRAS, MAP2K1, MAPK1, MET, MIR4728, ERBB2, MLH1, MPL, MYCN, MYD88, NCOA4, NF1, NF2, NFE2L2, NOTCHI, NPM1, NRAS, PBRM1, PDGFRA, PIK3CA, PTEN, PTPN11, RET, RHEB, RHOA, RIT1, ROS1, SETD2, SMAD4, SMO, SPOP, TERT, TMPRSS2, TP53, TPR, TSC1, and VHL. Embodiment 29. The library of any one of embodiments 1-28, wherein the sample polynucleotide set is substantially free of biological contamination. Embodiment 30. The library of embodiment 29, wherein the biological contamination comprises cellular components or biomolecules derived from plasma. Embodiment 31. The library of any one of embodiments 1-30, wherein the library further comprises a buffer. Embodiment 32. The library of any one of embodiments 1-31, wherein the background polynucleotide set comprises wild-type regions corresponding to locations of the at least one variant. Embodiment 33. The library of embodiment 32, wherein the wild-type regions are represented within 10% of the variant frequency of the variant set. Embodiment 34. The library of any one of embodiments 1-33, wherein the background polynucleotide set comprises two or more polynucleotides. Embodiment 35. The library of any one of embodiments 1-34, wherein highest abundance of polynucleotides in the background set are 125-200 bases in length. Embodiment 36. The library of embodiment 35, wherein highest abundance of polynucleotides in the background set are 150-185 bases in length. Embodiment 37. The library of any one of embodiments 1-36, wherein at least 90% of the polynucleotides in the background set are mononucleosomal or dinucleosomal. Embodiment 38. The library of any one of embodiments 1-37, wherein the ratio of mononucleosomal to dinucleosomal is 70:30 to 90:10. Embodiment 39. The library of any one of embodiments 1-38, wherein the background polynucleotide set is derived from a healthy human. Embodiment 40. The library of embodiment 39, wherein the background polynucleotide set is isolated from a healthy human. Embodiment 41. The library of embodiment 40, wherein the human is male. Embodiment 42. The library of embodiment 41, wherein the human is no more than 30 years old. Embodiment 43. The library of any one of embodiments 1-42, wherein at least one background polynucleotide comprises a variant present at a frequency of 0.001%, 0.01%, 0.1% 0.25%, 0.5%, 1%, or 2% relative to a wild-type genomic sequence. Embodiment 44. A kit for measuring variant detection limits comprising: a) The library of any one of embodiments 1-43; b) instructions for use of the kit; and c) packaging configured to hold and describe the kit contents. Embodiment 45. The kit of embodiment 44, wherein the kit comprises at least two libraries of any one of embodiment 1-43. Embodiment 46. The kit of embodiment 44 or 45, wherein the at least two libraries each comprise variants present at a frequency of 0%, 0.1% 0.25%, 0.5%, 1%, or 2% relative to a wild-type genomic sequence. Embodiment 47. The kit of embodiment 46, wherein the kit comprises five libraries, each comprising variants present at a frequency of 0%, 0.1% 0.25%, 0.5%, 1%, or 2% relative to a wild-type genomic sequence. Embodiment 48. A method of preparing the library of any one of embodiments 1-43 comprising: a) providing the background polynucleotide set; b) synthesizing the sample polynucleotide set from predetermined sequences; and c) mixing the variant set and the background set in a buffer. Embodiment 49. The method of embodiment 48, wherein synthesizing comprises chemical synthesis. Embodiment 50. The method of embodiment 48 or 49, wherein synthesizing comprises synthesis on a surface. Embodiment 51. The method of any one of embodiments 48-50, wherein synthesizing comprises coupling of nucleoside phosphoramidites. Embodiment 52. The method of any one of embodiments 48-51, further comprising sequencing the library. Embodiment 53. The method of any one of embodiments 48-52, further comprising ddPCR measurement of the library. Embodiment 54. The method of any one of embodiments 48-53, further comprising fluorescence/UV DNA quantification and size distribution of the library. Embodiment 55. The method of any one of embodiments 48-54, further comprising determining the variant frequency in the background polynucleotide set, where the variants correspond to the at least one variant in the sample polynucleotide set. Embodiment 56. The method of any one of embodiments 48-55, further comprising fluorescence/UV DNA quantification of the sample polynucleotide set prior to mixing. Embodiment 57. The method of any one of embodiments 48-56, further comprising electrophoretic fragment analysis of the sample polynucleotide set prior to mixing. Embodiment 58. A method of preparing a nucleic acid test sample useful for determining the detection limit of genomic variants comprising: a) providing a library of any one of embodiments 1-43; b) obtaining at least one test sample from a patient suspected of having a disease or condition; c) detecting the presence or absence of the one or more variants in the library of any one of embodiments 1-43; and d) detecting the presence or absence of the one or more variants in the at least one test sample. Embodiment 59. The method of embodiment 58, wherein detecting comprises sequencing. Embodiment 60. The method of embodiment 59, wherein detecting comprises Next Generation Sequencing. Embodiment 61. The method of embodiment 59 or 60, wherein sequencing comprises sequencing by synthesis, nanopore sequencing, or SMRT sequencing. Embodiment 62. The method of embodiment 58, wherein detecting comprises ddPCR or specific hybridization to an array. Embodiment 63. The method of any one of embodiments 58-62, wherein the at least one test sample comprises a liquid biopsy. Embodiment 64. The method of any one of embodiments 58-63, wherein the at least one test sample comprises circulating tumor DNA (ctDNA). Embodiment 65. The method of any one of embodiments 58-64, wherein the at least one test sample is obtained from blood. Embodiment 66. The method of any one of embodiments 58-65, wherein the at least one test sample is substantially cell-free. Embodiment 67. The method of any one of embodiments 58-66, wherein the method comprises at least 5 test samples. Embodiment 68. The method of any one of embodiments 58-67, wherein the method further comprises detection of minimal residual disease (MRD). Embodiment 69. The method of any one of embodiments 58-68, wherein the patient is suspected of having a disease or condition. Embodiment 70. The method of embodiment 69, wherein the disease or condition is a proliferative disease. Embodiment 71. The method of embodiment 69, wherein the disease or condition is cancer. Embodiment 72. The method of any one of embodiments 58-71, wherein the patient was previously treated, is currently treated, or has received a clinical diagnosis for cancer. Embodiment 73. The method of any one of embodiments 58-72, wherein the method further comprises ligating sequencing adapters to at least some polynucleotides in the test sample, the library, or both. Embodiment 74. The method of any one of embodiments 58-73, wherein the method further comprises amplifying at least some polynucleotides in the test sample, the library, or both. Embodiment 75. The method of any one of embodiments 58-74, wherein if one or more variants are not detected in the library, then results obtained from the at least one test sample is discarded or re-analyzed. Embodiment 76. The method of any one of embodiments 58-75, wherein detecting comprises addition of one or more adapters to at least some sample polynucleotides in the library. Embodiment 77. The method of embodiment 76, wherein the adapters comprise at least one barcode. Embodiment 78. The method of embodiment 77, wherein the at least one barcode comprises one or more of a unique molecular identifier and a sample index. Embodiment 79. The method of embodiment 78, where the at least one adapter comprises a duplex adapter. Embodiment 80. The method of embodiment 78, wherein at least one adapter comprises at least two unique molecular identifiers. Embodiment 81. The method of embodiment 78, wherein at least one adapter comprises a first unique molecular identifier and a second unique molecular identifier. Embodiment 82. The method of embodiment 81, wherein the first unique molecular identifier or the second unique molecular identifier comprises a sequence of one or more of AAGGA, ACAAC, ATACG, CACTG, CATGA, CGATA, CGTGT, GCCAT, GCTGT, GTCAC, GTCGT, TACGA, TCCTA, TCGTG, TGTCG, TTGGC, AACAC, AATGC, ACTAG, AGCAT, AGTAC, ATCTC, CAGAC, CAGTA, CGAAT, CGGTT, CTTGG, GCATA, GCTAA, GTGAG, GTGTC, and TGTGC. Embodiment 83. The method of embodiment 81, wherein the first unique molecular identifier or the second unique molecular identifier comprises a sequences of 10 or more of

AAGGA,
ACAAC,
ATACG,
CACTG,
CATGA,
CGATA,
CGTGT,
GCCAT,
GCTGT,
GTCAC,
GTCGT,
TACGA,
TCCTA,
TCGTG,
TGTCG,
TTGGC,
AACAC,
AATGC,
ACTAG,
AGCAT,
AGTAC,
ATCTC,
CAGAC,
CAGTA,
CGAAT,
CGGTT,
CTTGG,
GCATA,
GCTAA,
GTGAG,
GTGTC,
and
TGTGC.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Functionalization of a Substrate Surface

A substrate was functionalized to support the attachment and synthesis of a library of polynucleotides. The substrate surface was first wet cleaned using a piranha solution comprising 90% H₂SO₄ and 10% H₂O₂ for 20 minutes. The substrate was rinsed in several beakers with DI water, held under a DI water gooseneck faucet for 5 minutes, and dried with N₂. The substrate was subsequently soaked in NH₄OH (1:100; 3 mL:300 mL) for 5 minutes, rinsed with DI water using a handgun, soaked in three successive beakers with DI water for 1 minute each, and then rinsed again with DI water using the handgun. The substrate was then plasma cleaned by exposing the substrate surface to O₂. A SAMCO PC-300 instrument was used to plasma etch O₂ at 250 watts for 1 minute in downstream mode.

The cleaned substrate surface was actively functionalized with a solution comprising N-(3-triethoxysilylpropyl)-4-hydroxybutyramide using a YES-1224P vapor deposition oven system with the following parameters: 0.5 to 1 torr, 60 minutes, 70° C., 135° C. vaporizer. The substrate surface was resist coated using a Brewer Science 200× spin coater. SPR™ 3612 photoresist was spin coated on the substrate at 2500 rpm for 40 seconds. The substrate was pre-baked for 30 minutes at 90° C. on a Brewer hot plate. The substrate was subjected to photolithography using a Karl Suss MA6 mask aligner instrument. The substrate was exposed for 2.2 seconds and developed for 1 minute in MSF 26A. Remaining developer was rinsed with the handgun and the substrate soaked in water for 5 minutes. The substrate was baked for 30 minutes at 100° C. in the oven, followed by visual inspection for lithography defects using a Nikon L200. A descum process was used to remove residual resist using the SAMCO PC-300 instrument to O₂ plasma etch at 250 watts for 1 minute.

The substrate surface was passively functionalized with a 100 μL solution of perfluorooctyltrichlorosilane mixed with 10 μL light mineral oil. The substrate was placed in a chamber, pumped for 10 minutes, and then the valve was closed to the pump and left to stand for 10 minutes. The chamber was vented to air. The substrate was resist stripped by performing two soaks for 5 minutes in 500 mL NMP at 70° C. with ultrasonication at maximum power (9 on Crest system). The substrate was then soaked for 5 minutes in 500 mL isopropanol at room temperature with ultrasonication at maximum power. The substrate was dipped in 300 mL of 200 proof ethanol and blown dry with N₂. The functionalized surface was activated to serve as a support for polynucleotide synthesis.

### Example 2: Synthesis of a 50-Mer Sequence on a Polynucleotide Synthesis Device

A two dimensional polynucleotide synthesis device was assembled into a flowcell, which was connected to a flowcell (Applied Biosystems (ABI394 DNA Synthesizer”). The polynucleotide synthesis device was uniformly functionalized with N-(3-TRIETHOXYSILYLPROPYL)-4-HYDROXYBUTYRAMIDE (Gelest) was used to synthesize an exemplary polynucleotide of 50 bp (“50-mer polynucleotide”) using polynucleotide synthesis methods described herein.

The sequence of the 50-mer was as described in SEQ ID NO.: 1. 5′AGACAATCAACCATTTGGGGTGGACAGCCTTGACCTCTAGACTTCGGCAT ##TTTTTTTTT T3′ (SEQ ID NO.: 1), where # denotes Thymidine-succinyl hexamide CED phosphoramidite (CLP-2244 from ChemGenes), which is a cleavable linker enabling the release of polynucleotides from the surface during deprotection.

The synthesis was done using standard DNA synthesis chemistry (coupling, capping, oxidation, and deblocking) and an ABI synthesizer.

The phosphoramidite/activator combination was delivered similar to the delivery of bulk reagents through the flowcell. No drying steps were performed as the environment stays “wet” with reagent the entire time.

The flow restrictor was removed from the ABI 394 synthesizer to enable faster flow. Without flow restrictor, flow rates for amidites (0.1M in ACN), Activator, (0.25M Benzoylthiotetrazole (“BTT”; 30-3070-xx from GlenResearch) in ACN), and Ox (0.02M I₂ in 20% pyridine, 10% water, and 70% THF) were roughly ˜100 uL/second, for acetonitrile (“ACN”) and capping reagents (1:1 mix of CapA and CapB, wherein CapA is acetic anhydride in THF/Pyridine and CapB is 16% 1-methylimidizole in THF), roughly ˜200 uL/second, and for Deblock (3% dichloroacetic acid in toluene), roughly ˜300 uL/second (compared to ˜50 uL/second for all reagents with flow restrictor). The time to completely push out Oxidizer was observed, the timing for chemical flow times was adjusted accordingly and an extra ACN wash was introduced between different chemicals. After polynucleotide synthesis, the chip was deprotected in gaseous ammonia overnight at 75 psi. Five drops of water were applied to the surface to recover polynucleotides. The recovered polynucleotides were then analyzed on a BioAnalyzer small RNA chip (data not shown).

### Example 3: Synthesis of a 100-Mer Sequence on a Polynucleotide Synthesis Device

The same process as described in Example 2 for the synthesis of the 50-mer sequence was used for the synthesis of a 100-mer polynucleotide (“100-mer polynucleotide”; 5′ CGGGATCCTTATCGTCATCGTCGTACAGATCCCGACCCATTTGCTGTCCACCAGTCATGCT AGCCATACCATGATGATGATGATGATGAGAACCCCGCAT ##TTTTTTTTTT3′, where #denotes Thymidine-succinyl hexamide CED phosphoramidite (CLP-2244 from ChemGenes); SEQ ID NO.: 2) on two different silicon chips, the first one uniformly functionalized with N-(3-TRIETHOXYSILYLPROPYL)-4-HYDROXYBUTYRAMIDE and the second one functionalized with 5/95 mix of 11-acetoxyundecyltriethoxysilane and n-decyltriethoxysilane, and the polynucleotides extracted from the surface were analyzed on a BioAnalyzer instrument (data not shown).

All ten samples from the two chips were further PCR amplified using a forward (5′ATGCGGGGTTCTCATCATC3′; SEQ ID NO.: 3) and a reverse (5′CGGGATCCTTATCGTCATCG3′; SEQ ID NO.: 4) primer in a 50 uL PCR mix (25 uL NEB Q5 master mix, 2.5 uL 10 uM Forward primer, 2.5 uL 10 uM Reverse primer, 1 uL polynucleotide extracted from the surface, and water up to 50 uL) using the following thermal cycling program:
98 C, 30 seconds
98 C, 10 seconds; 63C, 10 seconds; 72C, 10 seconds; repeat 12 cycles
72C, 2 minutes

The PCR products were also run on a BioAnalyzer (data not shown), demonstrating sharp peaks at the 100-mer position. Next, the PCR amplified samples were cloned, and Sanger sequenced. Table 7 summarizes the results from the Sanger sequencing for samples taken from spots 1-5 from chip 1 and for samples taken from spots 6-10 from chip 2.

TABLE 7

| Spot | Error rate | Cycle efficiency |
|---|---|---|
| 1 | 1/763 bp | 99.87% |
| 2 | 1/824 bp | 99.88% |
| 3 | 1/780 bp | 99.87% |
| 4 | 1/429 bp | 99.77% |
| 5 | 1/1525 bp | 99.93% |
| 6 | 1/1615 bp | 99.94% |
| 7 | 1/531 bp | 99.81% |
| 8 | 1/1769 bp | 99.94% |
| 9 | 1/854 bp | 99.88% |
| 10 | 1/1451 bp | 99.93% |

Thus, the high quality and uniformity of the synthesized polynucleotides were repeated on two chips with different surface chemistries. Overall, 89%, corresponding to 233 out of 262 of the 100-mers that were sequenced were perfect sequences with no errors.

Finally, Table 8 summarizes error characteristics for the sequences obtained from the polynucleotides samples from spots 1-10.

TABLE 8

| Sample | OSA_ | OSA_ | OSA_ | OSA_ | OSA_ | OSA_ | OSA_ | OSA_ | OSA_ | OSA_ |
|---|---|---|---|---|---|---|---|---|---|---|
| ID/Spot no. | 0046/1 | 0047/2 | 0048/3 | 0049/4 | 0050/5 | 0051/6 | 0052/7 | 0053/8 | 0054/9 | 055/10 |
| Total | 32 | 32 | 32 | 32 | 32 | 32 | 32 | 32 | 32 | 32 |
| Sequences | | | | | | | | | | |
| Sequencing | 25 of 28 | 27 of 27 | 26 of 30 | 21 of 23 | 25 of 26 | 29 of 30 | 27 of 31 | 29 of 31 | 28 of 29 | 25 of 28 |
| Quality | | | | | | | | | | |
| Oligo | 23 of 25 | 25 of 27 | 22 of 26 | 18 of 21 | 24 of 25 | 25 of 29 | 22 of 27 | 28 of 29 | 26 of 28 | 20 of 25 |
| Quality | | | | | | | | | | |
| ROI Match | 2500 | 2698 | 2561 | 2122 | 2499 | 2666 | 2625 | 2899 | 2798 | 2348 |
| Count | | | | | | | | | | |
| ROI | 2 | 2 | 1 | 3 | 1 | 0 | 2 | 1 | 2 | 1 |
| Mutation | | | | | | | | | | |
| ROI Multi | 0 | 0 | 0 | 0 | 0 | 0 | 0 | 0 | 0 | 0 |
| Base | | | | | | | | | | |
| Deletion | | | | | | | | | | |
| ROI Small | 1 | 0 | 0 | 0 | 0 | 0 | 0 | 0 | 0 | 0 |
| Insertion | | | | | | | | | | |
| ROI Single | 0 | 0 | 0 | 0 | 0 | 0 | 0 | 0 | 0 | 0 |
| Base | | | | | | | | | | |
| Deletion | | | | | | | | | | |
| Large | 0 | 0 | 1 | 0 | 0 | 1 | 1 | 0 | 0 | 0 |
| Deletion | | | | | | | | | | |
| Count | | | | | | | | | | |
| Mutation: | 2 | 2 | 1 | 2 | 1 | 0 | 2 | 1 | 2 | 1 |
| G > A | | | | | | | | | | |
| Mutation: | 0 | 0 | 0 | 1 | 0 | 0 | 0 | 0 | 0 | 0 |
| T > C | | | | | | | | | | |
| ROI Error | 3 | 2 | 2 | 3 | 1 | 1 | 3 | 1 | 2 | 1 |
| Count | | | | | | | | | | |
| ROI Error | Err: ~1 | Err: ~1 | Err: ~1 | Err: ~1 | Err: ~1 | Err: ~1 | Err: ~1 | Err: ~1 | Err: ~1 | Err: ~1 |
| Rate | in 834 | in 1350 | in 1282 | in 708 | in 2500 | in 2667 | in 876 | in 2900 | in 1400 | in 2349 |
| ROI Minus | MP | MP | MP | MP | MP | MP | MP | MP | MP | MP |
| Primer | Err: ~1 | Err: ~1 | Err: ~1 | Err: ~1 | Err: ~1 | Err: ~1 | Err: ~1 | Err: ~1 | Err: ~1 | Err: ~1 |
| Error Rate | in 763 | in 824 | in 780 | in 429 | in 1525 | in 1615 | in 531 | in 1769 | in 854 | in 1451 |

### Example 4: Parallel Assembly of 29,040 Unique Polynucleotides

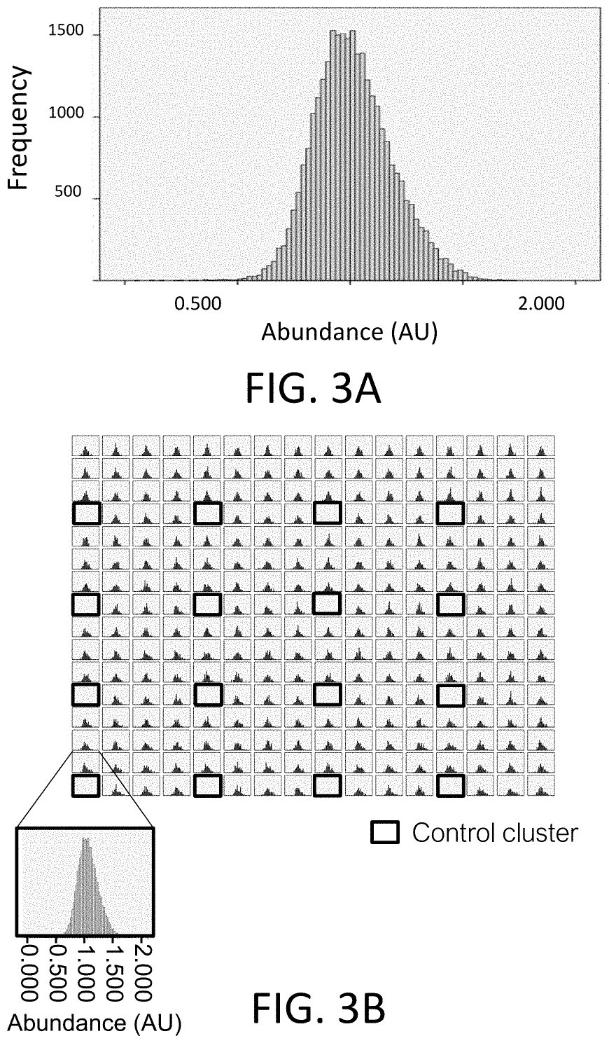

A structure comprising 256 clusters each comprising 121 loci on a flat silicon plate 201 was manufactured as shown in FIG. 2. An expanded view of a cluster is shown in 205 with 121 loci. Loci from 240 of the 256 clusters provided an attachment and support for the synthesis of polynucleotides having distinct sequences. Polynucleotide synthesis was performed by phosphoramidite chemistry using general methods from Example 3. Loci from 16 of the 256 clusters were control clusters. The global distribution of the 29,040 unique polynucleotides synthesized (240×121) is shown in FIG. 3A. Polynucleotide libraries were synthesized at high uniformity. 90% of sequences were present at signals within 4× of the mean, allowing for 100% representation. Distribution was measured for each cluster, as shown in FIG. 3B. On a global level, all polynucleotides in the run were present and 99% of the polynucleotides had abundance that was within 2× of the mean indicating synthesis uniformity. This same observation was consistent on a per-cluster level.

The error rate for each polynucleotide was determined using an Illumina MiSeq gene sequencer. The error rate distribution for the 29,040 unique polynucleotides averages around 1 in 500 bases, with some error rates as low as 1 in 800 bases. Distribution was measured for each cluster. The library of 29,040 unique polynucleotides was synthesized in less than 20 hours. Analysis of GC percentage versus polynucleotide representation across all of the 29,040 unique polynucleotides showed that synthesis was uniform despite GC content.

### Example 5. Design and Synthesis of a Synthetic cfDNA Variant Library

Using the general synthesis methods described in Example 3, a synthetic variant library was designed and synthesized. The total number of target variants represented was 458, and each polynucleotide in the library was 167 base pairs in length. Variants were present on 85 different human genes, and included SNVs (228), indels (215 total; 168 deletions, 47 insertions), fusions, and SVs (15). This included 147 clinically relevant variants (including all SVs). Variants were selected from Tables 1-6. Polynucleotides targeting a single variant were tiled using the general design of FIG. 1A, with an offset of 4 bases and with 32 polynucleotides targeting each variant. The distribution of indel sizes for the library is shown in FIG. 1B. The variant library was then mixed with a background cfDNA library obtained from plasma of a healthy male donor (less than 30 years old, shown in FIG. 1C). Libraries having a variant allele frequency (VAF) of 0% (wild-type), 0.1%, 0.25%, 0.5%, 1%, 2%, and 5% were generated. Accurate representation and distribution of polynucleotides in the library was further confirmed by Next Generation Sequencing (all variant sites) and ddPCR (for a subset of variant sites).

### Example 6. Variant Libraries as a Reference Standard

At least one sample from a patient suspected of having a disease or condition is obtained, such as a sample obtained via liquid biopsy. The patient may have been previously untreated, previously diagnosed/treated, or concurrently treated for a disease or condition. A library generated using the general methods of Example 5 (reference standard, includes mixture variant polynucleotides and background cfDNA) is analyzed on an instrument (sequencing or ddPCR) with the at least one patient sample. If the variants are not detected with the required confidence in the reference standard, the instrument may be adjusted/recalibrated, subjected to maintenance, or the patient sample may be re-analyzed or results discarded. From the sensitivity of the reference standard, the patient sample is analyzed and determined to contain or not contain one or more variants found in the reference standard. Based on this result, the patient may be diagnosed or treated appropriately by a healthcare professional.

### Example 7. Design of ctDNA Standards Using Restriction Site Adapter Cleavage

Sequences for approximately 500 variants were acquired comprising mostly SBS (single base substitutions) from a reference genome. Approximately 10,000 fragments were designed having a length of about 160 bp, with an 8 bp sliding window. About 20 fragments were tiled across each variant. Optionally, a 5 base identifier was added to label the fragments as synthetic. This identifier in some instances was a significant edit distance from the reference gene, or else it may just be called as a variant. Given a variant fasta file, fragments are designed by:
1. Selecting 162 bases (for 2 base “synthetic signatures” to the 5′ and 3′ of the variant base, for a total of 325 bases.
2. The 5′ 164 bases will be fragment 1.
3. Looping over a sliding window+8, each will be new fragment, 20 fragments to synthesize per variant.
4. For each fragment, change 5 bases at the 5′ end to encode the complement. i.e., AGATC . . . TCTAG . . . .
5. For each fragment, change 5 bases at the 3′ end to encode the complement as above.

If the variant is at the end of a molecule, in some instances it is soft-clipped. In one embodiment, the sliding window is at 7, but starts closer to the variant. This would result in 20 unique molecules per variant.

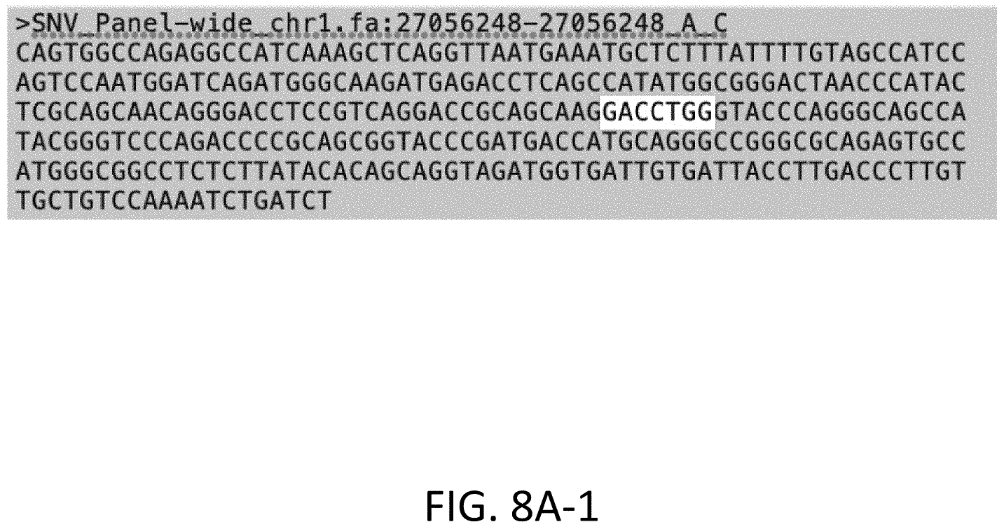

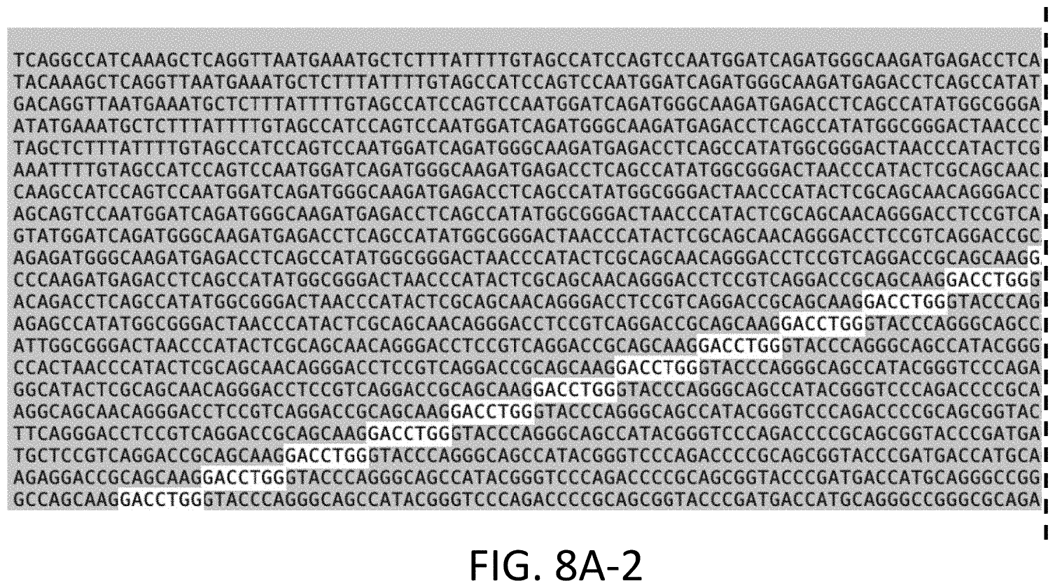

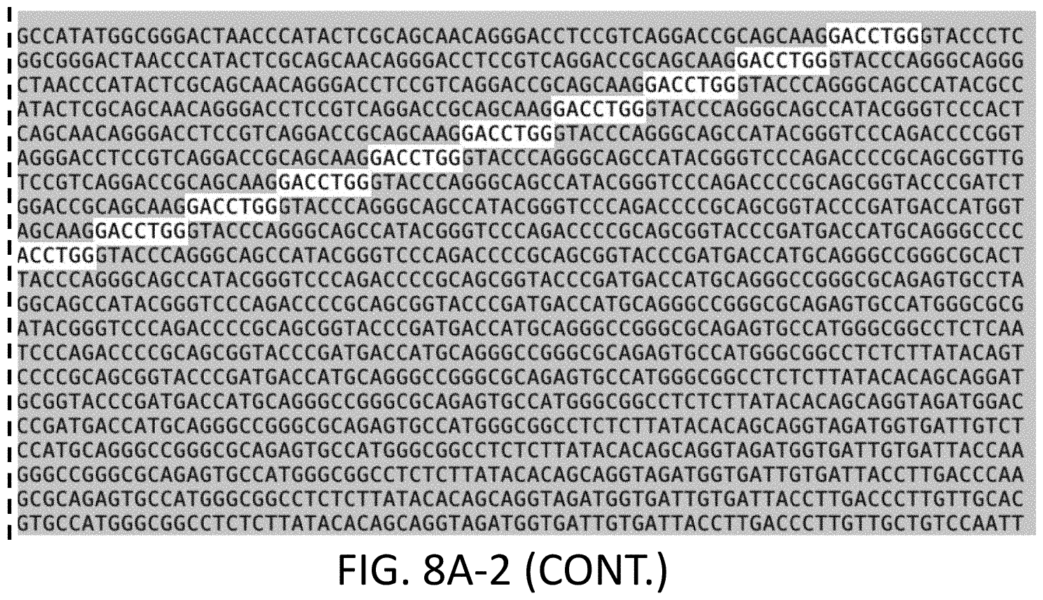

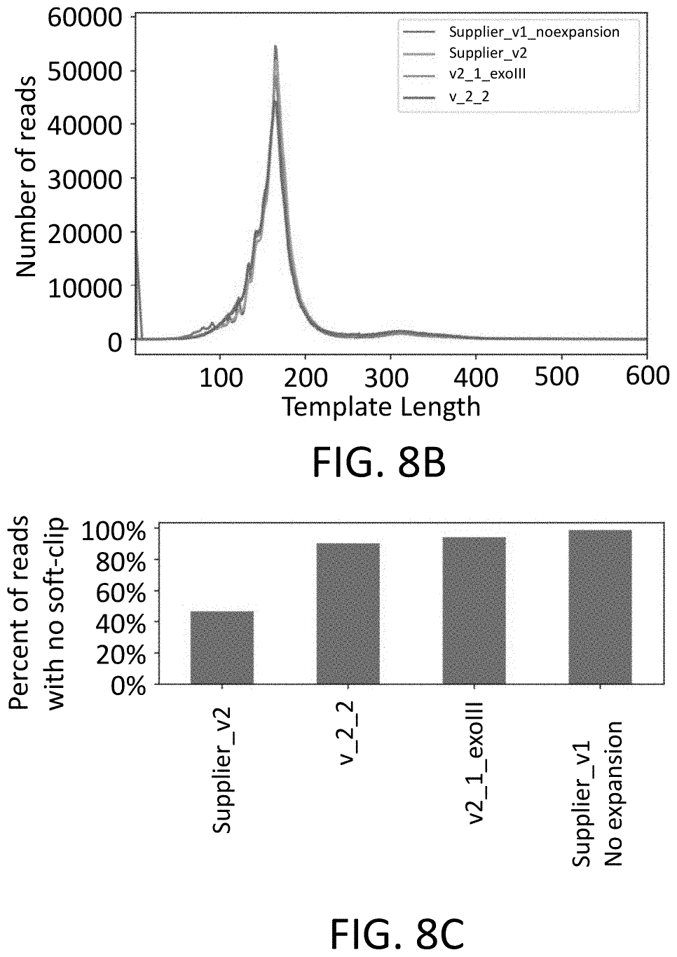

The length is 324 bp (for 2 bp on each end for barcoding). The variant is placed at position 161. In another embodiment, the sliding window is +7 (every 8th base), the variant is at base 161 in the original fasta at 171 in the expanded fasta, start at −150, fragment length is 164, 2 bp on each end is complemented, and flanks are added as described below. FIG. 8A depicts an example of 20 oligos to be synthesized, without the flanks added, to show the location of each of the variants across each molecule. The top is the original variant. In the bottom 20, each line is a unique molecule from the sliding window. The highlighted region contains the variant base. Within the GACCTGG, the bolded base is the variant. It is present within each molecule at least 8 bases within the end of the alignable. Flanks are added as below. Initial builds using this design resulted in 6760 oligos for the SNVs (333 variants with 20 oligos per variant). The oligos are screened for restriction sites:

TABLE 9

| | bspq1 | bsmb1 | bbs1 |
|---|---|---|---|
| number of oligos | 162 | 208 | 792 |
| with site | | | |
| % with site | 2.39538666 | 3.07555818 | 11.7107792 |

Bspq1 and bsmb1 (both 7 cutters) result in fewer oligos with cut sites; bbsl is a 6 cutter, and cuts more frequently. BSPQ1 cleaves at the fewest endogenous locations, so this is used to remove adapters; the cut sequences are:

GCTCTTC(N1)- 3′
CGAGAAG(N4)- 5′

There is a 3 base 5′ overhang after cutting. These are filled in with Klenow after cleanup. The N1 base is in ( ). The initial oligo has the sequence: 5′-GAAGTGCCATTCCGC GCTCTTC(A) (SEQ ID NO: 58)-2b complement—160b w/variant—2b complement—(T)GAAGAGC ATCGTACAG CTGCTCG-3′ (SEQ ID NO: 59)

In another embodiment, the oligo has the sequence: 5′-CCATTCCGC GCTCTTC(A) (SEQ ID NO: 60)-2b complement—160b w/variant—2b complement—(T)GAAGAGCATC GTACAGCT-3′ (SEQ ID NO: 61)

Exemplary primers include those described in Tables 10A and 10B.

TABLE 10A

| | |
|---|---|
| Uni9 | GAAGTGCCATTCCGCCTGACCT (SEQ ID |
| | NO: 62) |
| B1-BSPQ-M-AFR-1B | #-CGAGCAGCTGTACGATGCTCTTCA (SEQ |
| | ID NO: 63) |
| B2-BSPQ-M-AFR-1B | #-CGCTGACGATGTCAGTGCTCTTCA (SEQ |
| | ID NO: 64) |

TABLE 10B

| | Forward primer | Reverse primer | rev comp |
|---|---|---|---|
| | (Uni9 based) | (Bl based) | reverse |
| original | #- | #- | TGAAGAGCATCGTACAGCTG |
| | GAAGTGCCATTCCGCGCTC | CGAGCAGCTGTACGATGCTC | CTCG (SEQ ID NO: 59) |
| | TTCA (SEQ ID NO: 58) | TTCA (SEQ ID NO: 63) | |
| Q5 | 73° C. | 71° C. | |
| Tm | | | |
| | #-CCATTCCGCGCTCTTCA | #-AGCTGTACGATGCTCTTCA | TGAAGAGCATCGTACAGCT |
| | (SEQ ID NO: 60) | (SEQ ID NO: 65) | (SEQ ID NO: 61) |
| Q5 | 65° C. | 63° C. | |
| Tm | | | |

In some instances, primers are further shortened or comprise lower GC content. In some instances primers are no more than 200 bp. Primers are biotinylated for removal after cleavage. T4 DNA polymerase is used to fill-in 5′ overhangs. SPRI beads are also used to remove ends. If the primers misprime on each other (due to similar 3′ ends) primers will still introduce BSPQ1 and a biotinylated tail. Oligos are binned by GC to avoid bias during amplification, and printed to a matrixed pool at 60 oligos per cluster.

Primers are synthesized having the sequences:

cfDNA_BSPQ1F
(SEQ ID NO: 60)
#-CCATTCCGCGCTCTTCA
cfDNA_BSPQ1R
(SEQ ID NO: 65)
#-AGCTGTACGATGCTCTTCA

Genes are binned by GC to prevent competition. For these genes, any molecules with BSPQ1 sites are removed to prevent potential issues downstream.

An adapter-off process for this design in some instances uses restriction. Using Bsa1 may result in variance in cleavage by methylation status, as cfDNA in some instances have adapters with Bsa1 cut sites. These are methylation sensitive because the primers used are biotinylated on the 5′ end and unmethylated. Bsa1 cut side have the sequences:

GGTCTC(N1)-3′
CCAGAG(N4)-5′

In some instances, endogenous sites are protected by adding 5-methyl-dCTP to the PCR step. After digestion, uncleaved products and cleaved adapters are removed by streptavidin binding, then filled in with Klenow. In some instances, Bsmb1 is used as a restriction enzyme, resulting in sequences:

5′-CGTCTC(N1)-3′
3′-GCAGAG(N4)-5′

Bottom strand methylation results in protection from digestion. To evaluate how this effects adapter removal, 5m-dCTP is spiked in at various ratios in a range from 10-100%. Both forward and reverse primers are biotinylated. Primers in some instances are designed to reduce homology and dimerization, as shown in Table 11.

TABLE 11

| | Forward primer | Reverse primer | 3′ rev_comp adapter |
|---|---|---|---|
| Primer | #- | #-AGTCAGGATGTCGTCTCG | CGAGACGACATCCTGACT |
| | CCATGTGCTCACGTCTCA | (SEQ ID NO: 67) | (SEQ ID NO: 68) |
| | (SEQ ID NO: 66) | | |
| Q5 Tm | 65° C. | 63° C. | |

A design utilizing the adapters of Table 11 is synthesized at 40 oligos per cluster binned by GC:. The 5′ overhang is filled in at the end with Klenow. Optionally, a PTO (phosphorothioate oligonucleotide) modification at the most 3′ of the primer is introduced which may protect the full length DNA from exonuclease digestion. In some cases, multiple PTO modifications are employed.

### Example 8. cfDNA Expansion with Uracil Adapter Cleavage

A cfDNA library was prepared using uracil as a terminal nucleotide of primers to enable facile cleavage of adapters sequences after amplification. In some instances, use of uracil results in fewer cleavage events in cfDNA libraries relative to a restriction enzyme digestion. Two cfDNA replicates were generated of 30 ng of cfDNA, amplified using UNI9 FWD/REV v2.1 (single uracil primers), a cfDNA expansion workflow performed comprising a) overhang digestion using Klenow and b) Overhang digestion using (non-HotStart) KAPA Hifi, and whole genome sequencing performed. A cfDNA sample was used to evaluate cleavage protocols.

cfDNA was obtained from commercial samples, or alternatively isolated from cell lines by nucleosome preparation. Briefly, Expi293 cells were harvested and diluted to 1×10⁶ cells per mL in 1×PBS, spun down, and the cells lysed. Isolated nuclei were treated with a nuclease and incubated, then treated with Proteinase K treatment. The product was then purified using spin columns.

Library preparation. 30 ng of input cfDNA was dissolved in 30 microliters EB buffer, and combined with 5 microliters water, 5 microliters 10× fragmentation buffer, and 10 microliters 5× fragmentation enzyme. The reaction was incubated for 30 minutes, the held at 4 degrees C., and mixed with 5 microliters of adapter solution. Ligation master mix was prepared from water (15 microliters), DNA ligation buffer (20 microliters), and DNA ligation mix (10 microliters), followed by incubation at 20 degrees C. for 15 minutes. Cleanup was then performed using 0.8× SPRI, and products eluted with 20 microliters EB buffer. The adapter library (20 microliters), forward and reverse primers (2.5 microliters each at 20 uM), and KAPA Hifi U+ master mix (25 microliters) were used to amplify the library. The thermocycler program was initialization (98C, 45 s, 1 cycle); denaturation (98C, 15 s), annealing (70C, 30 s), and extension (72C, 30 s)-3 cycles; final extension (72C, 1 min); and hold at 4C. After amplification, the products were cleaned up with 1× SPRI, and eluted with 30 microliters EB buffer. Amplicon size was approximately 150-500 bases, with most fragments about 234 bases in length. After fragmentation. of the cfDNA sample, ligation of adapters, and amplification with uracil-containing primers, the cfDNA library comprised the sequences (SEQ ID NOS 69 and 70, respectively, in order of appearance).

```
5′ (B) GAAGTGCCATTCCGCCTGACCTGCTCTTCCGUNNNNNNNNNNACGGAAGAGCTCCGATCCACCTCCGAGTCAC
              |||||||||||||||||||||||||||||||||||||||||||||||||||||||||||||||||||||||||
        3′ CTTCACGGTAAGGCGGACTGGACGAGAAGGCANNNNNNNNNNUGCCTTCTCGAGGCTAGGTGGAGGCTCAGTG (B)
```

The library was next digested with USER to cleave the adapters. 1 microgram of cfDNA was incubated with USER (1000 U/mL, 2.5 microliters), 10× cutsmart buffer (5 microliters), and water to 50 microliters at 37C for 1 hour. 3′ overhangs were removed by Klenow (1 microliter), 10× NEB buffer 2 (5 microliters), dNTPs (10 mM, 1 microliter), and water (5 microliter) incubated at 25C for 1 hour. Alternatively, 5× KAPA Hifi was used (5× KAPA Hifi Buffer, 10 microliters; KAPA Hifi Enzyme, 1 microliter; and dNTPs, 10 mM, 1 microliter) incubated at 72C for 1 hour. Products were purified by streptavidin binding to beads, and SPRI cleanup. Alternatively, primers were removed by Prep Streptavidin beads with Cutsmart (50 ul beads, wash 2 times with 1× Cutsmart buffer; Elute 20 ul 1× Cutsmart buffer); Bind sample to beads (Add beads to 500 ng of library ˜30 ul; Incubate in thermocycler 20° C. 30 min); USER digestion (Add 2.5 ul USER enzyme, Advance thermocycler 37° C. 1 hr); Strand disassociation (Advance thermocycler 70° C. 30m); Collect flow-through (Put tubes on magnetic rack, collect flow through); End blunting (Add 6 ul of 10× NEB Buffer 2; Add 1 ul of Klenow; Add 3 ul of nuclease free water; Incubate 25° C. 30 min); SPRI cleanup (2× SPRI cleanup; Elute 30 ul EB buffer). Alternatively, the following protocol changes were made: Bind to beads 20° C. 1 hr (500 ng); Add 5 ul USER, digest 37° C. 2 hr; Incubate 80° C. for 30 minutes (immediate magnetization to minimize potential re-annealing); Use KAPA Hifi for end digestion (14 ul 5× KAPA Buffer, lul KAPA Hifi (70 ul reaction total), Incubate 72° C. 1 hr); 2× SPRI cleanup (Elute 35 ul EB buffer).

After cleavage/exoIII digestion, the library had sequences (SEQ ID NOS 71 and 72, respectively, in order of appearance):

```
5′ (B) GAAGTGCCATTCCGCCTGACCTGCTCTTCCG NNNNNNNNNNACGGAAGAGCTCCGATCCACCTCCGAGTCAC
              ||||||||||||||||||||||||||||||||||||||||||||||||||||||||||||||||||||||||
        3′ CTTCACGGTAAGGCGGACTGGACGAGAAGGCANNNNNNNNNN GCCTTCTCGAGGCTAGGTGGAGGCTCAGTG (B)
```

After cleanup was performed with streptavidin beads and strand dissociation to generate sequences (SEQ ID NOS 73 and 74, respectively, in order of appearance):

5′ NNNNNNNNNNACGGAAGAGCTCCGATCCACCTCCGAGTCAC
||||||||||
3′ CTTCACGGTAAGGCGGACTGGACGAGAAGGCANNNNNNNNNN

Lastly, cfDNA repair and extension using polymerase are used to generate the cfDNA library:

5′ NNNNNNNNNN
||||||||||
3′ NNNNNNNNNN

Sequencing results of the library are shown in FIGS. 8B-8C.

### Example 9. cfDNA Expansion Using Phosphorothioates

Following the general methods of Example 8, cfDNA expansion libraries were generated using either no phosphorothioate at the 3′ uracil, 1 phosphorothioate bond at the 3′ uracil, or 3 phosphorothioate bonds at the 3′ uracil. Primer sequences were:

cfDNA_Exp_v2.1_FWD
(SEQ ID NO: 75)
/5Biosg/GA AGT GCC ATT CCG CCT GAC CTG CTC TTC CG/3deoxyU/
cfDNA_Exp_v2.1_REV
(SEQ ID NO: 76)
/5Biosg/GT GAC TCG GAG GTG GAT CGG AGC TCT TCC G/3deoxyU/
cfDNA_Exp_v2.1_1PTO_FWD
(SEQ ID NO: 77)
/5Biosg/GA AGT GCC ATT CCG CCT GAC CTG CTC TTC CG*/3deoxyU/
cfDNA_Exp_v2.1_1PTO_REV
(SEQ ID NO: 78)
/5Biosg/GT GAC TCG GAG GTG GAT CGG AGC TCT TCC G*/3deoxyU/
cfDNA_Exp_v2.1_3PTO_FWD
(SEQ ID NO:79)
/5Biosg/GA AGT GCC ATT CCG CCT GAC CTG CTC TTC* C*G*/3deoxyU/
cfDNA_Exp_v2.1_3PTO_REV
(SEQ ID NO: 80)
/5Biosg/GT GAC TCG GAG GTG GAT CGG AGC TCT TC*C* G*/3deoxyU/

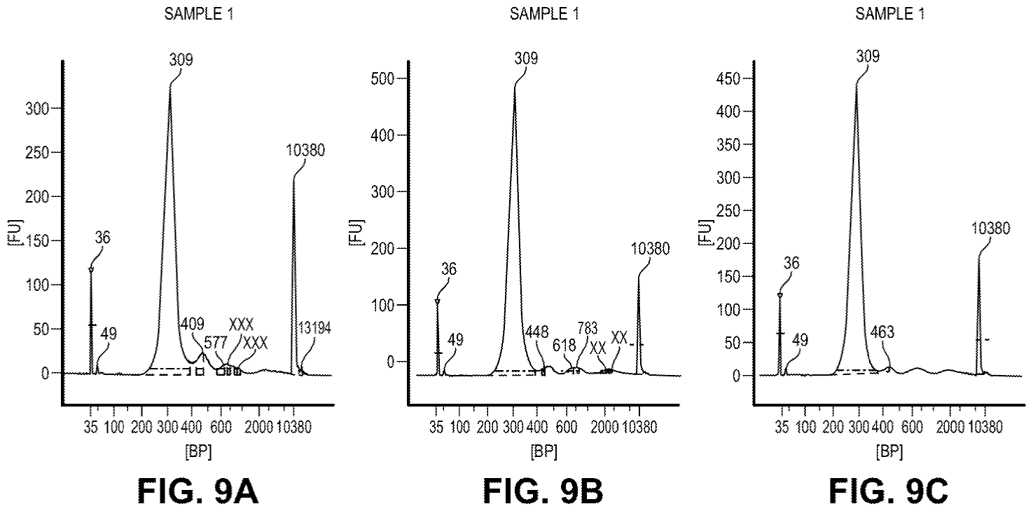

Libraries were evaluated using a bioanalyzer as shown in FIGS. 9A-9C.

Use of phosphorothioate bonds led to increased yields. Without being bound by theory, use of the phosphorothioate preserved the terminal uracil via preventing exonucleolytic removal of the U by the polymerase. After fragmentation. of the cfDNA sample, ligation of adapters, and amplification with uracil-containing primers, the cfDNA library comprised the sequences (SEQ ID NOS 69 and 70, respectively, in order of appearance):

```
5′ (B) GAAGTGCCATTCCGCCTGACCTGCTCTTCCGUNNNNNNNNNNACGGAAGAGCTCCGATCCACCTCCGAGTCAC
              |||||||||||||||||||||||||||||||||||||||||||||||||||||||||||||||||||||||||
        3′ CTTCACGGTAAGGCGGACTGGACGAGAAGGCANNNNNNNNNNUGCCTTCTCGAGGCTAGGTGGAGGCTCAGTG (B)
```

Phosphorothioate bonds are shown between G and U bases (bolded, underlined).

### Example 10. cfDNA Analysis Using UMIs for Cancer Detection

Early detection can significantly improve the clinical outcome for a number of cancers, but many of the best current screening methods require invasive procedures. A promising alternative approach is to perform a liquid biopsy of cell-free DNA (cfDNA) from patient plasma. Because tumors generally shed relatively large amounts of DNA into the circulation, cancer can potentially be detected by identifying oncogenic variants in cfDNA. This process generally requires extremely deep sequencing, and is in some cases limited by the accuracy of next-generation sequencing (NGS).

One approach to overcoming this limitation is to use unique molecular identifiers (UMIs), which are short sequences that uniquely tag each input DNA molecule prior to preparing NGS libraries. The approach can further be improved by tagging each original strand of the DNA molecule, in a technique termed duplex sequencing, which allows for correction of early PCR errors and/or single-strand DNA damage events.

Following the general procedures of Example 6, a contrived sample was designed and synthesized to simulate a fraction of tumor DNA in a healthy background and ligated to polynucleotide “duplex” UMI-containing adapters. UMI sequences were optimized to maximize sequence distances for error correction. The library was then subjected to sequencing analysis.

The rate at which input DNA is converted into sequencing libraries was determined. Using contrived samples to simulate a fraction of tumor DNA in a healthy background, both high sensitivity and specificity towards oncogenic variants was demonstrated. The baseline error rate using unmodified human cell-free DNA was evaluated, and mutation frequency in synthetic biology applications were determined.

### Example 11. Variant Analysis of cfDNA Analysis Using UMIs

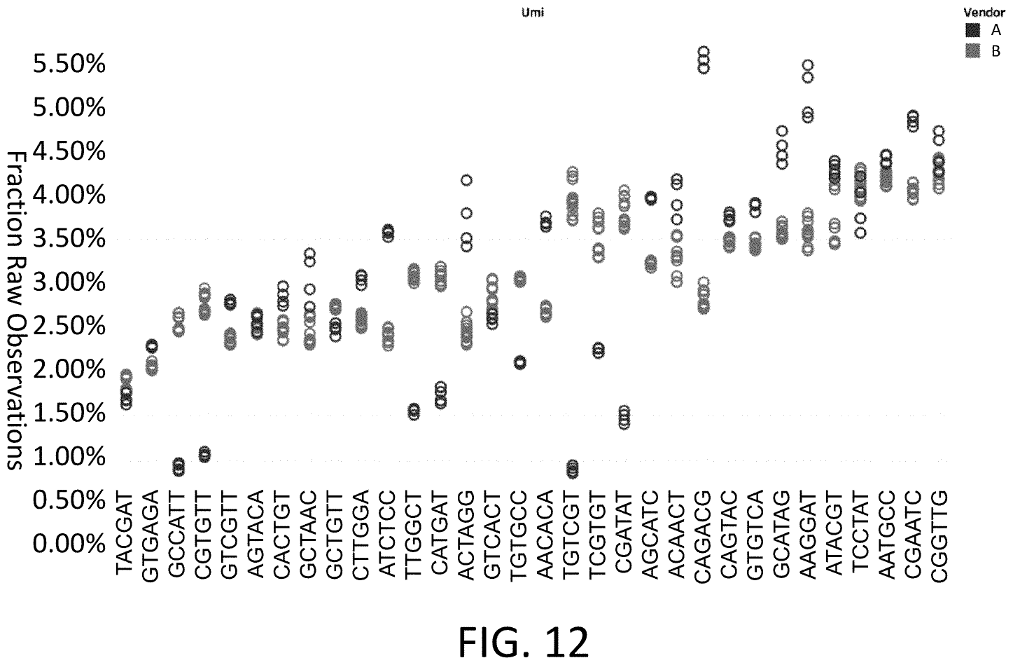

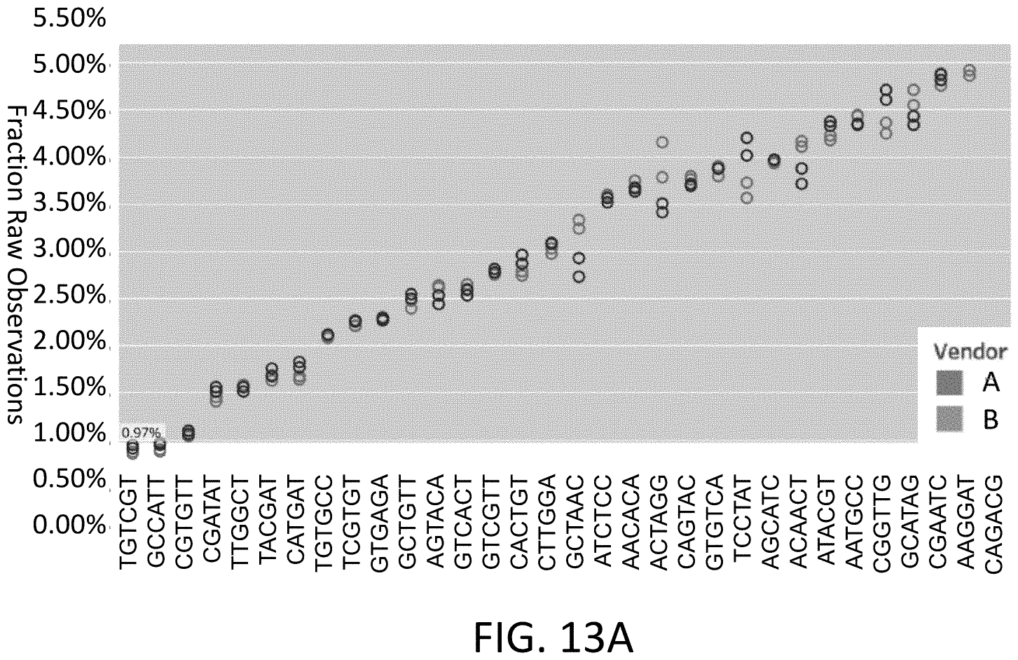

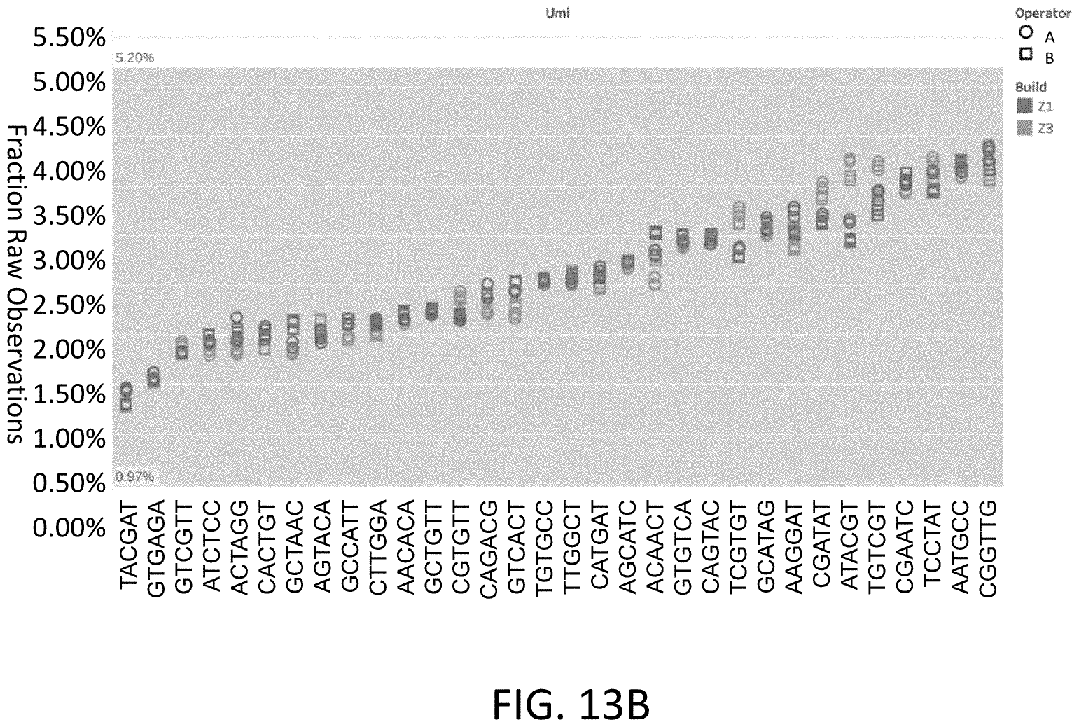

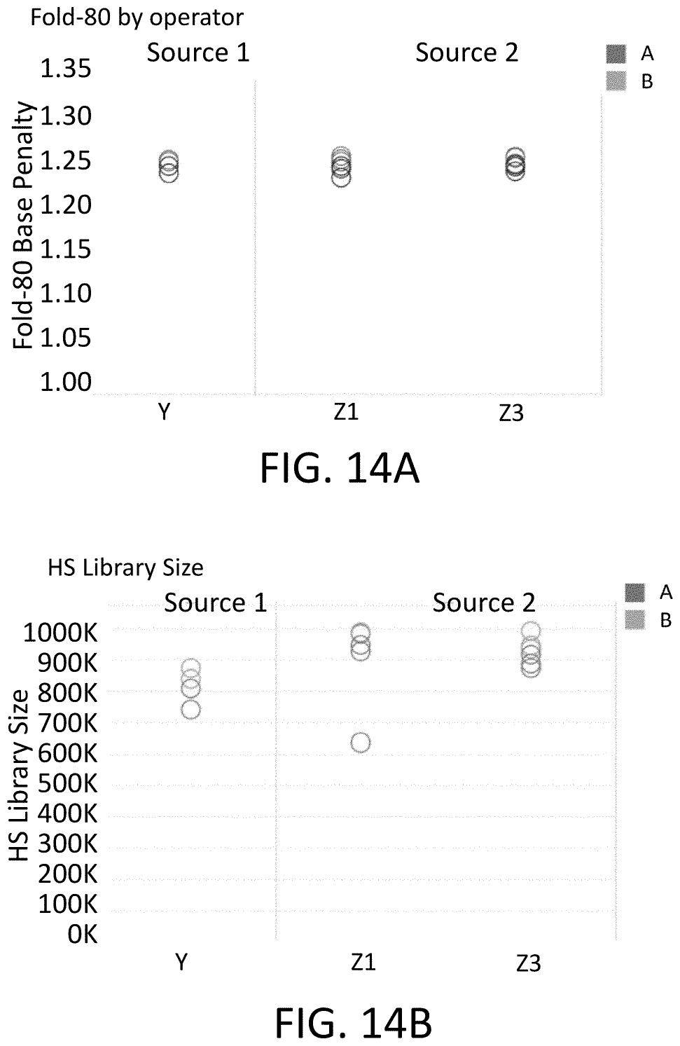

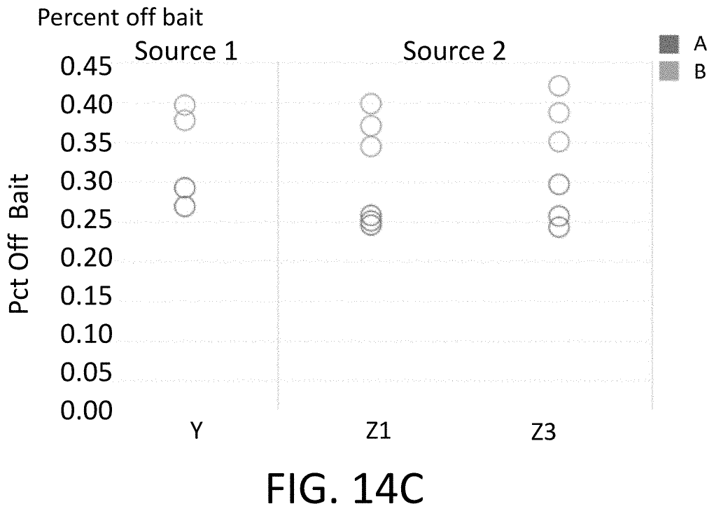

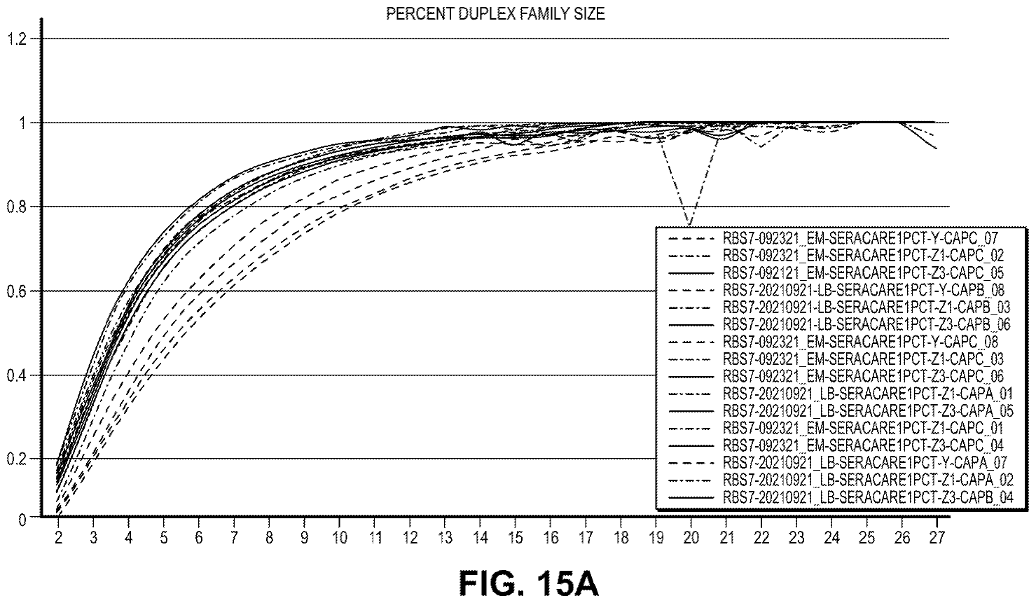

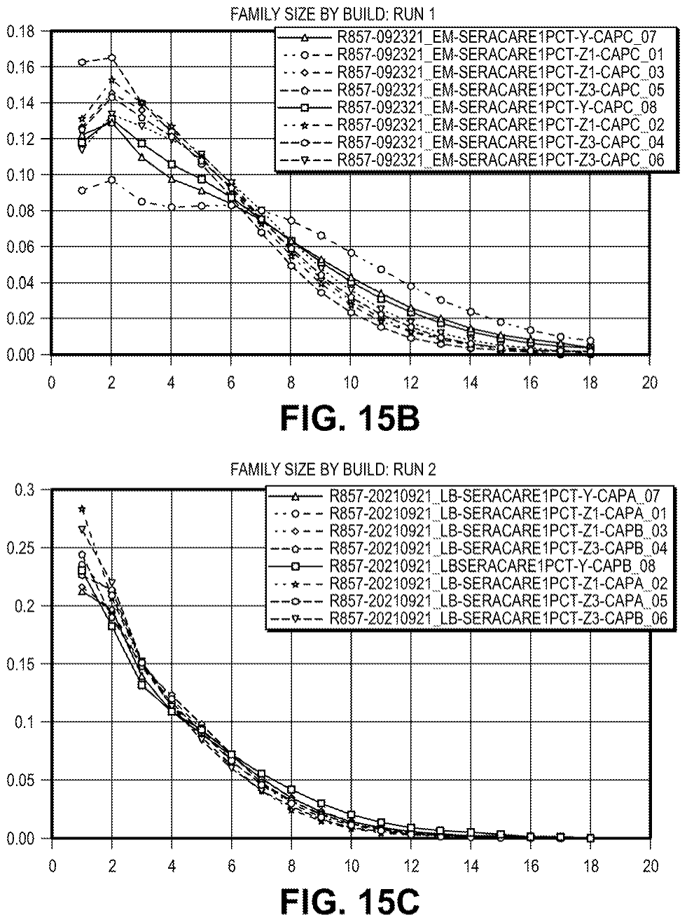

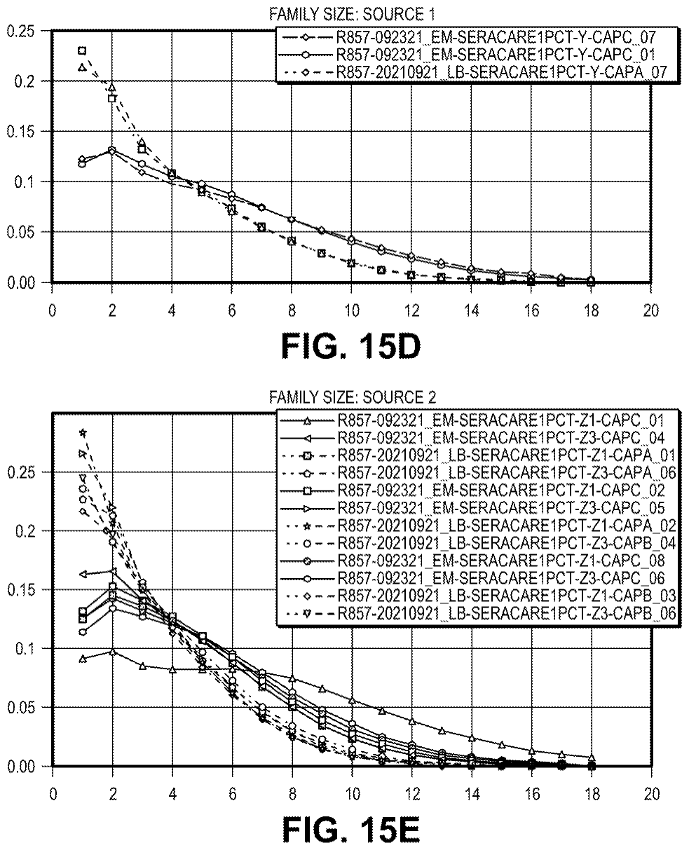

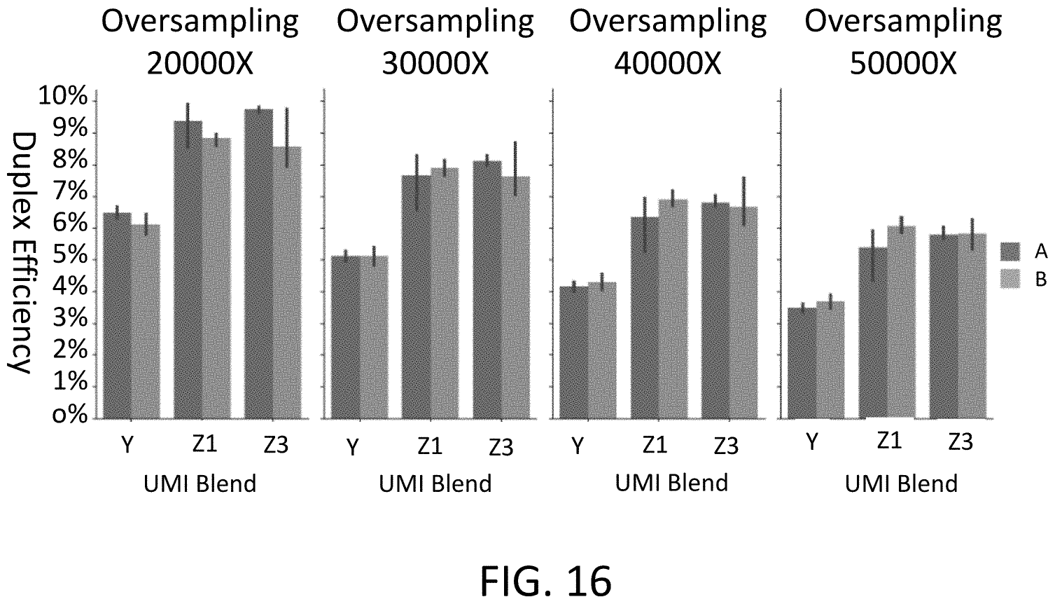

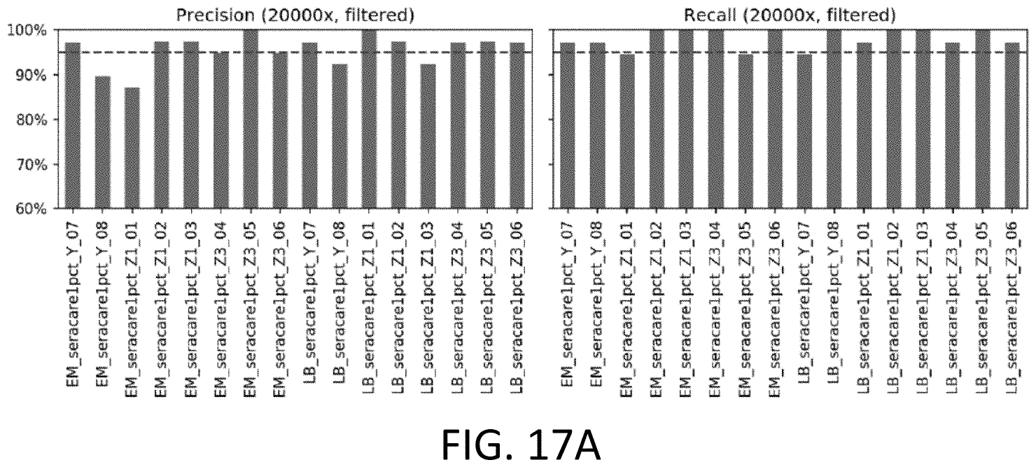

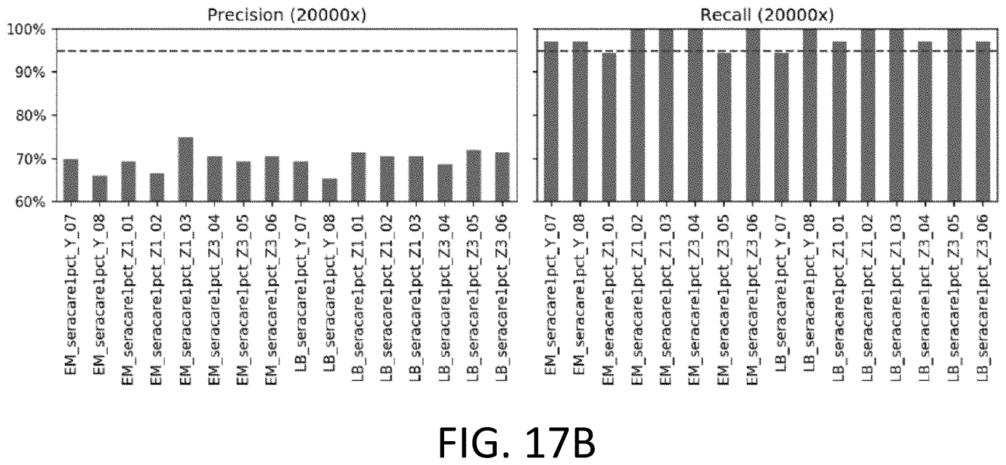

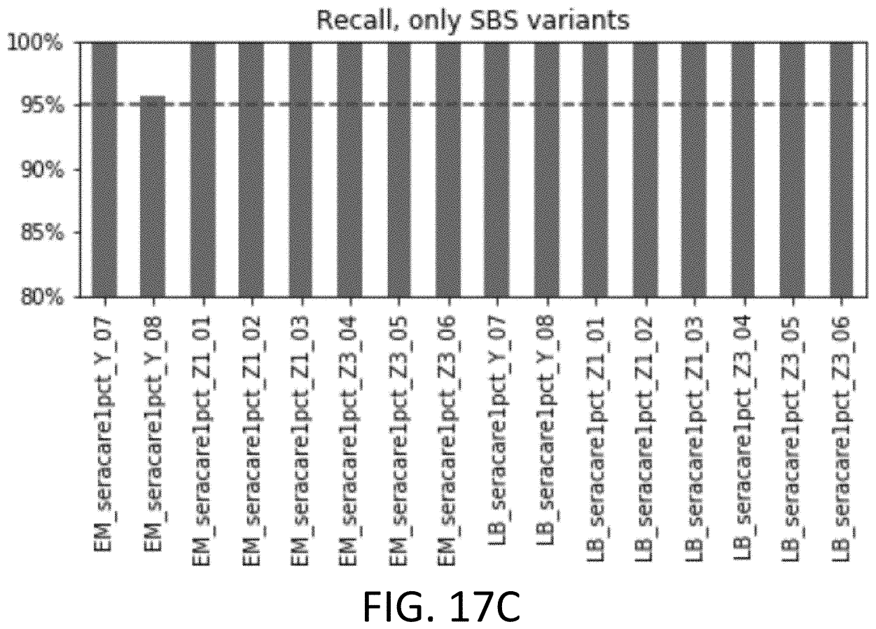

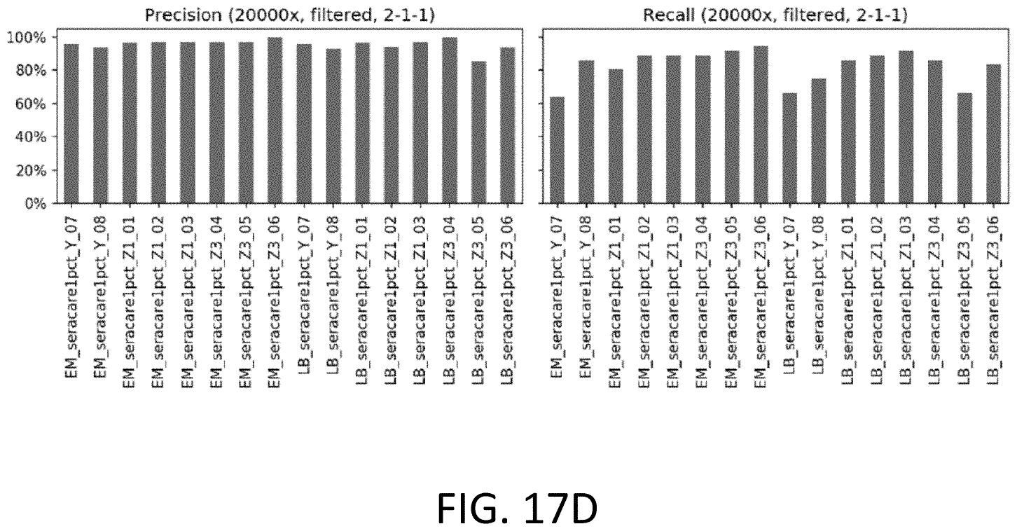

Following the general procedures of Example 10, 30 ng of ctDNA (Seracare) AF1l %, 3 μl of 10 μM adapter solution, followed by amplification (Equinox MM, 9 cycles PCR). Standard capture was performed using a 37 kb variant-targeting panel, with a hybridization time of 16 hrs (1 plex). 50 ng of input material was used and subjected to 16 cycles PCR prior to sequencing. Sequencing metrics are shown in FIGS. 12-17D. Duplex efficiency is shown below in Table 12.

TABLE 12

| Run | blend | 20000× | 30000x | 40000x | 50000x |
|---|---|---|---|---|---|
| 1 | Y | 6.3% | 5.0% | 4.0% | 3.4% |
| 1 | Y | 6.7% | 5.3% | 4.3% | 3.6% |
| 1 | Z1 | 8.6% | 6.6% | 5.3% | 4.4% |
| 1 | Z1 | 9.7% | 8.2% | 6.9% | 5.9% |
| 1 | Z1 | 9.9% | 8.3% | 6.9% | 5.9% |
| 1 | Z3 | 9.7% | 8.3% | 7.0% | 6.0% |
| 1 | Z3 | 9.7% | 8.0% | 6.7% | 5.7% |
| 1 | Z3 | 9.8% | 8.1% | 6.7% | 5.7% |
| 2 | Y | 5.8% | 4.9% | 4.1% | 3.5% |
| 2 | Y | 6.4% | 5.4% | 4.6% | 3.9% |
| 2 | Z1 | 8.6% | 7.7% | 6.7% | 5.9% |
| 2 | Z1 | 9.0% | 8.1% | 7.2% | 6.3% |
| 2 | Z1 | 9.0% | 7.9% | 6.9% | 6.0% |
| 2 | Z3 | 8.0% | 7.1% | 6.1% | 5.3% |
| 2 | Z3 | 9.8% | 8.7% | 7.6% | 6.7% |
| 2 | Z3 | 8.0% | 7.2% | 6.3% | 5.5% |

### Example 12. Variant Analysis of Pan Cancer Controls

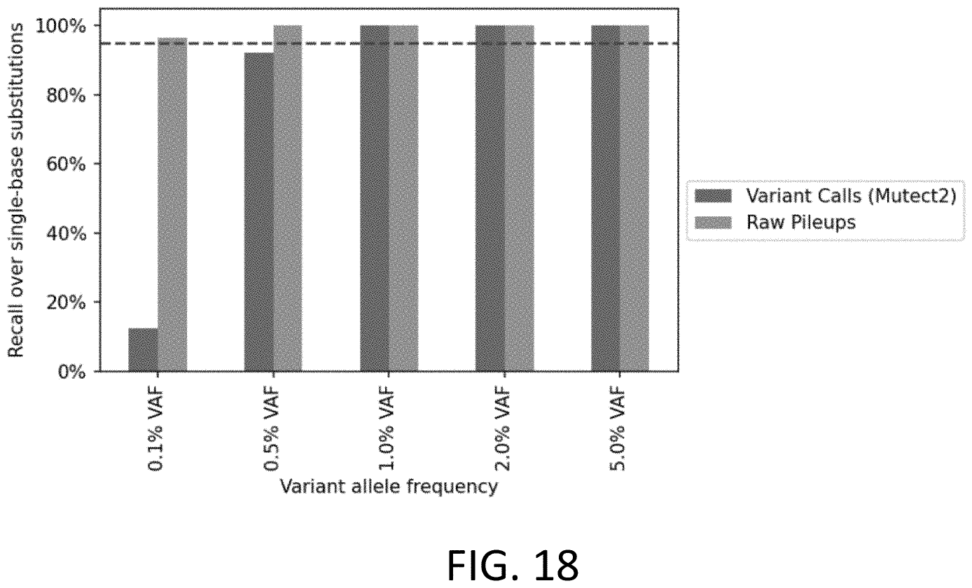

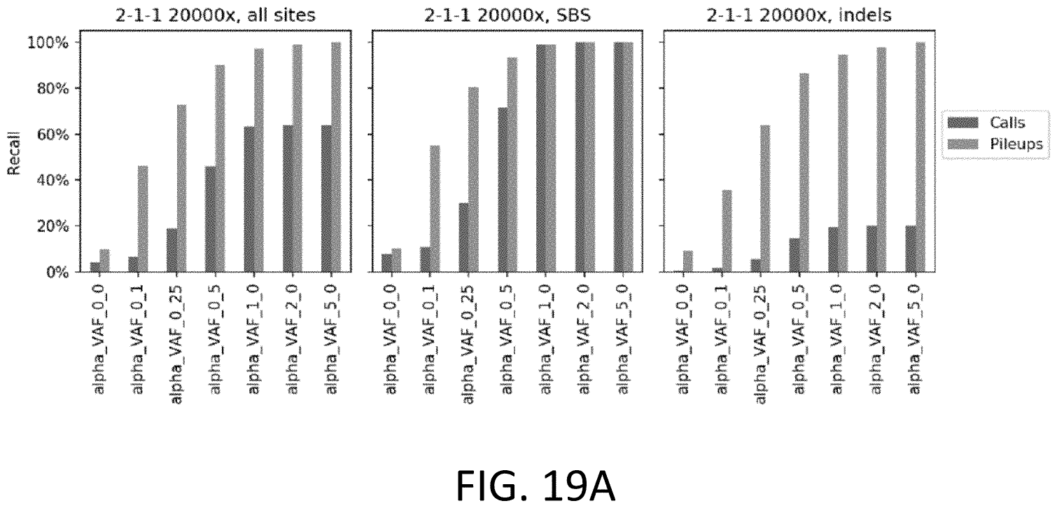

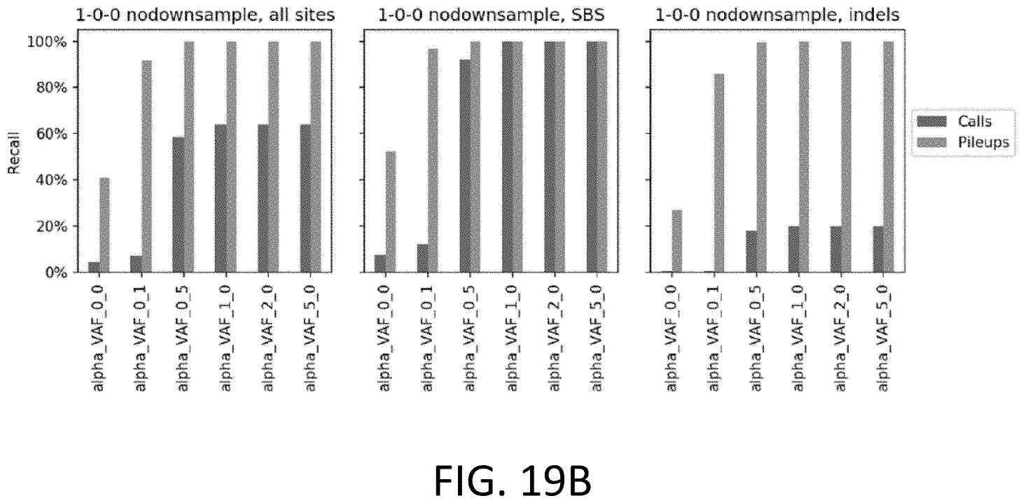

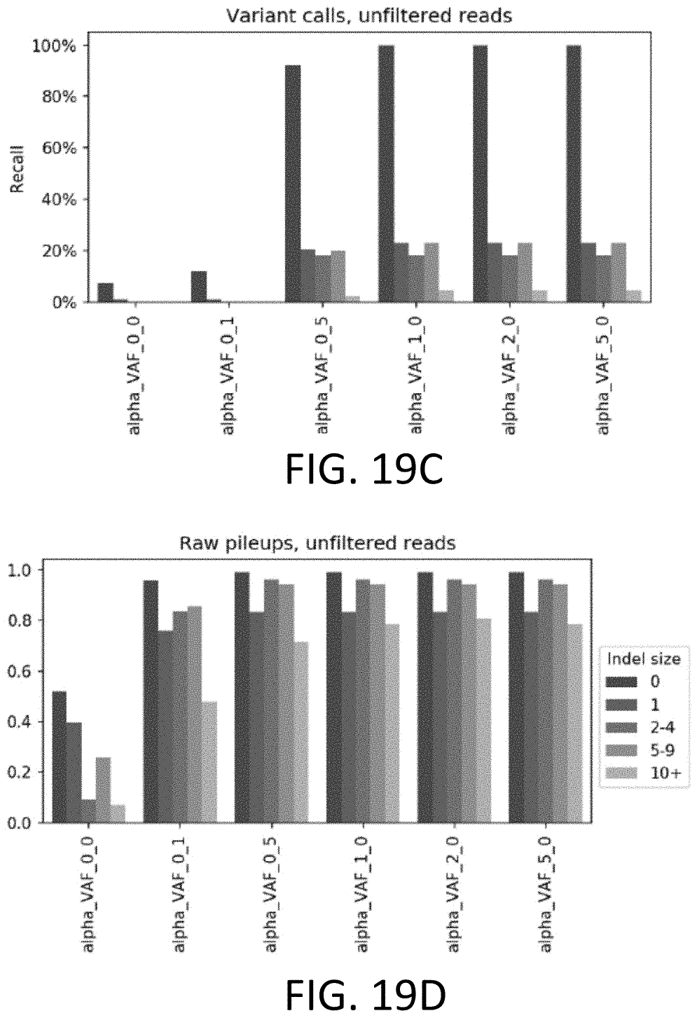

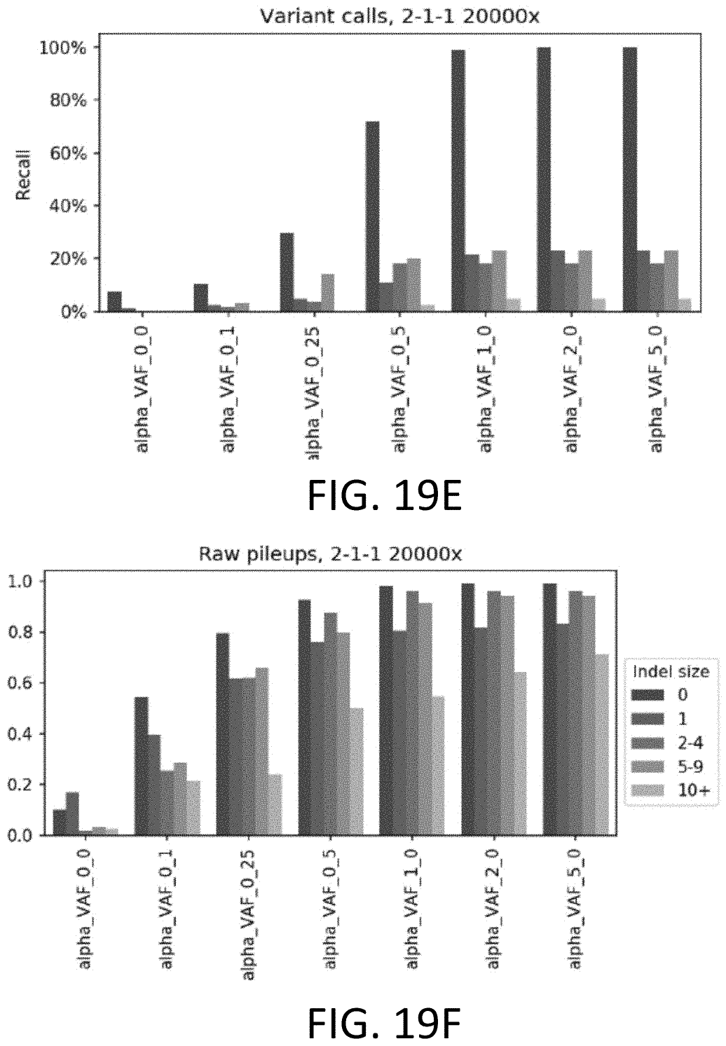

Following the general procedures of Examples 6 and 10, a 458 member pan-cancer cfDNA standard was designed, ligated to UMI-containing adapters, and sequenced. Results with and without downsampling and/or filtering are shown in FIGS. 18-19F.

What is claimed is:

1. A polynucleotide library comprising:
a sample polynucleotide set comprising at least 100 polynucleotides derived from genomic sequences; and
a background set comprising background polynucleotides, wherein the background set comprises cell-free DNA (cfDNA),
wherein each of the least 100 polynucleotides of the sample polynucleotide set comprises at least one variant, wherein the at least one variant comprises one or more changes compared to a background polynucleotide; and
at least 2 polynucleotides of the at least 100 polynucleotides are tiled across each of the at least one variant.

2. (canceled)

3. The library of claim 1, wherein the sample polynucleotide set comprises at least 150 variants.

4.-9. (canceled)

10. The library of claim 1, wherein the sample polynucleotide set comprises no more than 10% of the total amount of polynucleotides in the library.

11. The library of claim 1, wherein the at least one variant is present at a frequency of 0.01-5% relative to a wild-type genomic sequence.

12. (canceled)

13. (canceled)

14. The library of claim 1, wherein at least 90% of the at least one variants is present at a frequency of no more than 10% relative to the frequency of other variants.

15.-19. (canceled)

20. The library of claim 1, wherein the at least one variant comprises an insertion, deletion, fusion, duplication, frameshift, repeat expansion, or substitution.

21.-26. (canceled)

27. The library of claim 1, wherein the at least one variant is located in one or more of genes ABL1, ABL2, AKT1, ALK, APC, AR, ARAF, ARID1A, ATM, ATR, BAP1, BRAF, BRCA1, BRCA2, CCND1, CDC6, CDH1, CDK12, CDK4, CDX2, CTNNB1, DDR2, EGFR, EML4, ERBB2, ERBB3, ERG, ESR1, EZH2, FBXW7, FGFR1, FGFR2, FGFR3, FLT3, FOXA1, FOXL2, GATA3, GNA11, GNAQ, GNAS, HNF1A, HRAS, IDH1, IDH2, JAK2, KDM5C, KDM6A, KIF5B, KIT, KRAS, MAP2K1, MAPK1, MET, MIR4728, ERBB2, MLH1, MPL, MYCN, MYD88, NCOA4, NF1, NF2, NFE2L2, NOTCHI, NPM1, NRAS, PBRM1, PDGFRA, PIK3CA, PTEN, PTPN11, RET, RHEB, RHOA, RIT1, ROS1, SETD2, SMAD4, SMO, SPOP, TERT, TMPRSS2, TP53, TPR, TSC1, and VHL.

28. The library of claim 27, wherein the at least one variant is located in ten or more of genes ABL1, ABL2, AKT1, ALK, APC, AR, ARAF, ARID1A, ATM, ATR, BAP1, BRAF, BRCA1, BRCA2, CCND1, CDC6, CDH1, CDK12, CDK4, CDX2, CTNNB1, DDR2, EGFR, EML4, ERBB2, ERBB3, ERG, ESR1, EZH2, FBXW7, FGFR1, FGFR2, FGFR3, FLT3, FOXA1, FOXL2, GATA3, GNA11, GNAQ, GNAS, HNF1A, HRAS, IDH1, IDH2, JAK2, KDM5C, KDM6A, KIF5B, KIT, KRAS, MAP2K1, MAPK1, MET, MIR4728, ERBB2, MLH1, MPL, MYCN, MYD88, NCOA4, NF1, NF2, NFE2L2, NOTCHI, NPM1, NRAS, PBRM1, PDGFRA, PIK3CA, PTEN, PTPN11, RET, RHEB, RHOA, RIT1, ROS1, SETD2, SMAD4, SMO, SPOP, TERT, TMPRSS2, TP53, TPR, TSC1, and VHL.

29.-31. (canceled)

32. The library of claim 1, wherein the background polynucleotide set comprises wild-type regions corresponding to locations of the at least one variant.

33. The library of claim 32, wherein the wild-type regions are represented within 10% of the variant frequency of the variant set.

34.-36. (canceled)

37. The library of claim 1, wherein at least 90% of the polynucleotides in the background set are mononucleosomal or dinucleosomal.

38.-42. (canceled)

43. The library of claim 1, wherein at least one background polynucleotide comprises a variant present at a frequency of 0.001%, 0.01%, 0.1% 0.25%, 0.5%, 1%, or 2% relative to a wild-type genomic sequence.

44. A kit for measuring variant detection limits comprising:
a. The library of claim 1;
b. instructions for use of the kit; and
c. packaging configured to hold and describe the kit contents.

45.-47. (canceled)

48. A method of preparing the library of claim 1 comprising:
a. providing the background polynucleotide set;
b. synthesizing the sample polynucleotide set from predetermined sequences; and
c. mixing the variant set and the background set in a buffer.

49.-54. (canceled)

55. The method of claim 48, further comprising determining the variant frequency in the background polynucleotide set, where the variants correspond to the at least one variant in the sample polynucleotide set.

56.-57. (canceled)

58. A method of preparing a nucleic acid test sample useful for determining the detection limit of genomic variants comprising:
a. providing a library of claim 1;
b. obtaining at least one test sample from a patient suspected of having a disease or condition;
c. detecting the presence or absence of the one or more variants in the library of claim 1; and
d. detecting the presence or absence of the one or more variants in the at least one test sample.

59.-60. (canceled)

61. The method of claim 58, wherein detecting comprises sequencing by synthesis, nanopore sequencing, or SMRT sequencing.

62.-75. (canceled)

76. The method of claim 58, wherein detecting comprises addition of one or more adapters to at least some sample polynucleotides in the library.

77. The method of claim 76, wherein the one or more adapters comprise at least one barcode.

78. (canceled)

79. The method of claim 77, where at least one adapter of the one or more adaptors comprises a duplex adapter.

80.-83. (canceled)
